(19) United States
(12) Patent Application Publication
(10) Pub. No.: US 2026/0086458 A1
(43) Pub. Date: Mar. 26, 2026

(54) RESIST COMPOSITION, METHOD FOR FORMING RESIST PATTERN, COMPOUND, AND POLYMER COMPOUND

(71) Applicant: TOKYO OHKA KOGYO CO., LTD., Kawasaki-shi (JP)

(72) Inventors: Issei SUZUKI, Kawasaki-shi (JP); Akiya FUJINO, Kawasaki-shi (JP); Ryota KOBAYASHI, Kawasaki-shi (JP); Tetsuya MATSUSHITA, Kawasaki-shi (JP); KhanhTin NGUYEN, Kawasaki-shi (JP); Tetsuo FUJINAMI, Kawasaki-shi (JP); Seiji TODOROKI, Kawasaki-shi (JP); Yohei ADACHI, Kawasaki-shi (JP)

(21) Appl. No.: 19/112,828

(22) PCT Filed: Sep. 21, 2023
(86) PCT No.: PCT/JP2023/034311

§ 371 Date: Mar. 18, 2025

(30) Foreign Application Priority Data
Oct. 5, 2022 (JP) 2022-160709

(51) Int. Cl. G03F 7/004; C07C 69/88; C08F 220/38; G03F 7/20

(52) U.S. Cl. CPC G03F 7/0045; C07C 69/88; C08F 220/38; G03F 7/2004; G03F 7/2059

(57) ABSTRACT

A resist composition in which a polymer compound having a constitutional unit derived from a compound represented by General Formula (a0-m) is used as a base resin. In the formula, W represents a polymerizable group-containing group; R^{Ar1} and R^{Ar2} represent an aromatic group; R⁰¹ and R⁰² represent an iodinated alkyl group, an iodine atom, or a hydrogen atom; L¹¹ to L¹⁵ represent a linking group; q represents 0 or 1; when q represents 0, R⁰² represents an iodinated alkyl group or an iodine atom, and j2 represents an integer of 1 to 5; when q represents 1, the number of iodine atoms in R⁰¹ and R⁰² is 1 or more; and k1 and k2 represent 0 or 1; when q represents 0, k2 represents 1; when q represents 1, k1+k2 is 1; and M^{m+} represents an m-valent onium cation.

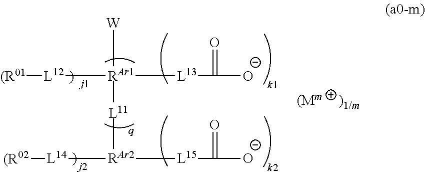

### TECHNICAL FIELD

The present invention relates to a resist composition, a method for forming a resist pattern, a compound, and a polymer compound.

Priority is claimed on Japanese Patent Application No. 2022-160709, filed Oct. 5, 2022, the content of which is incorporated herein by reference.

### BACKGROUND ART

In recent years, in the manufacture of semiconductor elements and liquid crystal display elements, advances in lithography technologies have led to a rapid progress in the field of pattern fining. These pattern miniaturization techniques typically involve shortening the wavelength (increasing the energy) of the exposure light source.

Resist materials are required to have lithography characteristics such as sensitivity to these exposure light sources and resolution that enables reproduction of patterns with minute dimensions.

As a resist material that satisfies these requirements, a chemically amplified resist composition containing a base material component whose solubility in a developing solution is changed by an action of an acid and an acid generator component that generates an acid upon light exposure has been used.

In the chemically amplified resist composition, a resin having a plurality of constitutional units is typically used as the base material component in order to improve lithography characteristics and the like.

Further, in the resist pattern formation, the behavior of an acid generated by an acid generator component upon light exposure serves as an element that greatly affects the lithography characteristics.

As the acid generator that is used in the chemically amplified resist composition, a wide variety of acid generators have been suggested in the related art. Examples of such known acid generators include an onium salt-based acid generator such as an iodonium salt or a sulfonium salt, an oxime sulfonate-based acid generator, a diazomethane-based acid generator, a nitrobenzyl sulfonate-based acid generator, an imino sulfonate-based acid generator, and a disulfone-based acid generator.

In addition, as the resist material, a chemically amplified resist composition containing a combination of an acid generator component and an acid diffusion control agent that controls diffusion of an acid to be generated from the acid generator component upon light exposure has been suggested in the related art.

For example, Patent Document 1 discloses a resist composition containing a resin that has a constitutional unit containing an acid dissociable group having a specific structure, a sulfonium salt, and a photodecomposable base that has an anion moiety and a cation moiety with a specific structure.

### CITATION LIST

### Patent Document

Patent Document 1: Japanese Unexamined Patent Application, First Publication No. 2020-091312

### SUMMARY OF INVENTION

### Technical Problem

With the further progress of the lithography technologies and resist pattern miniaturization, for example, the goal in lithography using extreme ultraviolet (EUV) rays or electron beams (EB) is to form a minute pattern with a size of several tens of nanometers. As the pattern dimensions decrease, high sensitivity to an exposure light source and lithography characteristics such as roughness reduction are required.

However, in the resist composition of the related art as described in Patent Document 1, further improvement is required in terms of achieving both the sensitivity and the lithography characteristics.

The present invention has been made in consideration of the above-described circumstances, and an object thereof is to provide a resist composition capable of achieving higher sensitivity and having improved lithography characteristics such as roughness reduction, a method for forming a resist pattern using the resist composition, a polymer compound useful for the resist composition, and a compound that can be used for synthesis of the polymer compound.

### Solution to Problem

In order to solve the above-described problems, the present invention has adopted the following configurations.

That is, according to a first aspect of the present invention, there is provided a resist composition which generates an acid upon light exposure and whose solubility in a developing solution is changed by an action of the acid, the resist composition including: a resin component (A1) whose solubility in a developing solution is changed by the action of the acid, in which the resin component (A1) has a constitutional unit (a0) derived from a compound represented by General Formula (a0-m).

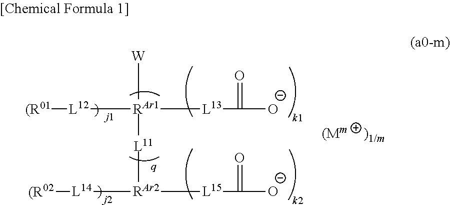

[In the formula, W represents a polymerizable group-containing group. R^{Ar1} and R^{Ar2} each independently represent an aromatic group which may have a substituent. R^{Ar1}-L¹¹- represents R^{Ar1}—O—, R^{Ar1}—S—, R^{Ar1}—NH—, R^{Ar1}-Ak-, R^{Ar1}—C(═O)—O—, R^{Ar1}—C(═O)—NH—, R^{Ar1}—O—C(═O)—, R^{Ar1}—NH—C(═O)—, R^{Ar1}-Ak-O—, R^{Ar1}-Ak-NH—, R^{Ar1}—O—C(═O)-Ak-, or R^{Ar1}— (in a case where L¹¹ represents a single bond). Ak represents an alkylene group or a fluorinated alkylene group (the same applies hereinafter). R^{Ar1}-L¹²- represents R^{Ar1}-Ak-, R^{Ar1}—C(═O)—O-Ak-, R^{Ar1}—C(═O)—NH-Ak-, R^{Ar1}—O—C(═O)-Ak-, R^{Ar1}—NH—C(═O)-Ak-, R^{Ar1}—O—C(═O)—, or R^{Ar1}— (in a case where L¹² represents a single bond). R^{Ar1}-L¹³-represents R^{Ar1}—O—, R^{Ar1}—S—, R^{Ar1}—NH—, R^{Ar1}-Ak-, R^{Ar1}—O-Ak-, or R^{Ar1}— (in a case where L¹³ represents a single bond). R^{Ar2}-L¹⁴- represents R^{Ar2}-Ak-, R^{Ar2}—C(═O)—O-Ak-, R^{Ar2}—C(═O)—NH-Ak-, R^{Ar2}—O—C(═O)-Ak-, R^{Ar2}—NH—C(═O)-Ak-, R^{Ar2}—O—C(═O)—, or R^{Ar2}— (in a case where L¹⁴ represents a single bond). R^{Ar2}-L¹⁵- represents R^{Ar2}—O—, R^{Ar2}—S—, R^{Ar2}—NH—, R^{Ar2}-Ak-, R^{Ar2}—O-Ak-, or R^{Ar2}— (in a case where L¹⁵ represents a single bond). q represents 0 or 1. R⁰¹ and R⁰² each independently represent an iodinated alkyl group, an iodine atom, or a hydrogen atom. j1 and j2 each independently represent an integer of 0 to 5. Here, in a case where q represents 0, R⁰² represents an iodinated alkyl group or an iodine atom, and j2 represents an integer of 1 to 5. In a case where q represents 1, the number of iodine atoms in R⁰¹ and R⁰² is 1 or more. k1 and k2 each independently represent 0 or 1. Here, in a case where q represents 0, k2 represents 1. In a case where q represents 1, k1+k2 is 1. M⁺ represents an m-valent onium cation. m represents an integer of 1 or greater.]

According to a second aspect of the present invention, there is provided a method for forming a resist pattern, including: a step of forming a resist film on a support using the resist composition according to the first aspect; a step of exposing the resist film to light; and a step of developing the resist film exposed to light to form a resist pattern.

According to a third aspect of the present invention, there is provided a compound which is represented by General Formula (a0-m).

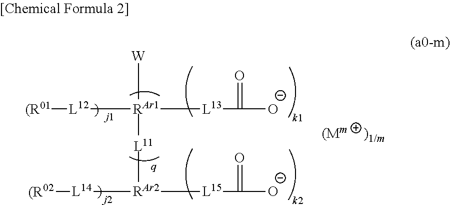

[In the formula, W represents a polymerizable group-containing group. R^{Ar1} and R^{Ar2} each independently represent an aromatic group which may have a substituent. R^{Ar1}-L¹¹- represents R^{Ar1}—O—, R^{Ar1}—S—, R^{Ar1}—NH—, R^{Ar1}-Ak-, R^{Ar1}—C(═O)—O—, R^{Ar1}—C(═O)—NH—, R^{Ar1}—O—C(═O)—, R^{Ar1}—NH—C(═O)—, R^{Ar1}-Ak-O—, R^{Ar1}-Ak-NH—, R^{Ar1}—O—C(═O)-Ak-, or R^{Ar1}— (in a case where L¹¹ represents a single bond). Ak represents an alkylene group or a fluorinated alkylene group (the same applies hereinafter). R^{Ar1}-L¹²- represents R^{Ar1}-Ak-, R^{Ar1}—C(═O)—O-Ak-, R^{Ar1}—C(═O)—NH-Ak-, R^{Ar1}—O—C(═O)-Ak-, R^{Ar1}—NH—C(═O)-Ak-, R^{Ar1}—O—C(═O)—, or R^{Ar1}— (in a case where L¹² represents a single bond). R^{Ar1}-L¹³-represents R^{Ar1}—O—, R^{Ar1}—S—, R^{Ar1}—NH—, R^{Ar1}-Ak-, R^{Ar1}—O-Ak-, or R^{Ar1}— (in a case where L¹³ represents a single bond). R^{Ar2}-L¹⁴- represents R^{Ar2}-Ak-, R^{Ar2}—C(═O)—O-Ak-, R^{Ar2}—C(═O)—NH-Ak-, R^{Ar2}—O—C(═O)-Ak-, R^{Ar2}—NH—C(═O)-Ak-, R^{Ar2}—O—C(═O)—, or R^{Ar2}— (in a case where L¹⁴ represents a single bond). R^{Ar2}-L¹⁵- represents R^{Ar2}—O—, R^{Ar2}—S—, R^{Ar2}—NH—, R^{Ar2}-Ak-, R^{Ar2}—O-Ak-, or R^{Ar2}— (in a case where L¹⁵ represents a single bond). q represents 0 or 1. R⁰¹ and R⁰² each independently represent an iodinated alkyl group, an iodine atom, or a hydrogen atom. j1 and j2 each independently represent an integer of 0 to 5. Here, in a case where q represents 0, R⁰² represents an iodinated alkyl group or an iodine atom, and j2 represents an integer of 1 to 5. In a case where q represents 1, the number of iodine atoms in R⁰¹ and R⁰² is 1 or more. k1 and k2 each independently represent 0 or 1. Here, in a case where q represents 0, k2 represents 1. In a case where q represents 1, k1+k2 is 1. M^{m+} represents an m-valent onium cation. m represents an integer of 1 or greater.]

According to a fourth aspect of the present invention, there is provided a polymer compound which has a constitutional unit derived from the compound according to the third aspect.

### Advantageous Effects of Invention

According to the present invention, it is possible to provide a resist composition capable of achieving higher sensitivity and having improved lithography characteristics such as roughness reduction, a method for forming a resist pattern using the resist composition, a polymer compound useful for the resist composition, and a compound that can be used for synthesis of the polymer compound.

### DESCRIPTION OF EMBODIMENTS

In the present specification and the scope of the present claims, the term “aliphatic” is a relative concept used in relation to the term “aromatic” and defines a group, a compound, or the like that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of “halogen atom” include a fluorine atom, a chlorine atom, a bromine atom, and an iodine atom.

The term “constitutional unit” indicates a monomer unit constituting a polymer compound (a resin, a polymer, or a copolymer).

The expression “may have a substituent” includes both a case where a hydrogen atom (—H) is substituted with a monovalent group and a case where a methylene group (—CH₂—) is substituted with a divalent group.

The term “light exposure” is a general concept for irradiation with radiation.

The term “acid decomposable group” indicates a group having acid decomposability in which at least a part of a bond in the structure of the acid decomposable group can be cleaved by the action of an acid.

Examples of the acid decomposable group whose polarity is increased by the action of an acid include groups which are decomposed by the action of an acid to generate a polar group.

Examples of the polar group include a carboxy group, a hydroxyl group, an amino group, and a sulfo group (—SO₃H).

More specific examples of the acid decomposable group include a group in which the above-described polar group has been protected by an acid dissociable group (such as a group in which a hydrogen atom of the OH-containing polar group has been protected by an acid dissociable group).

Here, the term “acid dissociable group” indicates both a group (i) having an acid dissociation property in which a bond between the acid dissociable group and an atom adjacent to the acid dissociable group can be cleaved by the action of an acid and a group (ii) in which some bonds are cleaved by the action of an acid, a decarboxylation reaction occurs, and thus the bond between the acid dissociable group and the atom adjacent to the acid dissociable group can be cleaved.

It is necessary that the acid dissociable group that constitutes the acid decomposable group is a group which exhibits a lower polarity than that of the polar group generated by the dissociation of the acid dissociable group. Thus, in a case where the acid dissociable group is dissociated by the action of an acid, a polar group exhibiting a higher polarity than that of the acid dissociable group is generated so that the polarity is increased. As a result, the polarity of an entire component (A1) is increased. Due to the increase in the polarity, the solubility in a developing solution is relatively changed such that the solubility is increased in a case where the developing solution is an alkali developing solution and the solubility is decreased in a case where the developing solution is an organic developing solution.

The term “base material component” denotes an organic compound having a film-forming ability. Organic compounds used as the base material component are classified into non-polymers and polymers. As the non-polymers, typically non-polymers having a molecular weight of 500 or greater and less than 4000 (hereinafter, referred to as “low-molecular-weight compounds”) are used. Hereinafter, “resin”, “polymer compound”, or “polymer” indicates a polymer having a molecular weight of 1000 or greater. As the molecular weight of the polymer, the weight-average molecular weight in terms of polystyrene according to gel permeation chromatography (GPC) is used.

The expression “constitutional unit to be derived” denotes a constitutional unit formed by cleavage of a multiple bond between carbon atoms, for example, an ethylenic double bond.

In “acrylic acid ester”, the hydrogen atom bonded to the carbon atom at the α-position may be substituted with a substituent. The substituent (R^{αx}) that substitutes the hydrogen atom bonded to the carbon atom at the α-position is an atom other than the hydrogen atom or a group. Further, itaconic acid diester in which the substituent (R^{αx}) has been substituted with a substituent having an ester bond or α-hydroxyacryl ester in which the substituent (R^{αx}) has been substituted with a hydroxyalkyl group or a group obtained by modifying a hydroxyl group thereof can be described as acrylic acid ester. Further, the carbon atom at the α-position of acrylic acid ester indicates the carbon atom to which the carbonyl group of acrylic acid is bonded, unless otherwise specified.

Hereinafter, acrylic acid ester in which the hydrogen atom bonded to the carbon atom at the α-position has been substituted with a substituent is also referred to as α-substituted acrylic acid ester.

The concept “derivative” includes those obtained by substituting a hydrogen atom at the α-position of a target compound with another substituent such as an alkyl group or a halogenated alkyl group, and derivatives thereof. Examples of the derivatives thereof include those obtained by substituting a hydrogen atom of a hydroxyl group of a target compound, in which the hydrogen atom at the α-position may be substituted with a substituent is substituted, with an organic group; and those obtained by bonding a substituent other than a hydroxyl group to a target compound in which the hydrogen atom at the α-position may be substituted with a substituent. In addition, the α-position denotes the first carbon atom adjacent to the functional group unless otherwise specified.

Examples of the substituent that substitutes a hydrogen atom at the α-position of hydroxystyrene include the same groups as those for R^{αx}, such as an alkyl group and a halogenated alkyl group.

In the present specification and the scope of the present claims, asymmetric carbons may be present and enantiomers or diastereomers may be present depending on the structures of the chemical formulae. In this case, these isomers are represented by one chemical formula. These isomers may be used alone or in the form of a mixture.

### (Resist Composition)

The resist composition according to the present embodiment is a resist composition which generates an acid upon light exposure and whose solubility in a developing solution is changed by the action of the acid.

The resist composition contains a base material component (A) (hereinafter, also referred to as “component (A)”) whose solubility in a developing solution is changed by the action of the acid. In the resist composition of the present embodiment, the component (A) includes a resin component which generates an acid upon exposure and whose solubility in a developing solution is changed by the action of the acid.

In a case where a resist film is formed using the resist composition according to the present embodiment and the formed resist film is subjected to selective exposure, an acid is generated at least from the component (A) at exposed portions of the resist film, and the generated acid acts on the component (A) to change the solubility of the component (A) in a developing solution, whereas the solubility of the component (A) in a developing solution is not changed at unexposed portions of the resist film, and thus a difference in solubility in the developing solution occurs between the exposed portions and the unexposed portions of the resist film. Therefore, in a case where the resist film is developed, the exposed portion of the resist film is dissolved and removed to form a positive-tone resist pattern in a case where the resist composition is of a positive-tone, whereas the unexposed portion of the resist film is dissolved and removed to form a negative-tone resist pattern in a case where the resist composition is of a negative-tone.

The resist composition of the present embodiment may be a positive-tone resist composition or a negative-tone resist composition. Further, in the formation of a resist pattern, the resist composition according to the present embodiment may be used for an alkali developing process using an alkali developing solution in the developing treatment, or used for a solvent developing process using a developing solution containing an organic solvent (organic developing solution) in the developing treatment.

### <Base Material Component (A)>

In the resist composition according to the present embodiment, the component (A) contains a resin component (A1) (hereinafter, also referred to as “component (A1)”) whose solubility in a developing solution is changed by the action of an acid.

Since the polarity of the base material component before and after the light exposure is changed by using the component (A1), an excellent development contrast can be obtained not only in an alkali developing process but also in a solvent developing process.

As the component (A), at least one of other polymer compounds or low-molecular-weight compounds may be used in combination with the component (A1).

In the resist composition according to the present embodiment, the component (A) may be used alone or in combination of two or more kinds thereof.

### In Regard to Resin Component (A1)

The component (A1) is a resin component whose solubility in a developing solution is changed by the action of an acid. The component (A1) has a constitutional unit (a0) derived from a compound represented by General Formula (a0-m).

Further, the component (A1) may have other constitutional units as necessary in addition to the constitutional unit (a0).

<<Constitutional Unit (a0)>>

The constitutional unit (a0) is a constitutional unit derived from a compound represented by General Formula (a0-m).

The constitutional unit (a0) acts as a quencher (acid diffusion control agent) which traps an acid generated upon light exposure in the resist composition. Further, the constitutional unit (a0) functions as a photodecomposable base that is decomposed upon light exposure and loses acid diffusion controllability. Therefore, in a case where the component (A1) has the constitutional unit (a0), the contrast between the exposed portion and the unexposed portion of the resist film can be further improved in the resist pattern formation.

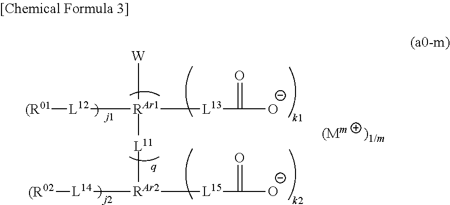

[In the formula, W represents a polymerizable group-containing group. R^{Ar1} and R^{Ar2} each independently represent an aromatic group which may have a substituent. R^{Ar1}-L¹¹- represents R^{Ar1}—O—, R^{Ar1}—S—, R^{Ar1}—NH—, R^{Ar1}-Ak-, R^{Ar1}—C(═O)—O—, R^{Ar1}—C(═O)—NH—, R^{Ar1}—O—C(═O)—, R^{Ar1}—NH—C(═O)—, R^{Ar1}-Ak-O—, R^{Ar1}-Ak-NH—, R^{Ar1}—O—C(═O)-Ak-, or R^{Ar1}— (in a case where L¹¹ represents a single bond). Ak represents an alkylene group or a fluorinated alkylene group (the same applies hereinafter). R^{Ar1}-L¹²- represents R^{Ar1}-Ak-, R^{Ar1}—C(═O)—O-Ak-, R^{Ar1}—C(═O)—NH-Ak-, R^{Ar1}—O—C(═O)-Ak-, R^{Ar1}—NH—C(═O)-Ak-, R^{Ar1}—O—C(═O)—, or R^{Ar1}— (in a case where L¹² represents a single bond). R^{Ar1}-L¹³ represents R^{Ar1}—O—, R^{Ar1}—S—, R^{Ar1}—NH—, R^{Ar1}-Ak-, R^{Ar1}—O-Ak-, or R^{Ar1}— (in a case where L¹³ represents a single bond). R^{Ar2}-L¹⁴- represents R^{Ar2}-Ak-, R^{Ar2}—C(═O)—O-Ak-, R^{Ar2}—C(═O)—NH-Ak-, R^{Ar2}—O—C(═O)-Ak-, R^{Ar2}—NH—C(═O)-Ak-, R^{Ar2}—O—C(═O)—, or R^{Ar2}— (in a case where L¹⁴ represents a single bond). R^{Ar2}-L¹⁵- represents R^{Ar2}—O—, R^{Ar2}—S—, R^{Ar2}—NH—, R^{Ar2}-Ak-, R^{Ar2}—O-Ak-, or R^{Ar2}— (in a case where L¹⁵ represents a single bond). q represents 0 or 1. R⁰¹ and R⁰² each independently represent an iodinated alkyl group, an iodine atom, or a hydrogen atom. j1 and j2 each independently represent an integer of 0 to 5. Here, in a case where q represents 0, R⁰² represents an iodinated alkyl group or an iodine atom, and j2 represents an integer of 1 to 5. In a case where q represents 1, the number of iodine atoms in R⁰¹ and R⁰² is 1 or more. k1 and k2 each independently represent 0 or 1. Here, in a case where q represents 0, k2 represents 1. In a case where q represents 1, k1+k2 is 1. M^{m+} represents an m-valent onium cation. m represents an integer of 1 or greater.]

### [Anion Moiety]

In Formula (a0-m), the term “polymerizable group” in the polymerizable group-containing group as W is a group that enables polymerization of a compound containing a polymerizable group by radical polymerization or the like and, for example, is a group having multiple bonds between carbon atoms such as an ethylenic double bond.

Examples of the polymerizable group include a vinyl group, an allyl group, acryloyl group, a methacryloyl group, a fluorovinyl group, a difluorovinyl group, a trifluorovinyl group, a difluorotrifluoromethylvinyl group, a trifluoroallyl group, a perfluoroallyl group, a trifluoromethylacryloyl group, a nonylfluorobutylacryloyl group, a vinyl ether group, a fluorine-containing vinyl ether group, an allyl ether group, a fluorine-containing allyl ether group, a styryl group, and a vinylnaphthyl group, a fluorine-containing styryl group, a fluorine-containing vinylnaphthyl group, a norbornyl group, a fluorine-containing norbornyl group, and a silyl group.

The polymerizable group-containing group may be a group formed of only a polymerizable group, or a group formed of a polymerizable group and a group other than the polymerizable group. Examples of the group other than the polymerizable group include a divalent hydrocarbon group which may have a substituent and a divalent linking group containing a heteroatom.

Suitable examples of the polymerizable group-containing group include a group represented by Formula: C(R^{X11})(R^{X12})═C(R^{x13})-Ya^{x0}-. In the formulae, R^{X11}, R^{X12}, and R^{X13} each independently represent a hydrogen atom, an alkyl group having 1 to 5 carbon atoms, or a halogenated alkyl group having 1 to 5 carbon atoms, and Ya^{x0} represents a single bond or a divalent linking group. Examples of the divalent linking group as Ya^{x0} include a divalent hydrocarbon group which may have a substituent and a divalent linking group having a heteroatom. Examples of the divalent linking group as Ya^{x0} include an ester bond (—C(═O)—O—), an oxycarbonyl group (—O—C(═O)—), an ether bond (—O—), —C(═O)—NH—, —NH—C(═O)—, a linear or branched alkylene group, and a combination thereof.

It is preferable that W in Formula (a0-m) represents a polymerizable group-containing group represented by General Formula (a0-w).

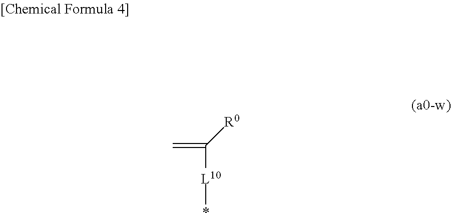

[In the formula, R⁰ represents an alkyl group having 1 to 5 carbon atoms, a halogenated alkyl group having 1 to 5 carbon atoms, or a hydrogen atom. -L¹⁰-* represents —C(═O)—O—*, —C(═O)—NH—*, or -*(in a case where L¹⁰ represents a single bond). * represents a bonding site that is bonded to R^{Ar1} in General Formula (a0-m) or a bonding site that is bonded to R^{Ar2} in a case where q represents 0.]

In Formula (a0-w), as the alkyl group having 1 to 5 carbon atoms as R⁰, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group.

The halogenated alkyl group having 1 to 5 carbon atoms as R⁰ is a group obtained by substituting some or all hydrogen atoms of the above-described alkyl group having 1 to 5 carbon atoms with halogen atoms. As the halogen atom, a fluorine atom is particularly preferable.

R⁰ represents preferably a hydrogen atom, an alkyl group having 1 to 5 carbon atoms, or a fluorinated alkyl group having 1 to 5 carbon atoms, and from the viewpoint of industrial availability, more preferably a hydrogen atom, a methyl group, or trifluoromethyl group, and still more preferably a hydrogen atom or a methyl group.

In Formula (a0-m), the aromatic group as R^{Ar1} may be a group obtained by removing two or more hydrogen atoms from an aromatic hydrocarbon ring or a group obtained by removing two or more hydrogen atoms from an aromatic heterocyclic ring in which one or more carbon atoms constituting the aromatic hydrocarbon ring are substituted with heteroatoms, or may be monocyclic or polycyclic. Examples of the heteroatoms include a nitrogen atom and a sulfur atom.

The aromatic group has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms.

Examples of the aromatic hydrocarbon ring include benzene, naphthalene, anthracene, and phenanthrene. Examples of the aromatic heterocyclic ring include azoles such as pyrrole, diazole, and triazole; and thiazole.

The aromatic group as R^{Ar1} is preferably a group obtained by removing two or more hydrogen atoms from benzene, a group obtained by removing two or more hydrogen atoms from naphthalene, or a group obtained by removing two or more hydrogen atoms from azole, more preferably a group obtained by removing two or more hydrogen atoms from benzene or a group obtained by removing two or more hydrogen atoms from naphthalene, and still more preferably a group obtained by removing two or more hydrogen atoms from benzene.

In Formula (a0-m), the aromatic group as R^{Ar2} may be a group obtained by removing one or more hydrogen atoms from an aromatic hydrocarbon ring or a group obtained by removing one or more hydrogen atoms from an aromatic heterocyclic ring in which one or more carbon atoms constituting the aromatic hydrocarbon ring are substituted with heteroatoms, or may be monocyclic or polycyclic. Examples of the heteroatoms include a nitrogen atom and a sulfur atom.

The aromatic group has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms.

Examples of the aromatic hydrocarbon ring include benzene, naphthalene, anthracene, and phenanthrene. Examples of the aromatic heterocyclic ring include azoles such as pyrrole, diazole, and triazole; and thiazole.

The aromatic group as R^{Ar2} is preferably a group obtained by removing two or more hydrogen atoms from benzene, a group obtained by removing two or more hydrogen atoms from naphthalene, or a group obtained by removing two or more hydrogen atoms from azole and more preferably a group obtained by removing two or more hydrogen atoms from benzene, a group obtained by removing two or more hydrogen atoms from pyrrole, or a group obtained by removing two or more hydrogen atoms from diazole.

From the viewpoint of improving the sensitivity to an exposure light source, suitable examples of the substituent that the aromatic group as R^{Ar1} and R^{Ar2} may have include a hydroxy group, an alkoxy group, an alkyl group, and an amino group.

In Formula (a0-m), for R^{Ar1}-L¹¹-, the alkylene group in Ak may be linear or branched, and the number of carbon atoms is preferably 1 to 10, more preferably 1 to 6, still more preferably 1 to 4, particularly preferably 1 to 3, and most preferably 1. The fluorinated alkylene group as Ak is a group in which some or all hydrogen atoms of the alkylene group as Ak are substituted with fluorine atoms.

R^{Ar1}-L¹¹- is preferably selected from the group consisting of R^{Ar1}—O—C(═O)—, R^{Ar1}—C(═O)—O—, R^{Ar1}—C(═O)—NH—, R^{Ar1}-Ak-O—, R^{Ar1}-Ak-NH—, and R^{Ar1}—O—C(═O)-Ak-.

In Formula (a0-m), the description of the alkylene group and the fluorinated alkylene group as Ak in R^{Ar1}-L¹²- is the same as that for Ak in R^{Ar1}-L¹¹-.

R^{Ar1}-L¹²- is preferably R^{Ar1}— (in a case where L¹² represents a single bond).

In Formula (a0-m), the description of the alkylene group and the fluorinated alkylene group as Ak in R^{Ar2}-L¹⁴- is the same as that for Ak in R^{Ar1}L¹¹-.

R^{Ar2}-L¹⁴- is preferably R^{Ar2}— (in a case where L¹⁴ represents a single bond).

In Formula (a0-m), the description of the alkylene group and the fluorinated alkylene group as Ak in R^{Ar1}-L¹³- is the same as that for Ak in R^{Ar1}-L¹¹-.

R^{Ar1}-L¹³- is preferably selected from the group consisting of R^{Ar1}— (in a case where L¹³ represents a single bond), R^{Ar1}-Ak-, R^{Ar1}—NH—, and R^{Ar1}—O-Ak- and more preferably R^{Ar1}-Ak-.

In Formula (a0-m), the description of the alkylene group and the fluorinated alkylene group as Ak in R^{Ar2}-L¹⁵- is the same as that for Ak in R^{Ar1}-L¹¹-.

R^{Ar2}-L⁵- is preferably R^{Ar2}— (in a case where L¹⁵ represents a single bond).

In Formula (a0-m), examples of the iodinated alkyl group as R⁰¹ and R⁰² include an iodinated alkyl group having 1 to 5 carbon atoms. Among these, an iodinated alkyl group having 1 to 3 carbon atoms is preferable, and an iodinated methyl group or an iodinated ethyl group is more preferable. It is preferable that R⁰¹ and R⁰² represent an iodine atom or a hydrogen atom.

In Formula (a0-m), j1 represents preferably 1, 2, or 3 and more preferably 1 or 2.

j2 represents preferably an integer of 1 to 4 and more preferably 2, 3, or 4.

Here, in a case where q represents 0, R⁰² represents an iodinated alkyl group or an iodine atom and preferably an iodine atom, and j2 represents preferably an integer of 1 to 4 and more preferably 2, 3, or 4. In a case where q represents 1, the number of iodine atoms in R⁰¹ and R⁰² is 1 or more, preferably 1 to 6, and more preferably 1 to 4.

Preferred examples of the constitutional unit (a0) include a constitutional unit (a01), a constitutional unit (a02), a constitutional unit (a03), and a constitutional unit (a04), which will be described below.

In Regard to Constitutional Unit (a01)

The constitutional unit (a01) is a constitutional unit derived from a compound in which, in General Formula (a0-m), W represents a polymerizable group-containing group represented by General Formula (a0-w), q represents 1, k1 represents 1, k2 represents 0, R⁰² represents an iodinated alkyl group or an iodine atom, j2 represents an integer of 1 to 5, and L¹⁰ in General Formula (a0-w) represents a single bond. That is, the constitutional unit (a01) is a constitutional unit derived from a compound represented by General Formula (a01-m).

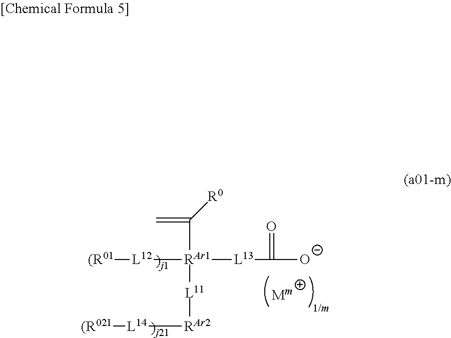

[In the formula, R⁰ has the same definition as that for R⁰ in General Formula (a0-w). R^{Ar1}, R^{Ar2}, R^{Ar1}-L¹¹-, R^{Ar1}-L¹²-, R^{Ar1}-L¹³-, R^{Ar2}-L¹⁴-, R⁰¹, and j1 each have the same definition as that for R^{Ar1}, R^{Ar2}, R^{Ar1}-L¹¹-, R^{Ar1}-L¹²-, R^{Ar1}-L¹³-, R^{Ar2}-L¹⁴-, R^{0l}, and j1 in Formula (a0-m). R⁰²¹ represents an iodinated alkyl group or an iodine atom. j21 represents an integer of 1 to 5. M^{m+} represents an m-valent onium cation. m represents an integer of 1 or greater.]

In Formula (a01-m), examples of the iodinated alkyl group as R⁰²¹ include an iodinated alkyl group having 1 to 5 carbon atoms, and an iodinated alkyl group having 1 to 3 carbon atoms is preferable, and an iodinated methyl group or an iodinated ethyl group is more preferable. It is preferable that R⁰²¹ represents an iodine atom. j21 represents preferably an integer of 1 to 4 and more preferably 2, 3, or 4.

Hereinafter, preferred specific examples of the anion moiety of the compound represented by General Formula (a01-m) will be shown.

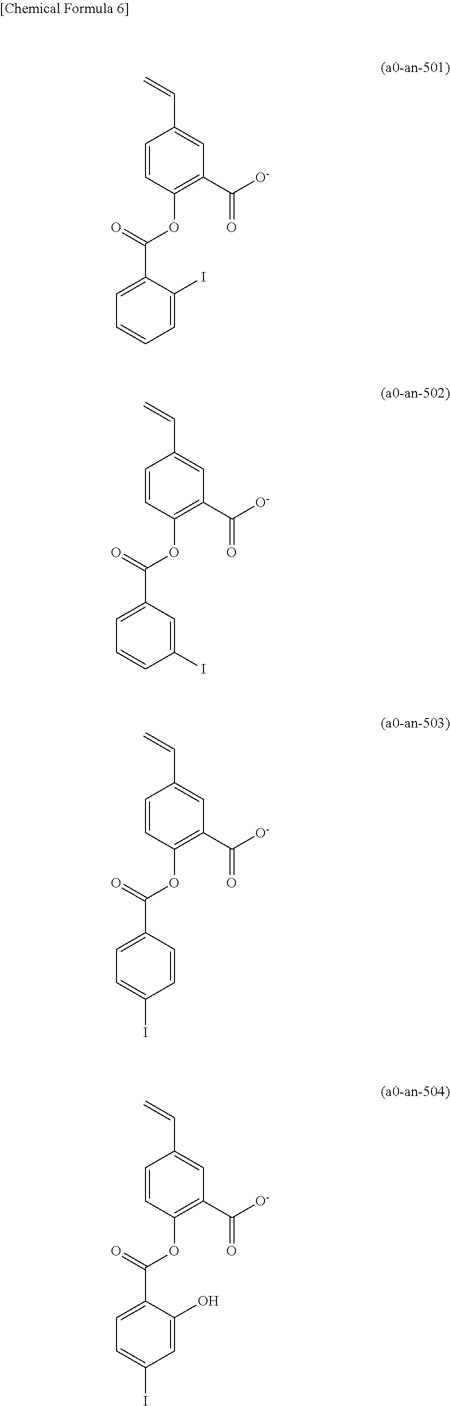

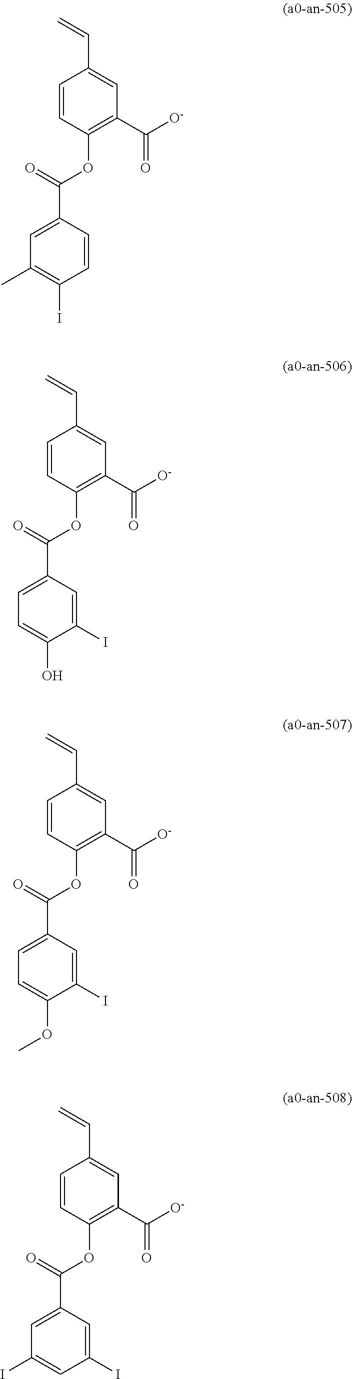

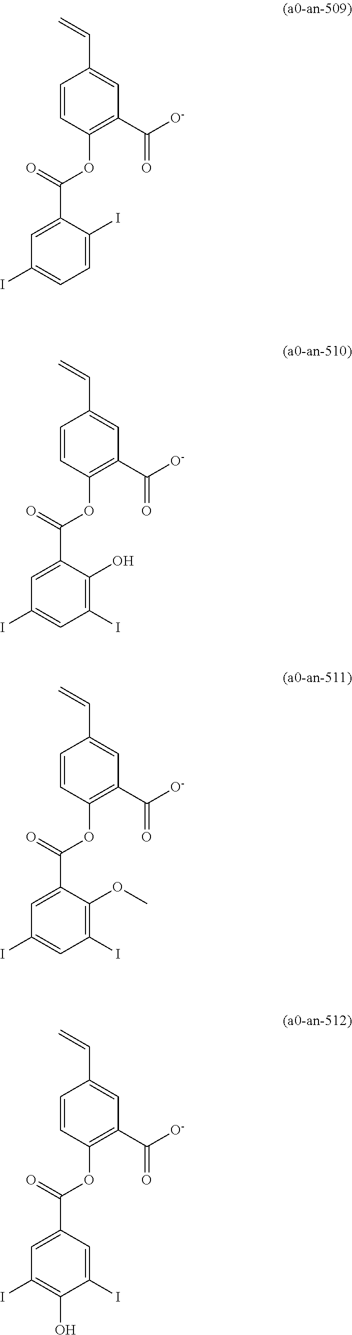

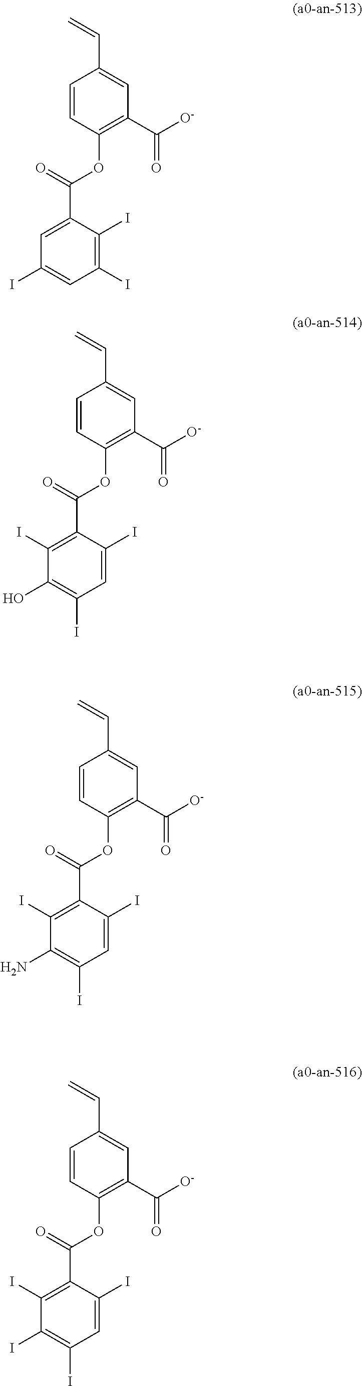

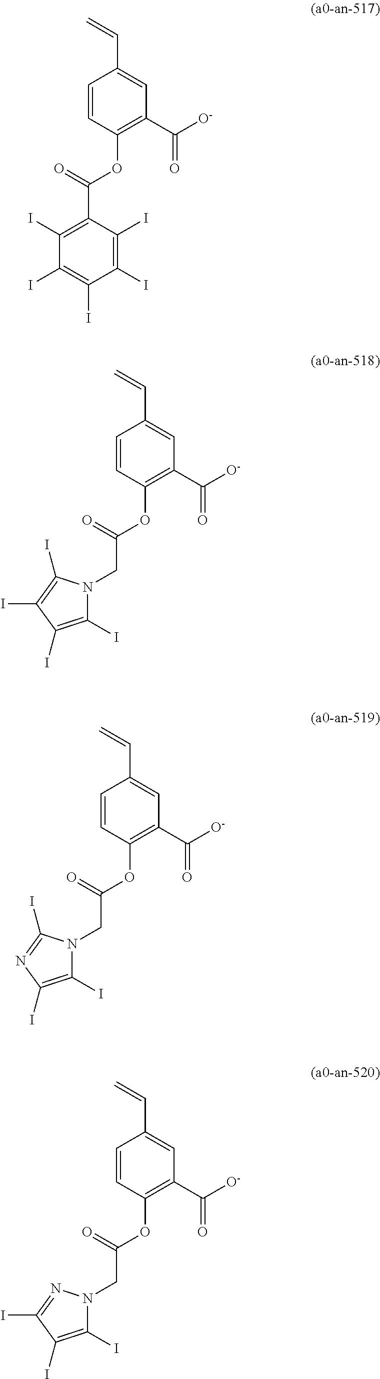

The anion moiety of the compound represented by General Formula (a01-m) is preferably at least one selected from the group consisting of anions each represented by Chemical Formulae (a0-an-501) to (a0-an-520).

In Regard to Constitutional Unit (a02):

The constitutional unit (a02) is a constitutional unit derived from a compound in which, in General Formula (a0-m), W represents a polymerizable group-containing group represented by General Formula (a0-w), q represents 1, k1 represents 0, k2 represents 1, R⁰² represents an iodinated alkyl group or an iodine atom, j2 represents an integer of 1 to 5, and L¹⁰ in General Formula (a0-w) represents a single bond. That is, the constitutional unit (a02) is a constitutional unit derived from a compound represented by General Formula (a02-m).

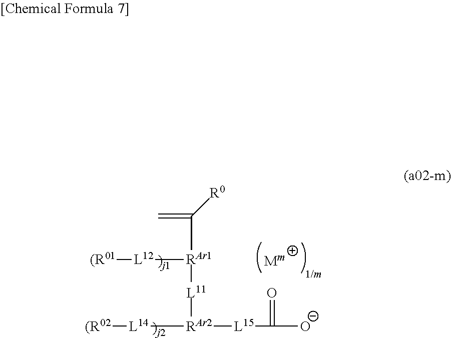

[In the formula, R⁰ has the same definition as that for R⁰ in General Formula (a0-w). R^{Ar1}, R^{Ar2}, R^{Ar1}-L¹¹-, R^{Ar1}-L¹²-, R^{Ar2}-L¹⁴-, R^{Ar2}-L¹⁵, R⁰¹, R⁰², j1, and j2 each have the same definition as that for R^{Ar1}, R^{Ar2}, R^{Ar1}-L¹¹-, R^{Ar1}-L¹²-, R^{Ar2}-L¹⁴-, R^{Ar2}-L¹⁵-, R⁰¹, R⁰², j1, and j2 in Formula (a0-m). M^{m+} represents an m-valent onium cation. m represents an integer of 1 or greater.]

Hereinafter, preferred specific examples of the anion moiety of the compound represented by General Formula (a02-m) will be shown.

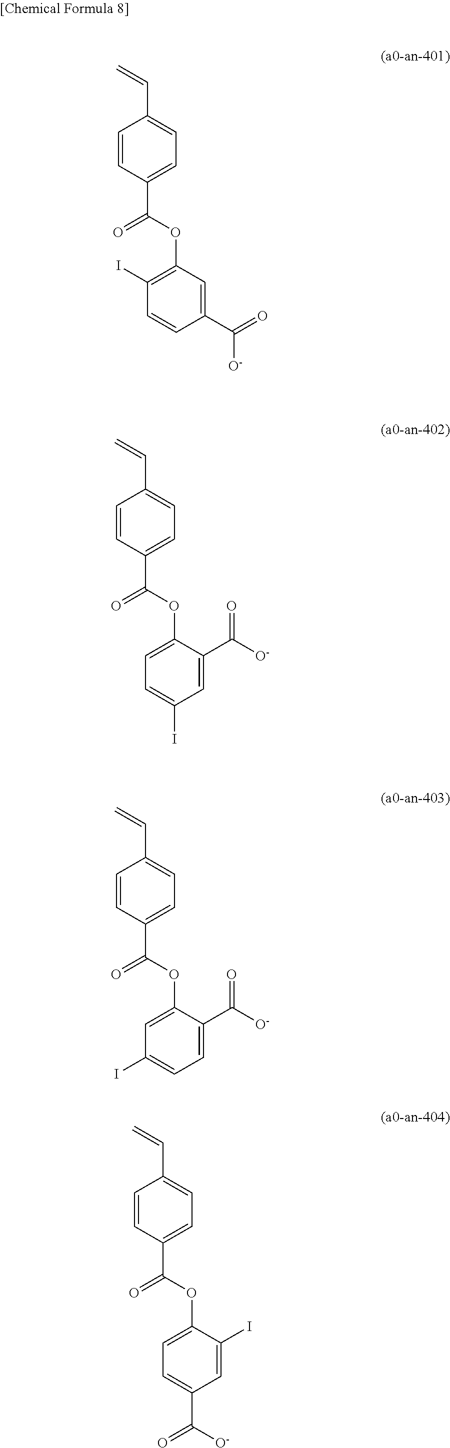

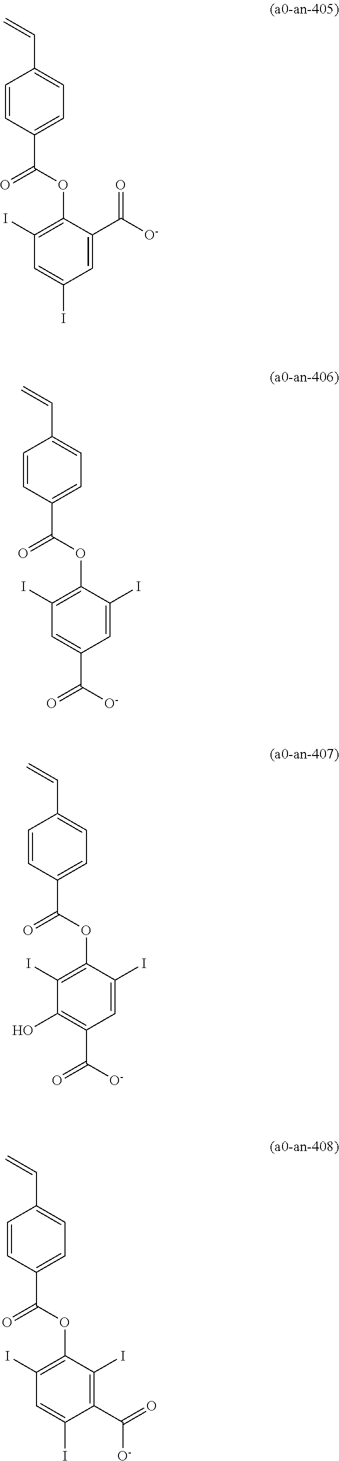

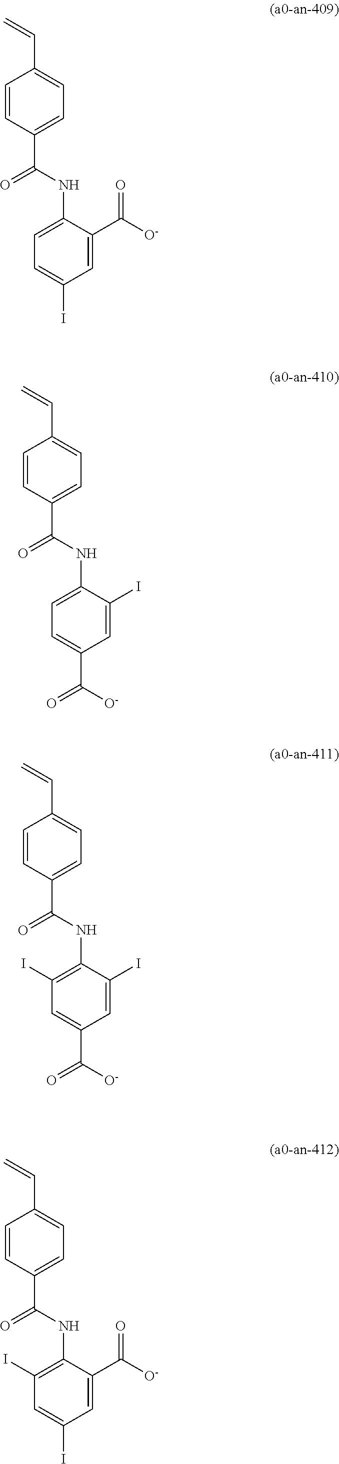

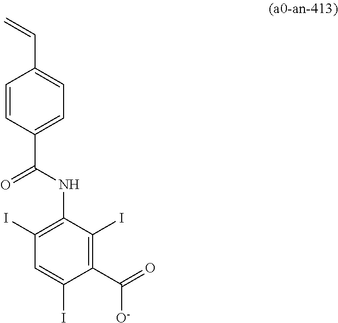

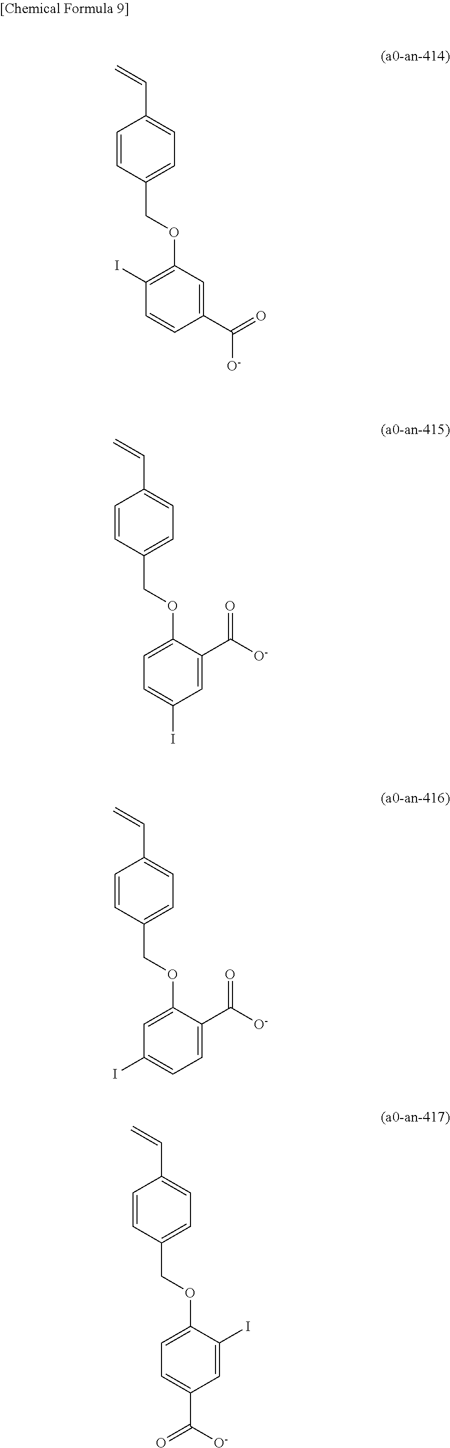

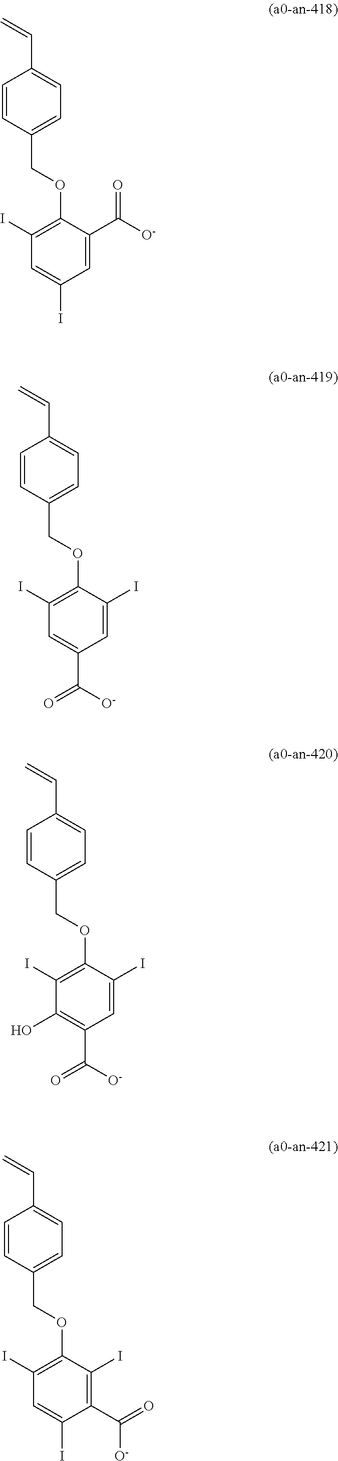

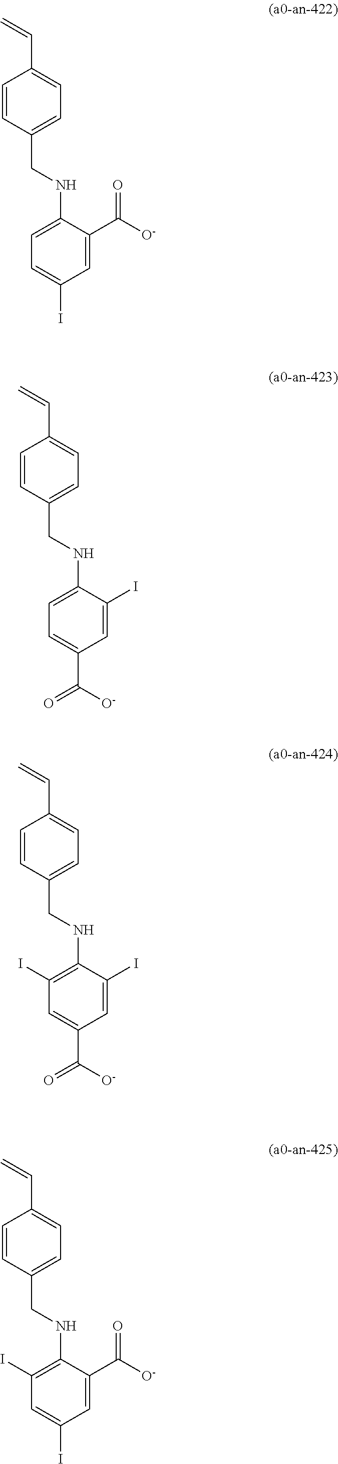

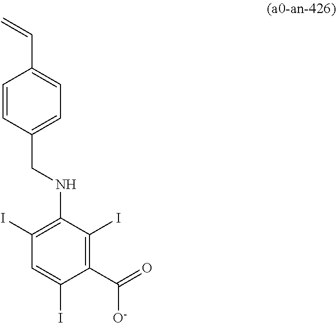

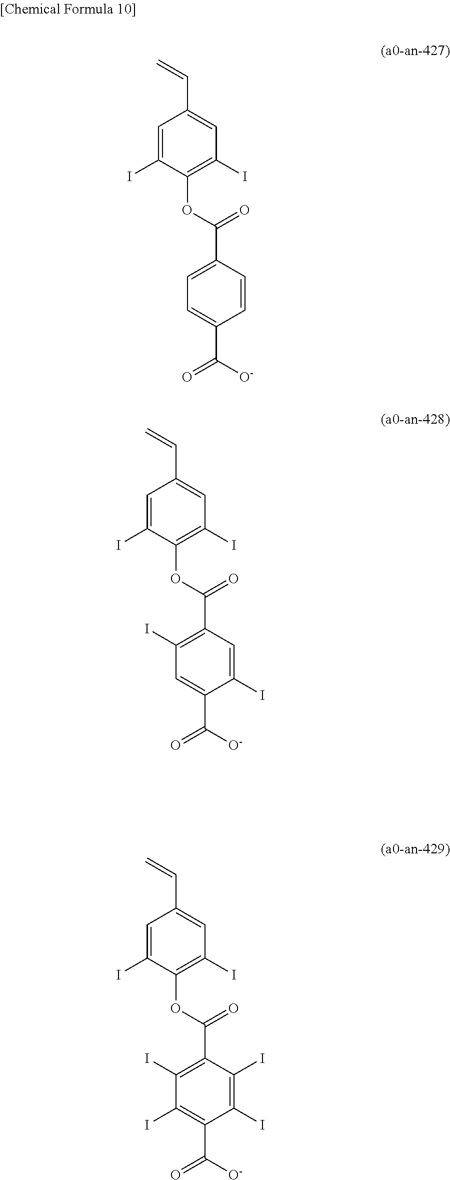

The anion moiety of the compound represented by General Formula (a02-m) is more preferably an anion in which j2 represents an integer of 1 to 5 and R⁰²² represents an iodinated alkyl group or an iodine atom. Among these, an anion in a case where j1 in the formula represents 0 is particularly preferable.

The anion moiety of the compound represented by General Formula (a02-m) is preferably at least one selected from the group consisting of anions each represented by Chemical Formulae (a0-an-401) to (a0-an-429), more preferably at least one selected from the group consisting of anions each represented by Chemical Formulae (a0-an-401) to (a0-an-426), (a0-an-428), and (a0-an-429), and particularly preferably at least one selected from the group consisting of anions each represented by Chemical Formulae (a0-an-401) to (a0-an-426).

In Regard to Constitutional Unit (a03):

The constitutional unit (a03) is a constitutional unit derived from a compound in which, in General Formula (a0-m), W represents a polymerizable group-containing group represented by General Formula (a0-w), q represents 0, k2 represents 1, R⁰² represents an iodinated alkyl group or an iodine atom, j2 represents an integer of 1 to 5, and L¹⁰ in General Formula (a0-w) represents a single bond. That is, the constitutional unit (a03) is a constitutional unit derived from a compound represented by General Formula (a03-m).

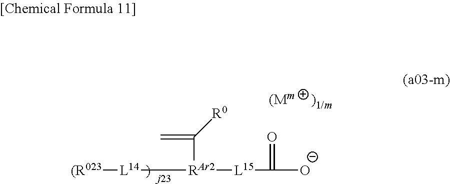

[In the formula, R⁰ has the same definition as that for R⁰ in General Formula (a0-w). R^{Ar2}, R^{Ar2}-L¹⁴-, and R^{Ar2}-L¹⁵- each have the same definition as that for R^{Ar2}, R^{Ar2}-L¹⁴-, and R^{Ar2}-L¹⁵- in Formula (a0-m). R⁰²³ represents an iodinated alkyl group or an iodine atom. j23 represents an integer of 1 to 5. M^{m+} represents an m-valent onium cation. m represents an integer of 1 or greater.]

In Formula (a03-m), examples of the iodinated alkyl group as R⁰²³ include an iodinated alkyl group having 1 to 5 carbon atoms, and an iodinated alkyl group having 1 to 3 carbon atoms is preferable, and an iodinated methyl group or an iodinated ethyl group is more preferable. It is preferable that R⁰²³ represents an iodine atom. j23 represents preferably an integer of 1 to 4 and more preferably 2, 3, or 4.

Hereinafter, preferred specific examples of the anion moiety of the compound represented by General Formula (a03-m) will be shown.

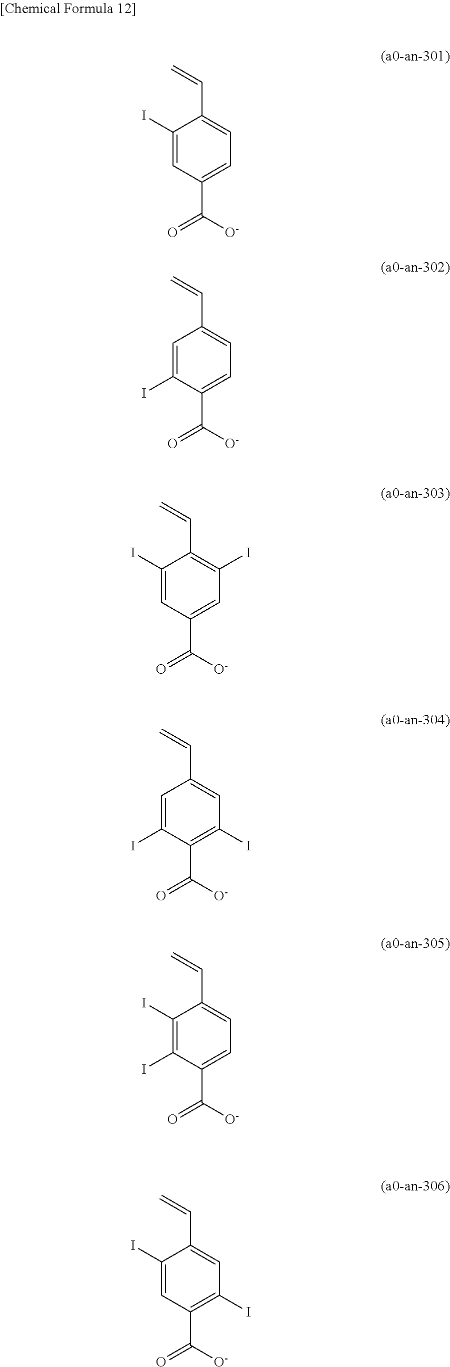

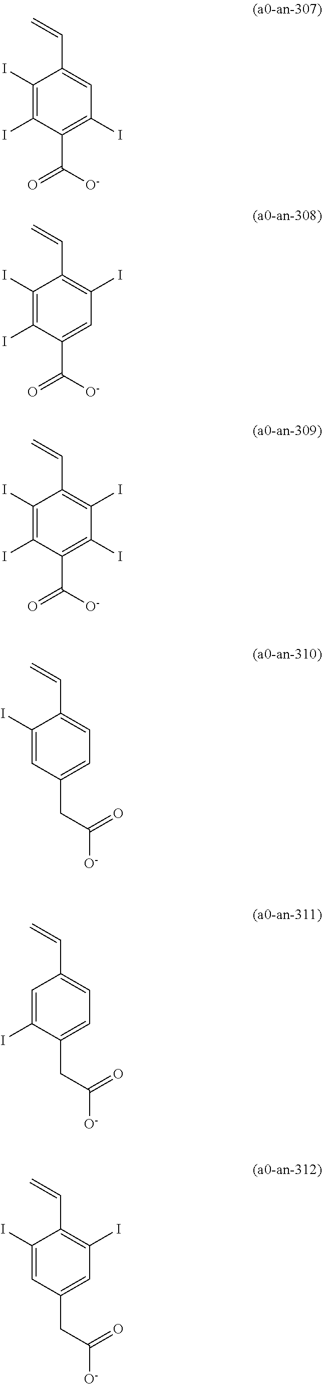

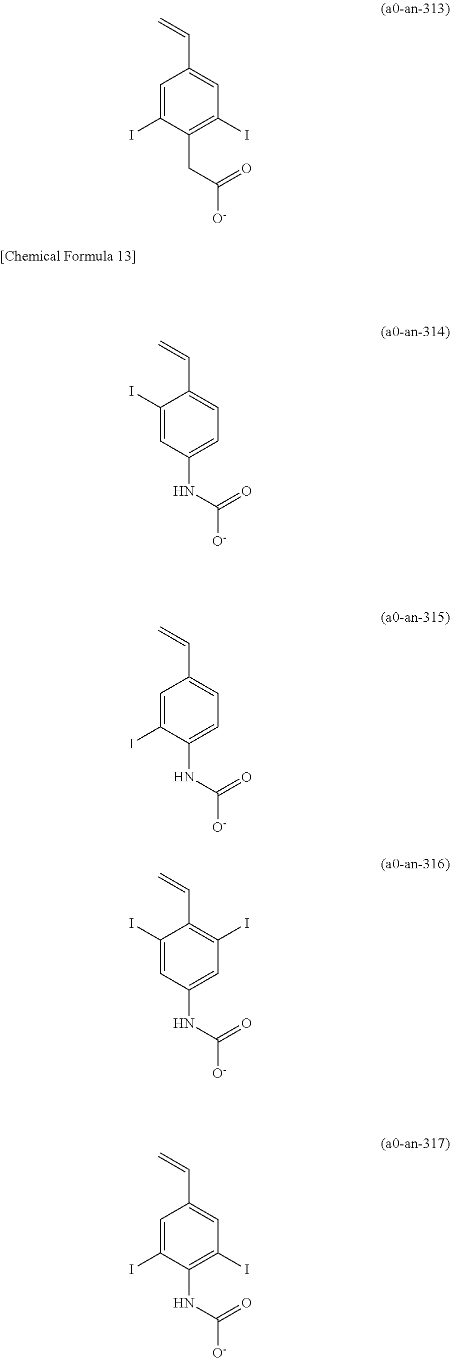

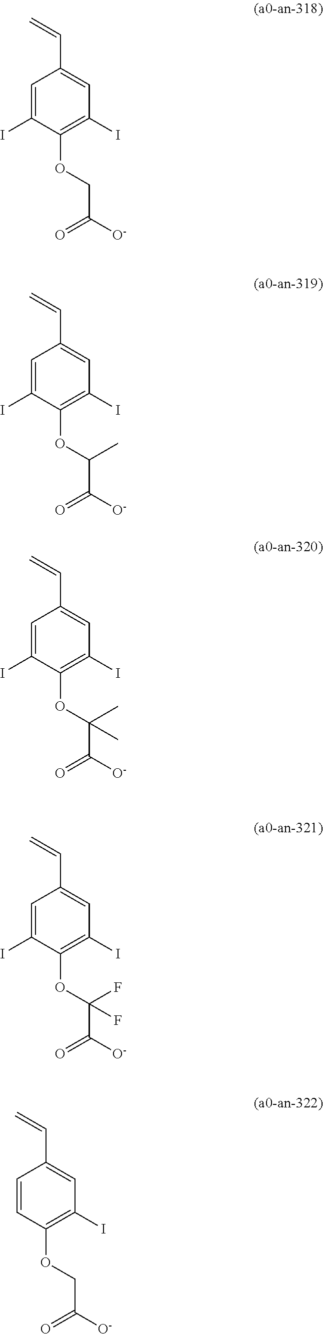

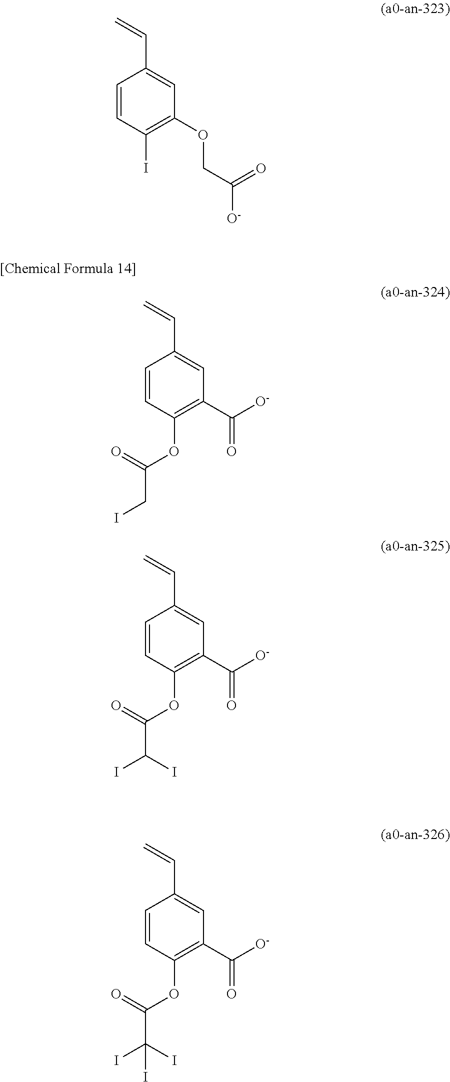

As the anion moiety of the compound represented by General Formula (a03-m), an anion in a case where R⁰²³ in the formula represents an iodine atom is more preferable.

The anion moiety of the compound represented by General Formula (a03-m) is preferably at least one selected from the group consisting of anions each represented by Chemical Formulae (a0-an-301) to (a0-an-326) and more preferably at least one selected from the group consisting of anions each represented by Chemical Formulae (a0-an-301) to (a0-an-323).

In Regard to Constitutional Unit (a04)

The constitutional unit (a04) is a constitutional unit derived from a compound in which, in General Formula (a0-m), W represents a polymerizable group-containing group represented by General Formula (a0-w), q represents 0, k2 represents 1, R⁰² represents an iodinated alkyl group or an iodine atom, j2 represents an integer of 1 to 5, and -L¹⁰-* in General Formula (a0-w) represents —C(═O)—O—* or —C(═O)—NH—* [here, * represents a bonding site that is bonded to R^{Ar2} in General Formula (a0-m)]. That is, the constitutional unit (a04) is a constitutional unit derived from a compound represented by General Formula (a04-m).

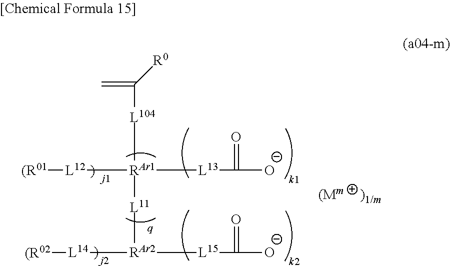

[In the formula, R⁰ has the same definition as that for R⁰ in General Formula (a0-w). -L¹⁰⁴- represents —C(═O)—O— or —C(═O)—NH—. R^{Ar1}, R^{Ar2}, R^{Ar1}-L¹¹-, R^{Ar1}-L¹²-, R^{Ar1}-L¹³-, R^{Ar2}-L¹⁴-, R^{Ar2}-L¹⁵, R⁰¹, R⁰², j1, j2, k1, k2, and q each have the same definition as that for R^{Ar1}, R^{Ar2}, R^{Ar1}-L¹¹-, R^{Ar1}-L¹², R^{Ar1}-L¹³, R^{Ar2}-L¹⁴, R^{Ar2}-L¹⁵, R⁰¹, R⁰², j1, j2, k1, k2, and q in Formula (a0-m). M^{m+} represents an m-valent onium cation. m represents an integer of 1 or greater.]

Among these, the constitutional unit (a04) is more preferably a constitutional unit derived from an anion in a case where q in General Formula (a04-m) represents 0, that is, a compound represented by General Formula (a04″-m).

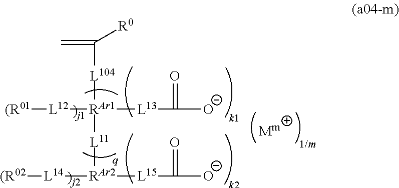

[In the formula, R⁰ has the same definition as that for R⁰ in General Formula (a0-w). -L¹⁰⁴- represents —C(═O)—O— or —CO)—NH—. R^{Ar2}, R^{Ar2}-L¹⁴-, and R^{Ar2}-L¹⁵- each have the same definition as that for R^{Ar2}, R^{Ar2}-L¹⁴-, and R^{Ar2}-L¹⁵- in Formula (a0-m). R⁰²⁴ represents an iodinated alkyl group or an iodine atom. j24 represents an integer of 1 to 5. M^{m+} represents an m-valent onium cation. m represents an integer of 1 or greater.]

In Formula (a04″-m), examples of the iodinated alkyl group as R⁰²⁴ include an iodinated alkyl group having 1 to 5 carbon atoms, and an iodinated alkyl group having 1 to 3 carbon atoms is preferable, and an iodinated methyl group or an iodinated ethyl group is more preferable. It is preferable that R⁰²⁴ represents an iodine atom. j24 represents preferably an integer of 1 to 4 and more preferably 2, 3, or 4.

Hereinafter, preferred specific examples of the anion moiety of the compound represented by General Formula (a04-m) will be shown.

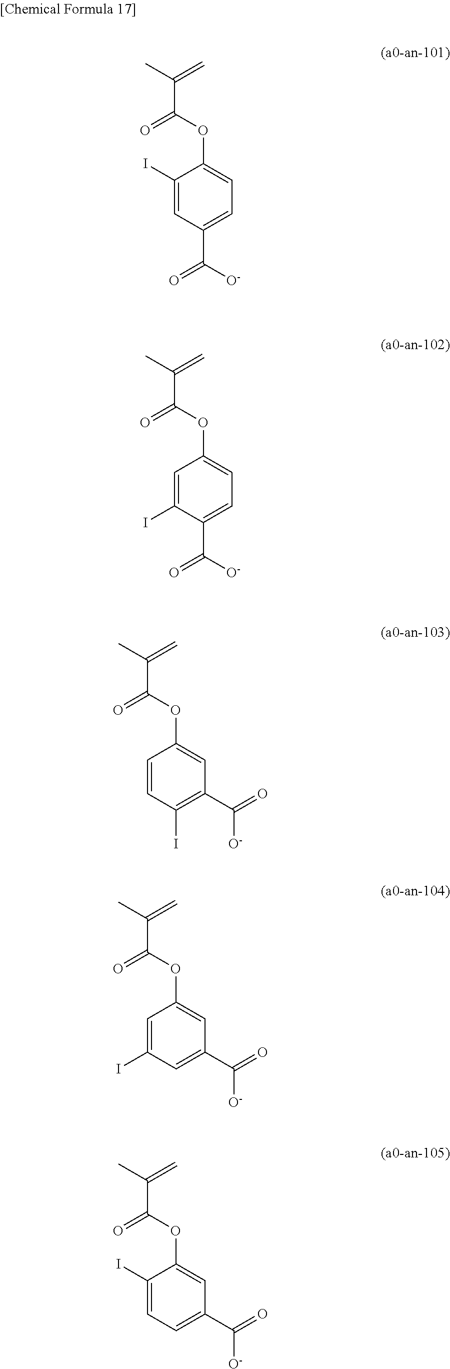

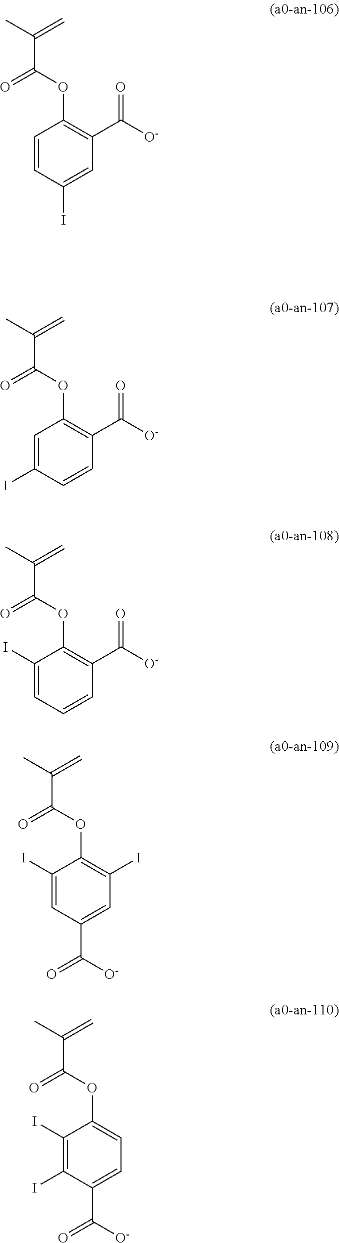

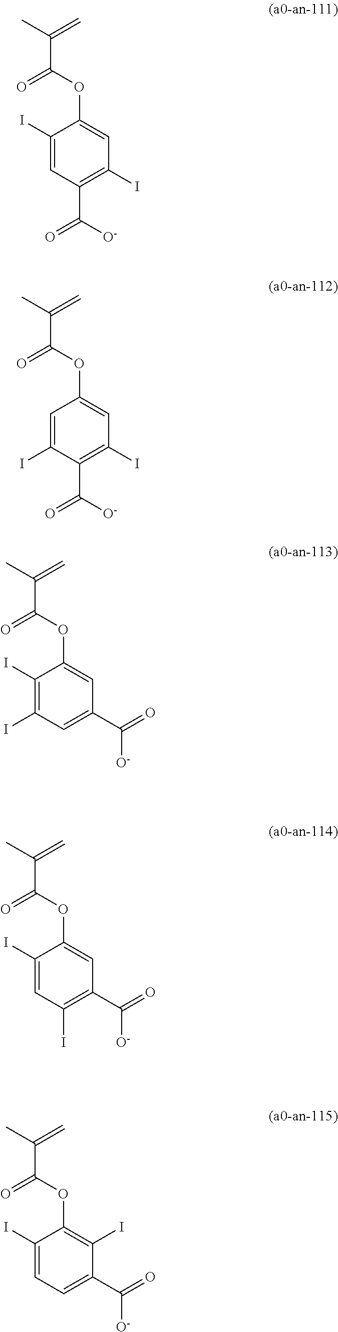

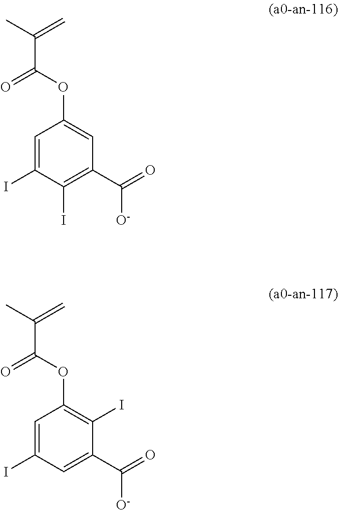

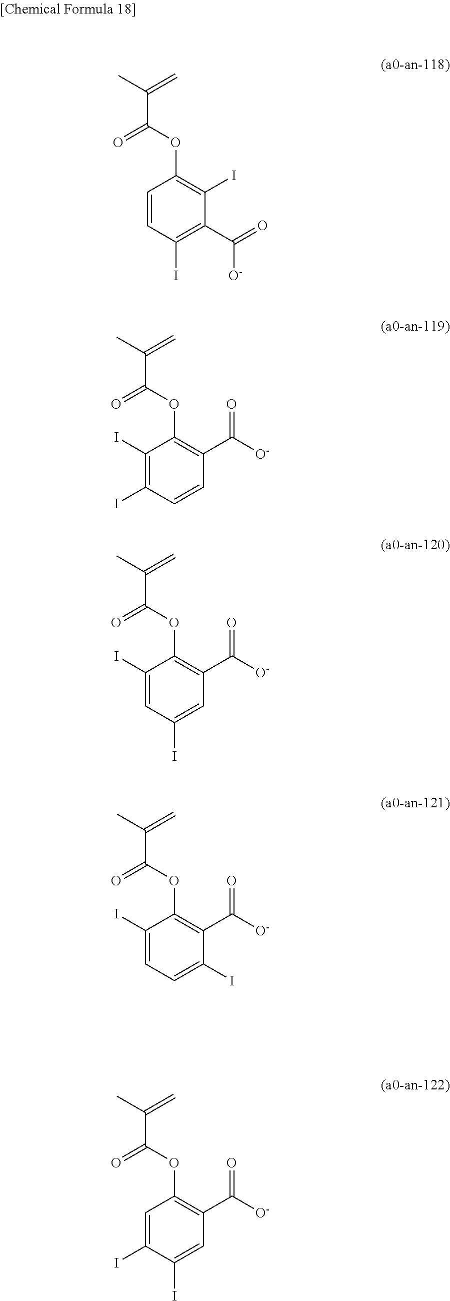

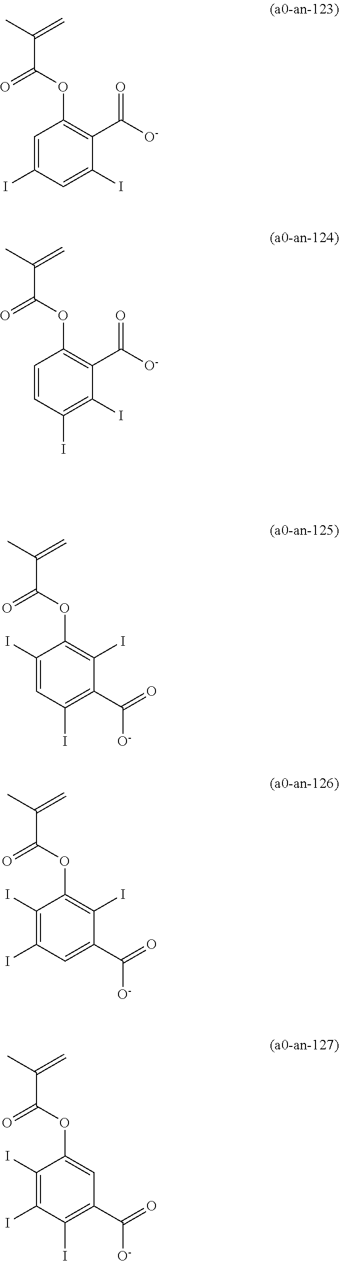

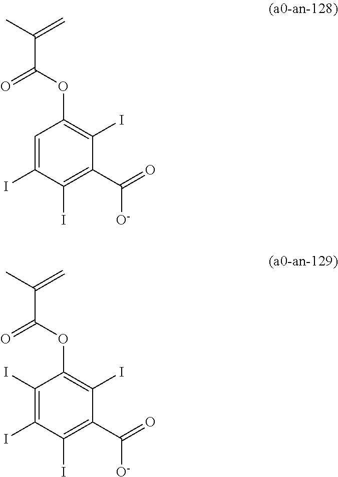

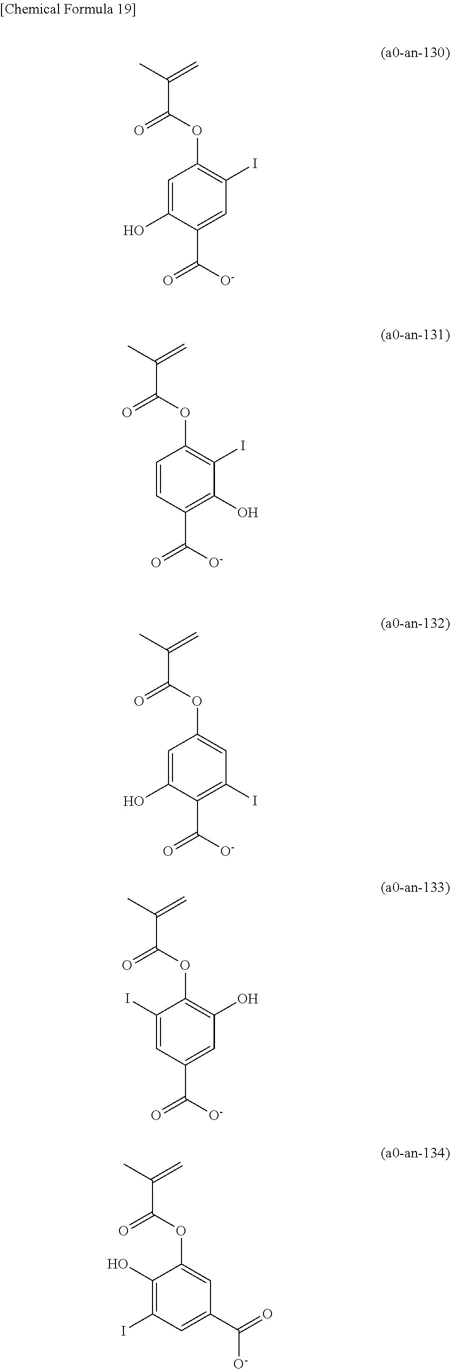

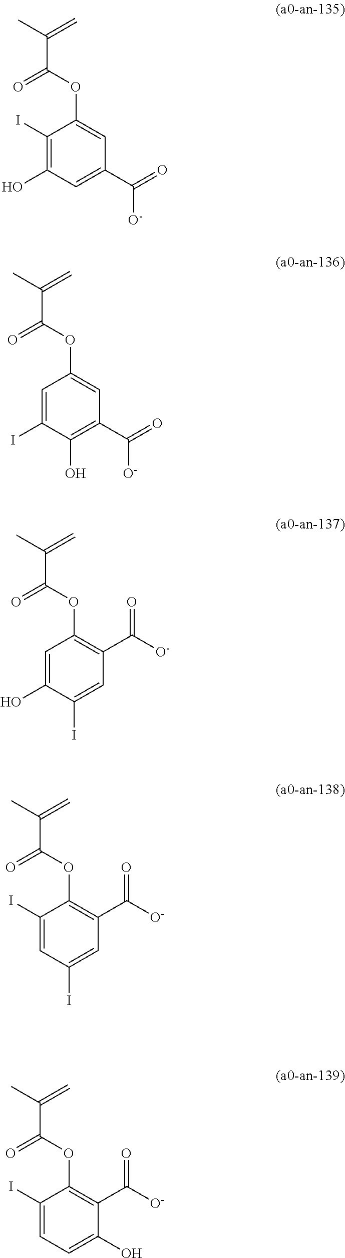

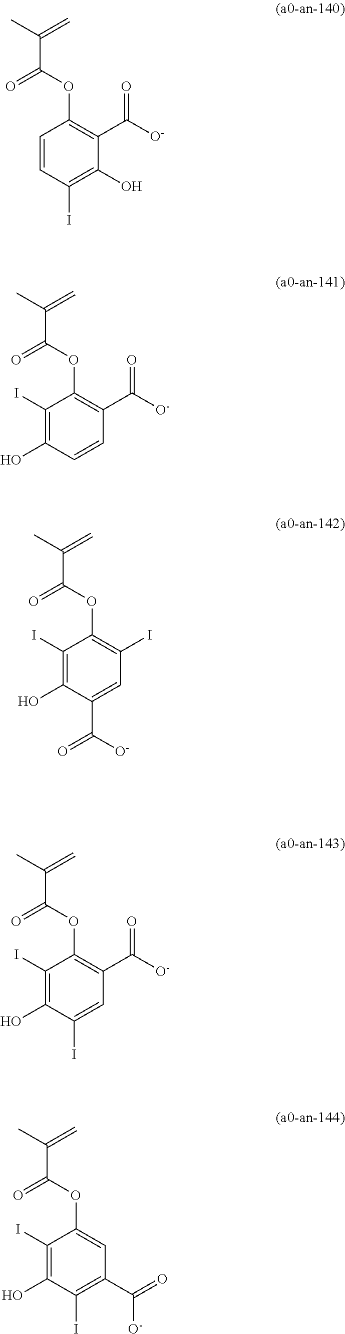

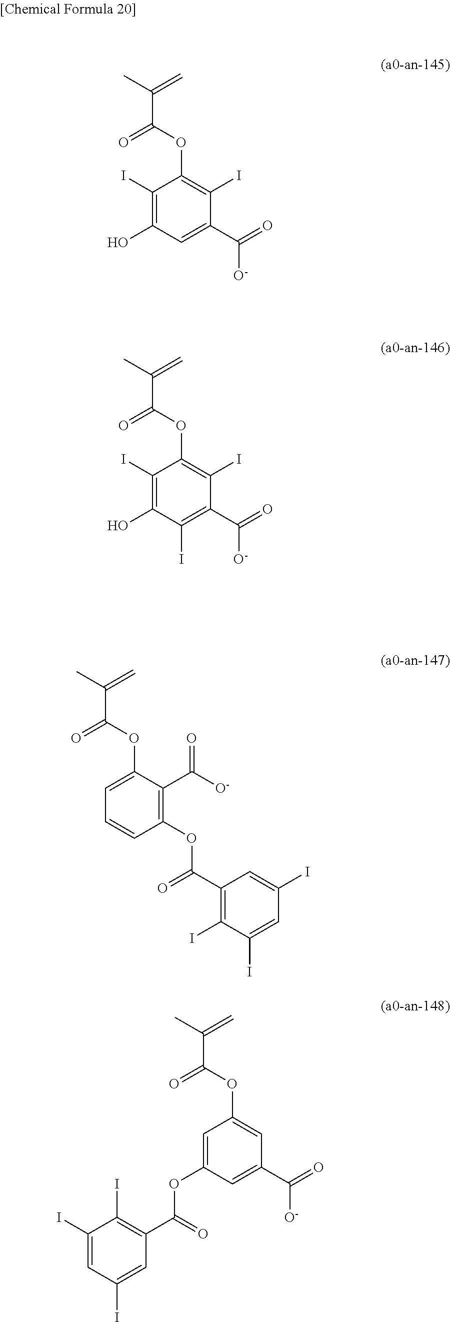

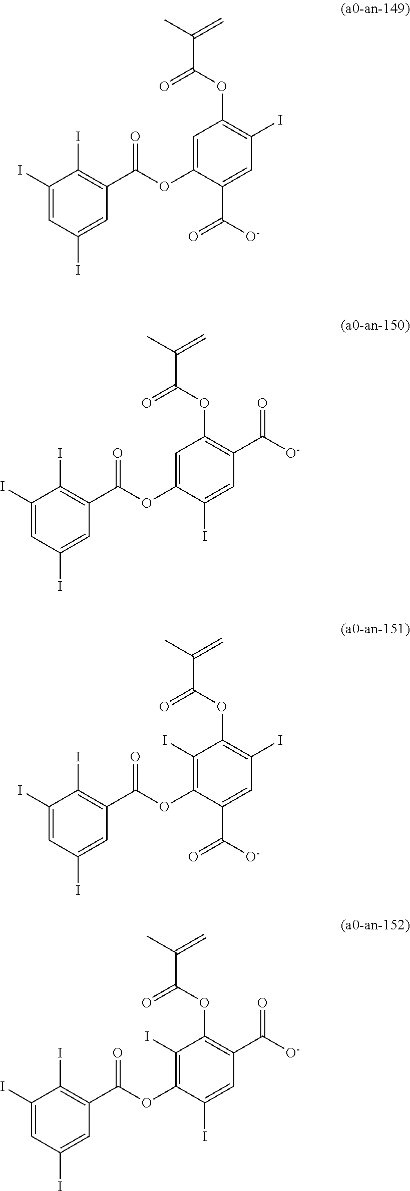

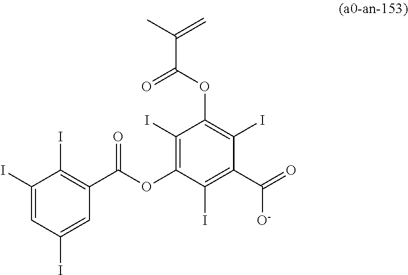

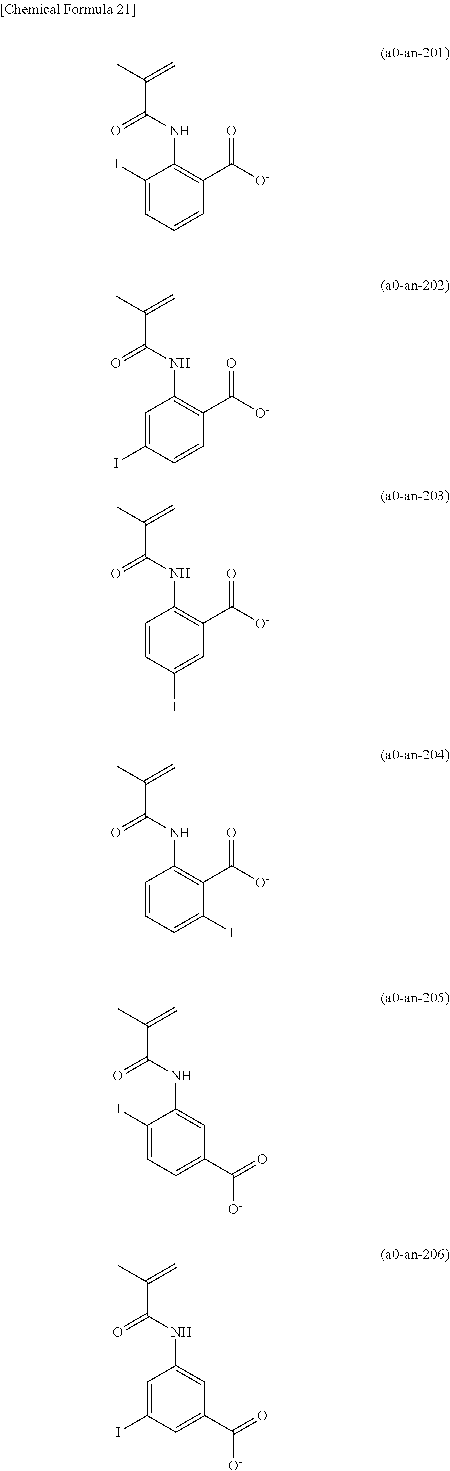

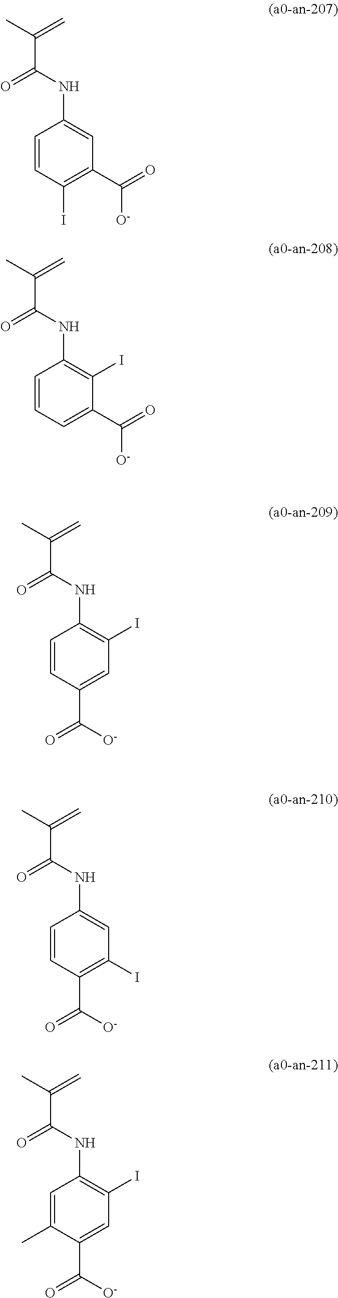

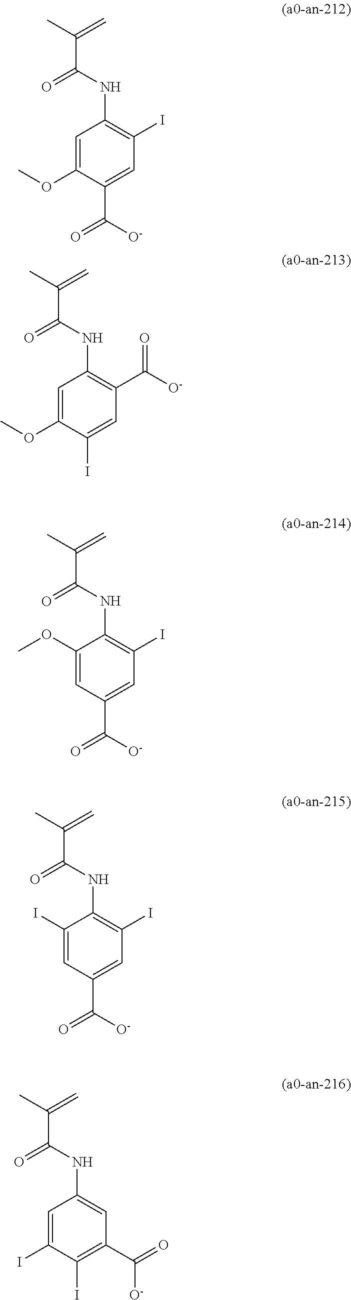

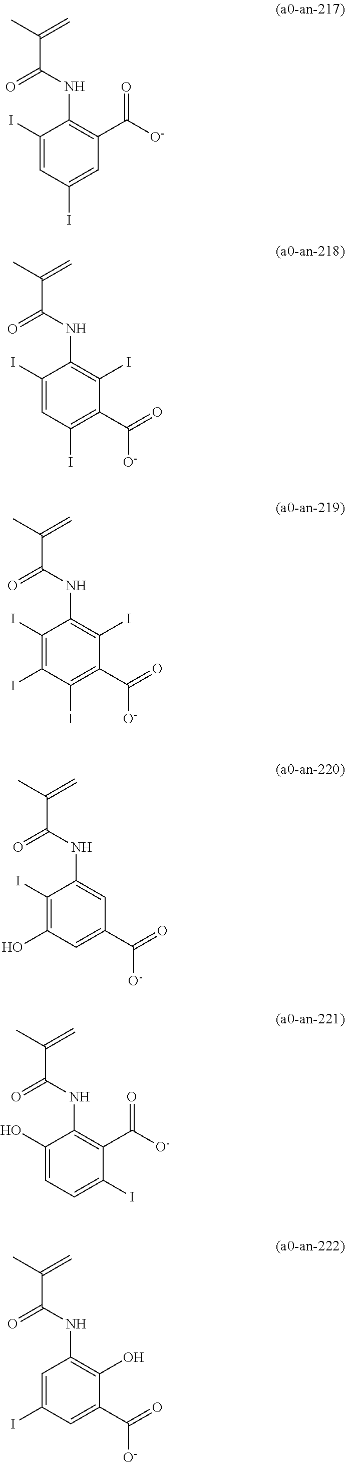

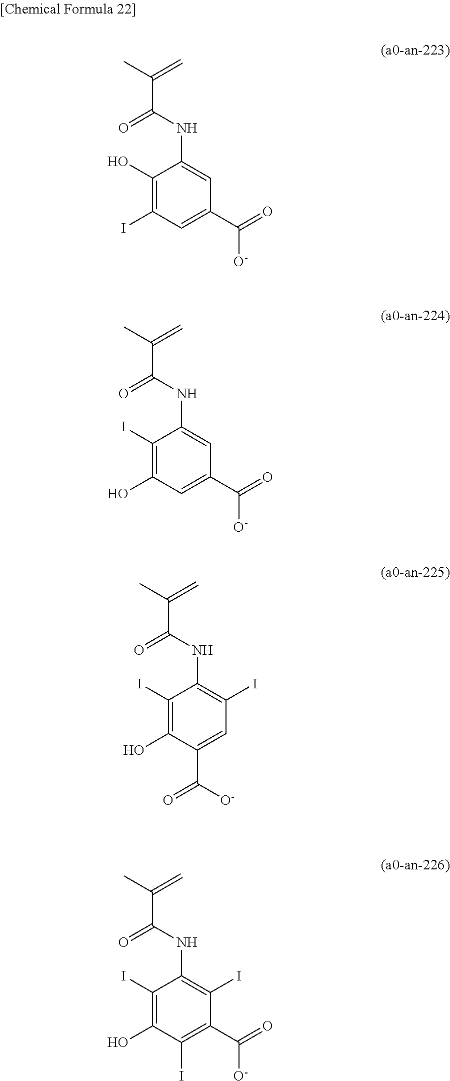

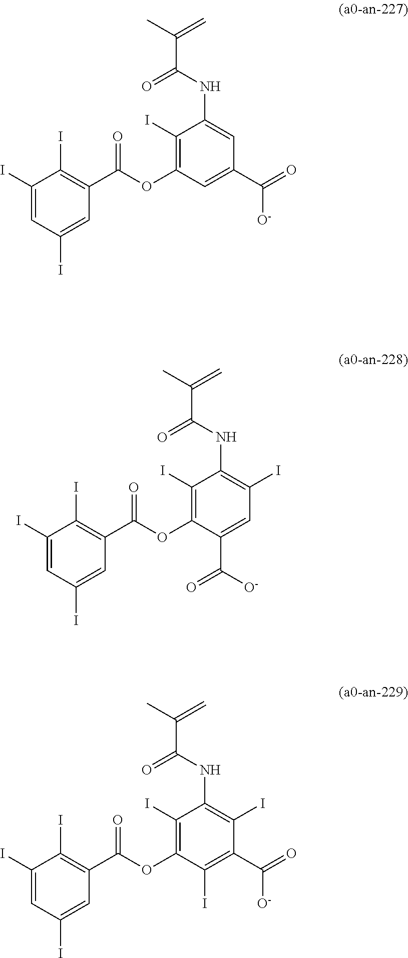

The anion moiety of the compound represented by General Formula (a04-m) is more preferably an anion in which j2 represents an integer of 1 to 5 and R⁰²² represents an iodinated alkyl group or an iodine atom. Among these, an anion in a case where j1 in the formula represents 0 is still more preferable, and an anion in a case where q represents 0 is particularly preferable.

The anion moiety of the compound represented by General Formula (a04-m) is preferably at least one selected from the group consisting of anions each represented by Chemical Formulae (a0-an-101) to (a0-an-153) and (a0-an-201) to (a0-an-229) and more preferably at least one selected from the group consisting of anions each represented by Chemical Formulae (a0-an-101) to (a0-an-146) and (a0-an-201) to (a0-an-226).

### [Cation Moiety]

In Formula (a0-m), M^{m+} represents an m-valent onium cation. m represents an integer of 1 or greater.

Examples of the onium cation as M^{m+} include a sulfonium cation, an iodonium cation, and an ammonium cation. Among these, a sulfonium cation or an iodonium cation is preferable.

Preferred examples of the cation moiety (M^{m+})_{1/m} include onium cations each represented by General Formulae (ca-1) to (ca-3).

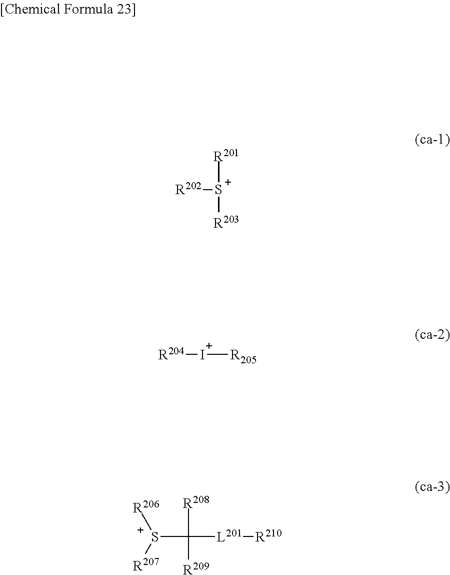

[In the formulae, R²⁰¹ to R²⁰⁷ each independently represent an aryl group, an alkyl group, or an alkenyl group, each of which may have a substituent. R²⁰¹ to R²⁰³, and R²⁰⁶ and R²⁰⁷ may be bonded to each other to form a ring together with the sulfur atoms in the formulae. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a —SO₂-containing cyclic group which may have a substituent. L²⁰¹ represents —C(═O)— or —C(═O)—O—.]

In General Formulae (ca-1) to (ca-3), examples of the aryl group as R²⁰¹ to R²⁰⁷ include an unsubstituted aryl group having 6 to 20 carbon atoms, where a phenyl group or a naphthyl group is preferable.

The alkyl group as R²⁰¹ to R²⁰⁷ is preferably a chain-like or cyclic alkyl group which has 1 to 30 carbon atoms.

The alkenyl group as R²⁰¹ to R²⁰⁷ preferably has 2 to 10 carbon atoms.

Examples of the substituent which may be contained in R²⁰1 to R²⁰⁷ and R²¹⁰ include an alkyl group, a halogen atom, a halogenated alkyl group, a carbonyl group, a cyano group, an amino group, an aryl group, and groups each represented by General Formulae (ca-r-1) to (ca-r-7).

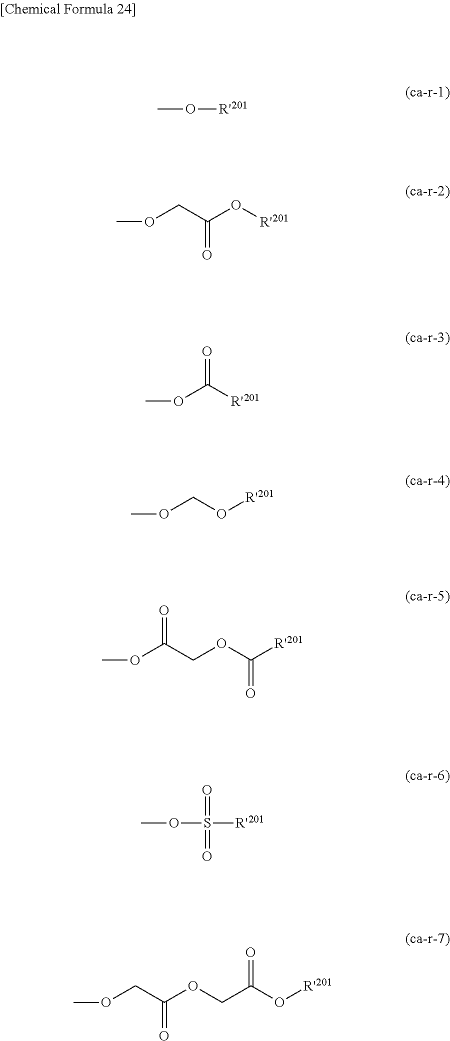

[In the formulae, R′²⁰¹'s each independently represents a hydrogen atom, a cyclic group which may have a substituent, a chain-like alkyl group which may have a substituent, or a chain-like alkenyl group which may have a substituent.]

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group is saturated.

The aromatic hydrocarbon group as R′²⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R′⁰, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Further, the cyclic hydrocarbon group as R′²⁰¹ may have a heteroatom such as a heterocyclic ring. Examples thereof include a lactone-containing cyclic group, an —SO₂-containing cyclic group, and another heterocyclic group represented by each of Chemical Formulae (r-hr-1) to (r-hr-16) described below.

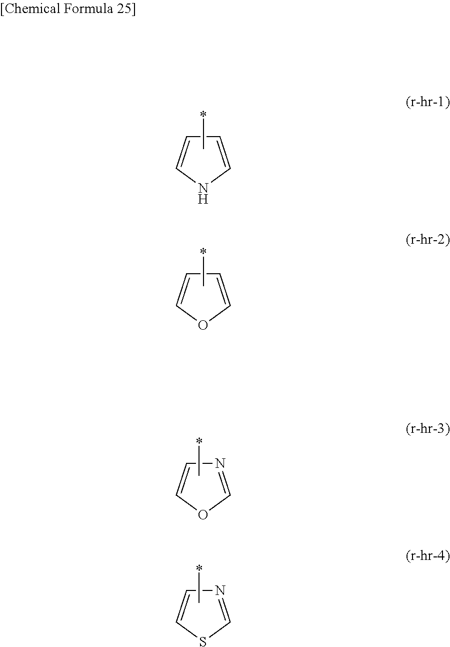

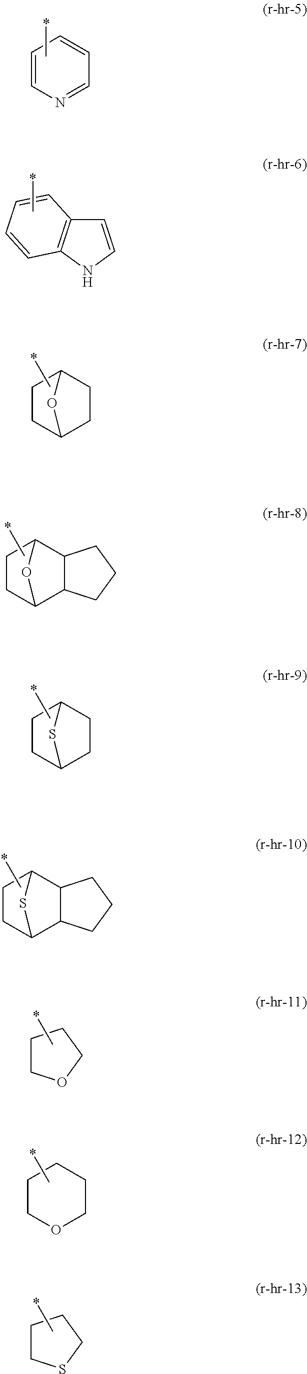

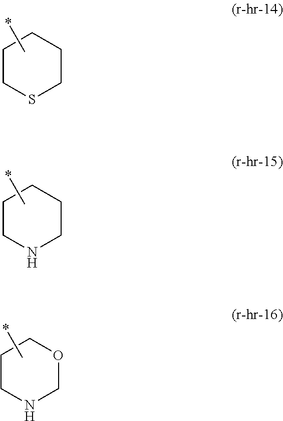

Examples of the substituent for the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as a substituent, a fluorine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

Chain-Like Alkyl Group which May have Substituent:

The chain-like alkyl group as R²⁰¹ may be linear or branched.

The linear alkyl group has preferably 1 to 20 carbon atoms, more preferably 1 to carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R²⁰¹ may be linear or branched, and has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, and the chain-like alkenyl group which may have a substituent as R′²⁰¹ include the same groups as those for the acid dissociable group represented by Formula (a1-r-2) described as the cyclic group which may have a substituent and the chain-like alkyl group which may have a substituent, in addition to those described above.

Among the examples, R′²⁰¹ represents preferably a cyclic group which may have a substituent, and more preferably a cyclic hydrocarbon group which may have a substituent. More specifically, it is, for example, preferably a group obtained by removing one or more hydrogen atoms from a phenyl group, a naphthyl group, or a polycycloalkane; a lactone-containing cyclic group; —SO₂-containing cyclic group; or the like.

In General Formulae (ca-1) to (ca-3), in a case where R²⁰¹ to R²⁰³ and R²⁰⁶ and R²⁰⁷ are bonded to each other to form a ring with a sulfur atom in the formula, these groups may be bonded to each other via a heteroatom such as a sulfur atom, an oxygen atom, or a nitrogen atom, or a functional group such as a carbonyl group, —SO—, —SO₂—, —SO₃—, —COO—, —CONH—, or —N(R_{N})— (here, R_{N} represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and preferably a hydrogen atom or an alkyl group having 1 to 3 carbon atoms. In a case where R²⁰⁸ and R²⁰⁹ each represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a —SO₂-containing cyclic group which may have a substituent.

Examples of the aryl group as R²¹⁰ include an unsubstituted aryl group having 6 to 20 carbon atoms, and a phenyl group or a naphthyl group is preferable.

As the alkyl group as R²¹⁰, a chain-like or cyclic alkyl group having 1 to 30 carbon atoms is preferable.

It is preferable that the alkenyl group as R²¹⁰ has 2 to 10 carbon atoms.

The —SO₂-containing cyclic group which may have a substituent, as R²¹⁰, is preferably “—SO₂-containing polycyclic group”.

Specific examples of suitable cations represented by Formula (ca-1) include cations each represented by Chemical Formulae (ca-1-1) to (ca-1-75).

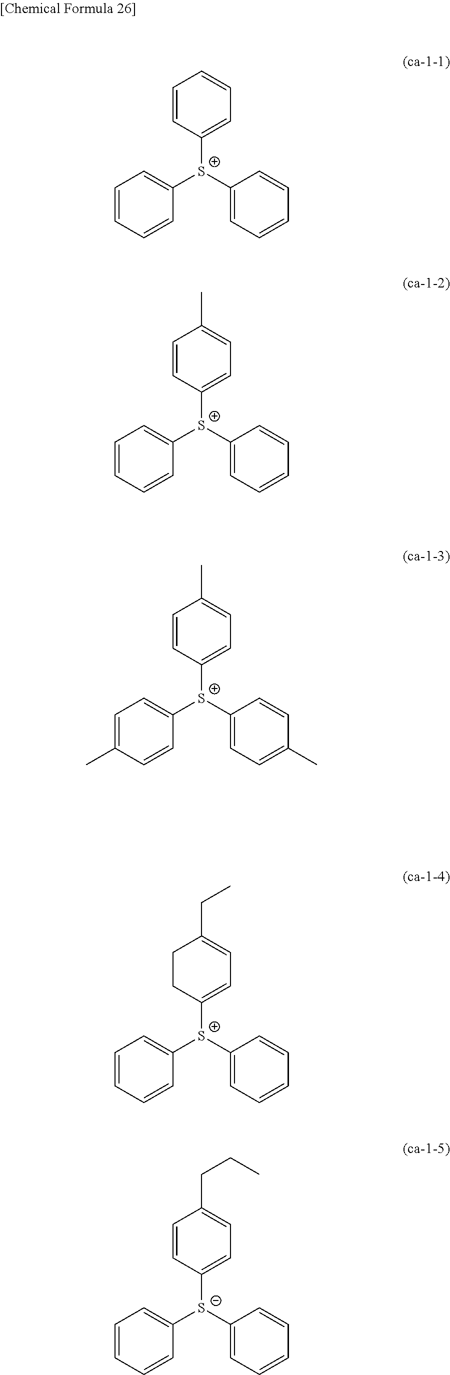

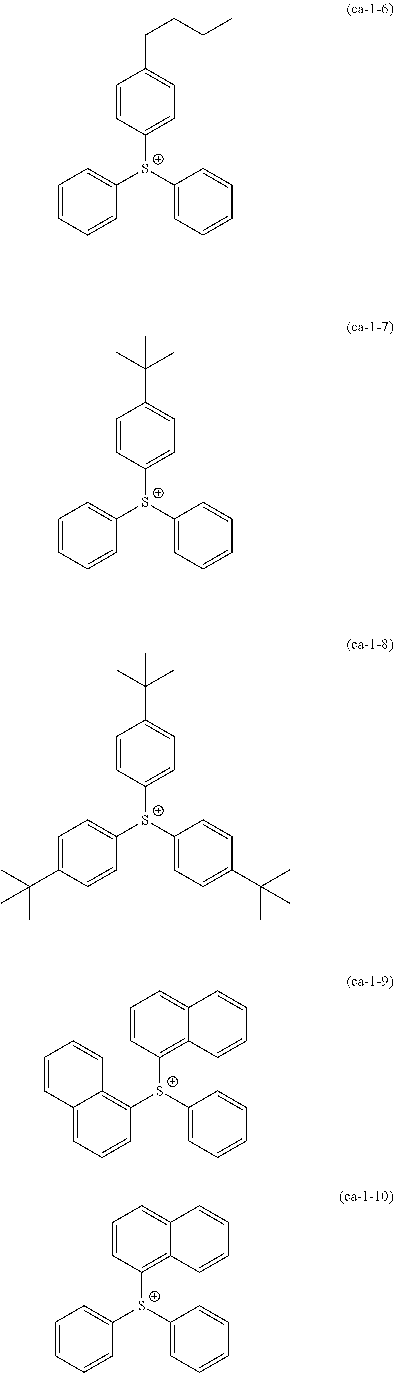 [SMILES: CCCCc1ccc([S+](c2ccccc2)c2ccccc2)cc1 ; CC(C)(C)c1ccc([S+](c2ccccc2)c2ccccc2)cc1 ; CC(C)(C)c1ccc([S+](c2ccc(C(C)(C)C)cc2)c2ccc(C(C)(C)C)cc2)cc1 ; c1ccc([S+](c2cccc3ccccc23)c2cccc3ccccc23)cc1 ; c1ccc([S+](c2ccccc2)c2cccc3ccccc23)cc1]

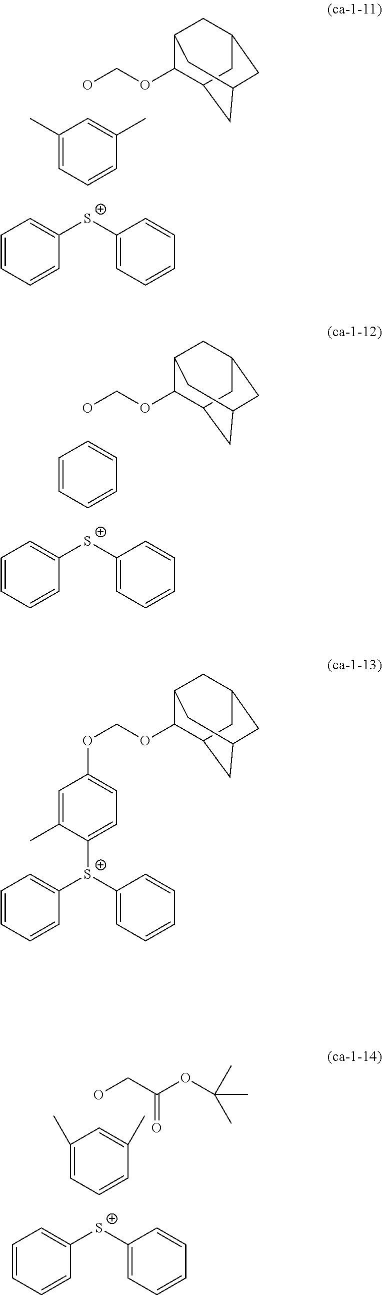

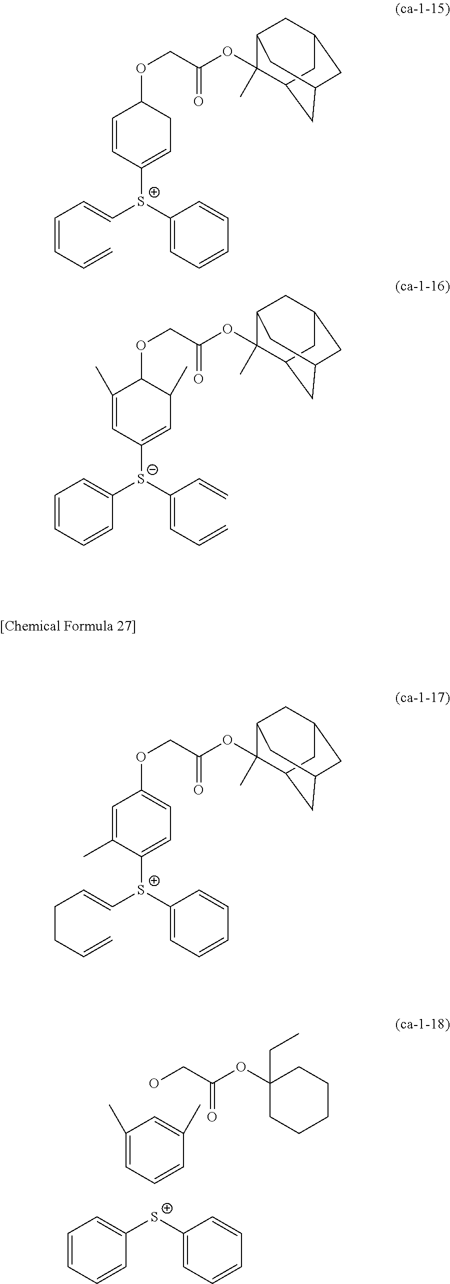

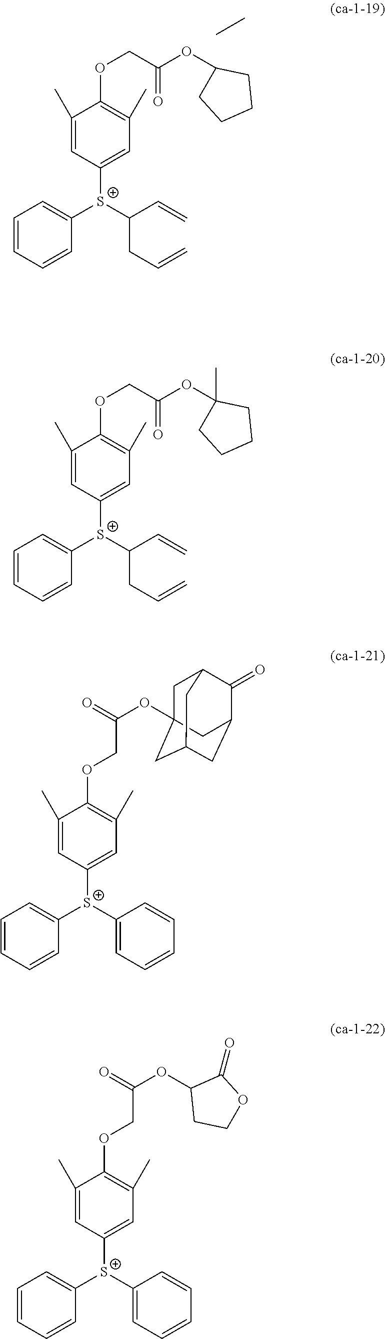

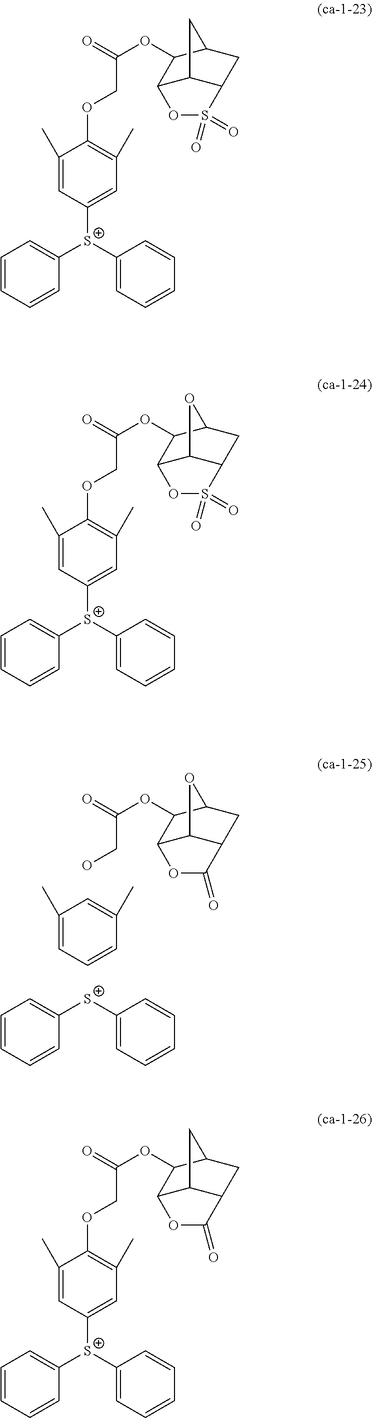

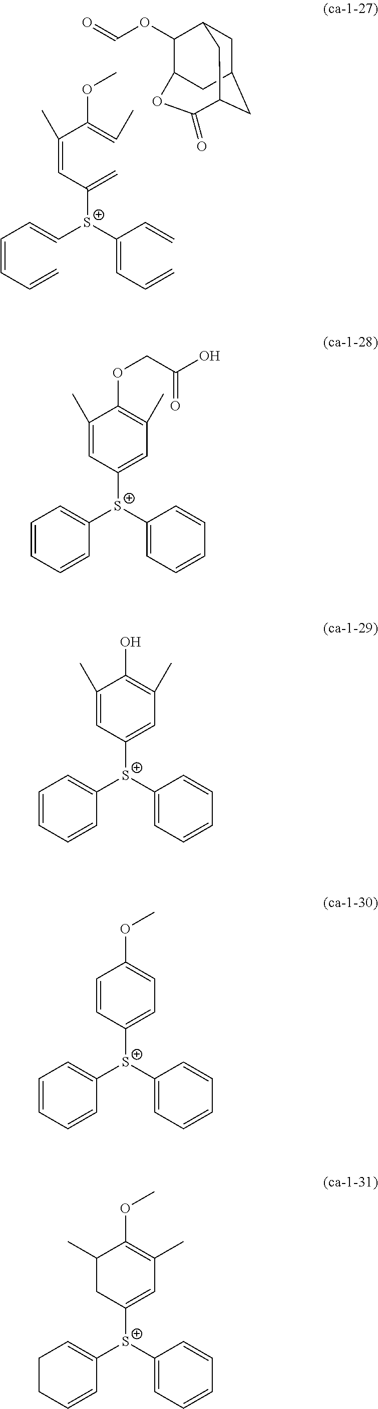 [SMILES: C=C/C=C\C=C\[S+](C(=C)/C=C(C)\C(=C\C)OC)/C(C=C)=C/C=C ; O=COC1C2CC3CC(C2)C(=O)OC1C3 ; Cc1cc([S+](c2ccccc2)c2ccccc2)cc(C)c1OCC(=O)O ; Cc1cc([S+](c2ccccc2)c2ccccc2)cc(C)c1O ; COc1ccc([S+](c2ccccc2)c2ccccc2)cc1 ; COC1=C(C)C=C([S+](C2=CCCC=C2)c2ccccc2)CC1C]

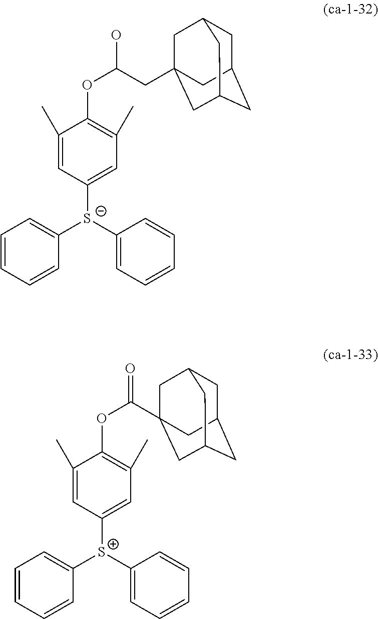 [SMILES: Cc1cc([S-](c2ccccc2)c2ccccc2)cc(C)c1OC(O)CC12CC3CC(CC(C3)C1)C2 ; Cc1cc([S+](c2ccccc2)c2ccccc2)cc(C)c1OC(=O)C12CC3CC(CC(C3)C1)C2]

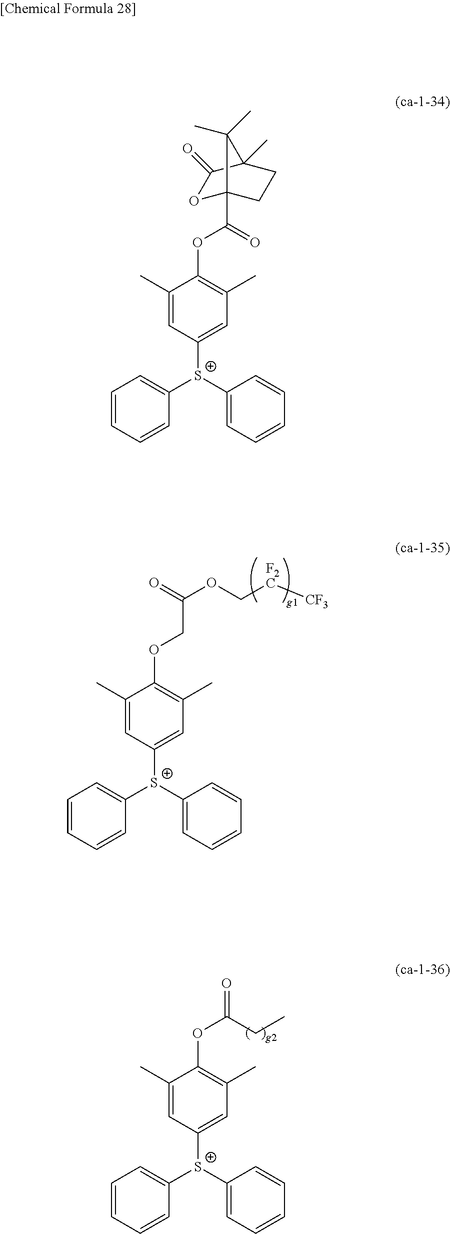

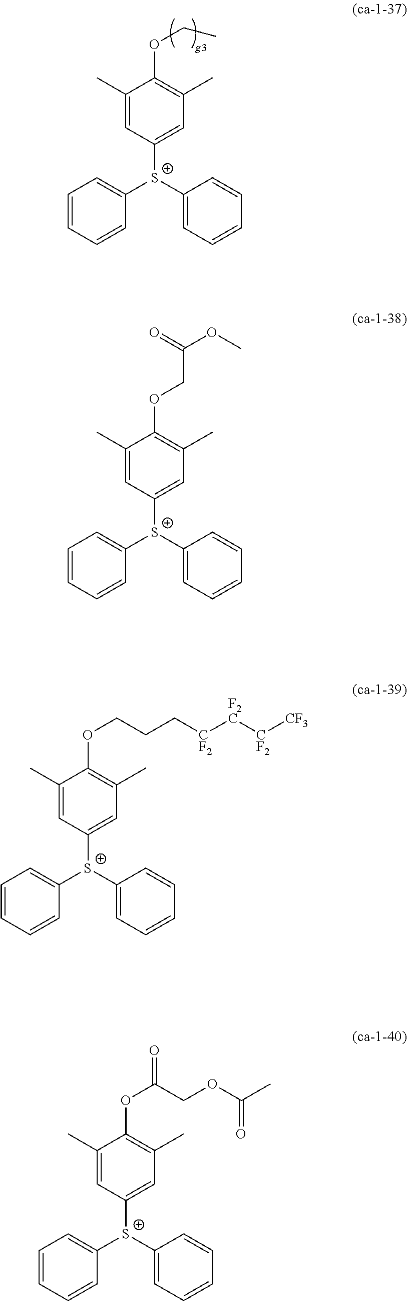

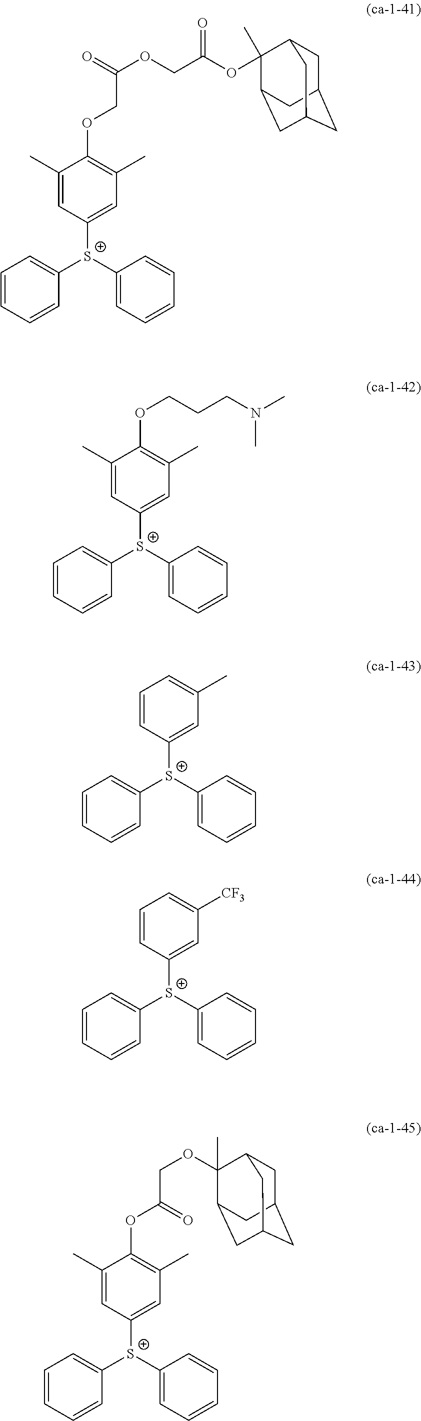 [SMILES: Cc1cc([S+](c2ccccc2)c2ccccc2)cc(C)c1OCC(=O)OCC(=O)OC1(C)C2CC3CC(C2)CC1C3 ; Cc1cc([S+](c2ccccc2)c2ccccc2)cc(C)c1OCCCN(C)C ; Cc1cccc([S+](c2ccccc2)c2ccccc2)c1 ; FC(F)(F)c1cccc([S+](c2ccccc2)c2ccccc2)c1 ; Cc1cc([S+](c2ccccc2)c2ccccc2)cc(C)c1OC(=O)COC1(C)C2CC3CC(C2)CC1C3]

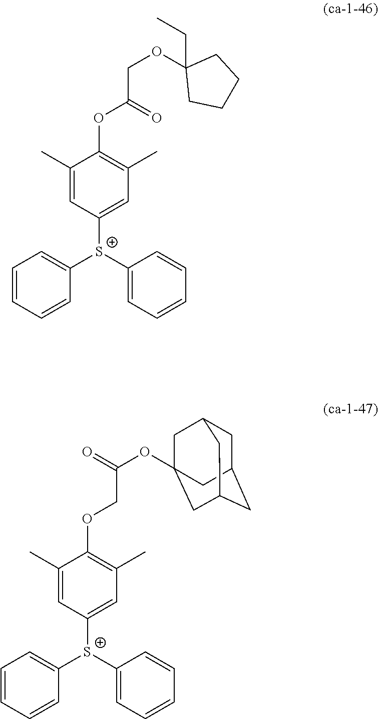 [SMILES: CCC1(OCC(=O)Oc2c(C)cc([S+](c3ccccc3)c3ccccc3)cc2C)CCCC1 ; Cc1cc([S+](c2ccccc2)c2ccccc2)cc(C)c1OCC(=O)OC12CC3CC(CC(C3)C1)C2]

[In the formulae, g1, g2, and g3 represent a repeating number, g1 represents an integer of 1 to 5, g2 represents an integer of 0 to 20, and g3 represents an integer of 0 to 20.]

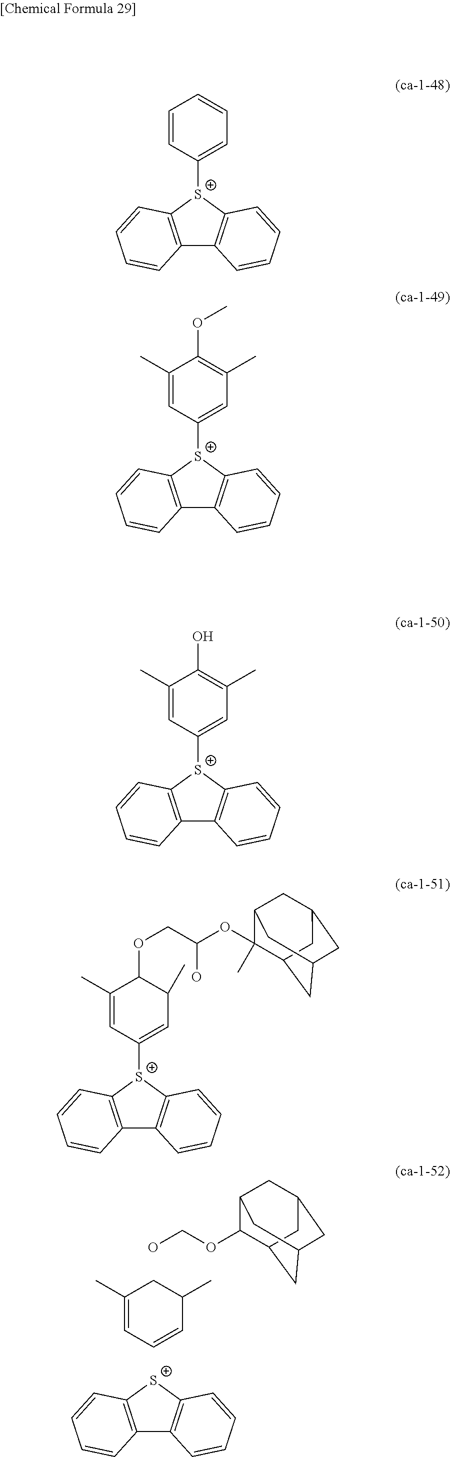

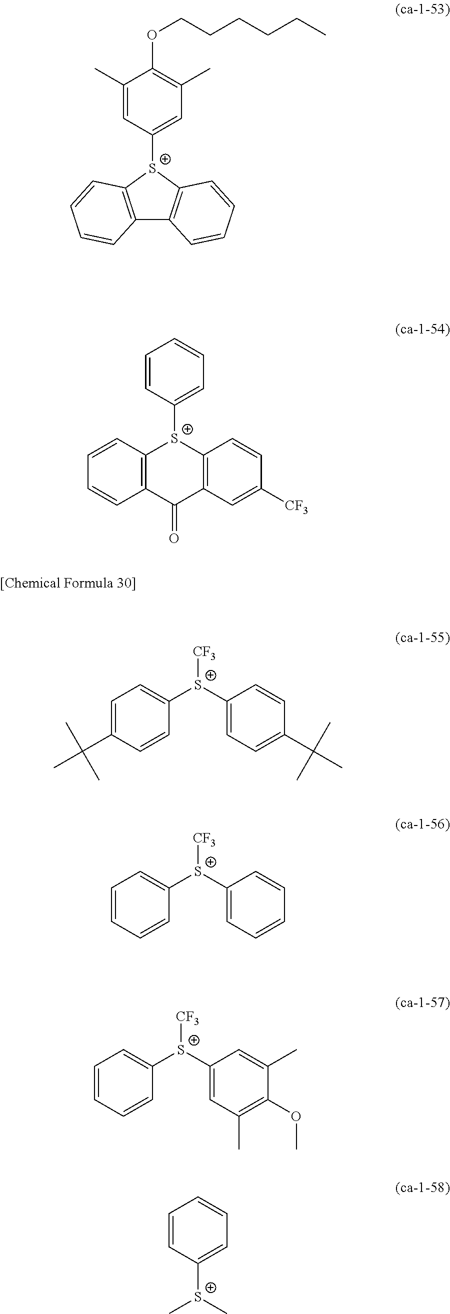

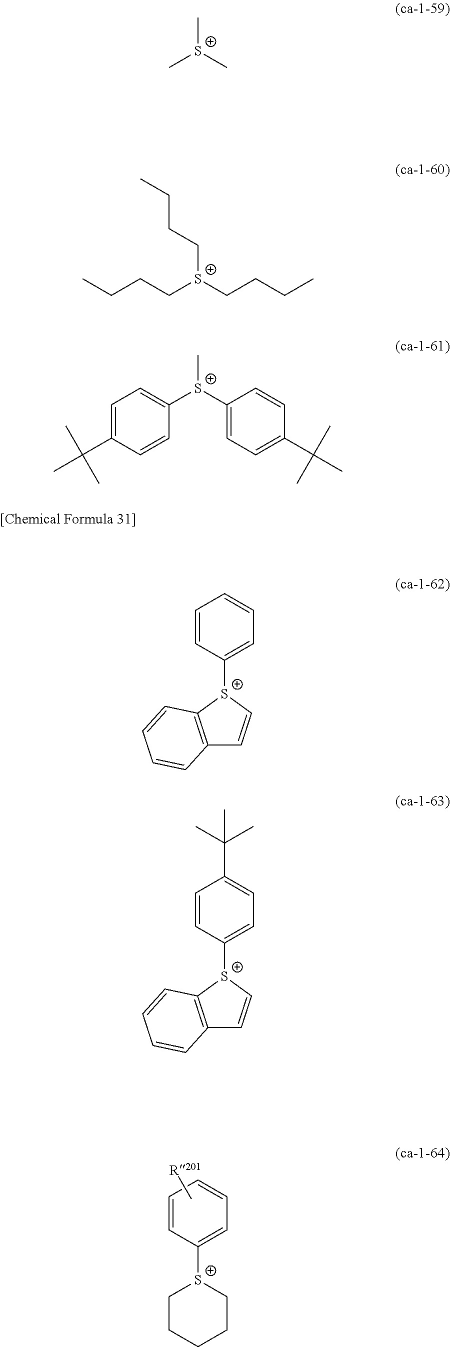

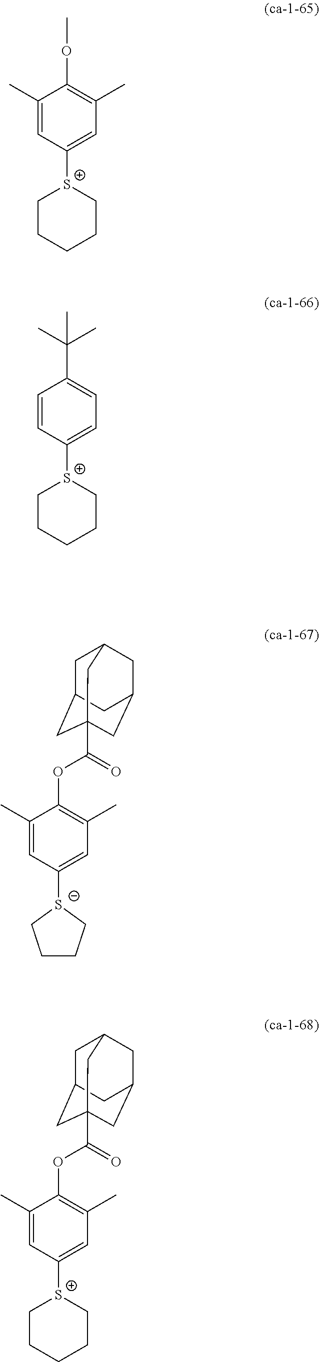

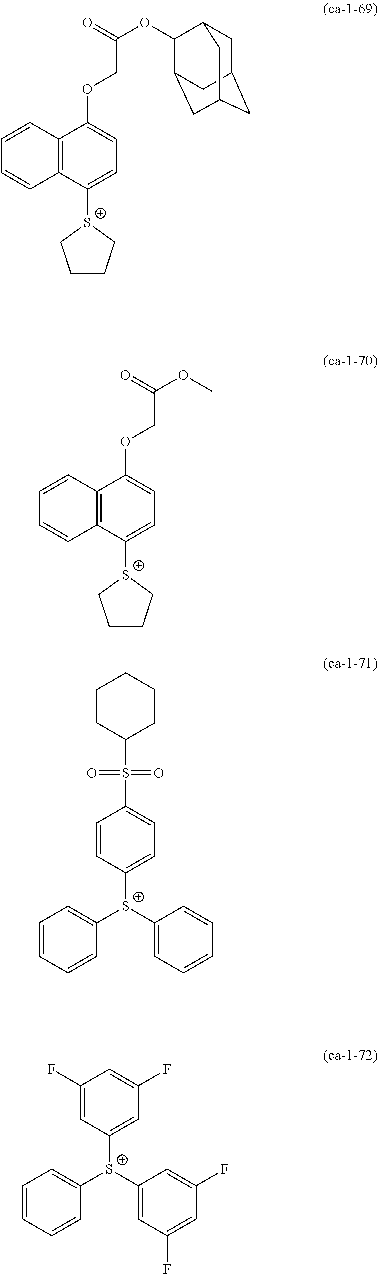 [SMILES: O=C(COc1ccc([S+]2CCCC2)c2ccccc12)OC1C2CC3CC(C2)CC1C3 ; COC(=O)COc1ccc([S+]2CCCC2)c2ccccc12 ; O=S(=O)(c1ccc([S+](c2ccccc2)c2ccccc2)cc1)C1CCCCC1 ; Fc1cc(F)cc([S+](c2ccccc2)c2cc(F)cc(F)c2)c1]

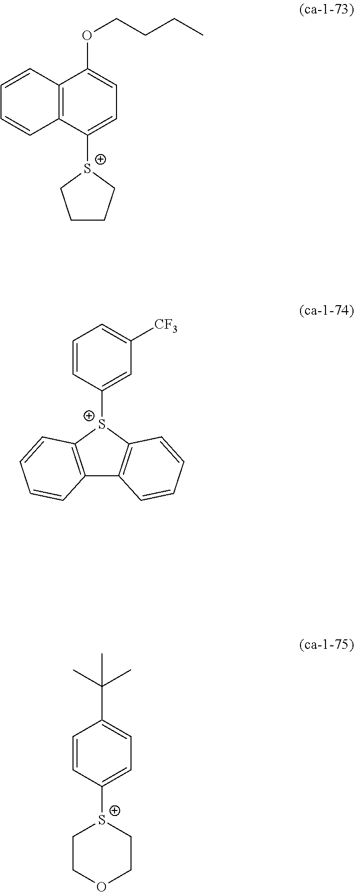 [SMILES: CCCCOc1ccc([S+]2CCCC2)c2ccccc12 ; FC(F)(F)c1cccc(-[s+]2c3ccccc3c3ccccc32)c1 ; CC(C)(C)c1ccc([S+]2CCOCC2)cc1]

[In the formulae, R″²⁰¹ represents a hydrogen atom or a substituent, and examples of the substituent include the same groups as those for the substituents which may be included in R²⁰¹ to R²⁰⁷ and R²¹⁰ to R²¹².]

Specific examples of suitable cations represented by Formula (ca-2) include a diphenyliodonium cation and a bis(4-tert-butylphenyl)iodonium cation.

Specific examples of suitable cations represented by Formula (ca-3) include cations each represented by Formulae (ca-3-1) to (ca-3-6).

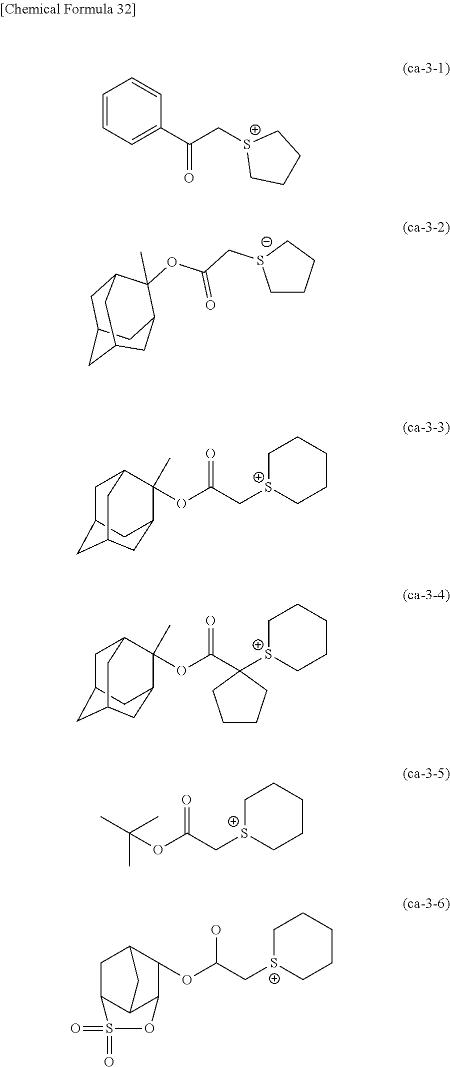 [SMILES: O=C(C[S+]1CCCC1)c1ccccc1 ; CC1(OC(=O)C[S-]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=S1(=O)OC2C3CC(CC31)C2OC(O)C[S+]1CCCCC1]

In addition, examples of the cation moiety ((M^{m+})_{1/m}) include an organic ammonium cation represented by General Formula (ca-4).

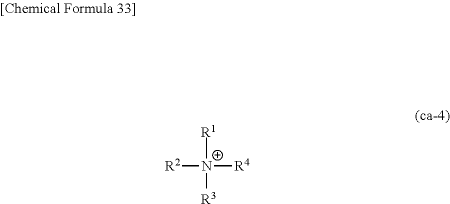

[In the formula, R¹ to R⁴ each independently represent a hydrocarbon group which may have a substituent. Alternatively, at least two of R¹ to R⁴ may be bonded to each other to form an alicyclic structure together with the nitrogen atom in the formula.]

In General Formula (ca-4), the hydrocarbon groups as R¹ to R⁴ are each independently preferably a hydrocarbon group having 1 to 15 carbon atoms and more preferably a hydrocarbon group having 1 to 10 carbon atoms. Further, the total number of carbon atoms in the hydrocarbon group as R¹ to R⁴ is preferably in a range of 1 to 20, more preferably in a range of 3 to 18, and still more preferably in a range of 4 to 15.

Examples of the hydrocarbon group as R¹ to R⁴ include a linear or branched alkyl group and a cyclic hydrocarbon group.

The linear or branched alkyl group is preferably a linear or branched alkyl group having 1 to 10 carbon atoms and more preferably a linear or branched alkyl group having 1 to 5 carbon atoms.

The cyclic hydrocarbon group may be an alicyclic hydrocarbon group or an aromatic hydrocarbon group.

The alicyclic hydrocarbon group is preferably a group obtained by removing one hydrogen atom from a monocycloalkane. The monocycloalkane has preferably 3 to 6 carbon atoms, and specific examples thereof include cyclopentane and cyclohexane. The aromatic hydrocarbon group is preferably a phenyl group or a benzyl group.

Examples of the substituent which may be included in the hydrocarbon group as R¹ to R⁴ include an alkoxy group, a hydroxyl group, an oxo group (═O), and an amino group.

Specific examples of the suitable cation represented by General Formula (ca-4) include cations each represented by Chemical Formulae (ca-4-1) to (ca-4-4) described below.

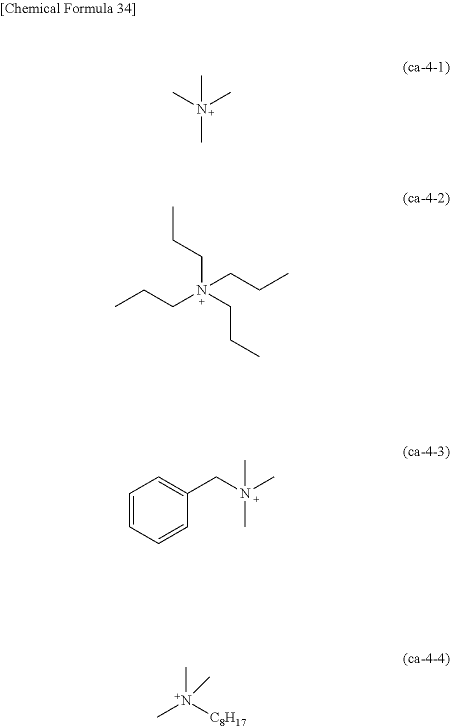

Among the examples, M^{m+} represents preferably an m-valent sulfonium cation and more preferably the cation represented by Formula (ca-1).

In addition, from the viewpoint of increasing the sensitivity, it is preferable that M^{m+} represents an m-valent onium cation having a fluorine atom. It is preferable that M^{m+} represents a cation represented by Formula (ca-1-1).

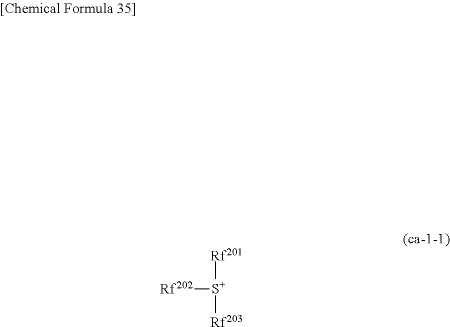

[In the formula, Rf²⁰¹ to Rf²⁰³ each independently represent an aryl group which may have a substituent, an alkyl group which may have a substituent, or an alkenyl group which may have a substituent. Rf²⁰¹ to Rf²⁰³ may be bonded to each other to form a ring together with the sulfur atoms in the formula. Here, at least one of Rf²⁰¹ to Rf²⁰³ contains at least one fluorine atom.]

Rf²⁰¹ to Rf²⁰³ in Formula (ca-1-1) are each the same as R²⁰¹ to R²⁰³ in Formula (ca-1). Here, at least one of Rf²⁰¹ to Rf²⁰³ contains at least one fluorine atom. The cation represented by Formula (ca-1-1) preferably contains three or more fluorine atoms. Any one of Rf²⁰¹ to Rf²⁰³ may have three or more fluorine atoms, or the total number of fluorine atoms contained in Rf²⁰¹ to Rf²⁰³ may be three or more.

As the constitutional unit (a0), it is preferable to use at least one selected from the group consisting of the constitutional unit (a01), the constitutional unit (a02), the constitutional unit (a03), and the constitutional unit (a04), more preferable to use at least one selected from the group consisting of the constitutional unit (a01), the constitutional unit (a02), and the constitutional unit (a03), still more preferable to use at least one selected from the group consisting of the constitutional unit (a01) and the constitutional unit (a02), and particularly preferable to use at least one selected from the group consisting of the constitutional unit (a01).

The cation moiety of each of the constitutional units (a01) to (a04) is preferably an m-valent sulfonium cation.

Specific examples of the constitutional unit (a0) are shown below, but the examples are not limited thereto.

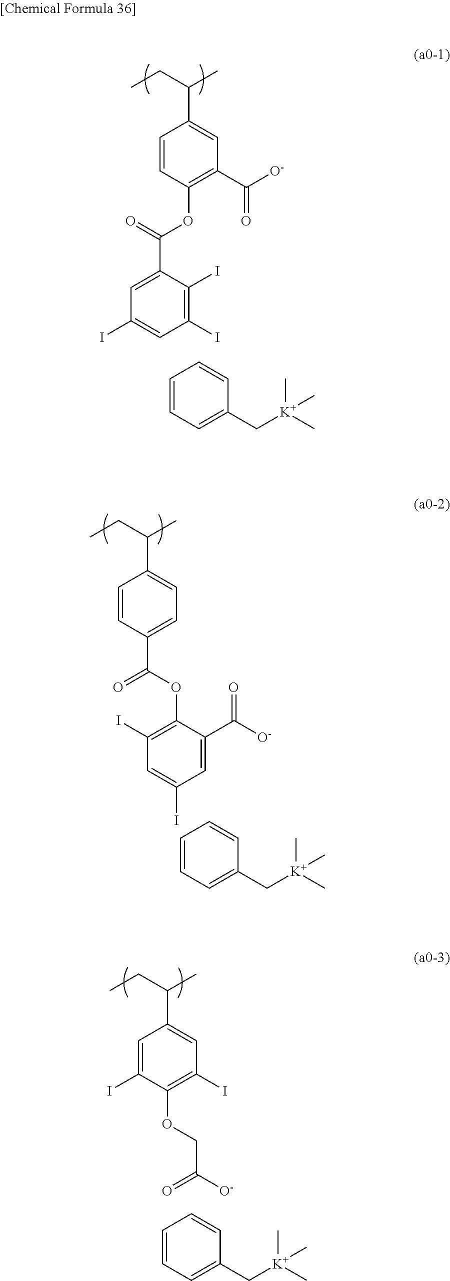

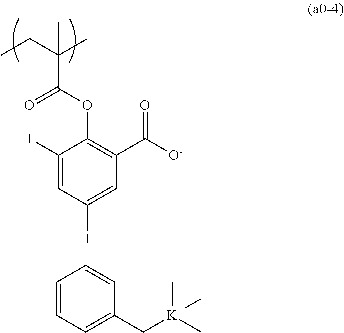

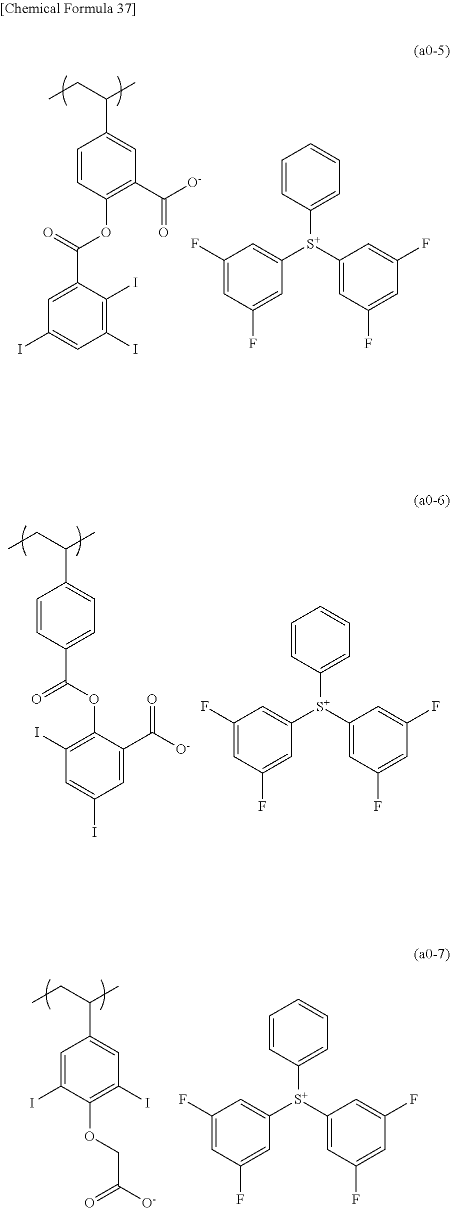

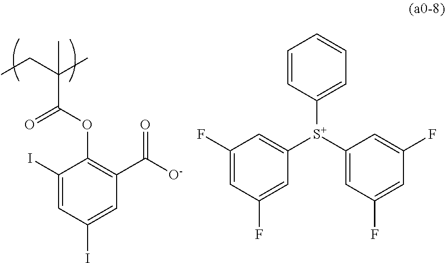

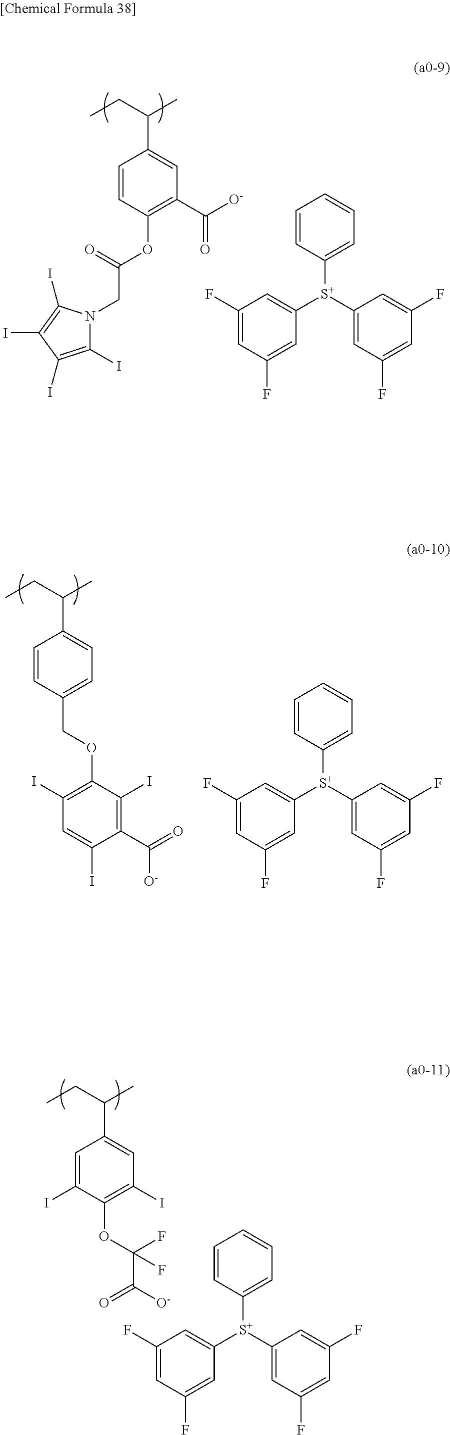

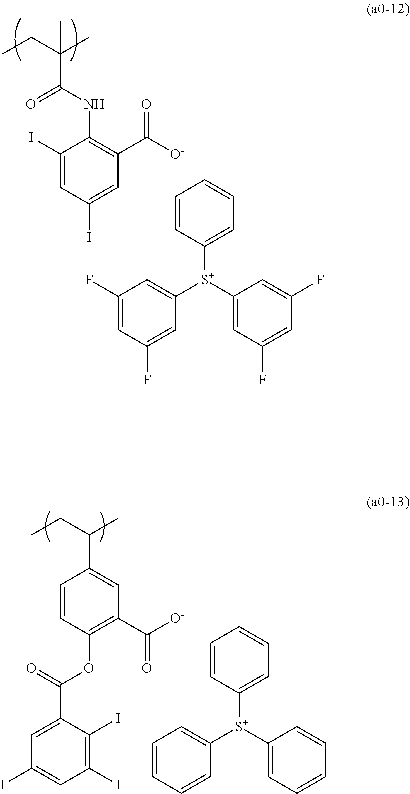

The constitutional unit (a0) contained in the component (A1) may be one kind or may be two or more kinds.

The content proportion of the constitutional unit (a0) in the component (A1) is preferably in a range of 1% to 50% by mole, more preferably in a range of 1% to 40% by mole, still more preferably in a range of 2% to 20% by mole, and particularly preferably in a range of 2% to 10% by mole with respect to the total amount (100% by mole) of all constitutional units constituting the component (A1).

In a case where the proportion of the constitutional unit (a0) is set to be greater than or equal to the lower limits of the above-described preferable ranges, the sensitivity is further improved. In a case where the proportion of the constitutional unit (a0) is less than or equal to the upper limits of the above-described preferable ranges, the lithography characteristics such as roughness reduction are further improved.

### <<Other Constitutional Units>>

The component (A1) may have other constitutional units as necessary in addition to the constitutional unit (a0) described above.

Examples of the other constitutional units include a constitutional unit (a1) containing an acid decomposable group whose polarity is increased by the action of an acid; a constitutional unit (a10) represented by General Formula (a10-1) described below; a constitutional unit (a5) which generates an acid upon light exposure (here, a constitutional unit corresponding to the constitutional unit (a0) is excluded); a constitutional unit (a2) containing a lactone-containing cyclic group; and a constitutional unit (a8) derived from a compound represented by General Formula (a8-1) described below.

### In Regard to Constitutional Unit (a1):

The constitutional unit (a1) is a constitutional unit that contains an acid decomposable group whose polarity is increased by the action of an acid.

Examples of the acid decomposable group include a group in which a polar group is protected by an acid dissociable group (for example, a group in which a hydrogen atom of an OH-containing polar group is protected by an acid dissociable group).

Examples of the acid dissociable group are the same as those which have been suggested as the acid dissociable groups of the base resin for a chemically amplified resist composition.

Specific examples of the acid dissociable group of the base resin suggested for a chemically amplified resist composition include “acetal type acid dissociable group”, “tertiary alkyl ester type acid dissociable group”, “tertiary alkyloxycarbonyl acid dissociable group”, and “secondary alkyl ester type acid dissociable group” described below.

### Acetal Type Acid Dissociable Group:

Examples of the acid dissociable group that protects a carboxy group or a hydroxyl group in the polar groups include an acid dissociable group represented by General Formula (a1-r-1) (hereinafter, also referred to as “acetal type acid dissociable group”).

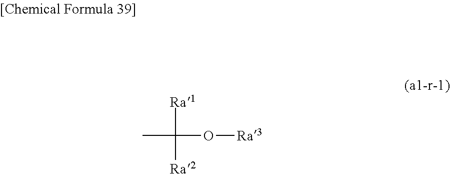

[In the formula, Ra′¹ and Ra′² represent a hydrogen atom or an alkyl group. Ra′³ represents a hydrocarbon group, and Ra′³ may be bonded to any of Ra′¹ and Ra′² to form a ring.]

In Formula (a1-r-1), it is preferable that at least one of Ra′¹ and Ra′² represents a hydrogen atom and more preferable that both Ra′¹ and Ra′² represent a hydrogen atom.

In a case where Ra′¹ or Ra′² represents an alkyl group, an alkyl group having 1 to 5 carbon atoms is preferable as the alkyl group. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′³ represents a cyclic hydrocarbon group, the hydrocarbon group may be an alicyclic hydrocarbon group or an aromatic hydrocarbon group and may be a polycyclic group or a monocyclic group.

The alicyclic hydrocarbon group which is a monocyclic group is preferably a group in which one hydrogen atom has been removed from a monocycloalkane. The monocycloalkane has preferably 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane. The polycycloalkane preferably has 7 to 12 carbon atoms, and specific examples thereof include adamantane, norbornane, isobornane, tricyclodecane, and tetracyclododecane.

In a case where the cyclic hydrocarbon group as Ra′³ becomes an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specifically, as the aromatic ring, an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms are exemplary examples. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

The cyclic hydrocarbon group as Ra′³ may have a substituent. Examples of the substituent include —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN, and —R^{P2}—COOH (hereinafter, these substituents will also be collectively referred to as “Ra^{x5}”)

Here, R^{P1} represents a chain-like monovalent saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. Further, R¹² represents a single bond, a chain-like divalent saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Some or all hydrogen atoms in the chain-like saturated hydrocarbon group, the aliphatic cyclic saturated hydrocarbon group, and the aromatic hydrocarbon group as R^{P1} and R^{P2} may be substituted with fluorine atoms. The aliphatic cyclic hydrocarbon group may have one or more of one kind of substituents or one or more of each of plural kinds of the substituents.

Examples of the chain-like monovalent saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, or an adamantyl group.

Examples of the monovalent aromatic hydrocarbon group having 6 to 30 carbon atoms include a group formed by removing one hydrogen atom from an aromatic hydrocarbon ring such as benzene, biphenyl, fluorene, naphthalene, anthracene, or phenanthrene.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

### Tertiary Alkyl Ester Type Acid Dissociable Group:

Examples of the acid dissociable group that protects a carboxy group among the polar groups include an acid dissociable group represented by General Formula (a1-r-2).

Among examples of the acid dissociable group represented by Formula (a1-r-2), a group formed of an alkyl group is referred to as “tertiary alkyl ester type acid dissociable group” for convenience.

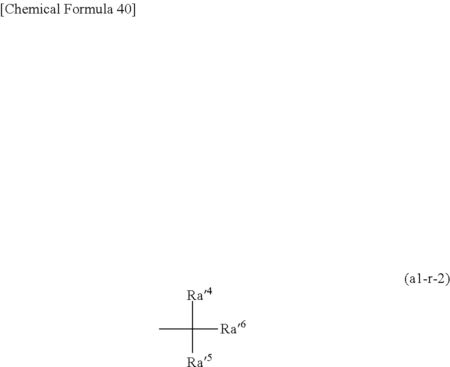

[In the formula, Ra′⁴ to Ra′⁶ each represents a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, a chain-like alkynyl group, and a cyclic hydrocarbon group.

Examples of the linear or branched alkyl group and the cyclic hydrocarbon group (an alicyclic hydrocarbon group which is a monocyclic group, an alicyclic hydrocarbon group which is a polycyclic group, or an aromatic hydrocarbon group) as Ra′⁴ include the same groups as those for Ra′³.

As the chain-like or cyclic alkenyl group as Ra′⁴, an alkenyl group having 2 to 10 carbon atoms is preferable.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁶ represent an independent hydrocarbon group without being bonded to one another, suitable examples thereof include a group represented by General Formula (a1-r2-4).

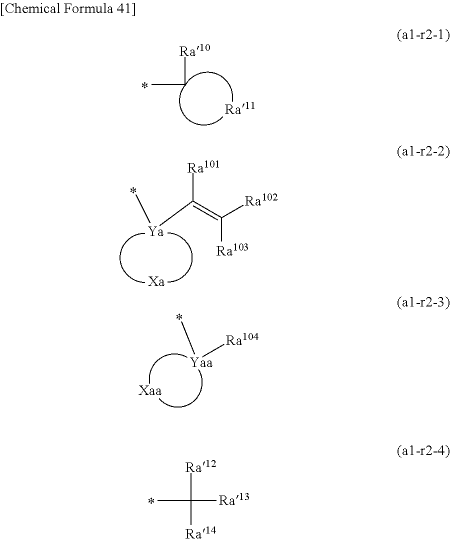

[In Formula (a1-r2-1), Ra′¹⁰ represents a linear or branched alkyl group having 1 to 12 carbon atoms, in which a part thereof may be substituted with a halogen atom or a heteroatom-containing group. Ra′¹¹ represents a group that forms an aliphatic cyclic group with the carbon atom to which Ra′¹⁰ has been bonded. In Formula (a1-r2-2), Ya represents a carbon atom. Xa represents a group that forms a cyclic hydrocarbon group with Ya. Some or all hydrogen atoms in this cyclic hydrocarbon group may be substituted. Ra¹⁰¹⁰ to Ra¹⁰³ each independently represent a hydrogen atom, a chain-like monovalent saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to one another to form a cyclic structure. In Formula (a1-r2-3), Yaa represents a carbon atom. Xaa represents a group that forms an aliphatic cyclic group with Yaa. Ra¹⁰⁴ represents an aromatic hydrocarbon group which may have a substituent. In Formula (a1-r2-4), Ra′¹² and Ra′¹³ each independently represent a chain-like monovalent saturated hydrocarbon group having 1 to 10 carbon atoms. Some or all hydrogen atoms contained in the chain-like saturated hydrocarbon group may be substituted. Ra′¹⁴ represents a hydrocarbon group which may have a substituent. * represents a bonding site (the same applies hereinafter).]

In Formula (a1-r2-1), Ra′¹⁰ represents a linear or branched alkyl group having 1 to 12 carbon atoms, in which a part thereof may be substituted with a halogen atom or a heteroatom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include those for Ra′³ described above.

The alkyl group in Ra′¹⁰ may be partially substituted with a halogen atom or a heteroatom-containing group. For example, some hydrogen atoms constituting the alkyl group may be substituted with a halogen atom or a heteroatom-containing group. Further, some carbon atoms (methylene group or the like) constituting the alkyl group may be substituted with a heteroatom-containing group.

Examples of the heteroatoms here include an oxygen atom, a nitrogen atom, and a sulfur atom. Examples of the heteroatom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In Formula (a1-r2-1), preferred examples of Ra′¹¹ (an aliphatic cyclic group that is formed together with a carbon atom to which Ra′¹⁰ is bonded) include the groups described as the alicyclic hydrocarbon group (alicyclic hydrocarbon group) which is a monocyclic group or a polycyclic group as Ra′³ in Formula (a1-r-1). Among these, it is preferably a monocyclic alicyclic hydrocarbon group, and specifically, it is more preferably a cyclopentyl group or a cyclohexyl group.

In Formula (a1-r2-2), examples of the cyclic hydrocarbon group that is formed by Xa together with Ya include a group in which one or more hydrogen atoms have been further removed from the cyclic monovalent hydrocarbon group (alicyclic hydrocarbon group) as Ra′³ in Formula (a1-r-1).

The cyclic hydrocarbon group that is formed by Xa together with Ya may have a substituent. Examples of the substituent include those which are the same as the substituents which may be included in the cyclic hydrocarbon group as Ra′³.

In Formula (a1-r2-2), examples of the chain-like monovalent saturated hydrocarbon group having 1 to 10 carbon atoms as Ra′⁰′ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, or an adamantyl group.

From the viewpoint of ease of synthesis, Ra¹⁰¹ to Ra¹⁰³ represent preferably a hydrogen atom or a chain-like monovalent saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ are the same as those for Ra^{x5}.

Examples of the group having a carbon-carbon double bond generated by two or more of Ra¹⁰¹ to Ra¹⁰³ being bonded to one another to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), as the aliphatic cyclic group that is formed by Xaa together with Yaa, the group described as the alicyclic hydrocarbon group which is a monocyclic group or a polycyclic group as Ra′³ in Formula (a1-r-1) is preferable.

In Formula (a1-r2-3), examples of the aromatic hydrocarbon group as Ra¹⁰4 include a group in which one or more hydrogen atoms have been removed from an aromatic hydrocarbon ring having 5 to 30 carbon atoms. Among the examples, Ra¹⁰⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent which may be included in Ra¹⁰⁴ in Formula (a1-r2-3) include a methyl group, an ethyl group, a propyl group, a hydroxyl group, a carboxyl group, a halogen atom, an alkoxy group (such as a methoxy group, an ethoxy group, a propoxy group, or a butoxy group), and an alkyloxycarbonyl group.

In Formula (a1-r2-4), Ra′¹² and Ra′¹³ each independently represent a chain-like monovalent saturated hydrocarbon group having 1 to 10 carbon atoms. Examples of the chain-like monovalent saturated hydrocarbon group having 1 to 10 carbon atoms as Ra′12 and Ra′¹³ include the same one as the chain-like monovalent saturated hydrocarbon group having 1 to 10 carbon atoms as Ra¹⁰¹ to Ra′⁰³ as described above. Some or all hydrogen atoms contained in the chain-like saturated hydrocarbon group may be substituted.

Ra′¹² and Ra′¹³ represent preferably an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′² and Ra′¹³ is substituted, examples of the substituent are those for Ra^{x5} described above.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an alicyclic hydrocarbon group or an aromatic hydrocarbon group and may be a polycyclic group or a monocyclic group.

The alicyclic hydrocarbon group which is a monocyclic group is preferably a group in which one hydrogen atom has been removed from a monocycloalkane. The monocycloalkane has preferably 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane. The polycycloalkane preferably has 7 to 12 carbon atoms, and specific examples thereof include adamantane, norbornane, isobornane, tricyclodecane, and tetracyclododecane.

Examples of the aromatic hydrocarbon group as Ra′¹⁴ include the same groups as those for the aromatic hydrocarbon group as Ra¹⁰⁴. Among these, Ra′¹⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent which may be included in Ra′¹⁴ include the same groups as those for the substituent which may be included in Ra¹⁰⁴.

In a case where Ra′¹⁴ in Formula (a1-r2-4) represents a naphthyl group, the position bonded to the tertiary carbon atom in Formula (a1-r2-4) may be the 1-position or the 2-position of the naphthyl group.

In a case where Ra′¹⁴ in Formula (a1-r2-4) represents an anthryl group, the position bonded to the tertiary carbon atom in Formula (a1-r2-4) may be the 1-position, the 2-position, or the 9-position of the anthryl group.

Specific examples of the group represented by Formula (a1-r2-1) are shown below.

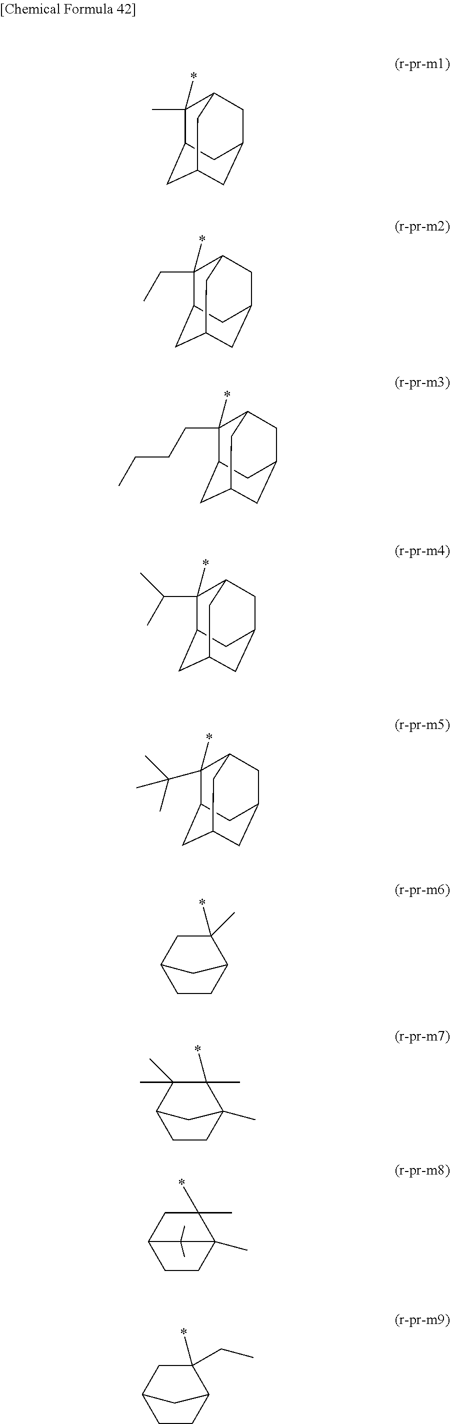

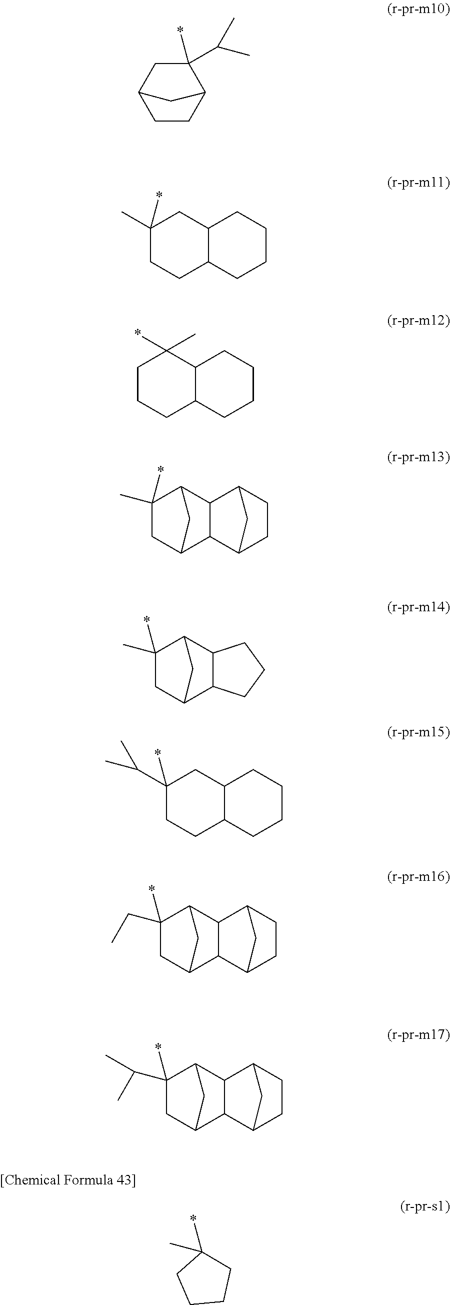

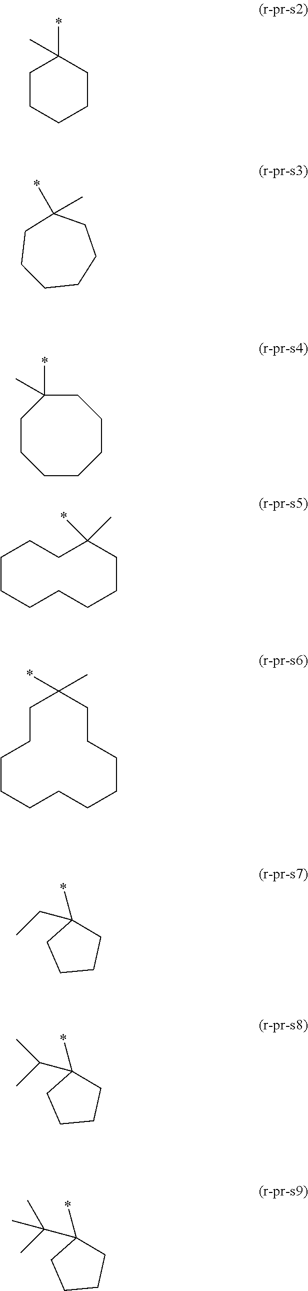

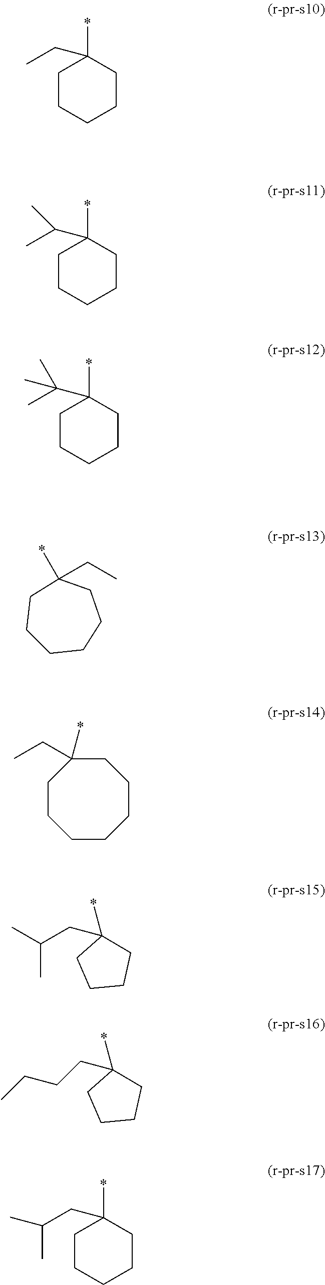

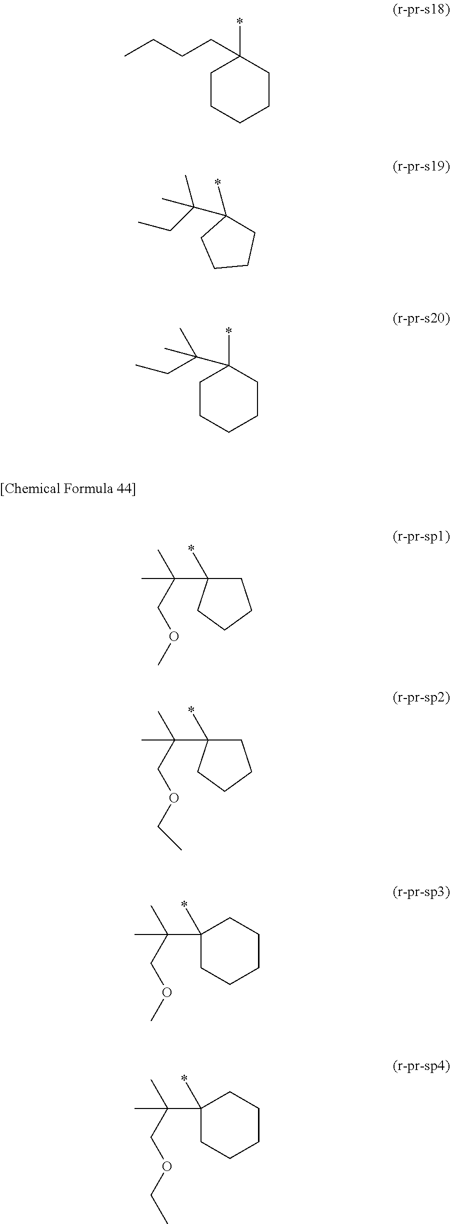

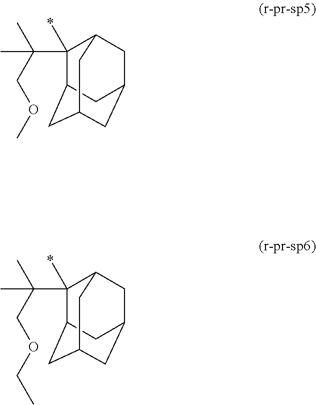 [SMILES: *C1(C(C)(C)COC)C2CC3CC(C2)CC1C3 ; *C1(C(C)(C)COCC)C2CC3CC(C2)CC1C3]

Specific examples of the group represented by Formula (a1-r2-2) are shown below.

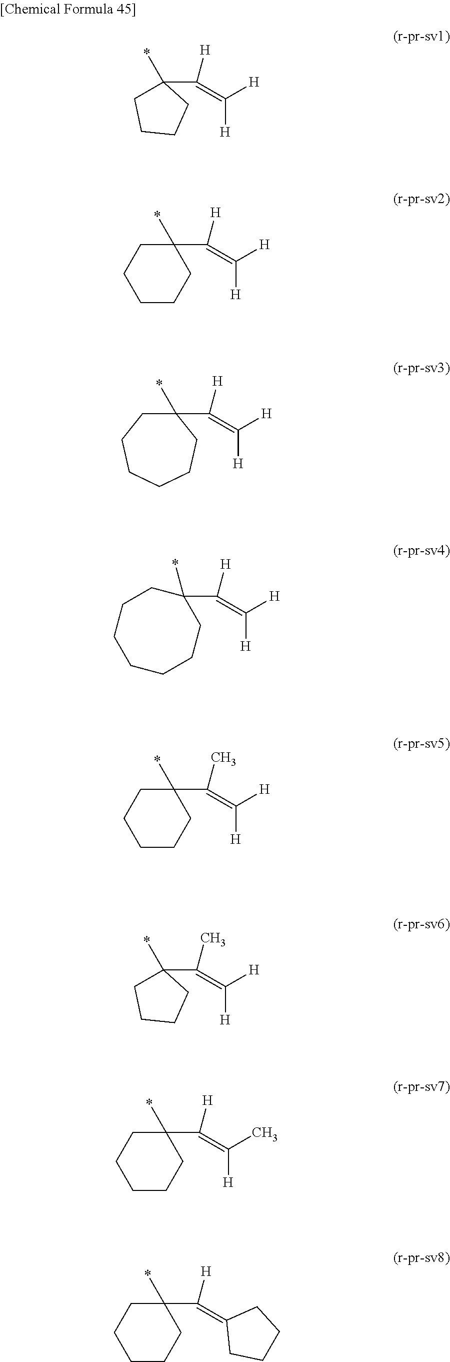

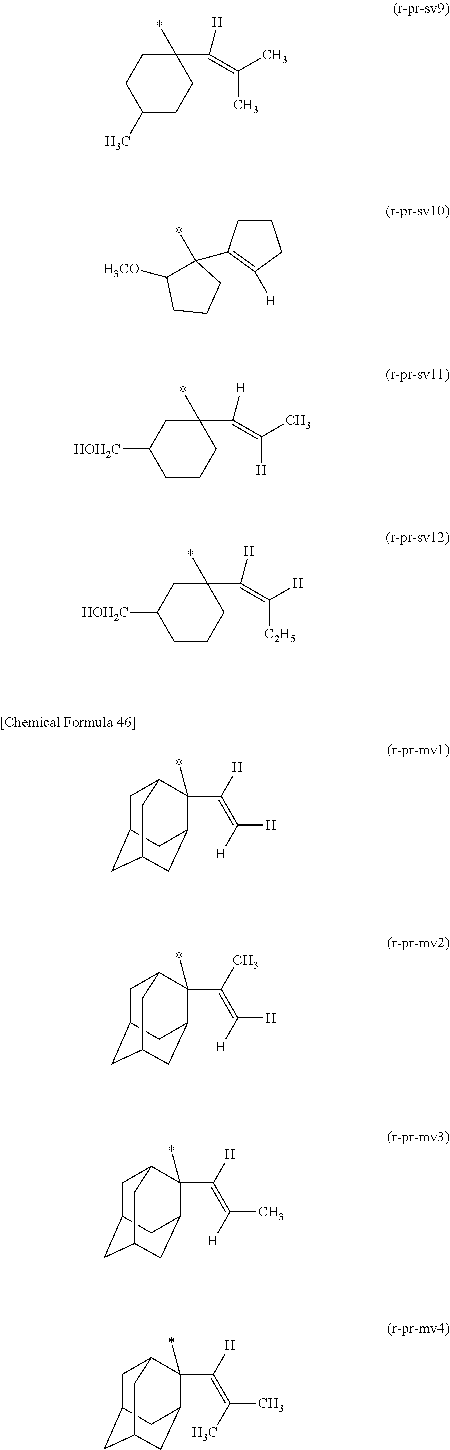

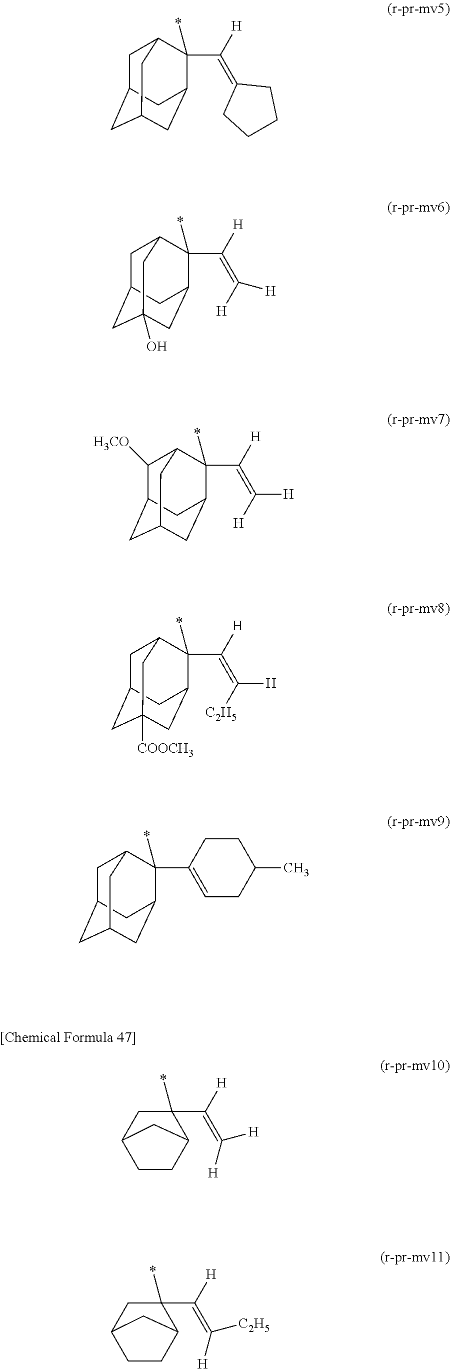

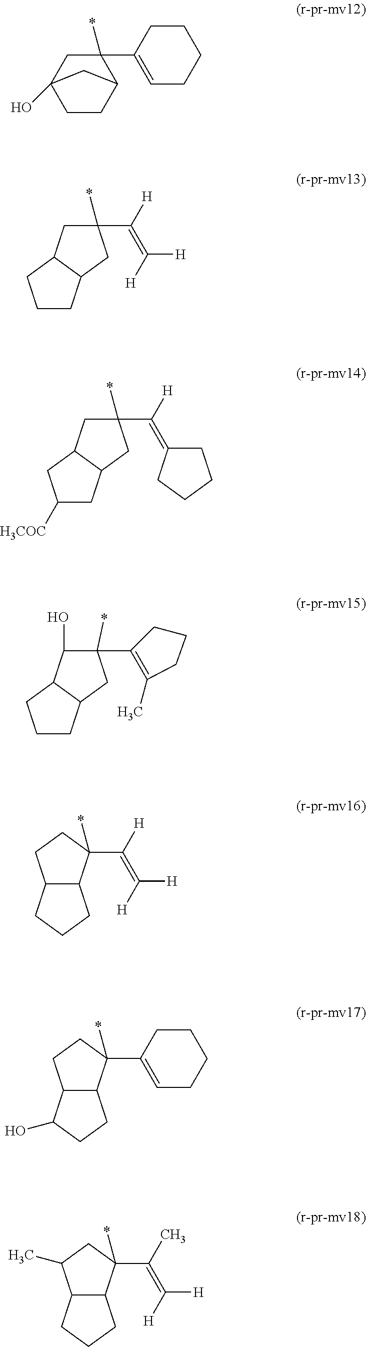

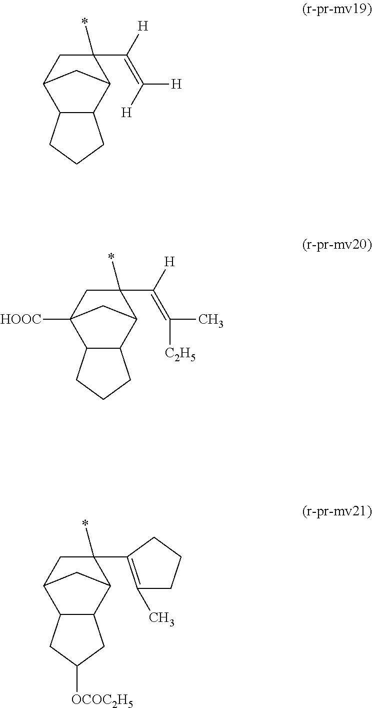

Specific examples of the group represented by Formula (a1-r2-3) are shown below.

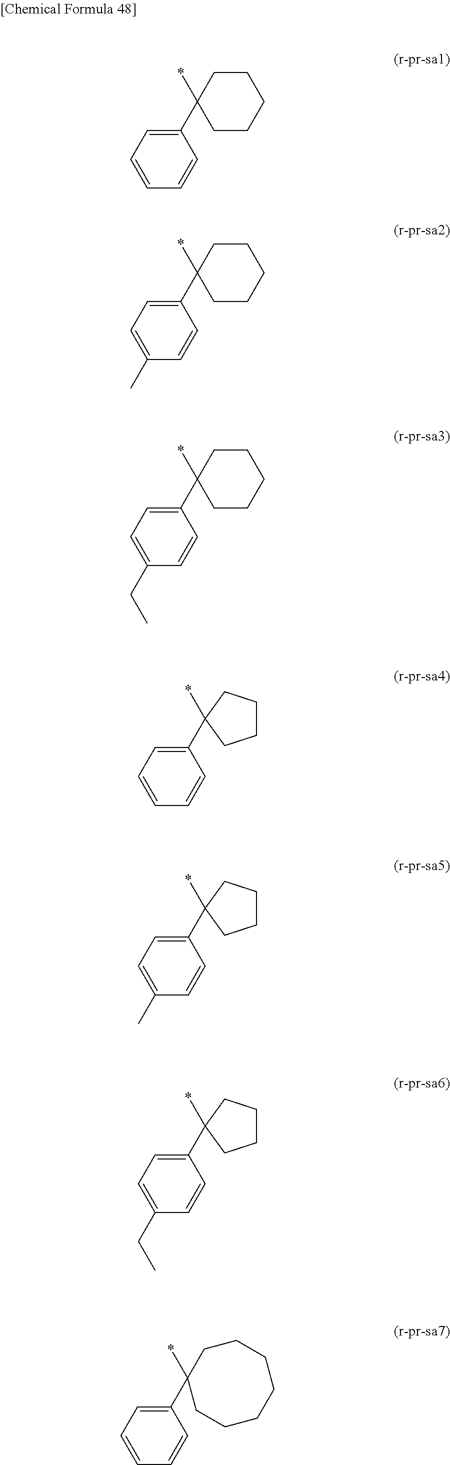

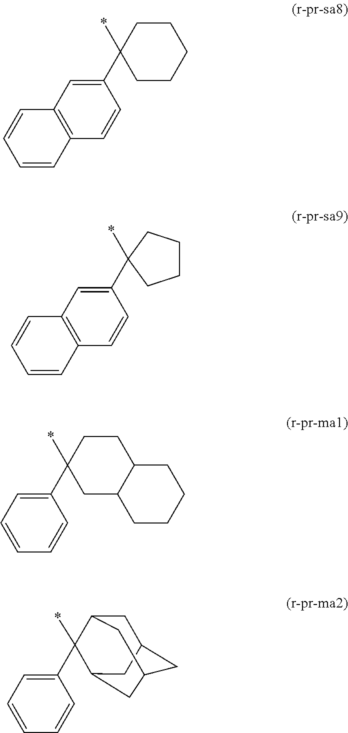 [SMILES: *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of the group represented by Formula (a1-r2-4) are shown below.

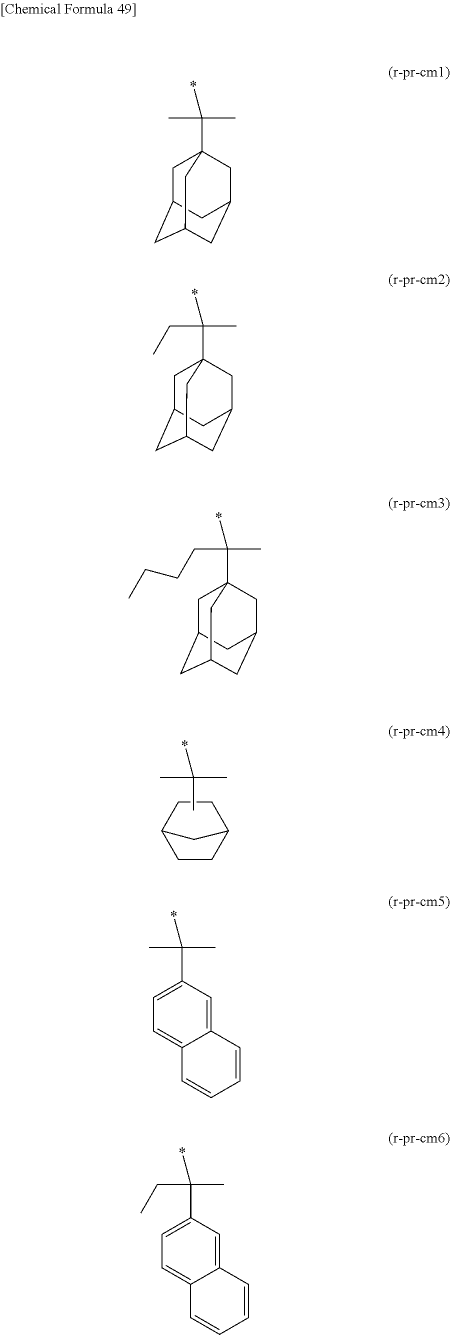

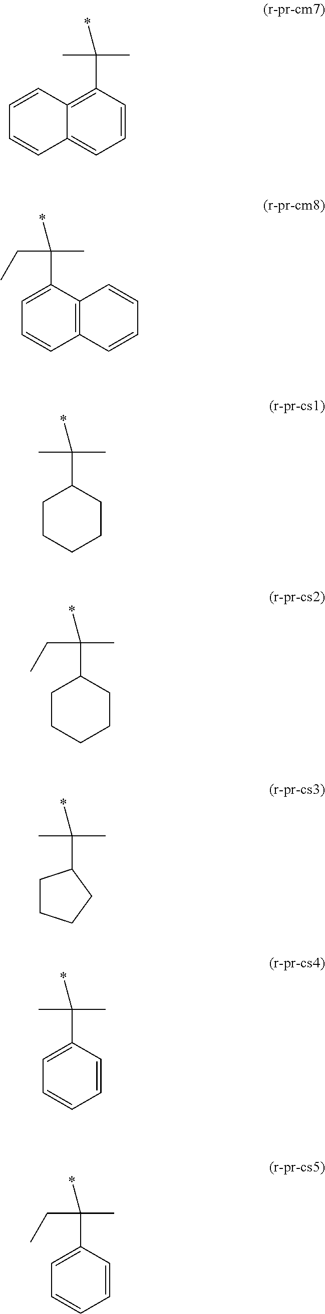

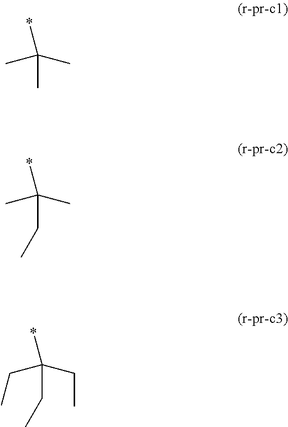

### Tertiary Alkyloxycarbonyl Acid Dissociable Group:

Examples of the acid dissociable group that protects a hydroxyl group among the polar groups include an acid dissociable group (hereinafter, also referred to as “tertiary alkyloxycarbonyl acid dissociable group” for convenience) represented by General Formula (a1-r-3).

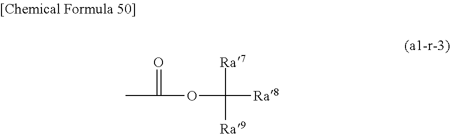

[In the formula, Ra′⁷ to Ra′⁹ each represent an alkyl group.]

In Formula (a1-r-3), Ra′⁷ to Ra′⁹ each represent preferably an alkyl group having 1 to 5 carbon atoms and more preferably an alkyl group having 1 to 3 carbon atoms.

Further, the total number of carbon atoms in each alkyl group is preferably in a range of 3 to 7, more preferably in a range of 3 to 5, and most preferably 3 or 4.

### Secondary Alkyl Ester Type Acid Dissociable Group:

Examples of the acid dissociable group that protects a carboxy group among the polar groups include an acid dissociable group represented by General Formula (a1-r-4).

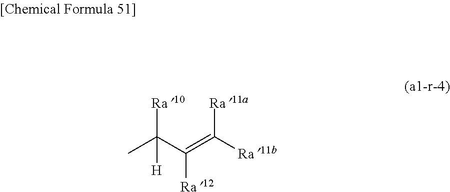

[In the formula, Ra′⁰ represents a hydrocarbon group. Ra′^{11a} and Ra′^{11b} each independently represent a hydrogen atom, a halogen atom, or an alkyl group. Ra′¹² represents a hydrogen atom or a hydrocarbon group. Ra′¹⁰ and Ra′^{11a} or Ra′^{11b} may be bonded to each other to form a ring. Ra′^{11a} or Ra′^{11b} and Ra′¹² may be bonded to each other to form a ring.]

Examples of the hydrocarbon group as Ra′¹⁰ or Ra′¹² in the formula include the same groups as those for Ra′³.

Examples of the alkyl group as Ra′^{11a} and Ra′^{11b} in the formula include the same groups as those for the alkyl group as Ra′¹.

In the formula, the hydrocarbon group as Ra′¹⁰ or Ra′¹² and the alkyl group as Ra′^{11a} and Ra′^{11b} may have a substituent. Examples of this substituent include Ra′ described above.

Ra′¹⁰ and Ra′^{11a} or Ra′^{11b} may be bonded to each other to form a ring. The ring may be a polycyclic ring or a monocyclic ring, and may be an alicyclic ring or an aromatic ring.

The alicyclic ring and the aromatic ring may have a heteroatom.

Among the examples described above, as the ring formed by Ra′¹⁰ and Ra′^{11a} or Ra′^{11b} being bonded to each other, monocycloalkene, a ring in which some carbon atoms of monocycloalkene are substituted with heteroatoms (such as an oxygen atom and a sulfur atom), or monocycloalkadiene is preferable, cycloalkene having 3 to 6 carbon atoms is preferable, and cyclopentene or cyclohexene is preferable.

The ring formed by Ra′¹⁰ and Ra′^{11a} or Ra′^{11b} being bonded to each other may be a condensed ring. Specific examples of the condensed ring include indane.

The ring formed by Ra′¹⁰ and Ra′^{11a} or Ra′^{11b} being bonded to each other may have a substituent. Examples of this substituent include Ra^{x5} described above.

Ra′^{11a} or Ra′^{11b} and Ra′¹² may be bonded to each other to form a ring, and examples of the ring include the rings formed by Ra′¹⁰ and Ra′^{11a} or Ra′^{11b} being bonded to each other.

Specific examples of the group represented by Formula (a1-r-4) are shown below.

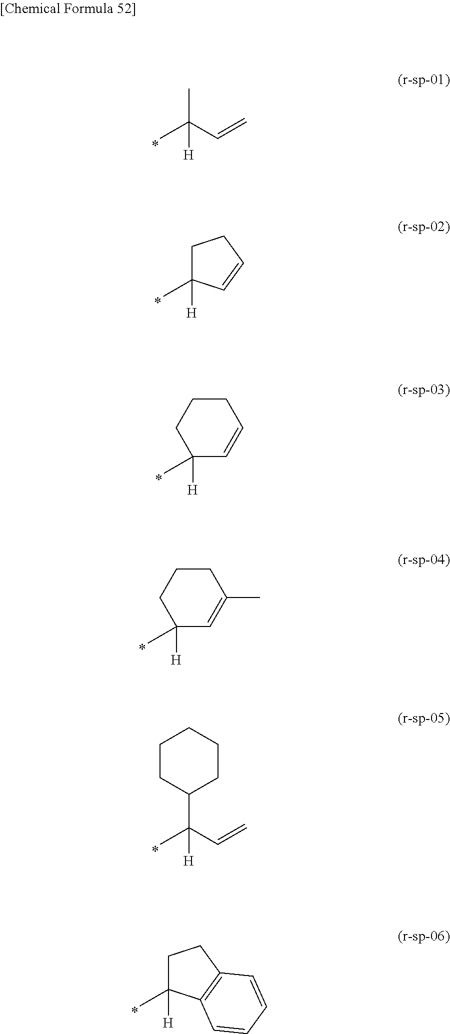

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid decomposable group; and a constitutional unit in which at least some hydrogen atoms in —C(═O)—OH of a constitutional unit derived from vinylbenzoic acid or a vinylbenzoic acid derivative are protected by a substituent containing the acid decomposable group.

Among the examples, as the constitutional unit (a1), a constitutional unit derived from acrylic acid ester in which the hydrogen atom bonded to the carbon atom at the α-position may be substituted with a substituent is preferable.

Specific preferred examples of such a constitutional unit (a1) include constitutional units represented by General Formula (a1-1) or (a1-2) shown below.

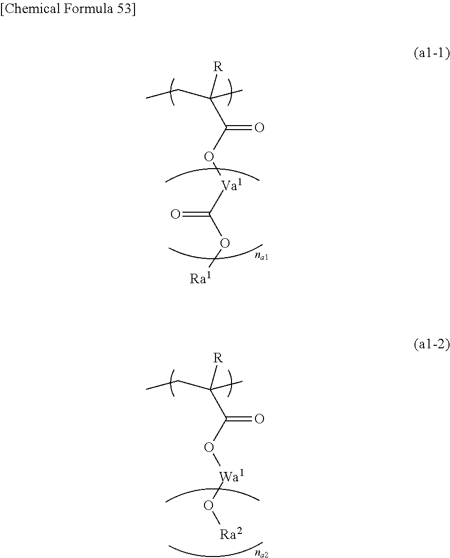

[In the formula, R represents a hydrogen atom, an alkyl group having 1 to 5 carbon atoms, or a halogenated alkyl group having 1 to 5 carbon atoms. Va¹ represents a divalent hydrocarbon group which may contain an ether bond. nₐ₁ represents an integer of 0 to 2. Ra¹ represents an acid dissociable group represented by General Formula (a1-r-1), (a1-r-2), or (a1-r-4). Wa¹ represents a (nₐ₂+1)-valent hydrocarbon group, nₐ₂ represents an integer of 1 to 3, and Ra² represents an acid dissociable group represented by General Formula (a1-r-1) or (a1-r-3)].

In Formula (a1-1), as the alkyl group having 1 to 5 carbon atoms as R, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R represents preferably a hydrogen atom, an alkyl group having 1 to 5 carbon atoms, or a fluorinated alkyl group having 1 to 5 carbon atoms, and most preferably a hydrogen atom or a methyl group from the viewpoint of the industrial availability.

In Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Va¹ may be saturated or unsaturated. In general, it is preferable that the aliphatic hydrocarbon group is saturated.

More specific examples of the aliphatic hydrocarbon group include a linear or branched aliphatic hydrocarbon group and an aliphatic hydrocarbon group having a ring in the structure thereof.

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Examples of the aliphatic hydrocarbon group having a ring in the structure thereof include an alicyclic hydrocarbon group (a group in which two hydrogen atoms have been removed from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the middle of the linear or branched aliphatic hydrocarbon group. Examples of the linear or branched aliphatic hydrocarbon group include the same groups as those for the linear aliphatic hydrocarbon group or the branched aliphatic hydrocarbon group.

The alicyclic hydrocarbon group has preferably 3 to 20 carbon atoms and more preferably 3 to 12 carbon atoms.

The alicyclic hydrocarbon group may be monocyclic or polycyclic. The monocyclic alicyclic hydrocarbon group is preferably a group obtained by removing two hydrogen atoms from a monocycloalkane. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples thereof include adamantane, norbornane, isobornane, tricyclodecane, and tetracyclododecane.

The aromatic hydrocarbon group as the divalent hydrocarbon group represented by Va¹ is a hydrocarbon group having an aromatic ring.

The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Here, the number of carbon atoms in a substituent is not included in the number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom.

Specific examples of the aromatic hydrocarbon group include a group in which two hydrogen atoms have been removed from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group formed by further removing one more hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (a1-1), Ra¹ represents an acid dissociable group represented by General Formula (a1-r-1), (a1-r-2), or (a1-r-4).

In Formula (a1-2), the (nₐ₂+1)-valent hydrocarbon group as Wa¹ may be an aliphatic hydrocarbon group or an aromatic hydrocarbon group. The aliphatic hydrocarbon group indicates a hydrocarbon group that has no aromaticity and may be saturated or unsaturated. In general, it is preferable that the aliphatic hydrocarbon group is saturated. Examples of the aliphatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof.

The valency of nₐ₂+1 is preferably divalent, trivalent, or tetravalent and more preferably divalent or trivalent.

In Formula (a1-2), Ra² represents an acid dissociable group represented by General Formula (a1-r-1) or (a1-r-3).

Specific examples of the constitutional unit (a1) are shown below. In the formulae shown below, R^{α} represents a hydrogen atom, a methyl group, or a trifluoromethyl group.

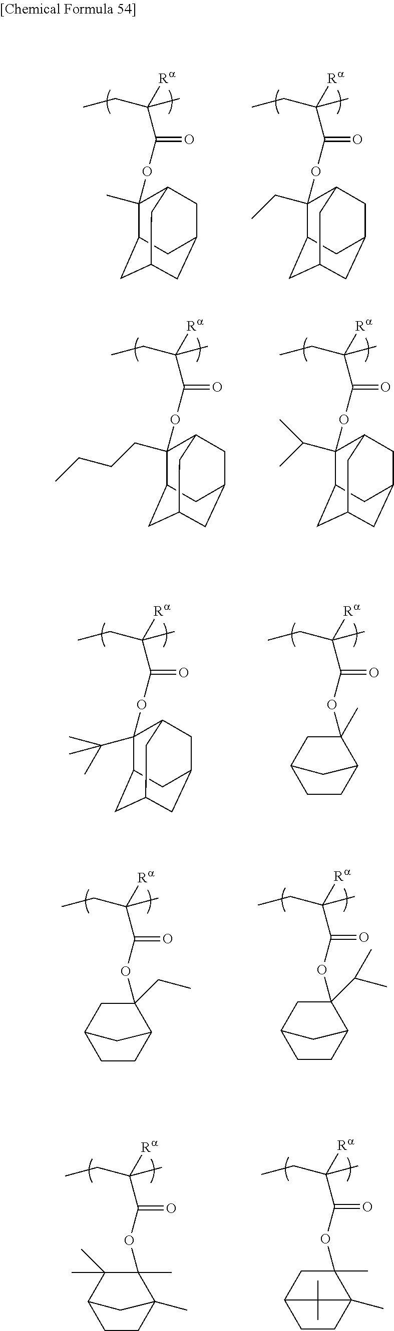

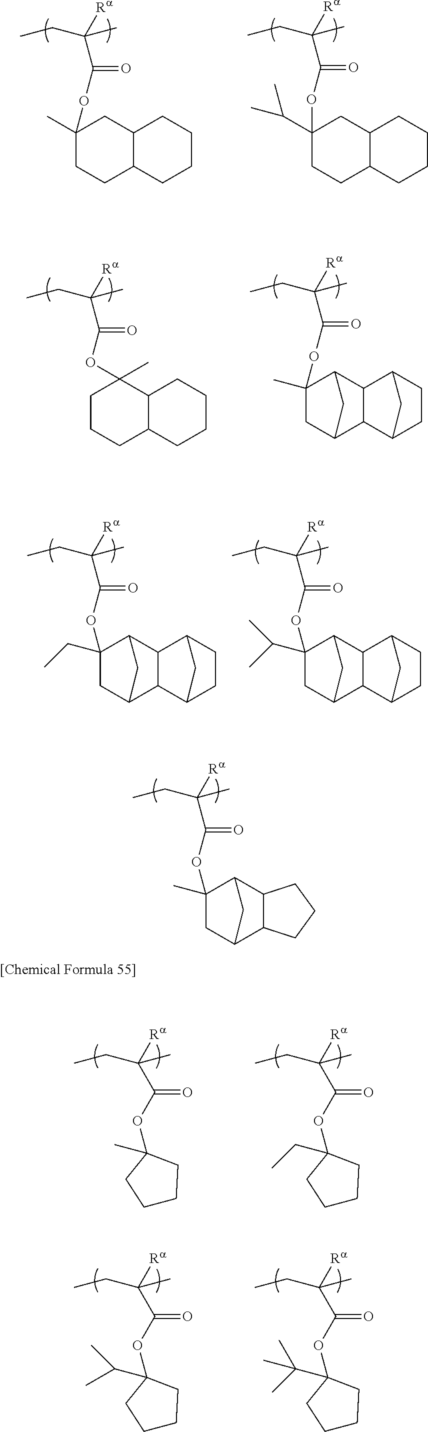

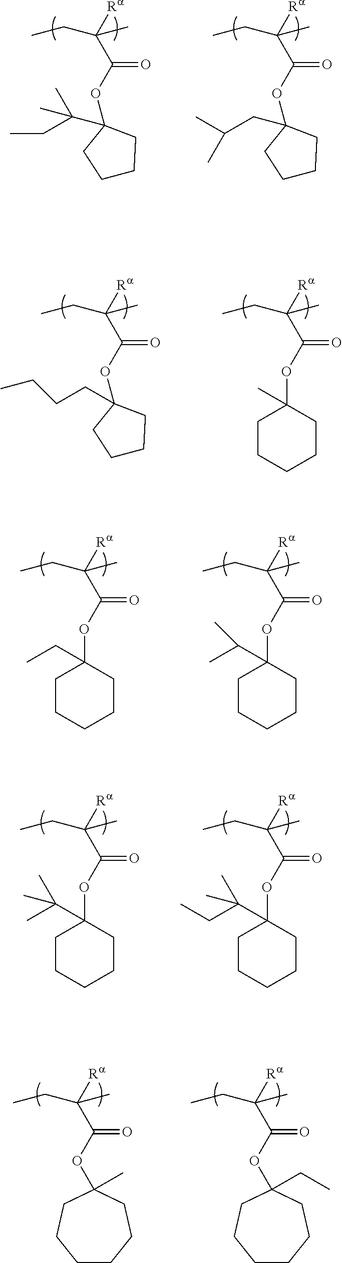

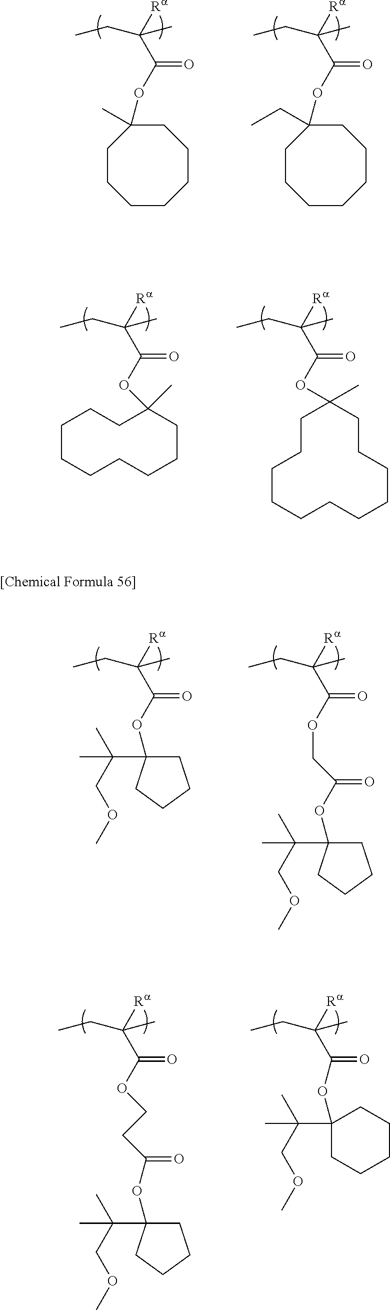

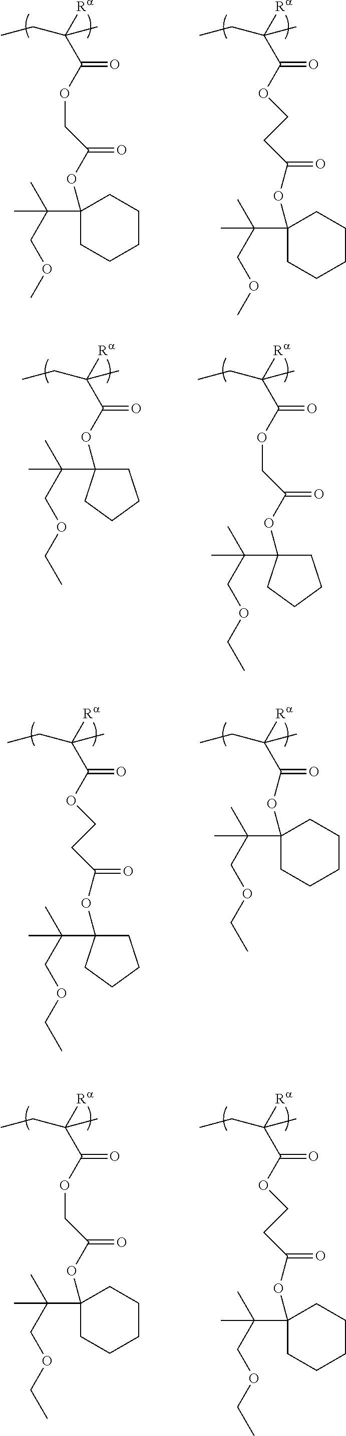

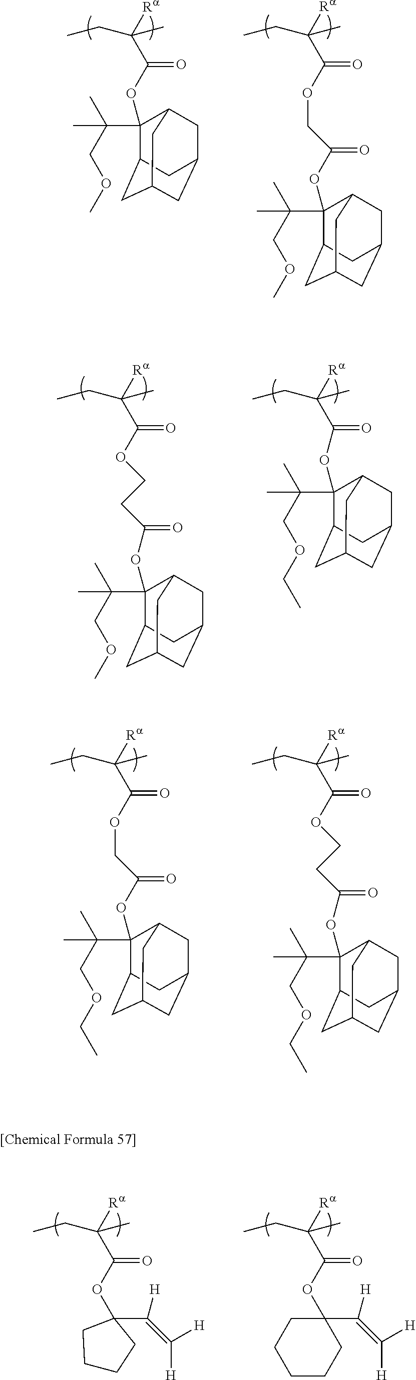

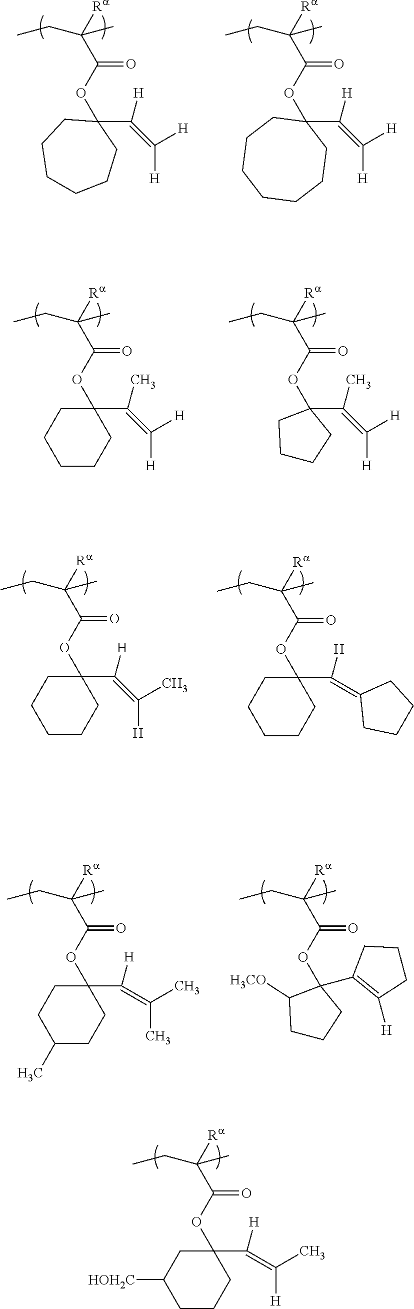

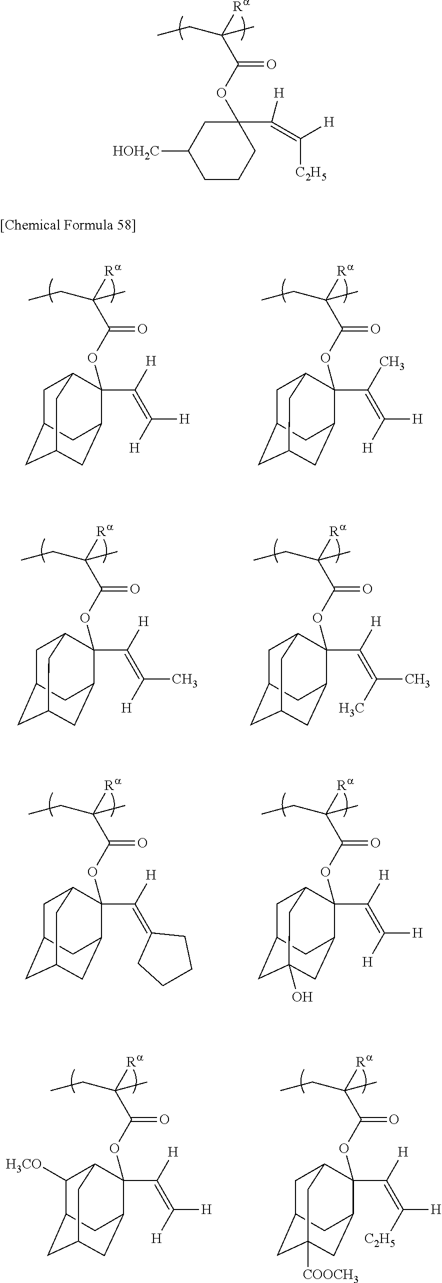

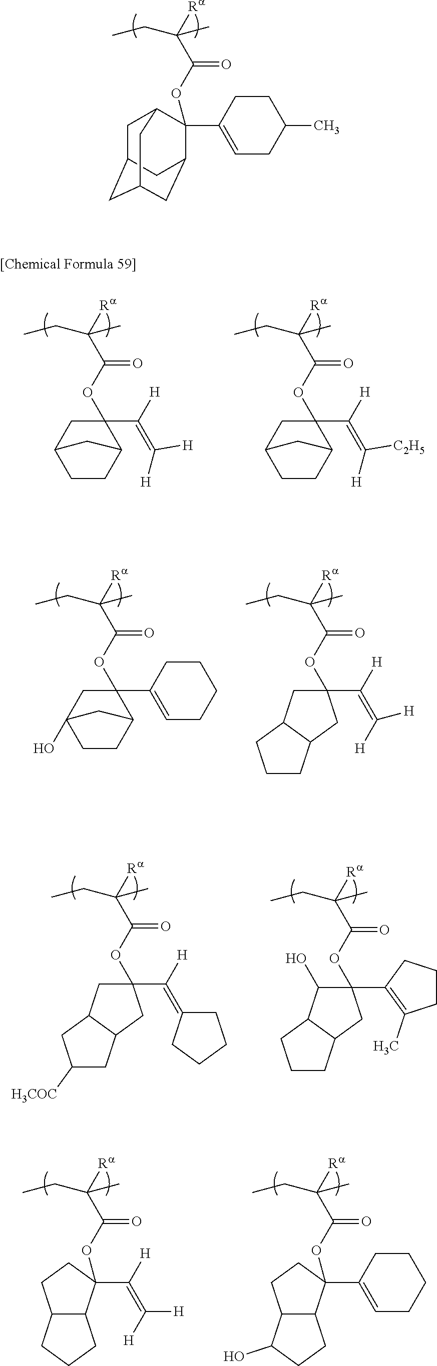

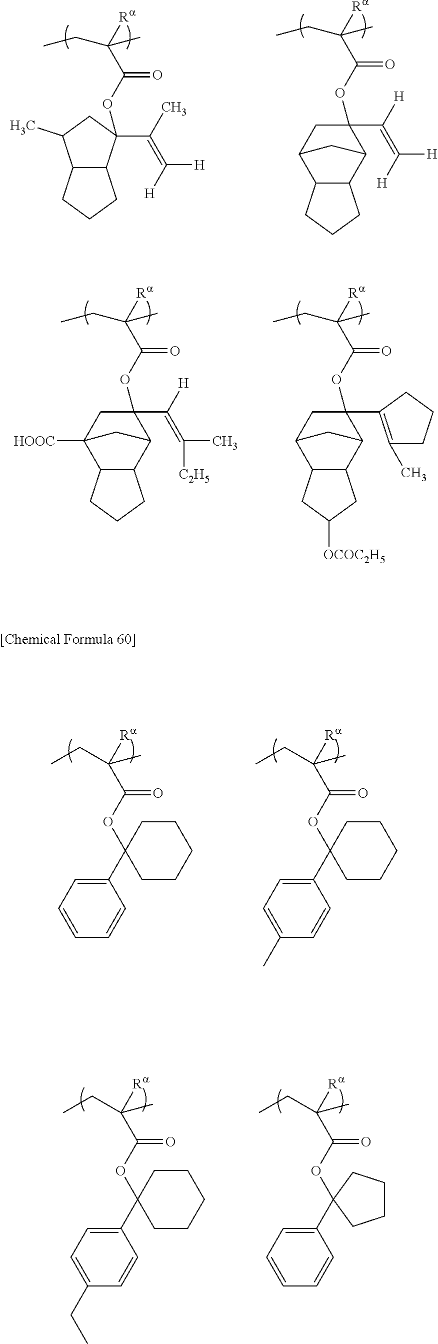

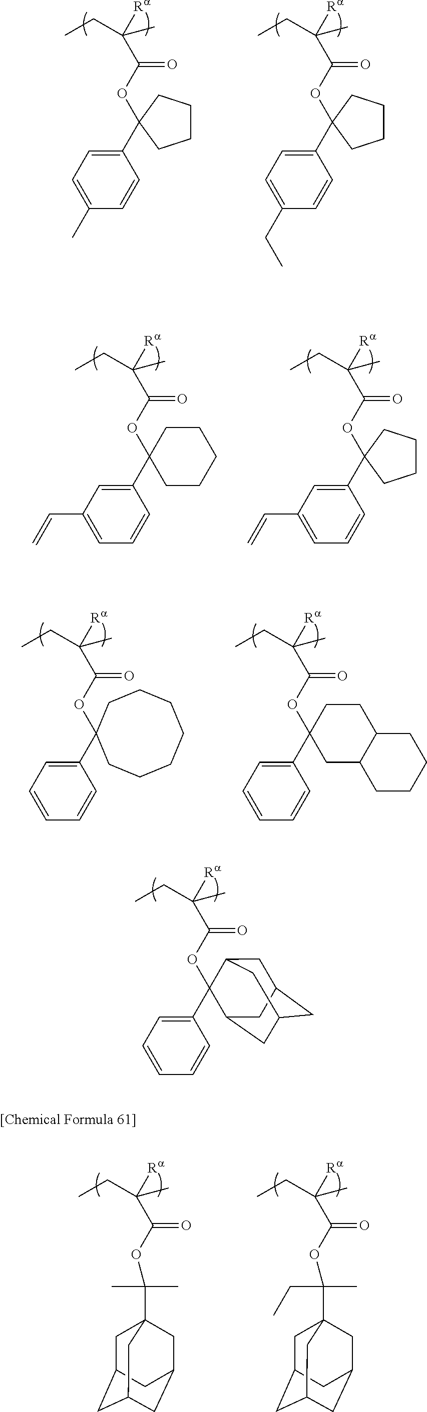

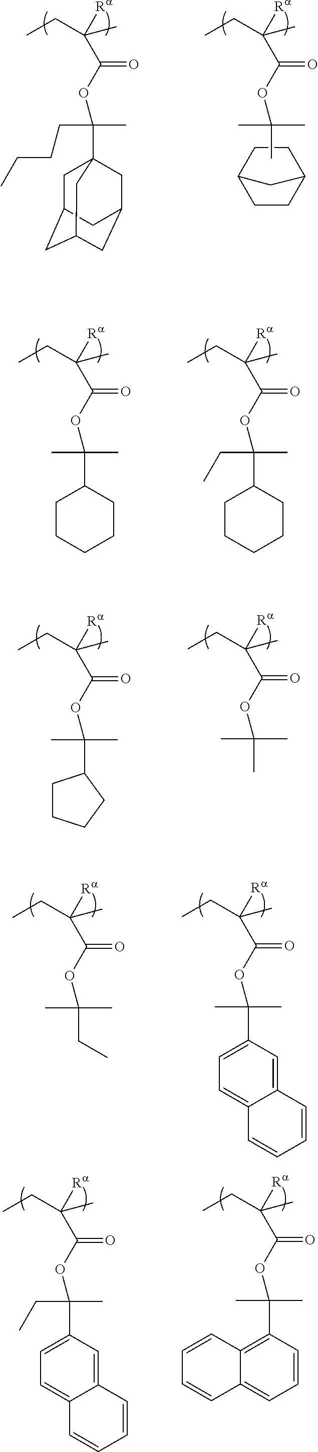

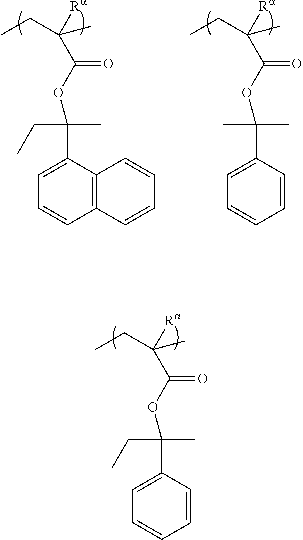

The constitutional unit (a1) included in the component (A1) may be used alone or two or more kinds thereof.

As the constitutional unit (a1), the constitutional unit represented by Formula (a1-1) is more preferable from the viewpoint that the lithography characteristics with electron beams or EUV are more likely to be enhanced.

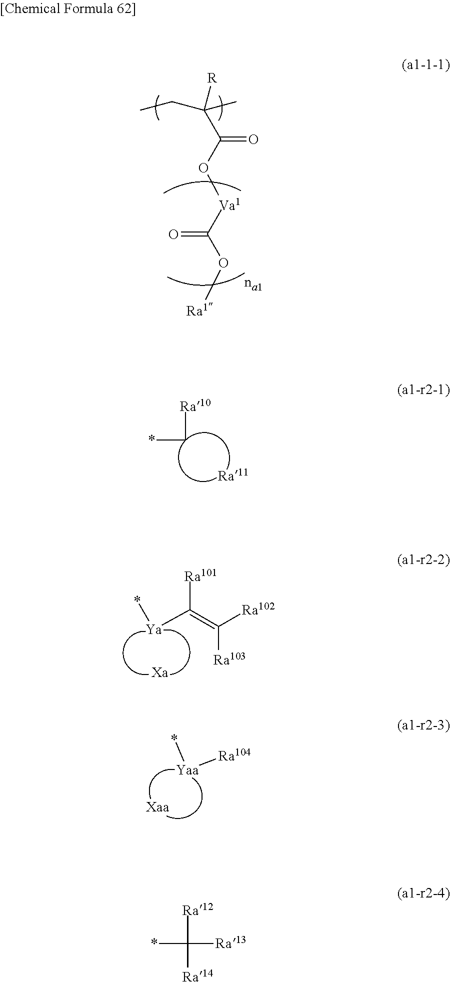

[In the formulae, Ra¹″ represents an acid dissociable group represented by General Formula (a1-r2-1), (a1-r2-2), (a1-r2-3), or (a1-r2-4)]. * represents a bonding site.]

In Formula (a1-1-1), R, Va¹, and nₐ₁ each have the same definition as that for R, Va¹, and nₐ₁ in Formula (a1-1).

The acid dissociable group represented by General Formula (a1-r2-1), (a1-r2-2), (a1-r2-3), or (a1-r2-4) is as described above. Among these, from the viewpoint of further increasing the reactivity for EB or EUV, an acid dissociable group in which the acid dissociable group is a cyclic group is preferably selected, an acid dissociable group represented by General Formula (a1-r2-1) or an acid dissociable group represented by General Formula (a1-r2-2) is more preferable, and an acid dissociable group represented by General Formula (a1-r2-1) is still more preferable.

The proportion of the constitutional unit (a1) in the component (A1) is preferably in a range of 25% to 70% by mole, more preferably in a range of 35% to 65% by mole, and still more preferably in a range of 45% to 60% by mole with respect to the total amount (100% by mole) of all constitutional units constituting the component (A1).

In a case where the proportion of the constitutional unit (a1) is set to be greater than or equal to the lower limits of the above-described preferable ranges, the lithography characteristics such as sensitivity, resolution, and pattern dimension uniformity are improved. Further, in a case where the proportion of the constitutional unit (a1) is less than or equal to the upper limits of the above-described preferable ranges, the constitutional unit (a1) and other constitutional units can be balanced, and the lithography characteristics are enhanced.

Constitutional Unit (a10):

The constitutional unit (a10) is a constitutional unit represented by General Formula (a10-1).

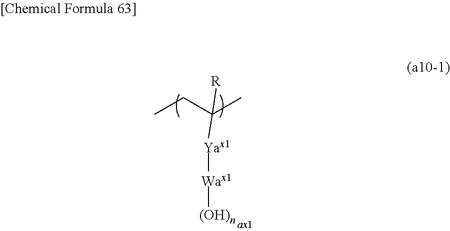

[In the formula, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent. nₐₓ₁ represents an integer of 1 or greater.]

In Formula (a10-1), R represents preferably a hydrogen atom, an alkyl group having 1 to 5 carbon atoms, or a fluorinated alkyl group having 1 to 5 carbon atoms, and from the viewpoint of industrial availability, particularly preferably a hydrogen atom or a methyl group.

In Formula (a10-1), Ya^{x1} represents a single bond or a divalent linking group.

The divalent linking group as Ya^{x1} is not particularly limited, and suitable examples thereof include a divalent hydrocarbon group which may have a substituent, and a divalent linking group having heteroatoms.

Ya^{x1} represents preferably a single bond, an ester bond [—C(═O)—O— or —O—C(═O)—], an ether bond (—O—), a linear or branched alkylene group, or a combination thereof and more preferably a single bond or an ester bond [—C(═O)—O— or —O—C(═O)—].

In Formula (a10-1), Wa^{x1} represents an aromatic hydrocarbon group which may have a substituent.

Examples of the aromatic hydrocarbon group as Wa^{x1} include a group obtained by removing (nₐₓ₁+1) hydrogen atoms from an aromatic ring which may have a substituent. The aromatic ring is not particularly limited as long as the aromatic ring is a cyclic conjugated system having (4n+2) π electrons. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a heteroatom. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Examples of the aromatic hydrocarbon group as Wa^{x1} also include a group obtained by removing (nₐₓ₁+1) hydrogen atoms from an aromatic compound including an aromatic ring (for example, biphenyl or fluorene) which may have two or more substituents.

Among these, Wa^{x1} represents preferably a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group as Wa^{x1} may have a substituent or may not have a substituent. Examples of substituents include an alkyl group, an alkoxy group, a halogen atom, and a halogenated alkyl group. Examples of the alkyl group, the alkoxy group, the halogen atom, and the halogenated alkyl group as the substituent include the same groups described as the substituent in the cyclic group as R′²⁰¹. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} has no substituent.

In Formula (a10-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In the formulae shown below, R^{α} represents a hydrogen atom, a methyl group, or a trifluoromethyl group.

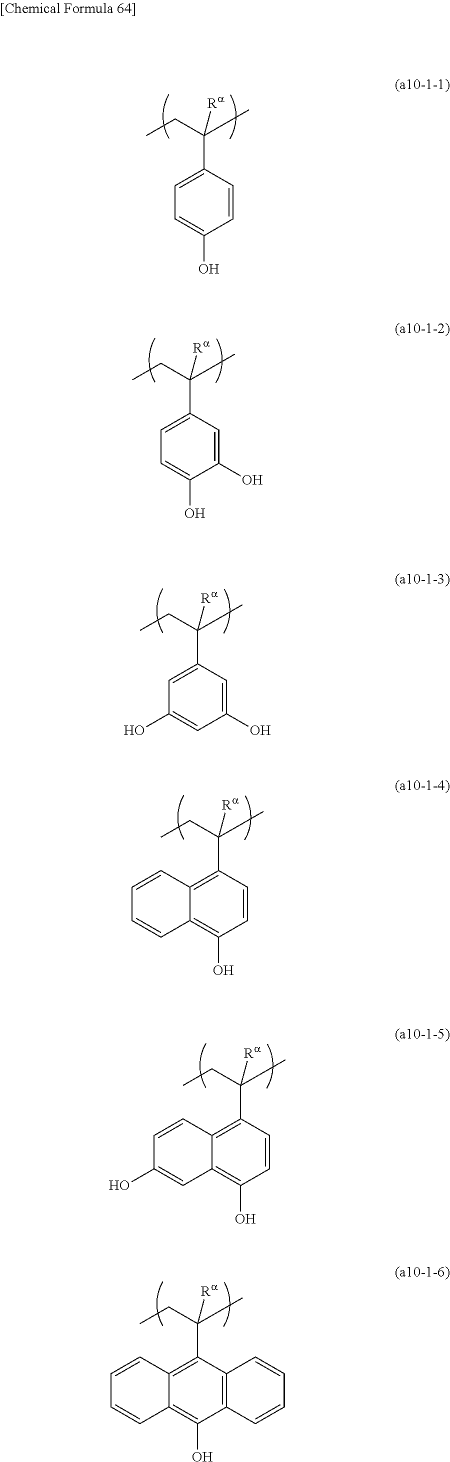

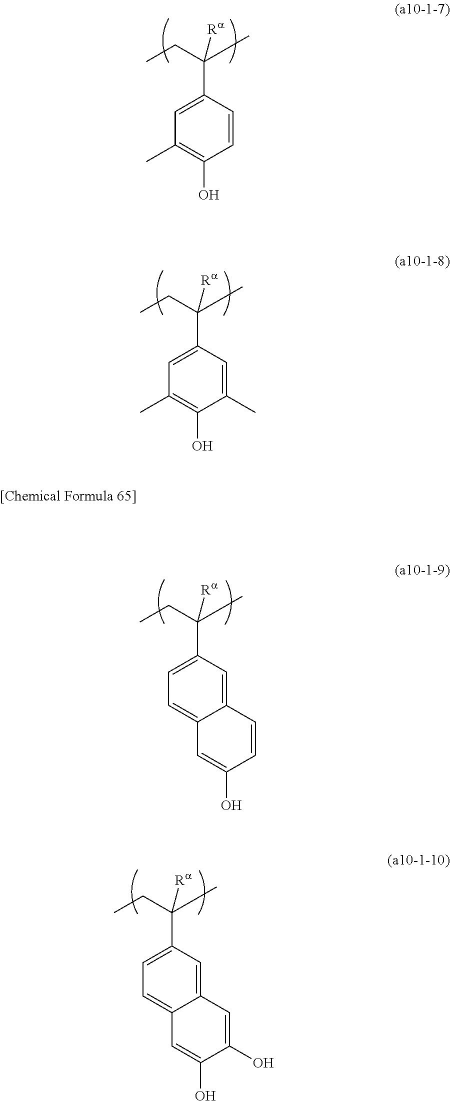

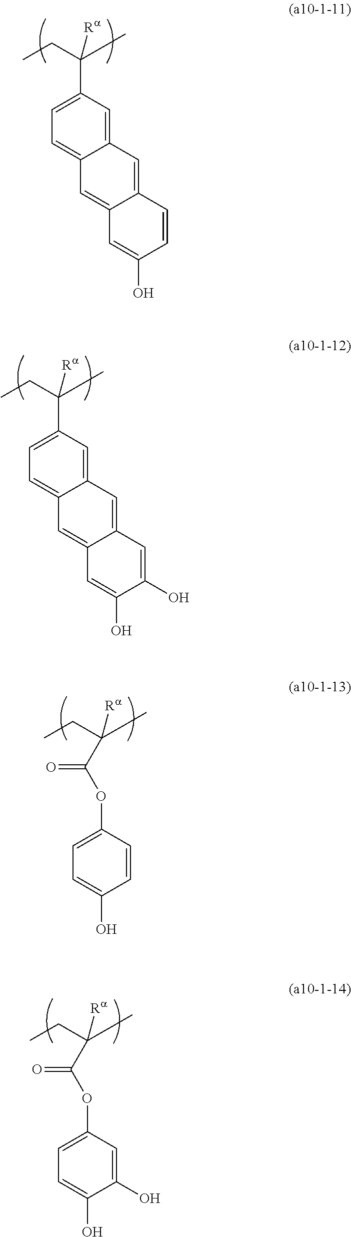

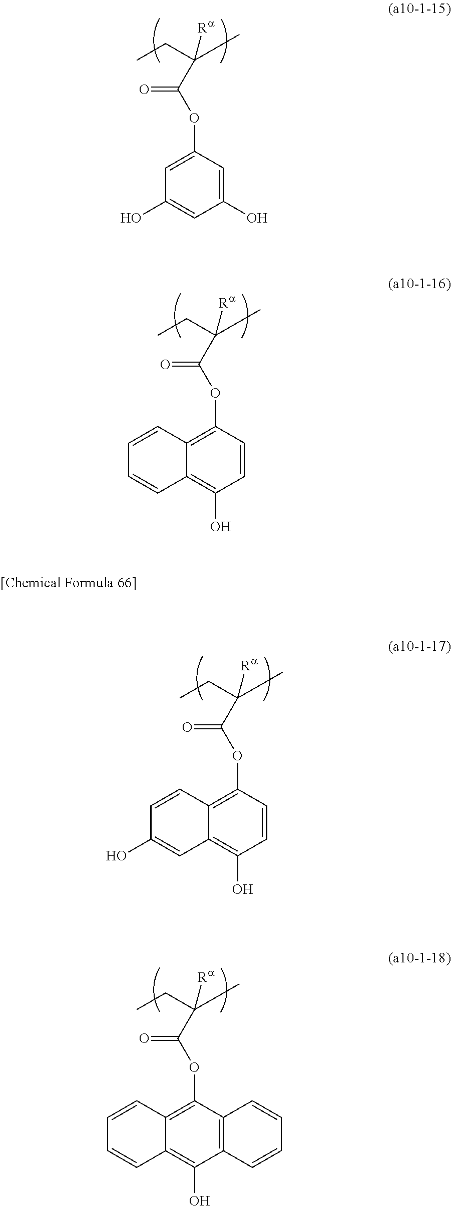

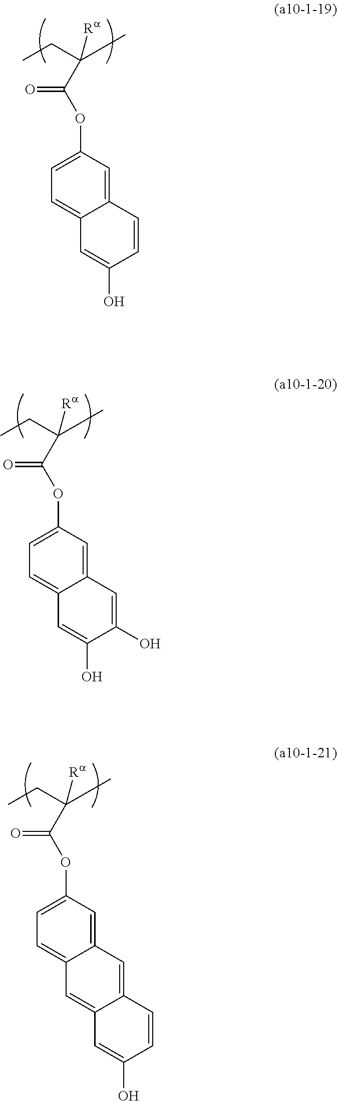

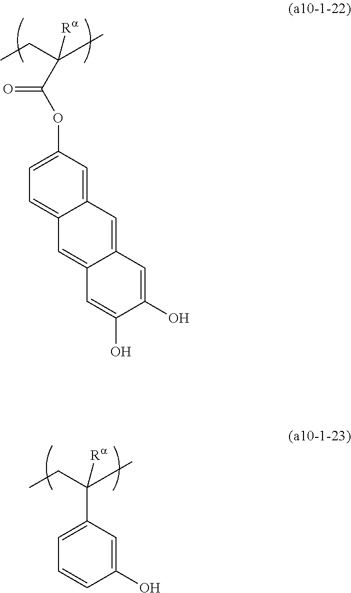

The constitutional unit (a10) included in the component (A1) may be used alone or two or more kinds thereof.

In a case where the component (A1) has the constitutional unit (a10), the proportion of the constitutional unit (a10) in the component (A1) is preferably in a range of 40% to 95% by mole, more preferably in a range of 15% to 50% by mole, still more preferably in a range of 20% to 45% by mole, and particularly preferably in a range of 25% to 40% by mole with respect to the total amount (100% by mole) of all constitutional units constituting the component (A1).

In a case where the proportion of the constitutional unit (a10) is greater than or equal to the lower limits of the above-described preferable ranges, the sensitivity is likely to be further enhanced. Meanwhile, in a case where the proportion thereof is set to be less than or equal to the above-described upper limits, the constitutional unit (a10) and other constitutional units are likely to be balanced.

### Constitutional Unit (a5):

In the present embodiment, the constitutional unit (a5) is a constitutional unit (here, constitutional units corresponding to the constitutional unit (a0) are excluded) that generates an acid upon light exposure, and a known constitutional unit can be used. In a case where the component (A1) has the constitutional unit (a5), the acid generated upon light exposure is likely to be uniformly distributed in the resist film.

Suitable examples of the constitutional unit (a5) include a constitutional unit represented by General Formula (a5-1).

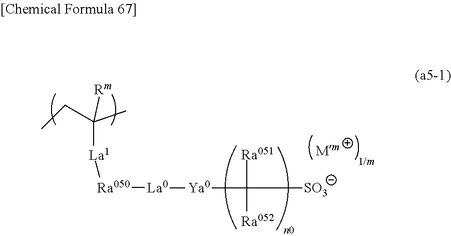

[In the formula, R^{m} represents an alkyl group having 1 to 5 carbon atoms, a halogenated alkyl group having 1 to 5 carbon atoms, a halogen atom, or a hydrogen atom. La¹ represents a divalent linking group or a single bond. Ra⁰⁵⁰ represents a divalent hydrocarbon group which may have a substituent. La⁰ represents a divalent linking group. Ya⁰ represents a divalent linking group which may have a heteroatom or a single bond. Ra⁰⁵¹ and Ra⁰⁵² each independently represent a hydrogen atom, a fluorine atom, or a fluorinated alkyl group. n0 represents an integer of 1 to 4. m represents an integer of 1 or greater, and M′^{m+} represents an m-valent onium cation.]

### {Anion Moiety}

In Formula (a5-1), R^{m} represents an alkyl group having 1 to 5 carbon atoms, a halogenated alkyl group having 1 to 5 carbon atoms, a halogen atom, or a hydrogen atom.

The alkyl group having 1 to 5 carbon atoms as R^{m} is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group.

The halogenated alkyl group having 1 to 5 carbon atoms is a group in which some or all hydrogen atoms in the alkyl group having 1 to 5 carbon atoms have been substituted with halogen atoms.

Examples of the halogen atom include a fluorine atom, a chlorine atom, a bromine atom, and an iodine atom. A fluorine atom is particularly preferable as the halogen atom in the halogenated alkyl group.

R^{m} represents preferably a hydrogen atom, an alkyl group having 1 to 5 carbon atoms, or a fluorinated alkyl group having 1 to 5 carbon atoms and, from the viewpoint of industrial availability, most preferably a hydrogen atom or a methyl group.

In Formula (a5-1), La¹ represents a divalent linking group or a single bond.

The divalent linking group as La¹ is not particularly limited, and suitable examples thereof include a divalent hydrocarbon group which may have a substituent and a divalent linking group having a heteroatom, each of which has the same definition as the divalent hydrocarbon group which may have a substituent and the divalent linking group having a heteroatom, described as the divalent linking group represented by Ya^{x1}.

Among these, it is preferable that La¹ represents an ester bond [—C(═O)—O— or —O—C(═O)—], an ether bond (—O—), a linear or branched alkylene group, an aromatic hydrocarbon group, or a combination thereof, or a single bond. Among these, La¹ represents more preferably an ester bond [—C(═O)—O— or —O—C(═O)—] or a single bond and still more preferably an ester bond [—C(═O)—O— or —O—C(═O)—].

In Formula (a5-1), Ra⁰⁵⁰ represents a divalent hydrocarbon group which may have a substituent.

The divalent hydrocarbon group as Ra⁰⁵⁰ may be an aliphatic hydrocarbon group or an aromatic hydrocarbon group.

### Aliphatic Hydrocarbon Group as Ra⁰⁵⁰

The aliphatic hydrocarbon group indicates a hydrocarbon group that has no aromaticity. The aliphatic hydrocarbon group may be saturated or unsaturated. In general, it is preferable that the aliphatic hydrocarbon group is saturated.

Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

The above linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a heteroatom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include the same groups as described above.

The cyclic aliphatic hydrocarbon group has preferably 3 to 20 carbon atoms and more preferably 3 to 12 carbon atoms.

The cyclic aliphatic hydrocarbon group may be a polycyclic group or a monocyclic group. The monocyclic alicyclic hydrocarbon group is preferably a group obtained by removing two hydrogen atoms from a monocycloalkane. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples of the polycycloalkane include adamantane, norbornane, isobornane, tricyclodecane, and tetracyclododecane.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

The alkyl group as the substituent is preferably an alkyl group having 1 to 5 carbon atoms, and most preferably a methyl group, an ethyl group, a propyl group, an n-butyl group, or a tert-butyl group.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Examples of the halogen atom for the substituent include a fluorine atom, a chlorine atom, a bromine atom, and an iodine atom, and a fluorine atom is preferable.

Examples of the halogenated alkyl group as the substituent include groups in which some or all hydrogen atoms in the above-described alkyl groups have been substituted with the above-described halogen atoms.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

### Aromatic Hydrocarbon Group as Ra⁰⁵⁰

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms. Specifically, as the aromatic ring, an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms are exemplary examples. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group (an arylene group or a heteroarylene group) obtained by removing two hydrogen atoms from the above-described aromatic hydrocarbon ring or the above-described aromatic heterocyclic ring; a group obtained by removing two hydrogen atoms from an aromatic compound (for example, biphenyl or fluorene) having two or more aromatic rings; and a group (for example, a group obtained by further removing one hydrogen atom from an aryl group in the arylalkyl group such as a benzyl group, a phenethyl group, a 1-naphthylmethyl group, a 2-naphthylmethyl group, a 1-naphthylethyl group, or a 2-naphthylethyl group) obtained by substituting one hydrogen atom of a group (an aryl group or a heteroaryl group) obtained by removing one hydrogen atom from the above aromatic hydrocarbon ring or the above aromatic heterocyclic ring, with an alkylene group. The above-described alkylene group bonded to the aryl group or heteroaryl group has preferably 1 to 4 carbon atoms, more preferably 1 or 2 carbon atoms, and particularly preferably 1 carbon atom.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

The alkyl group as the substituent is preferably an alkyl group having 1 to 5 carbon atoms, and most preferably a methyl group, an ethyl group, a propyl group, an n-butyl group, or a tert-butyl group.

Examples of the alkoxy group, the halogen atom, and the halogenated alkyl group as the substituents include those described as the substituent that substitutes a hydrogen atom in the cyclic aliphatic hydrocarbon group.

Among these, Ra⁰⁵⁰ represents preferably an aliphatic hydrocarbon group having a ring in the structure, more preferably a cyclic aliphatic hydrocarbon group which may have a substituent having a heteroatom in the ring structure, and still more preferably an alicyclic hydrocarbon group which may have a substituent, which is a polycyclic group or a monocyclic group.

In Formula (a5-1), La⁰ represents a divalent linking group.

Examples of the divalent linking group as La⁰ include a non-hydrocarbon oxygen atom-containing linking group such as an oxygen atom (ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon oxygen atom-containing linking groups with an alkylene group. Further, a sulfonyl group (—SO₂—) may be further linked to the combination.

Examples of such a divalent linking group include linking groups each represented by General Formulae (L-a1-1) to (L-a1-8). Further, in General Formulae (L-a1-1) to (L-a1-8), V′¹⁰¹ in General Formulae (L-a1-1) to (L-a1-8) is bonded to Ra⁰⁵⁰ in Formula (a5-1).

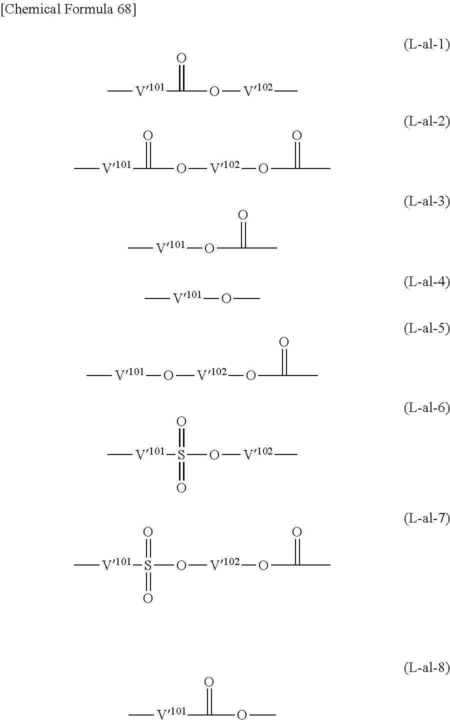

[In the formulae, V′¹⁰¹ represents a single bond or an alkylene group having 1 to 5 carbon atoms, and V′¹⁰² represents a divalent saturated hydrocarbon group having 1 to 30 carbon atoms.]

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

Further, a part of methylene group in the alkylene group as V′¹⁰¹ and V′¹⁰² may be substituted with a divalent aliphatic cyclic group having 5 to 10 carbon atoms. As the aliphatic cyclic group, a divalent group in which one hydrogen atom has been removed from the cyclic aliphatic hydrocarbon group (a monocyclic aliphatic hydrocarbon group or a polycyclic aliphatic hydrocarbon group) as Ra′³ in Formula (a1-r-1) is preferable, and a cyclohexylene group, a 1,5-adamantylene group, or a 2,6-adamantylene group is more preferable.

La⁰ represents preferably a divalent linking group having an ester bond or a divalent linking group having an ether bond, more preferably a linking group represented by any of Formulae (L-a1-1) to (L-a1-5) and (L-a1-8), and still more preferably a linking group represented by Formula (L-a1-3) or (L-a1-8).

In Formula (a5-1), Ya⁰ represents a divalent linking group which may have a heteroatom or a single bond.

The divalent linking group as Ya⁰ is not particularly limited, and suitable examples thereof include a divalent hydrocarbon group which may have a substituent, and a divalent linking group having a heteroatom.

The divalent hydrocarbon group which may have a substituent and the divalent linking group having a heteroatom as Ya⁰ each have the same definition as the divalent hydrocarbon group which may have a substituent and the divalent linking group having a heteroatom, described as the divalent linking group represented by Ya^{x1}.

Among the examples, Ya⁰ represents preferably a linear or branched alkylene group or a single bond and more preferably a single bond.

In Formula (a5-1), Ra⁰⁵¹ and Ra⁰⁵² each independently represent a hydrogen atom, a fluorine atom, or a fluorinated alkyl group.

The fluorinated alkyl groups as Ra⁰⁵¹ and Ra⁰⁵² are each preferably a linear or branched fluorinated alkyl group having 1 to 5 carbon atoms and more preferably a trifluoromethyl group.

In Formula (a5-1), it is preferable that at least one of Ra⁰⁵¹ and Ra⁰⁵² bonded to the carbon atom adjacent to SO₃⁻ represents a fluorine atom from the viewpoint of acid strength.

In Formula (a5-1), n0 represents an integer of 1 to 4 and preferably 1, 2, or 3.

### {Cation Moiety}

In Formula (a5-1), M′^{m+} represents an m-valent onium cation. Among these, it is preferable that M′^{m+} represents a sulfonium cation or an iodonium cation. m represents an integer of 1 or greater.

Preferred examples of the cation moiety ((M′^{m+})_{1/m}) include organic cations each represented by General Formulae (ca-1) to (ca-3). Among the examples, an organic cation represented by General Formula (ca-1) is more preferable as the cation moiety ((M′^{m+})_{1/m}).

Specific preferred examples of the constitutional unit (a5) are shown below.

In the formulae shown below, R^{α} represents a hydrogen atom, a methyl group, or a trifluoromethyl group. m and M′^{m+} each have the same definition as that for m and M′^{m+} in General Formula (a5-1).

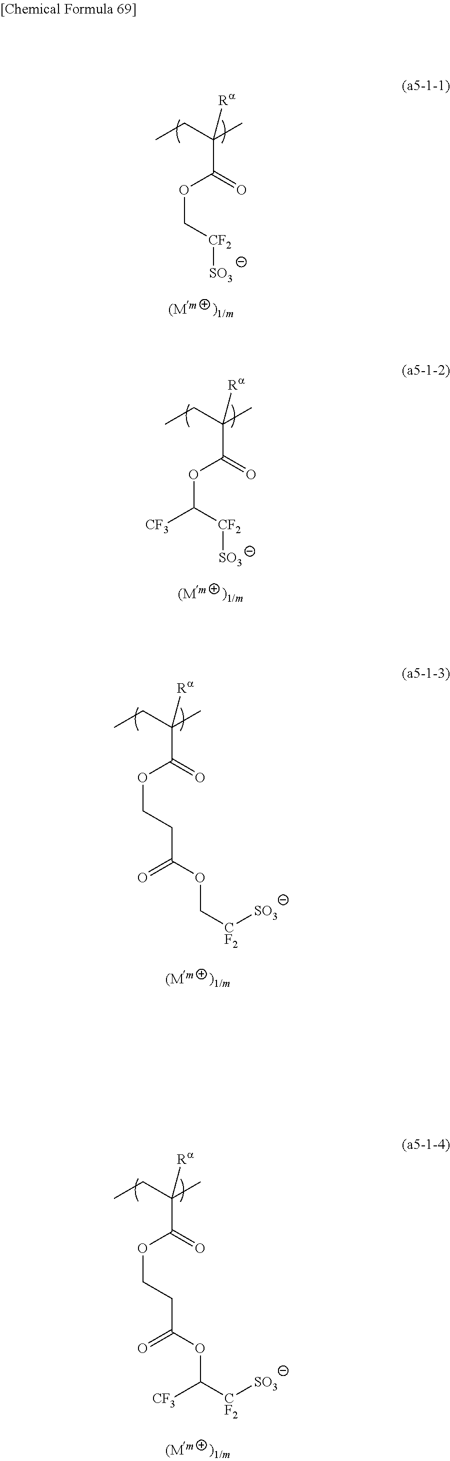

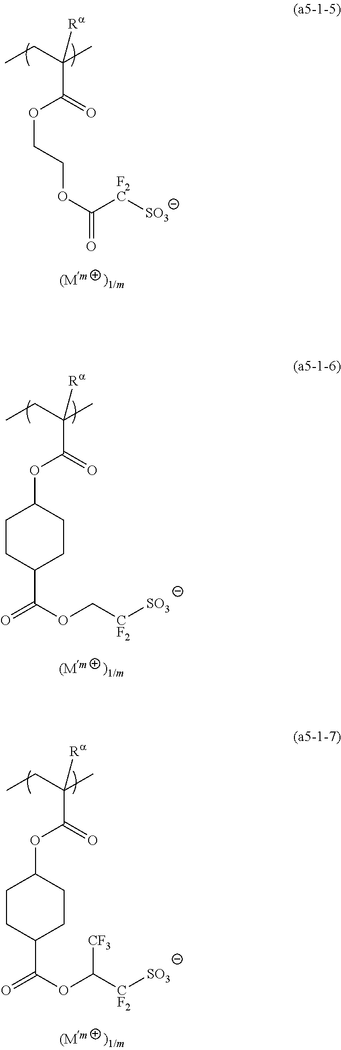

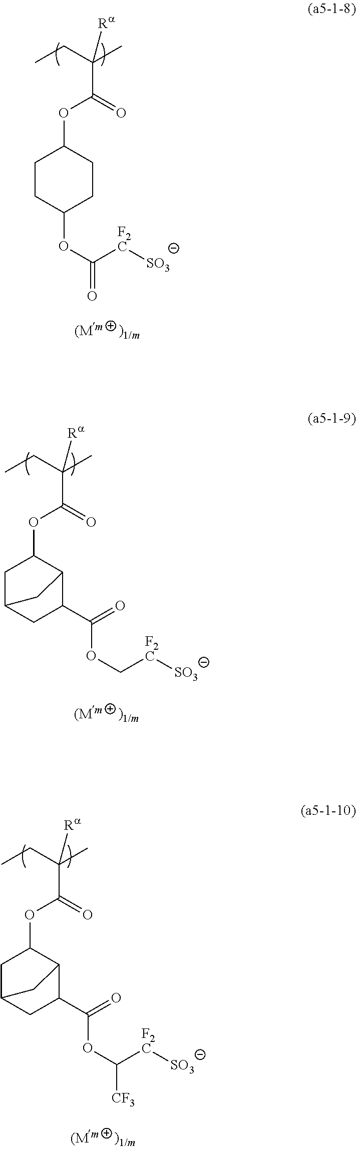

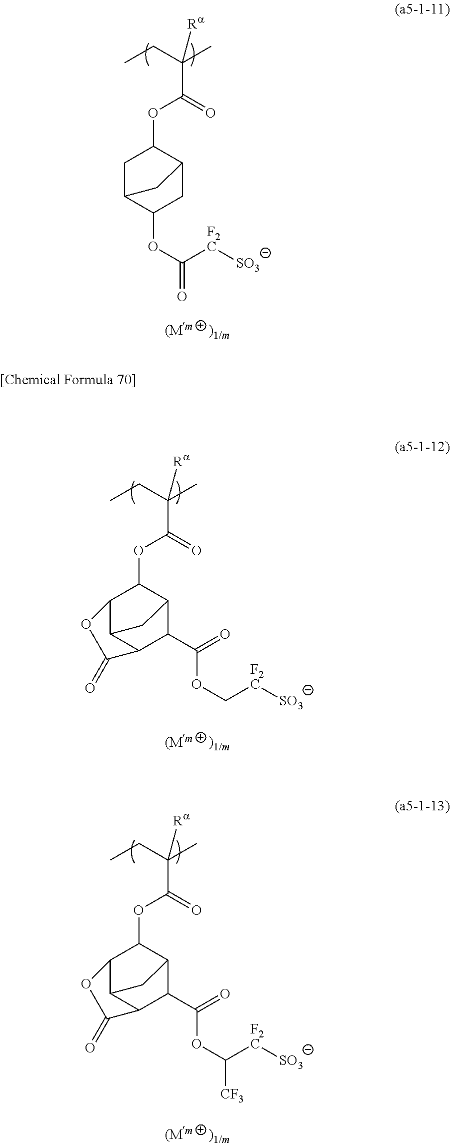

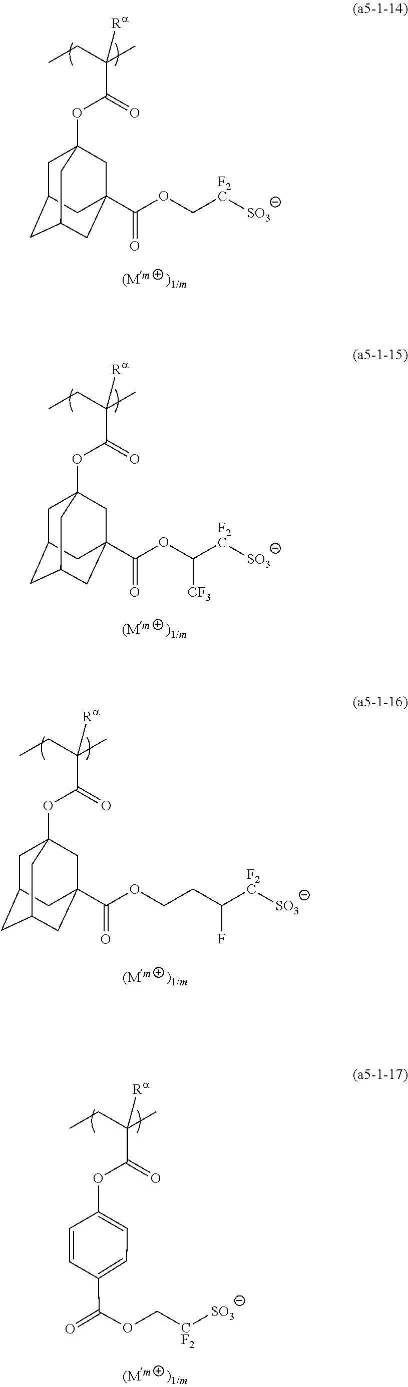

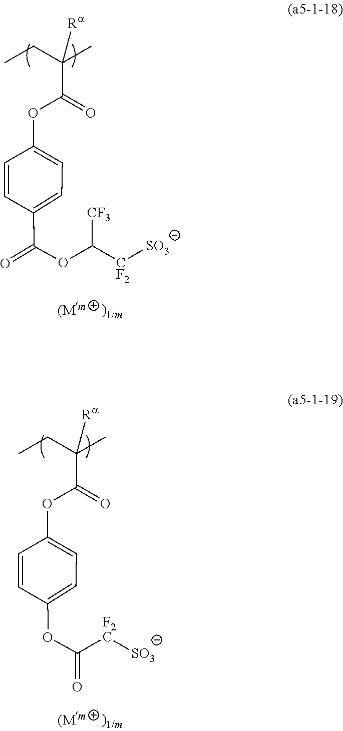

The constitutional unit (a5) of the component (A1) may be used alone or two or more kinds thereof.

In a case where the component (A1) has the constitutional unit (a5), the proportion of the constitutional unit (a5) in the component (A1) is preferably in a range of 5% to 25% by mole, more preferably in a range of 10% to 20% by mole, and still more preferably in a range of 15% to 20% by mole with respect to the total amount (100% by mole) of all constitutional units constituting the component (A1).

In a case where the proportion of the constitutional unit (a5) is greater than or equal to the lower limits of the above-described preferable ranges, the sensitivity is likely to be further increased. Meanwhile, in a case where the proportion thereof is less than or equal to the upper limits of the above-described preferable ranges, the constitutional unit (a5) and other constitutional units are likely to be balanced.

### Constitutional Unit (a2):

The component (A1) may have a constitutional unit (a2) containing a lactone-containing cyclic group (here, constitutional units corresponding to the constitutional unit (a1) are excluded).

In a case where the component (A1) is used to form a resist film, the lactone-containing cyclic group of the constitutional unit (a2) is effective for increasing the adhesiveness of the resist film to the substrate. Further, in a case where the component (A1) contains the constitutional unit (a2), the lithography characteristics and the like are enhanced due to the effects of appropriately adjusting the acid diffusion length, increasing the adhesiveness of the resist film to the substrate, and appropriately adjusting the solubility during the development.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional constitutional unit can be used. Specific examples thereof include groups each represented by General Formulae (a2-r-1) to (a2-r-7).

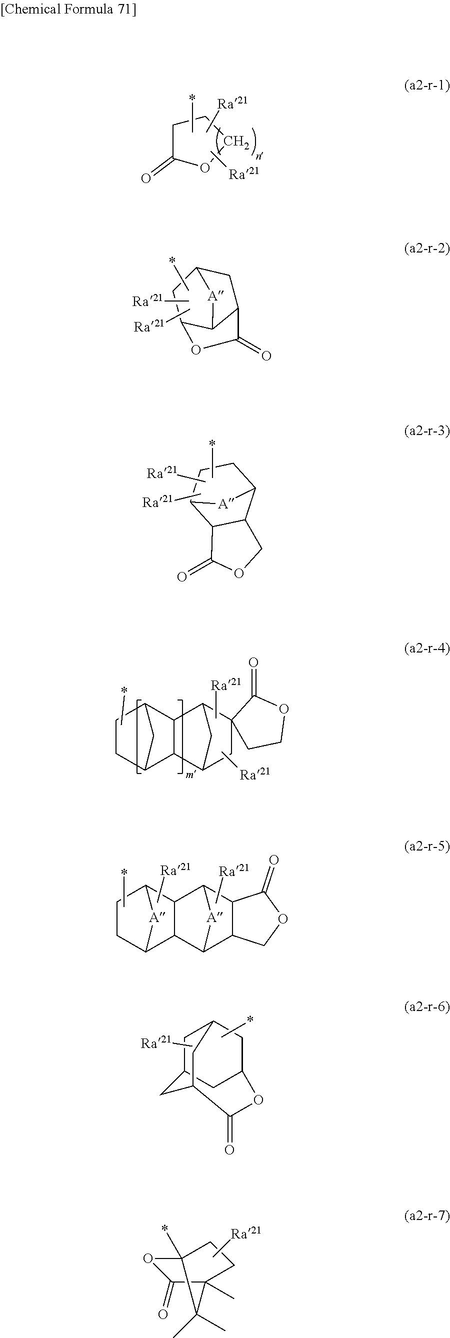

[In the formulae, Ra′²¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, or a lactone-containing cyclic group, A″ represents an alkylene group having 1 to 5 carbon atoms which may have an oxygen atom (—O—) or a sulfur atom (—S—), an oxygen atom, or a sulfur atom, n′ represents an integer of 0 to 2, and m′ represents 0 or 1. * represents a bonding site (the same applies hereinafter).]

In General Formulae (a2-r-1) to (a2-r-7), it is preferable that the alkyl group as Ra′²¹ is an alkyl group having 1 to 6 carbon atoms. Further, it is preferable that the alkyl group is linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ is an alkoxy group having 1 to 6 carbon atoms. Further, it is preferable that the alkoxy group is linear or branched. Specific examples of the alkoxy groups include a group formed by linking the above-described alkyl group described as the alkyl group represented by Ra′²¹ to an oxygen atom (—O—).

As the halogen atom as Ra′²¹, a fluorine atom is preferable.

Examples of the halogenated alkyl group as Ra′²¹ include groups in which some or all hydrogen atoms in the alkyl group as Ra′²¹ have been substituted with the halogen atoms. As the halogenated alkyl group, a fluorinated alkyl group is preferable, and a perfluoroalkyl group is particularly preferable.

In —COOR″ and —OC(═O)R″ as Ra′²¹, each R″ represents a hydrogen atom, an alkyl group, or a lactone-containing cyclic group.

The alkyl group as R″ may be linear, branched, or cyclic and has preferably 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane, or tetracyclododecane.

Examples of the lactone-containing cyclic group as R″ include the same groups as those for the groups each represented by General Formulae (a2-r-1) to (a2-r-7).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the examples, it is preferable that Ra′²¹'s each independently represent a hydrogen atom or a cyano group.

In General Formulae (a2-r-2), (a2-r-3), and (a2-r-5), as the alkylene group having 1 to 5 carbon atoms as A″, a linear or branched alkylene group is preferable, and examples thereof include a methylene group, an ethylene group, an n-propylene group, and an isopropylene group. Specific examples of the alkylene groups that contain an oxygen atom or a sulfur atom include a group obtained by interposing —O— or —S— in the terminal of the alkylene group or between the carbon atoms of the alkylene group, and examples thereof include —O—CH₂—, —CH₂—O—CH₂—, —S—CH₂—, and —CH₂—S—CH₂—. A″ represents preferably an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

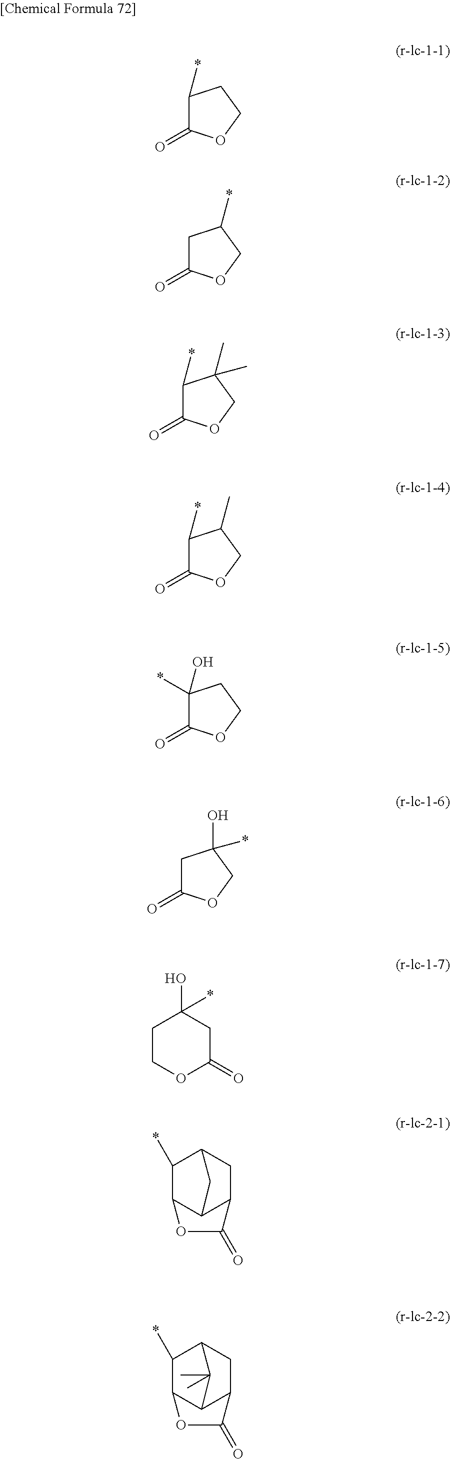

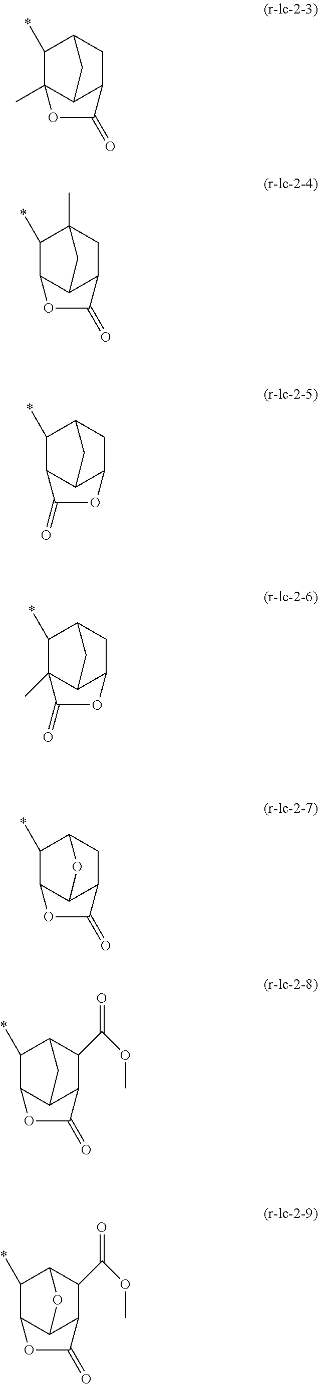

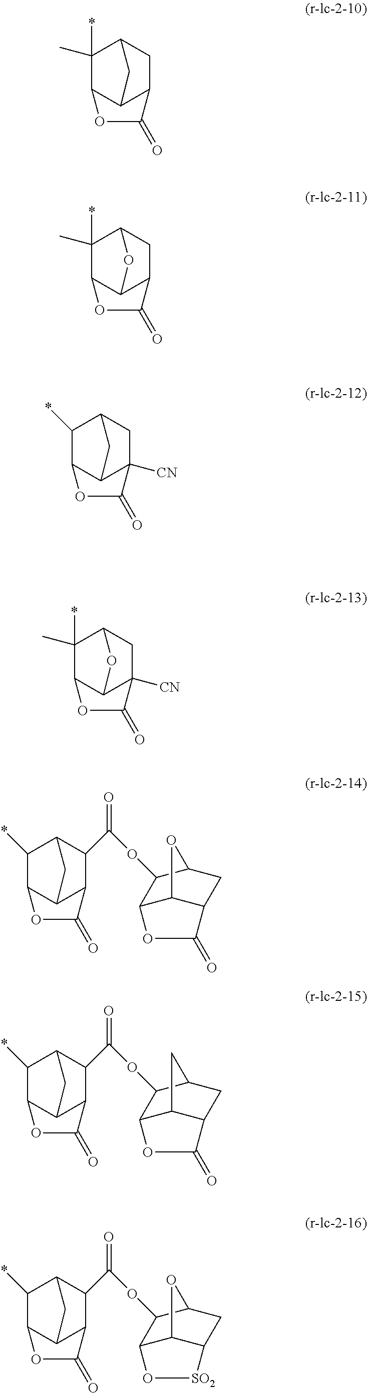

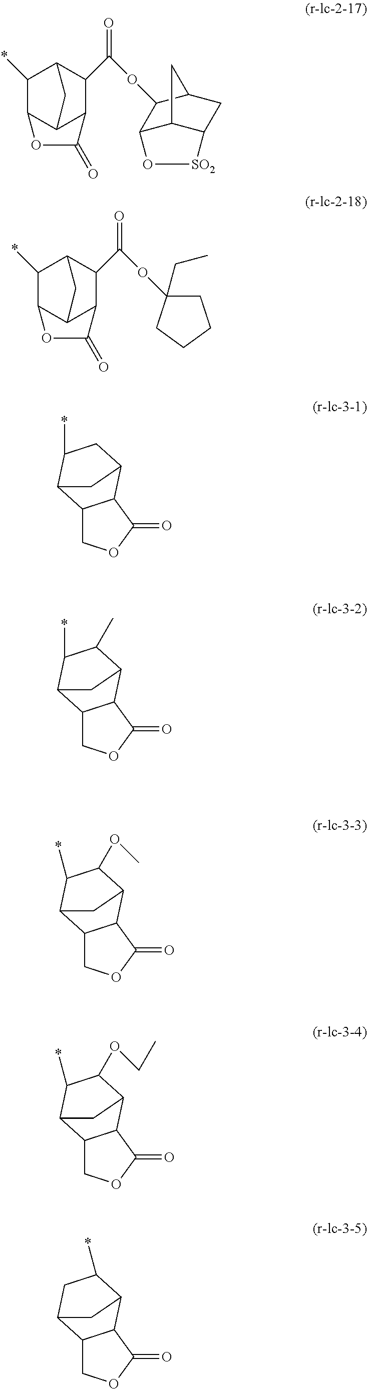

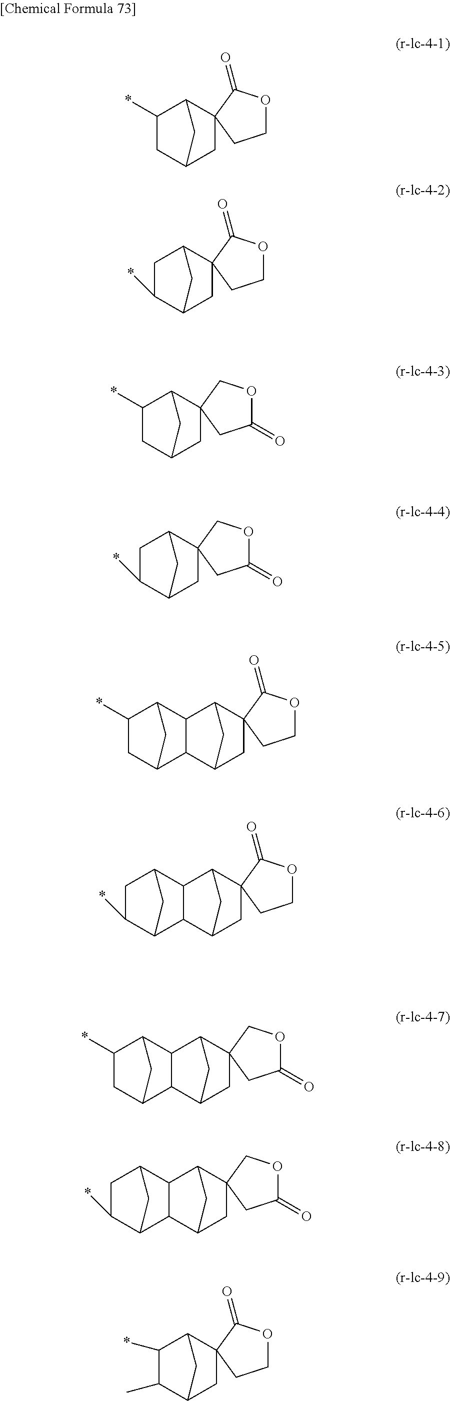

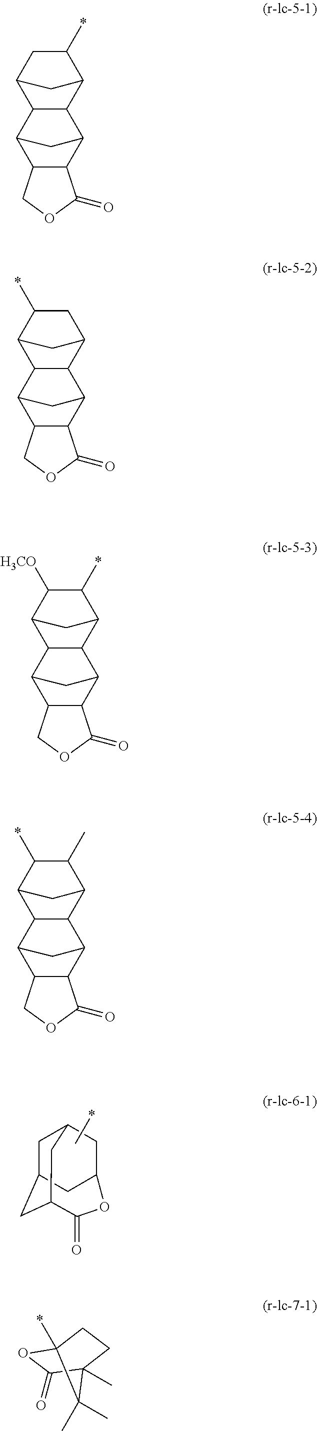

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) is a constitutional unit represented by General Formula (a2-1).

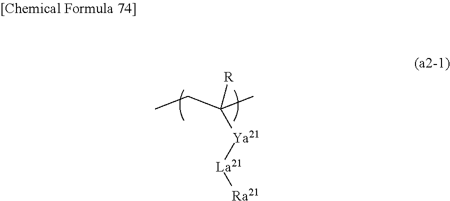

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R¹ represents a hydrogen atom or a methyl group. In a case where La²¹ represents —O—, Ya²¹ does not represent —CO—. Ra²¹ represents a lactone-containing cyclic group.]

In Formula (a2-1), R represents preferably a hydrogen atom, an alkyl group having 1 to 5 carbon atoms, or a fluorinated alkyl group having 1 to 5 carbon atoms, and from the viewpoint of industrial availability, particularly preferably a hydrogen atom or a methyl group.

In Formula (a2-1), the divalent linking group as Ya^{2l} is not particularly limited, and suitable examples thereof include a divalent hydrocarbon group which may have a substituent, and a divalent linking group having heteroatoms.

It is preferable that Ya²¹ represents a single bond, an ester bond [—C(═O)—O—], an ether bond (—O—), a linear or branched alkylene group, or a combination thereof.

In Formula (a2-1), it is preferable that Ya²¹ represents a single bond and La²′ represents —COO— or —OCO—.

In Formula (a2-1), Ra²¹ represents a lactone-containing cyclic group. Suitable examples of the lactone-containing cyclic group as Rau include groups each represented by General Formulae (a2-r-1) to (a2-r-7).

The constitutional unit (a2) included in the component (A1) may be used alone or two or more kinds thereof.

In a case where the component (A1) has the constitutional unit (a2), the proportion of the constitutional unit (a2) is preferably in a range of 1% to 20% by mole, more preferably in a range of 1% to 15% by mole, and still more preferably in a range of 1% to 10% by mole with respect to the total amount (100% by mole) of all constitutional units constituting the component (A1).

In a case where the proportion of the constitutional unit (a2) is set to be greater than or equal to the lower limits of the above-described preferable ranges, the effect to be obtained by allowing the component (A1) to have the constitutional unit (a2) is sufficiently obtained by the above-described effects. Further, in a case where the proportion thereof is set to be less than or equal to the upper limits of the above-described preferable ranges, the constitutional unit (a2) and other constitutional units can be balanced, and the lithography characteristics are enhanced.

### Constitutional Unit (a8):

The constitutional unit (a8) is a constitutional unit derived from a compound represented by General Formula (a8-1).

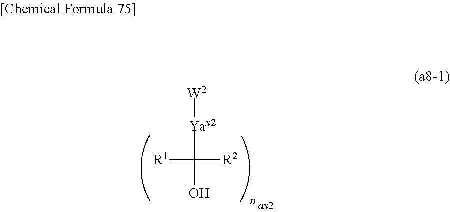

[In the formula, W² represents a polymerizable group-containing group. Ya^{x2} represents a single bond or an (nₐₓ₂+1)-valent linking group. Ya^{x2} and W² may form a condensed ring. R¹ represents a fluorinated alkyl group having 1 to 12 carbon atoms. R² represents an organic group having 1 to 12 carbon atoms which may have a fluorine atom, or a hydrogen atom. R² and Ya^{x2} may be bonded to each other to form a ring structure. nₐₓ₂ represents an integer of 1 to 3.]

The term “polymerizable group” in the polymerizable group-containing group as W² is a group that enables a compound having the polymerizable group to be polymerized by radical polymerization or the like, and refers to a group containing a multiple bond between carbon atoms, such as an ethylenic double bond.

The polymerizable group-containing group may be a group formed of only a polymerizable group or a group formed of a polymerizable group and a group other than the polymerizable group. Examples of the group other than the polymerizable group include a divalent hydrocarbon group which may have a substituent and a divalent linking group containing a heteroatom.

Suitable examples of the polymerizable group-containing group include a group represented by a chemical formula: C(R^{X11})(R^{X12})═C(R^{X13})—Ya^{x0}-.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

Examples of the condensed ring formed by Ya^{x2} and W² include a condensed ring formed by a polymerizable group of the W² moiety and by Ya^{x2} and a condensed ring formed by a group other than the polymerizable group of the W² moiety and by Ya^{x2}.

The condensed ring formed by Ya^{x2} and W² may have a substituent.

Specific examples of the constitutional unit (a8) are shown below.

In the following formulae, R^{α} represents a hydrogen atom, a methyl group, or a trifluoromethyl group.

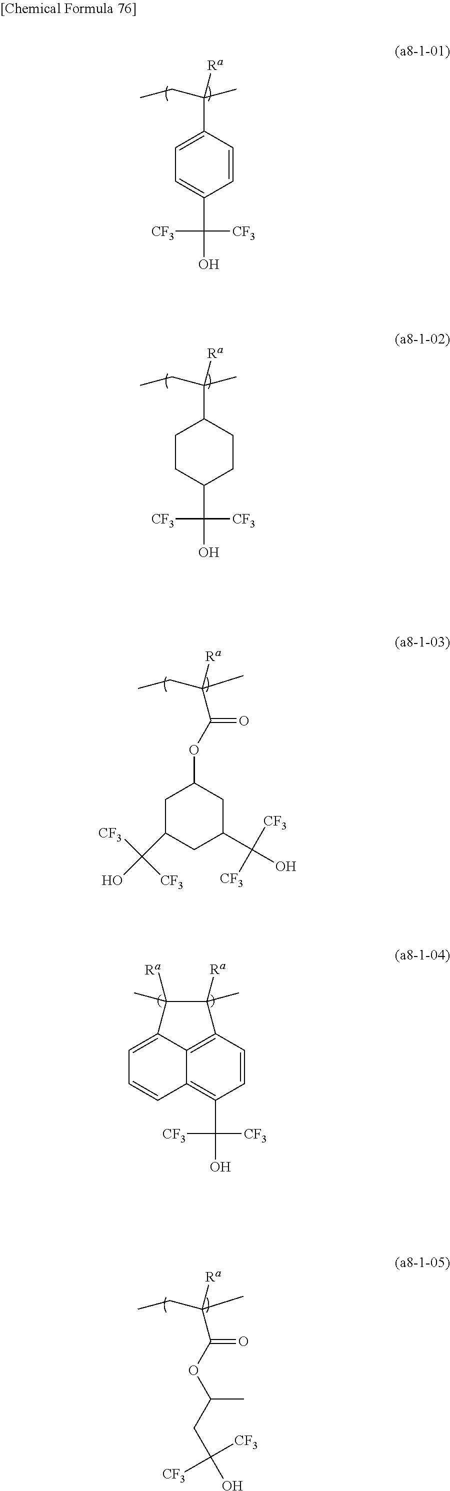

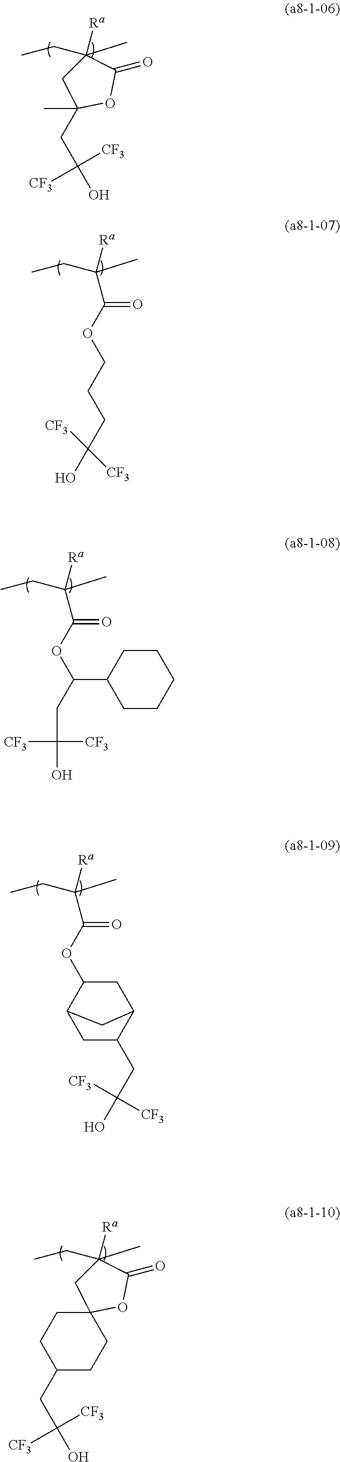

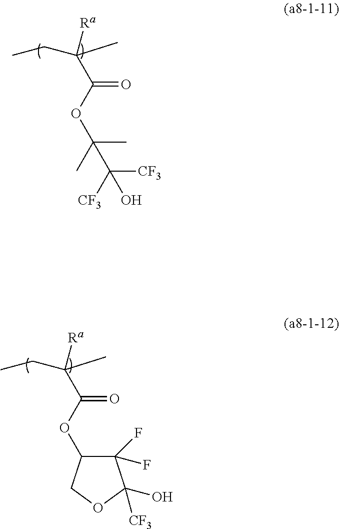

The constitutional unit (as) contained in the component (A1) may be used alone or two or more kinds thereof.

In a case where the component (A1) has the constitutional unit (as), the proportion of the constitutional unit (a8) is preferably in a range of 1% to 20% by mole, more preferably in a range of 1% to 15% by mole, and still more preferably in a range of 1% to 10% by mole with respect to the total amount (100% by mole) of all constitutional units constituting the component (A1).

The component (A1) contained in the resist composition may be used alone or in combination of two or more kinds thereof.

The component (A1) used in the present embodiment is a polymer compound having the constitutional unit (a0), and preferred examples thereof include a polymer compound having a repeating structure of the constitutional unit (a0), the constitutional unit (a1), and the constitutional unit (a10) and a polymer compound having a repeating structure of the constitutional unit (a0), the constitutional unit (a1), the constitutional unit (a10), and the constitutional unit (a5).

The content proportion of the constitutional unit (a0) in the component (A1) is preferably in a range of 1% to 50% by mole, more preferably in a range of 1% to 40% by mole, still more preferably in a range of 2% to 20% by mole, and particularly preferably in a range of 2% to 10% by mole with respect to the total amount (100% by mole) of all constitutional units constituting the component (A1).

The content proportion of the constitutional unit (a1) in the component (A1) is preferably in a range of 25% to 70% by mole, more preferably in a range of 35% to 65% by mole, and still more preferably in a range of 45% to 60% by mole with respect to the total amount (100% by mole) of all constitutional units constituting the component (A1).

In a case where the component (A1) has the constitutional unit (a10), the content proportion of the constitutional unit (a10) in the component (A1) is preferably in a range of 40% to 95% by mole, more preferably in a range of 15% to 50% by mole, still more preferably in a range of 20% to 45% by mole, and particularly preferably in a range of 25% to 40% by mole with respect to the total amount (100% by mole) of all constitutional units constituting the component (A1).

In a case where the component (A1) has the constitutional unit (a5), the content proportion of the constitutional unit (a5) in the component (A1) is preferably in a range of 5% to 25% by mole, more preferably in a range of 10% to 20% by mole, and still more preferably in a range of 15% to 20% by mole with respect to the total amount (100% by mole) of all constitutional units constituting the component (A1).

Such a component (A1) can be produced by dissolving a monomer, from which each constitutional unit is derived, in a polymerization solvent and adding a radical polymerization initiator such as azobisisobutyronitrile (AIBN) or dimethyl azobisisobutyrate (for example, V-601) to the solution so that the polymerization is carried out.

Alternatively, the component (A1) can be produced by dissolving, in a polymerization solvent, a monomer from which the constitutional unit (a0) is derived and a monomer from which the constitutional unit (a10) is derived, and adding the above-described radical polymerization initiator thereto to perform polymerization, and performing a deprotection reaction.

Further, a —C(CF₃)₂—OH group may be introduced into the terminal of the component (A1) during the polymerization using a chain transfer agent such as HS—CH₂—CH₂—CH₂—C(CF₃)₂—OH in combination. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably in a range of 1000 to 40000, more preferably in a range of 2000 to 20000, and still more preferably in a range of 5000 to 15000.

In a case where the Mw of the component (A1) is less than or equal to the upper limits of the above-described preferable ranges, the resist composition exhibits a satisfactory solubility in a resist solvent for a resist enough to be used as a resist. On the contrary, in a case where the Mw of the component (A1) is greater than or equal to the lower limits of the above-described preferable ranges, the dry etching resistance and the cross-sectional shape of the resist pattern are excellent.

The polydispersity (Mw/Mn) of the component (A1) is not particularly limited, but is preferably in a range of 1.0 to 4.0, more preferably in a range of 1.0 to 3.0, and particularly preferably in a range of 1.0 to 2.0. Further, Mn represents the number average molecular weight.

### In Regard to Component (A2)

In the resist composition of the present embodiment, a base material component (hereinafter, also referred to as “component (A2)”) which does not correspond to the component (A1) and whose solubility in a developing solution is changed by the action of an acid may be used in combination as the component (A).

The component (A2) is not particularly limited and may be optionally selected from a plurality of components of the related art which have been known as base material components for a chemically amplified resist composition and used.

As the component (A2), a polymer compound or a low-molecular-weight compound may be used alone or in combination of two or more kinds thereof.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having various excellent lithography characteristics such as high sensitivity, resolution, and roughness reduction in the pattern is likely to be formed.

In the resist composition of the present embodiment, the content of the component (A) may be adjusted according to the thickness of the resist film intended to be formed.

For example, the content of the component (A1) in the resist composition may be, for example, in a range of 0.5% to 10% by mass or in a range of 1% to 5% by mass.

### <Other Components>

The resist composition according to the present embodiment may further contain other components in addition to the component (A) described above. Examples of the other components include a component (B), a component (D), a component (E), a component (F), and a component (S), which are described below.

### <<Component (B): Acid Generator Component>>

The resist composition according to the present embodiment may further contain an acid generator component (B) which generates an acid upon light exposure.

The component (B) is not particularly limited, and those which have been suggested so far as an acid generator for a chemically amplified resist composition in the related art can be used.

Examples of the acid generator include various acid generators, for example, onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators, imino sulfonate-based acid generators, and disulfone-based acid generators.

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

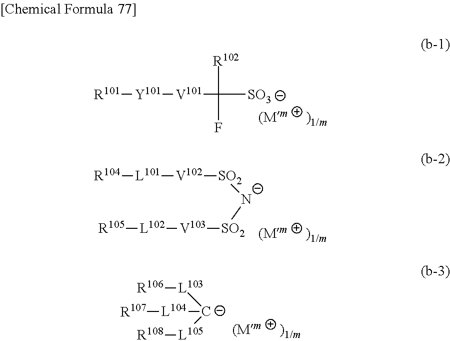

[In the formulae, R¹⁰¹ and R¹⁰⁴ to R′¹⁰⁸ each independently represent a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represent a single bond, an alkylene group, or a fluorinated alkylene group. L¹⁰¹ and L¹⁰² each independently represent a single bond or an oxygen atom. L¹⁰ to L¹⁰⁵ each independently represent a single bond, —CO—, or —SO₂—. m represents an integer of 1 or greater, and M′^{m+} represents an m-valent onium cation.]

### {Anion Moiety}

Anions in Component (b-1)

In Formula (b-1), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. In addition, it is preferable that the aliphatic hydrocarbon group is saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring of the aromatic hydrocarbon group as R¹⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R¹⁰¹ include a group obtained by removing one hydrogen atom from the aromatic ring (an aryl group such as a phenyl group or a naphthyl group) and a group in which one hydrogen atom of the aromatic ring is substituted with an alkylene group (for example, a benzyl group, a phenethyl group, or a 1-naphthylmethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Further, the cyclic hydrocarbon group as R¹⁰¹ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (b5-r-1) to (b5-r-4), and other heterocyclic groups each represented by Chemical Formulae (r-hr-1) to (r-hr-16).

In the formulae, * represents a bonding site bonded to Y¹⁰¹ in Formula (b-1).

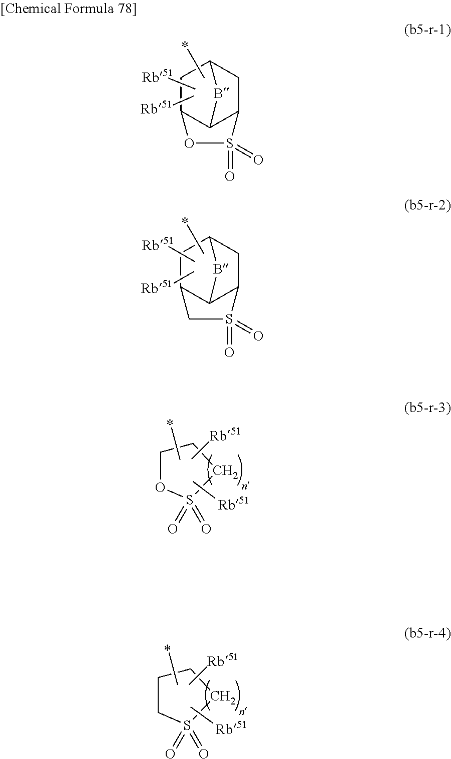

[In the formulae, Rb′⁵¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, or an —SO₂-containing cyclic group; B″ represents an oxygen atom, a sulfur atom, or an alkylene group having 1 to 5 carbon atoms, which may have an oxygen atom or a sulfur atom; and n′ represents an integer of 0 to 2. * represents a bonding site.]

Rb′⁵¹, B″, and n′ in General Formulae (b5-r-1) to (b5-r-4) each have the same definition as that for Ra′²¹, A″, and n′ in General Formulae (a2-r-1) to (a2-r-7).

Specific examples of the groups each represented by General Formulae (b5-r-1) to (b5-r-4) are shown below. In the formulae shown below, “Ac” represents an acetyl group.

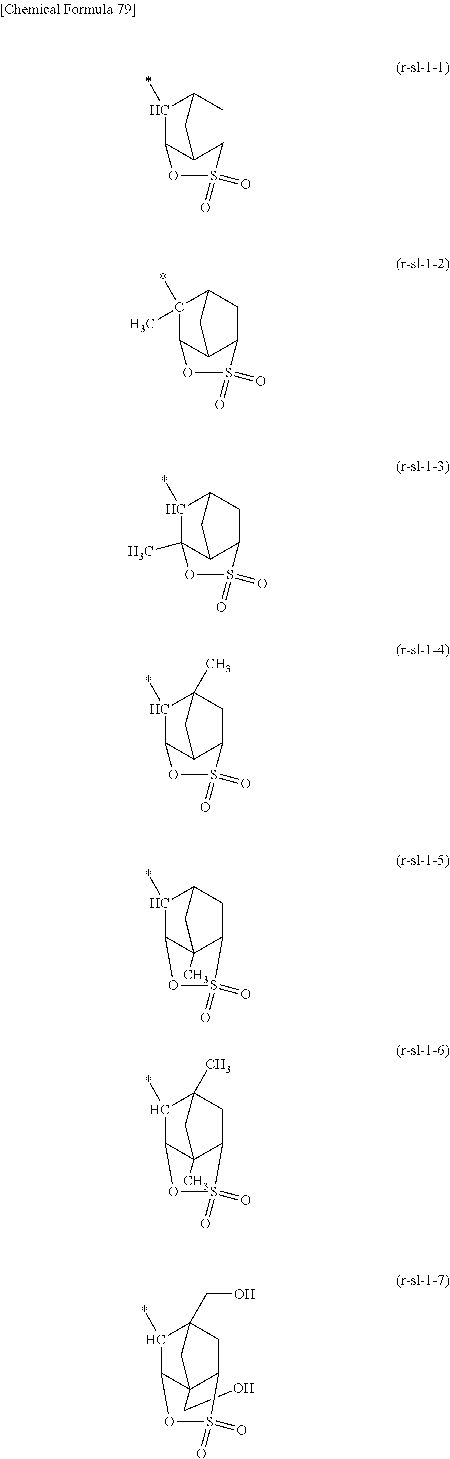

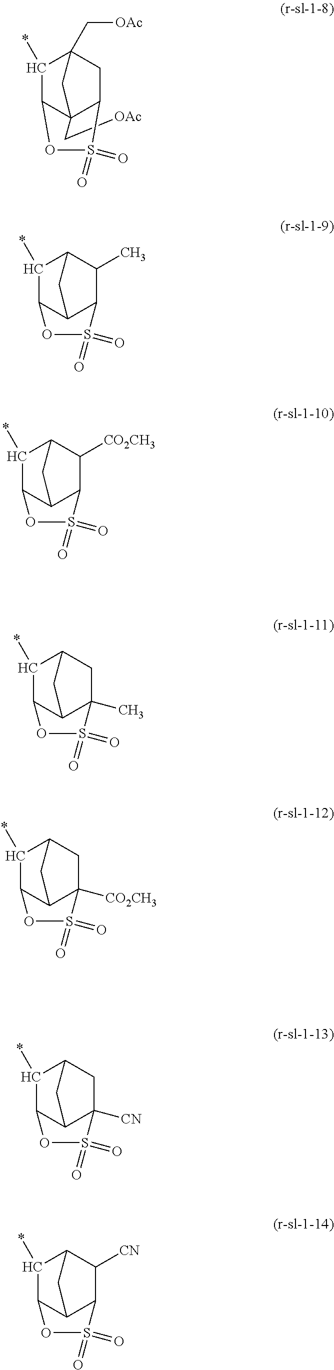

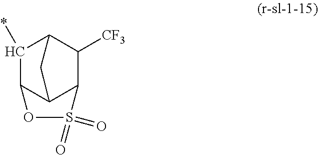

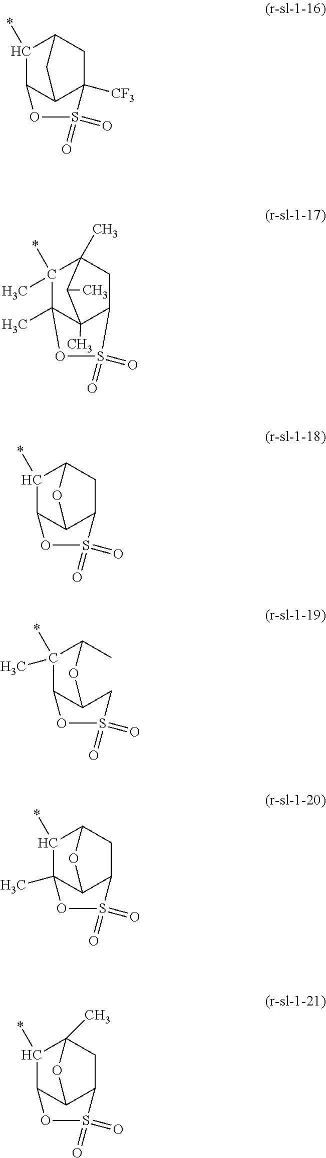

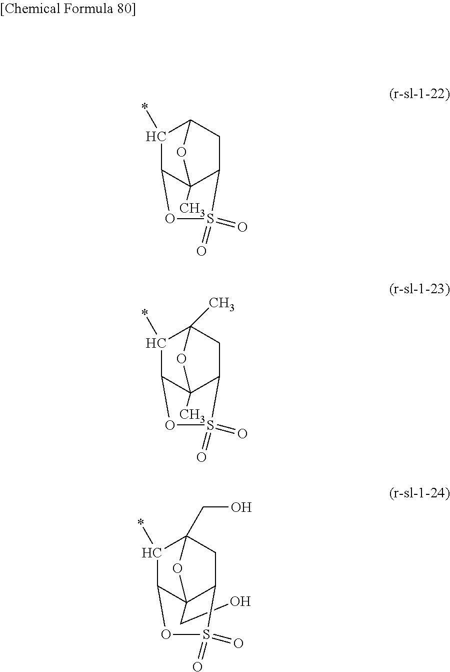

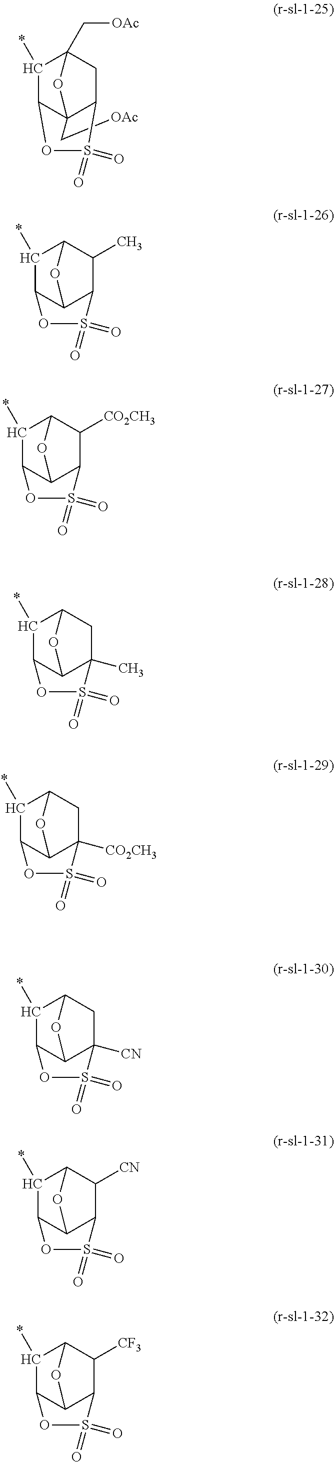

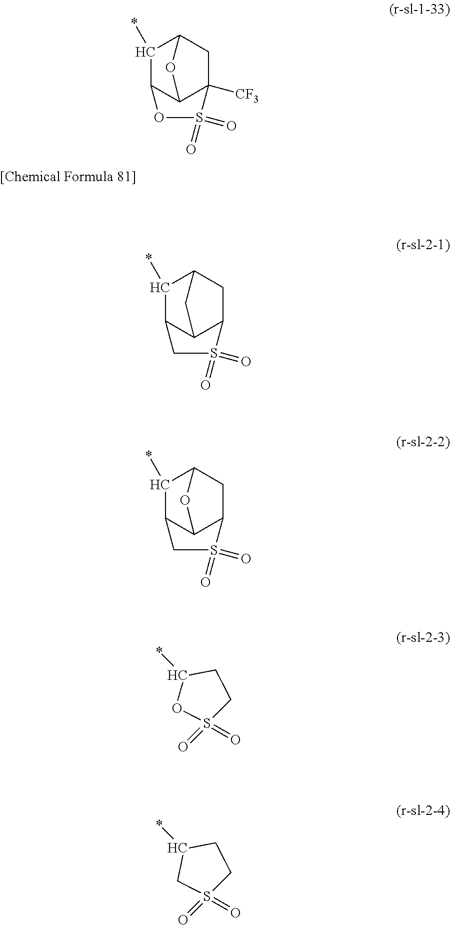

Examples of the substituent for the cyclic group as R¹⁰¹ include an alkyl group, an alkoxy group, a halogen atom, a halogenated alkyl group, a hydroxyl group, a carbonyl group, and a nitro group.

An alkyl group having 1 to 5 carbon atoms is preferable as the alkyl group serving as a substituent.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

A fluorine atom, a bromine atom, or an iodine atom is preferable as the halogen atom serving as a substituent.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group are substituted with the above-described halogen atoms.

The carbonyl group as the substituent is a group that substitutes a methylene group (—CH₂—) constituting the cyclic hydrocarbon group.

The cyclic hydrocarbon group as R¹⁰¹ may be a condensed cyclic group containing a condensed ring in which an aliphatic hydrocarbon ring and an aromatic ring are condensed. Examples of the condensed ring include those obtained by fusing one or more aromatic rings with a polycycloalkane having a crosslinked ring polycyclic skeleton. Specific examples of the crosslinked ring polycycloalkane include a bicycloalkane such as bicyclo[2.2.1]heptane (norbornane) and bicyclo[2.2.2]octane. As the condensed cyclic group, a group having a condensed ring in which two or three aromatic rings are condensed with a bicycloalkane is preferable, and a group having a condensed ring in which two or three aromatic rings are condensed with bicyclo[2.2.2]octane is more preferable. Specific examples of the condensed cyclic group as R¹⁰¹ include those represented by Formulae (r-br-1) and (r-br-2). In the formulae, * represents a bonding site bonded to Y¹⁰¹ in Formula (b-1).

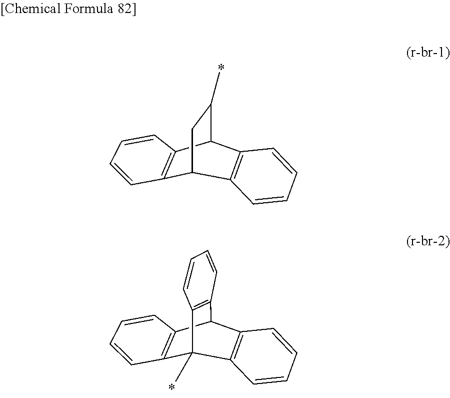

Examples of the substituent that the condensed cyclic group as R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed cyclic group include those described as the substituent of the cyclic group as R¹.

Examples of the aromatic hydrocarbon group as the substituent of the condensed cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Chemical Formulae (r-hr-1) to (r-hr-6).

Examples of the alicyclic hydrocarbon group as the substituent of the condensed cyclic group include a group in which one hydrogen atom has been removed from a monocycloalkane such as cyclopentane or cyclohexane, a group in which one hydrogen atom has been removed from a polycycloalkane such as adamantane, norbornane, isobornane, tricyclodecane, or tetracyclododecane, a lactone-containing cyclic group represented by any of General Formulae (a2-r-1) to (a2-r-7), a —SO₂-containing cyclic group represented by any of General Formulae (b5-r-1) to (b5-r-4), and a heterocyclic group represented by any of Formulae (r-hr-7) to (r-hr-16).

Chain-Like Alkyl Group which May have Substituent:

The chain-like alkyl group as R⁰ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R¹⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹.

Among the examples, R¹⁰¹ represents preferably a cyclic group which may have a substituent, and more preferably a cyclic hydrocarbon group which may have a substituent.

More specifically, as the cyclic hydrocarbon group, a group in which one or more hydrogen atoms have been removed from a phenyl group, a naphthyl group, or a polycycloalkane; a lactone-containing cyclic group represented by any of General Formulae (a2-r-1) to (a2-r-7); or a —SO₂-containing cyclic group represented by any of General Formulae (b5-r-1) to (b5-r-4) is preferable, a group in which one or more hydrogen atoms have been removed from a polycycloalkane is more preferable, and an adamantyl group is still more preferable.

In Formula (b-1), Y¹⁰¹ represents a single bond or a divalent linking group having an oxygen atom.

In a case where Y¹⁰¹ represents a divalent linking group having an oxygen atom, Y¹⁰¹ may have an atom other than the oxygen atom. Examples of atoms other than an oxygen atom include a carbon atom, a hydrogen atom, a sulfur atom, and a nitrogen atom.

Examples of the divalent linking group having an oxygen atom include linking groups each represented by General Formulae (y-a1-1) to (y-a1-8). Further, in General Formulae (y-a1-1) to (y-a1-8), V′¹⁰¹ in General Formulae (y-a1-1) to (y-a1-8) is bonded to R¹⁰¹ in Formula (b-1).

Y¹⁰¹ represents preferably a divalent linking group having an ester bond or a divalent linking group having an ether bond and more preferably a linking group represented by any of Formulae (y-a1-1) to (y-a1-6).

In Formula (b-1), V¹⁰¹ represents a single bond, an alkylene group, or a fluorinated alkylene group. It is preferable that the alkylene group and the fluorinated alkylene group as V¹⁰¹ have 1 to 4 carbon atoms. Among these, V¹⁰¹ represents preferably a single bond or a linear alkylene group having 1 to 4 carbon atoms.

Here, Y¹⁰¹ and V¹⁰¹ in Formula (b-1) do not simultaneously form a single bond.

In Formula (b-1), R¹⁰² represents a fluorine atom or a fluorinated alkyl group having 1 to 5 carbon atoms. R¹⁰² represents preferably a fluorine atom or a perfluoroalkyl group having 1 to 5 carbon atoms and more preferably a fluorine atom.

In a case where Y¹⁰¹ represents a single bond, specific example of the anion moiety represented by Formula (b-1) include a fluorinated alkylsulfonate anion such as a trifluoromethanesulfonate anion or a perfluorobutanesulfonate anion. Further, in a case where Y¹⁰¹ represents a divalent linking group having an oxygen atom, specific examples thereof include an anion represented by any of Formulae (an-1) to (an-3).

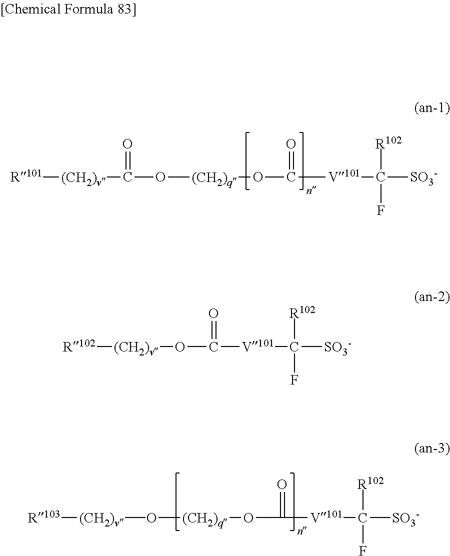

[In the formulae, R″¹⁰¹ represents an aliphatic cyclic group which may have a substituent, a monovalent heterocyclic group represented by any of Chemical Formulae (r-hr-1) to (r-hr-6), a condensed cyclic group represented by Formula (r-br-1) or (r-br-2), a chain-like alkyl group which may have a substituent, or an aromatic cyclic group which may have a substituent. R″¹⁰² represents an aliphatic cyclic group which may have a substituent, a condensed cyclic group represented by Formula (r-br-1) or (r-br-2), a lactone-containing cyclic group represented by any of General Formulae (a2-r-1) and (a2-r-3) to (a2-r-7), or a —S02-containing cyclic group represented by any of General Formulae (b5-r-1) to (b5-r-4). R″¹⁰³ represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer of 0 to 3, each q″ independently represents an integer of 0 to 20, and n″ represents 0 or 1.]

As the aliphatic cyclic group as R″¹⁰¹, R″¹⁰², and R″¹⁰³ which may have a substituent, the same groups as those for the cyclic aliphatic hydrocarbon group as R¹⁰¹ in Formula (b-1) are preferable. Examples of the substituent include the same groups as those for the substituent which may substitute the cyclic aliphatic hydrocarbon group as R¹⁰¹ in Formula (b-1).

The aromatic cyclic group which may have a substituent as R″¹⁰¹ and R″¹⁰³ is preferably the group described as the example of the aromatic hydrocarbon group for the cyclic hydrocarbon group as R¹⁰¹ in Formula (b-1). Examples of the substituent include the same groups as those for the substituent which may substitute the aromatic hydrocarbon group as R¹⁰¹ in Formula (b-1).

As the chain-like alkyl group as R″¹⁰¹ which may have a substituent, the same groups as those for the chain-like alkyl group as R¹⁰¹ in Formula (b-1) are preferable.

As the chain-like alkenyl group as R″¹⁰³ which may have a substituent, the same groups as those for the chain-like alkenyl group as R¹⁰¹ in Formula (b-1) are preferable.

The alkylene group and the fluorinated alkylene group as V″¹⁰1 have preferably 1 to 3 carbon atoms and more preferably 1 or 2 carbon atoms. Specific examples of V″¹⁰¹ include —CH₂—, —(CH₂)₂—, —CFH—, —CH₂CFH—, and —CH(CF₃)—.

As the anion moiety represented by Formula (b-1), an anion moiety represented by Formula (an-1) is preferable. Among these, R″¹⁰¹ in Formula (an-1) represents preferably an aromatic cyclic group which may have a substituent, and more preferably a phenyl group which may have a substituent. Examples of the substituent include a hydroxy group, an alkyl group, and a halogen atom. As the halogen atom, a bromine atom or an iodine atom is preferable, and an iodine atom is more preferable.

Anions in Component (b-2)

In Formula (b-2), R¹⁰⁴ and R¹⁰⁵ each independently represent a cyclic group which may have a substituent, a chain-like alkyl group which may have a substituent, or a chain-like alkenyl group which may have a substituent, and examples thereof include those for R¹⁰¹ in Formula (b-1). Here, R¹⁰⁴ and R¹⁰⁵ may be bonded to each other to form a ring.

R¹⁰⁴ and R¹⁰⁵ represent preferably a chain-like alkyl group which may have a substituent and more preferably a linear or branched alkyl group or a linear or branched fluorinated alkyl group.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decreases within the range of the number of carbon atoms because the solubility in a solvent for a resist is also satisfactory. Further, in the chain-like alkyl group as R¹⁰⁴ and R¹⁰⁵, it is preferable that the number of hydrogen atoms substituted with fluorine atoms is as large as possible because the acid strength increases and the transparency to high energy light with a wavelength of 250 nm or less or electron beams is improved. The proportion of fluorine atoms in the chain-like alkyl group, that is, the fluorination ratio is preferably in a range of 70% to 100% and more preferably in a range of 90% to 100%, and it is most preferable that the chain-like alkyl group is a perfluoroalkyl group in which all hydrogen atoms are substituted with fluorine atoms.

In Formula (b-2), V¹⁰² and V¹⁰³ each independently represent a single bond, an alkylene group, or a fluorinated alkylene group, and examples thereof include the same groups as those for V¹⁰¹ in Formula (b-1).

In Formula (b-2), L¹⁰¹ and L¹⁰² each independently represent a single bond or an oxygen atom.

Anions in Component (b-3)

In Formula (b-3), R¹⁰⁶ to R¹⁰⁸ each independently represent a cyclic group which may have a substituent, a chain-like alkyl group which may have a substituent, or a chain-like alkenyl group which may have a substituent, and examples thereof include those for R¹⁰¹ in Formula (b-1).

In Formula (b-3), L¹⁰³ to L¹⁰⁵ each independently represent a single bond, —CO—, or —SO₂—.

### {Cation Moiety}

In Formulae (b-1), (b-2), and (b-3) described above, M′^{m+} represents an m-valent onium cation. Among them, a sulfonium cation and an iodonium cation are preferable.

m represents an integer of 1 or greater.

Preferred examples of the cation moiety ((M′^{m+})_{1/m}) include onium cations each represented by General Formulae (ca-1) to (ca-3). As the cation moiety, a cation represented by General Formula (ca-1) is more preferable, and a cation represented by any of Formulae (ca-1-1) to (ca-1-75) is still more preferable.

In the resist composition according to the present embodiment, the component (B) may be used alone or in combination of two or more kinds thereof. In the resist composition of the present embodiment, since the above-described component (A1) has the constitutional unit (a0), the resist composition may contain no component (B).

Among the examples, the component (b-1) is preferable as the component (B).

The content of the component (B) in the resist composition is preferably in a range of 0 to 30 parts by mass, more preferably in a range of 0 to 20 parts by mass, and still more preferably in a range of 0 to 10 parts by mass with respect to 100 parts by mass of the component (A1).

It is preferable that the content of the component (B) is set to be in the above-described preferable ranges from the viewpoint that a uniform solution is likely to be obtained in a case where each component of the resist composition is dissolved in an organic solvent, and the storage stability of the resist composition is enhanced.

### <<Base Component (D)>>

It is preferable that the resist composition according to the present embodiment further contains a base component (hereinafter, also referred to as “component (D)”) that traps (that is, controls the acid diffusion) an acid that is generated upon light exposure, in addition to the component (A). The component (D) acts as a quencher (an acid diffusion control agent) which traps the acid generated in the resist composition upon light exposure.

Examples of the component (D) include a photodecomposable base (D1) having acid diffusion controllability (hereinafter, referred to as “component (D1)”) which is lost by the decomposition upon light exposure and a nitrogen-containing organic compound (D2) (hereinafter, referred to as “component (D2)”) which does not correspond to the component (D1). Among these, a photodecomposable base (component (D1)) is preferable from the viewpoint of easily enhancing the pattern dimension uniformity and the roughness reducing property. Further, in a case where the component (D1) is contained, both the characteristics of enhancing the sensitivity and suppressing the occurrence of coating defects are likely to be enhanced. The compound described as the component (D1) below may be used as the acid generator component (component (B)) depending on the combination with other compounds.

### In Regard to Component (D1)

In a case where a resist composition containing the component (D1) is obtained, the contrast between an exposed portion and an unexposed portion of the resist film can be further improved in a case of forming a resist pattern.

The component (D1) is not particularly limited as long as the component is decomposed upon light exposure and loses acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose the acid diffusion controllability (basicity), the components (d1-1) to (d1-3) do not act as a quencher at the exposed portion of the resist film, but act as a quencher at the unexposed portion of the resist film.

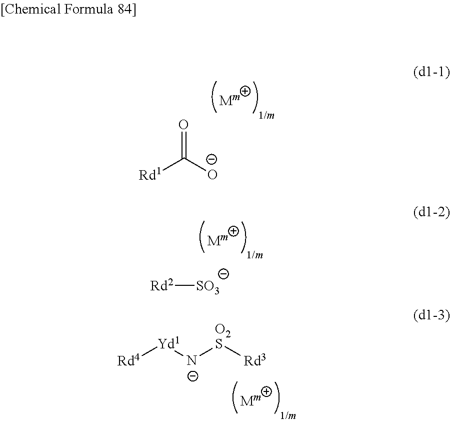

[In the formulae, Rd¹ to Rd⁴ represent a cyclic group which may have a substituent, a chain-like alkyl group which may have a substituent, or a chain-like alkenyl group which may have a substituent. Here, the carbon atom adjacent to the S atom as Rd² in Formula (d1-2) has no fluorine atom bonded thereto. Yd¹ represents a single bond or a divalent linking group. m represents an integer of 1 or greater, and M^{m+}'s each independently represent an m-valent onium cation.]

{Component (d1-1)}

### Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹.

Among these, it is preferable that the group as R^{d1} represents an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of General Formulae (a2-r-1) to (a2-r-7), an ether bond, an ester bond, and a combination thereof. In a case where the composition has an ether bond or an ester bond as the substituent, the substituent may be present through an alkylene group, and a linking group represented by any of Formulae (y-a1-1) to (y-a1-6) is preferable as the substituent in such a case. Further, in a case where the aromatic hydrocarbon group, the aliphatic cyclic group, or the chain-like alkyl group as Rd¹ contains a linking group represented by any of General Formulae (y-a1-1) to (y-a1-8) as a substituent, V′¹⁰¹ in General Formulae (y-a1-1) to (y-a1-8) is bonded to the carbon atom constituting the aromatic hydrocarbon group, the aliphatic cyclic group, or the chain-like alkyl group as Rd¹ in Formula (d3-1), in General Formulae (y-a1-1) to (y-a1-8).

Suitable examples of the aromatic hydrocarbon group include a phenyl group, a naphthyl group, and a polycyclic structure having a bicyclooctane skeleton (for example, a polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclodecane, or tetracyclododecane is more preferable.

It is preferable that the chain-like alkyl group has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-1) are described below.

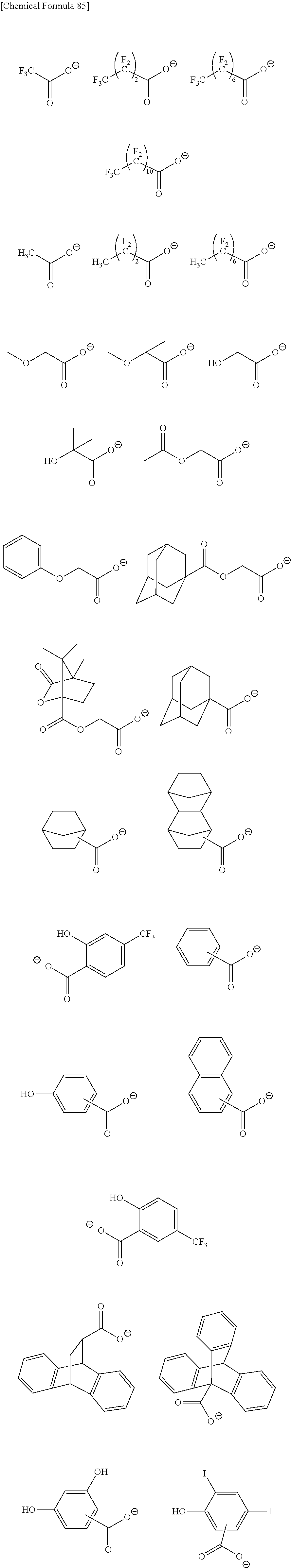

### Cation Moiety

In Formula (d1-1), M^{m+} represents an m-valent onium cation.

Suitable examples of the onium cation as M^{m+} include the same cations as those for the cations each represented by General Formulae (ca-1) to (ca-3). Among these, a cation represented by General Formula (ca-1) is more preferable, and cations each represented by Formulae (ca-1-1) to (ca-1-75) are still more preferable.

The component (d1-1) may be used alone or in combination of two or more kinds thereof.

{Component (d1-2)}

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Here, the carbon atom adjacent to the S atom in Rd² has no fluorine atom bonded thereto (the carbon atom is not substituted with a fluorine atom). As a result, the anion of the component (d1-2) becomes an appropriately weak acid anion, thereby improving the quenching ability of the component (D).

Rd² represents preferably a chain-like alkyl group which may have a substituent or an aliphatic cyclic group which may have a substituent, and more preferably an aliphatic cyclic group which may have a substituent.

The chain-like alkyl group preferably has 1 to 10 carbon atoms and more preferably 3 to 10 carbon atoms.

The aliphatic cyclic group is more preferably a group (which may have a substituent) obtained by removing one or more hydrogen atoms from adamantane, norbornane, isobornane, tricyclodecane, tetracyclododecane, or the like; and a group obtained by removing one or more hydrogen atoms from camphor.

The hydrocarbon group as Rd² may have a substituent, and examples of the substituent include the same groups as those for the substituent which may be included in the hydrocarbon group (such as an aromatic hydrocarbon group, an aliphatic cyclic group, or a chain-like alkyl group) as Rd¹ in Formula (d1-1).

Specific preferred examples of the anion moiety in the component (d1-2) are described below.

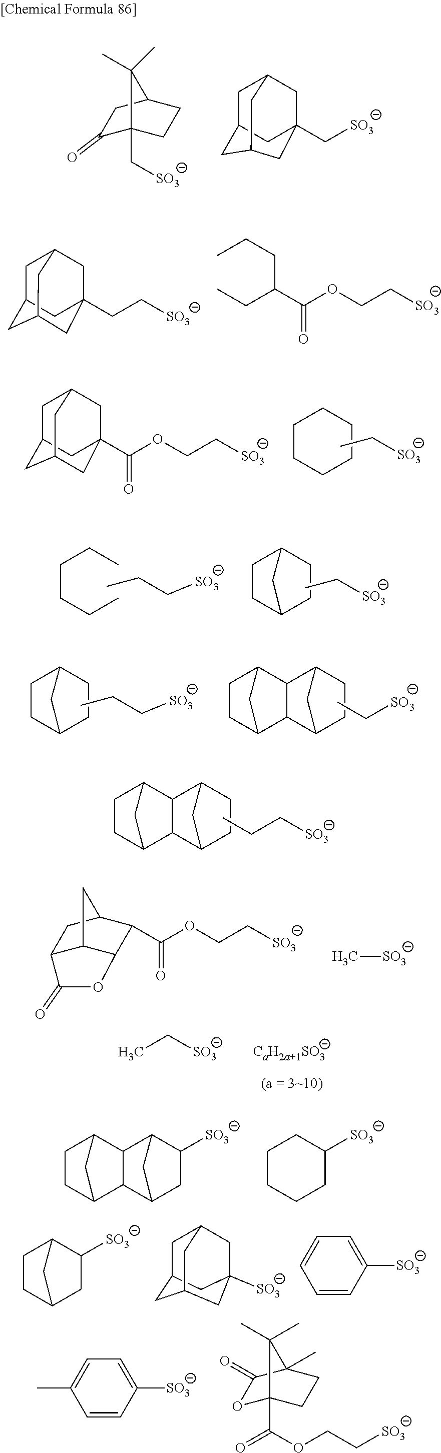

### Cation Moiety

In Formula (d1-2), M^{m+} represents an m-valent onium cation and has the same definition as that for M^{m+} in Formula (d1-1).

The component (d1-2) may be used alone or in combination of two or more kinds thereof.

{Component (d1-3)}

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R′²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ is a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ is an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may have an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclodecane or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are enhanced. Further, in a case where Rd⁴ represents an aromatic group, the resist composition has excellent light absorption efficiency in lithography using EUV or the like as an exposure light source, and thus the sensitivity and lithography characteristics are enhanced.

In Formula (d1-3), Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group having a heteroatom. Examples thereof include the same groups as those for the divalent hydrocarbon group which may have a substituent and the divalent linking group having a heteroatom described in the section of the divalent linking group as Ya^{x1} in Formula (a10-1).

It is preferable that Yd¹ represents a carbonyl group, an ester bond, an amide bond, an alkylene group, or a combination of these. As the alkylene group, a linear or branched alkylene group is more preferable, and a methylene group or an ethylene group is still more preferable.

Specific preferred examples of the anion moiety in the component (d1-3) are described below.

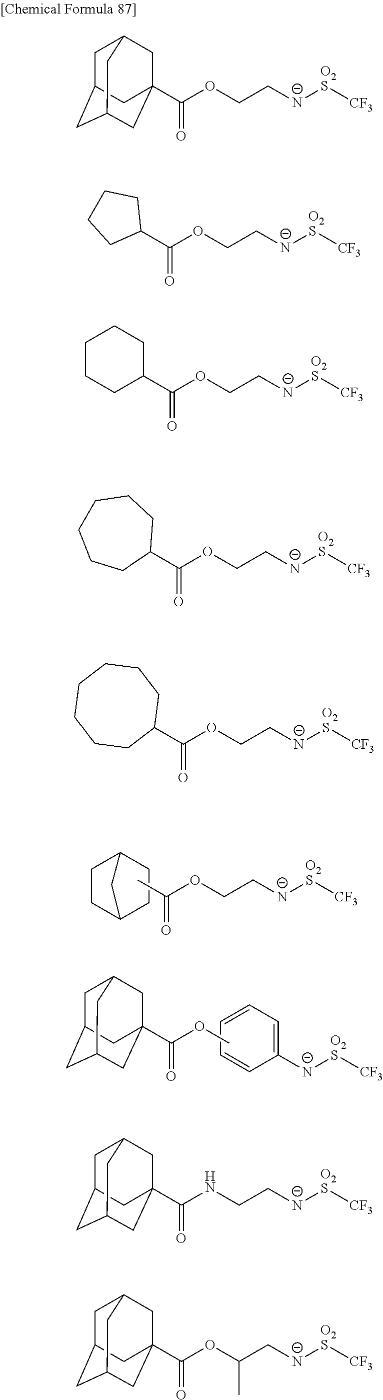

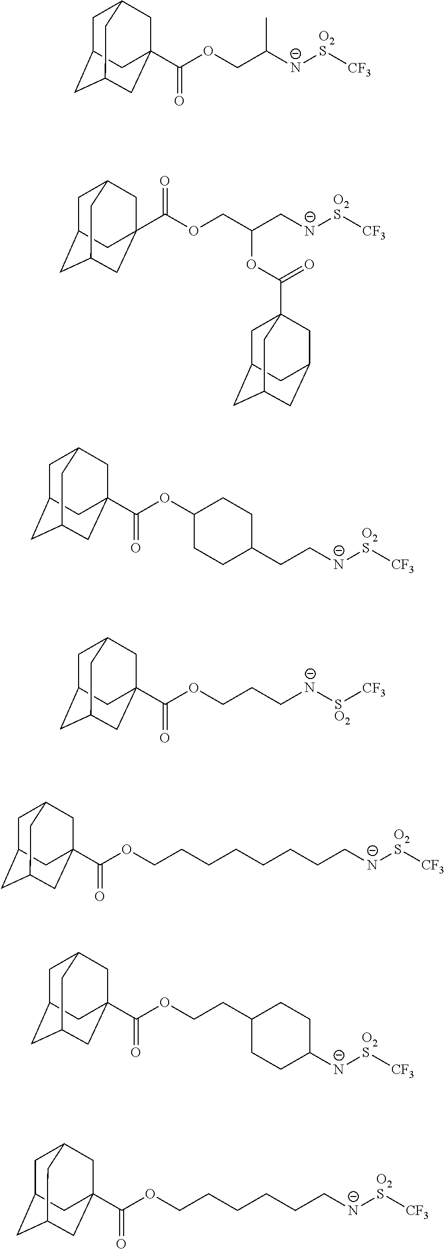

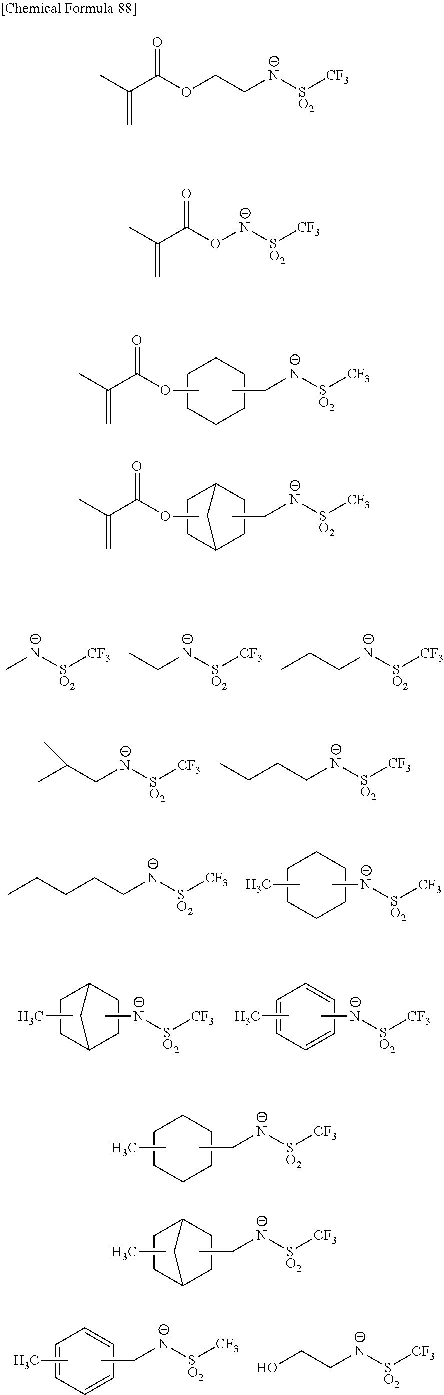

### Cation Moiety

In Formula (d1-3), M^{m+} represents an m-valent onium cation and has the same definition as that for M^{m+} in Formula (d1-1).

The component (d1-3) may be used alone or in combination of two or more kinds thereof.

As the component (D1), only one of the above-described components (d1-1) to (d1-3) or a combination of two or more kinds thereof may be used.

In the resist composition according to the present embodiment, it is preferable that the component (D1) contains the component (d1-1).

In a case where the resist composition contains the component (D1), the content of the component (D1) in the resist composition is preferably in a range of 0.5 to 15 parts by mass, more preferably in a range of 1 to 10 parts by mass, and still more preferably in a range of 2 to 9 parts by mass with respect to 100 parts by mass of the component (A1).

In a case where the content of the component (D1) is greater than or equal to the lower limits of the above-described preferable ranges, particularly excellent lithography characteristics and an excellent resist pattern shape are easily obtained. On the contrary, in a case where the content is less than or equal to the upper limits of the above-described ranges, the sensitivity can be satisfactorily maintained and the throughput is also excellent.

The content of the component (d1-1) in the total amount of the component (D) contained in the resist composition according to the present embodiment is preferably 50% by mass or greater, more preferably 70% by mass or greater, and still more preferably 90% by mass or greater, and the component (D) may be formed of only the component (d1-1).

### Method of Producing Component (D1):

The methods of producing the component (d1-1) and the component (d1-2) are not particularly limited, and these components can be produced by known methods.

Further, the method of producing the component (d1-3) is not particularly limited, and the component is produced by the same method as disclosed in United States Patent Application, Publication No. 2012-0149916.

### In Regard to Component (D2)

The component (D) may contain a nitrogen-containing organic compound component (hereinafter, referred to as “component (D2)”) that does not correspond to the component (D1) described above.

The component (D2) is not particularly limited as long as the component acts as an acid diffusion control agent and does not correspond to the component (D1), and an optional component may be selected from known components and then used. Among the examples, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of these aliphatic amines include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylanine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 6 to 30 carbon atoms is still more preferable, and tri-n-pentylamine or tri-n-octylamine is particularly preferable.

Examples of the cyclic amine include a heterocyclic compound having a nitrogen atom as a heteroatom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine, and triethanolamine triacetate. Among these, triethanolamine triacetate is preferable.

As the component (D2), an aromatic amine may be used.

Examples of the aromatic amine include 4-dimethylaminopyridine, pyrrole, indole, pyrazole, inidazole, and derivatives thereof, tribenzylamine, 2,6-diisopropylaniline, N-tert-butoxycarbonylpyrrolidine, 2,6-di-tert-butylpyridine, and 2,6-tert-butylpyridine.

Among the examples, the component (D2) is preferably an alkylamine and more preferably a trialkylamine having 6 to 30 carbon atoms.

The component (D2) may be used alone or in combination of two or more kinds thereof.

In a case where the resist composition contains the component (D2), the content of the component (D2) in the resist composition is preferably in a range of 0.01 to 5 parts by mass, more preferably in a range of 0.1 to 5 parts by mass, and still more preferably in a range of 0.5 to 5 parts by mass with respect to 100 parts by mass of the component (A1).

In a case where the content of the component (D2) is greater than or equal to the lower limits of the above-described preferable ranges, excellent lithography characteristics and an excellent resist pattern shape are easily obtained. On the contrary, in a case where the content is less than or equal to the upper limits of the above-described ranges, the sensitivity can be satisfactorily maintained and the throughput is also excellent.

<<At Least One Compound (E) Selected from Group Consisting of Organic Carboxylic Acids, Phosphorus Oxo Acids, and Derivatives Thereof>>

For the purpose of preventing any deterioration in sensitivity and improving the resist pattern shape and the post exposure temporal stability, the resist composition according to the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

In the resist composition of the present embodiment, the component (E) may be used alone or in combination of two or more kinds thereof.

In a case where the resist composition contains the component (E), the content of the component (E) is preferably in a range of 0.01 to 5 parts by mass and more preferably in a range of 0.05 to 3 parts by mass with respect to 100 parts by mass of the component (A1). In a case where the content thereof is in the above-described ranges, the lithography characteristics are further improved.

### <<Fluorine Additive Component (F)>>

The resist composition according to the present embodiment may further contain a fluorine additive component (hereinafter, referred to as “component (F)”) as a hydrophobic resin. Since the component (F) is used to impart water repellency to the resist film and used as a resin different from the component (A1), the lithography characteristics can be improved.

As the component (F), for example, the fluorine-containing polymer compounds described in Japanese Unexamined Patent Application, First Publication Nos. 2010-002870, 2010-032994, 2010-277043, 2011-13569, and 2011-128226 can be used.

Specific examples of the component (F) include a polymer having a constitutional unit (f1) represented by General Formula (f1-1). As the polymer, a polymer (homopolymer) formed of only the constitutional unit (f1) represented by Formula (f1-1); a copolymer of the constitutional unit (f1) and the constitutional unit

(a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

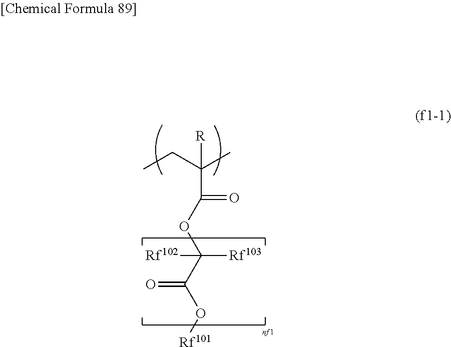

[In the formula, R has the same definition as described above, Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. nf¹ represents an integer of 0 to 5, and Rf¹⁰¹ represents an organic group having a fluorine atom.]

In Formula (f1-1), R bonded to the carbon atom at the α-position has the same definition as described above. It is preferable that R represents a hydrogen atom or a methyl group.

In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among the examples, a methyl group or an ethyl group is preferable. Specific examples of the halogenated alkyl group having 1 to 5 carbon atoms for Rf¹⁰² and Rf¹⁰³ include groups in which some or all hydrogen atoms of an alkyl group having 1 to 5 carbon atoms have been substituted with halogen atoms. Among these, a fluorine atom is preferable as the halogen atom. Among these, Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10.

In the hydrocarbon group having a fluorine atom, preferably 25% or greater of the hydrogen atoms in the hydrocarbon group are fluorinated, more preferably 50% or greater thereof are fluorinated, and still more preferably 60% or greater thereof are fluorinated from the viewpoint of increasing the hydrophobicity of the resist film during immersion exposure.

Among examples, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of polystyrene according to gel permeation chromatography) of the component (F) is preferably in a range of 1000 to 50000, more preferably in a range of 5000 to 40000, and most preferably in a range of 10000 to 30000. In a case where the weight-average molecular weight thereof is less than or equal to the upper limits of the above-described ranges, the resist composition exhibits a satisfactory solubility in a solvent for a resist enough to be used as a resist. Meanwhile, in a case where the weight-average molecular weight thereof is greater than or equal to the lower limits of the above-described ranges, water repellency of the resist film is improved.

Further, the polydispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or in combination of two or more kinds thereof.

In a case where the resist composition contains the component (F), the content of the component (F) is preferably in a range of 0.5 to 10 parts by mass and more preferably in a range of 1 to 10 parts by mass with respect to 100 parts by mass of the component (A1).

### <<Organic Solvent Component (S)>>

The resist composition according to the present embodiment may be produced by dissolving the resist materials in an organic solvent component (hereinafter, referred to as a “component (S)”).

In the resist composition of the present embodiment, the component (S) may be used alone or in the form of a mixed solvent of two or more kinds thereof. Among these, PGMEA, PGME, γ-butyrolactone, EL, or cyclohexanone is preferable.

Further, a mixed solvent obtained by mixing PGMEA with a polar solvent is also preferable as the component (S). The blending ratio (mass ratio) may be appropriately determined in consideration of the compatibility between PGMEA and the polar solvent.

A mixed solvent of at least one selected from PGMEA and EL, and γ-butyrolactone is also preferable as the (S) component. In this case, as the mixing ratio, the mass ratio between the former and the latter is preferably in a range of 70:30 to 95:5.

The amount of the component (S) to be used is not particularly limited and is appropriately set to have a concentration which enables coating a substrate or the like depending on the thickness of the coated film. In general, the component (S) is used such that the solid content concentration of the resist composition is in a range of 0.1% to 20% by mass, preferably in a range of 0.2% to 15% by mass, and more preferably in a range of 0.5% to 5% by mass.

After the resist material is dissolved in the component (S), impurities may be removed from the resist composition of the present embodiment using a porous polyimide film, a porous polyamide-imide film, or the like. For example, the resist composition may be filtered using a filter formed of a porous polyimide film, a filter formed of a porous polyamide-imide film, a filter formed of a porous polyimide film and a porous polyamide-imide film, or the like. Examples of the porous polyimide film and the porous polyamide-imide film include those described in Japanese Unexamined Patent Application, First Publication No. 2016-155121.

The resist composition of the present embodiment described above contains a polymer compound having a monomer unit formed of an anion containing a polymerizable group-containing group, an iodine atom, and a carboxylate group, and an onium cation, as a base resin which is a base material component.

Since the polymer compound which is the base resin contains an iodine atom, the light absorption property with respect to light exposure is enhanced. In addition, since the anion moiety containing a carboxylate group is introduced into the base resin as a repeating unit, a site where the quenching effect is exhibited is likely to be uniformly dispersed in the resist film. It is considered that in a case where such an action synergistically acts, the resist composition of the present embodiment can further achieve high sensitivity and improve lithography characteristics such as roughness reduction.

### (Method for Forming a Resist Pattern)

A method for forming a resist pattern according to the second aspect of the present invention is a method including a step of forming a resist film on a support using the resist composition according to the first aspect of the present invention described above, a step of exposing the resist film to light, and a step of developing the resist film exposed to light to form a resist pattern.

According to the embodiment of the method for forming a resist pattern, a method for forming a resist pattern, which is performed in the following manner is an exemplary example.

First, a support is coated with the resist composition of the present embodiment using a spinner or the like, and a bake (post applied bake (PAB)) treatment is performed under a temperature condition of 80° C. to 150° C. for 40 to 120 seconds and preferably 60 to 90 seconds to form a resist film.

Next, the selective light exposure is carried out on the resist film, for example, by light exposure through a mask (mask pattern) having a predetermined pattern formed on the mask by using an exposure apparatus such as an electron beam lithography apparatus or an EUV exposure apparatus, or direct irradiation of the resist film for drawing with an electron beam without using a mask pattern.

Thereafter, a bake treatment (post-exposure bake (PEB)) is performed, for example, under a temperature condition of 80° C. to 150° C. for 40 to 120 seconds and preferably 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, after the developing treatment or the rinse treatment, the developing solution or the rinse solution attached onto the pattern may be removed by a treatment using a supercritical fluid.

After the developing treatment or the rinse treatment, drying is conducted. As desired, a bake treatment (post bake) may be conducted after the developing treatment.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, copper, aluminum, nickel, or gold can be used.

The wavelength to be used for light exposure is not particularly limited and the exposure can be conducted using radiation such as an ArF excimer laser, a KrF excimer laser, an F₂ excimer laser, extreme ultraviolet (EUV) rays, vacuum ultraviolet rays (VUV), electron beams (EB), X-rays, and soft X-rays.

The method for forming a resist pattern according to the present embodiment is a useful method in which the resist film is exposed to extreme ultraviolet (EUV) rays or electron beams (EB) in the step of exposing the resist film to light.

The method of exposing the resist film to light can be general exposure (dry exposure) conducted in air or an inert gas such as nitrogen, or liquid immersion exposure (liquid immersion lithography).

The liquid immersion exposure is an exposure method in which the region between the resist film and the lens at the lowermost position of the exposure apparatus is filled with a solvent (liquid immersion medium) in advance that has a refractive index greater than the refractive index of air, and the exposure (immersion exposure) is conducted in this state.

As the liquid immersion medium, a solvent having a refractive index greater than the refractive index of air but less than the refractive index of the resist film to be exposed is preferable, and examples thereof include water, a fluorine-based inert liquid, a silicon-based solvent, and a hydrocarbon-based solvent.

As the liquid immersion medium, water is preferably used.

As the alkali developing solution used for the developing treatment in the alkali developing process, a 0.1 to 10 mass % tetramethylammonium hydroxide (TMAH) aqueous solution is an exemplary example.

The organic solvent contained in the organic developing solution used for the developing treatment in the solvent developing process may be any solvent that is capable of dissolving the component (A1) (the component (A1) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate.

Examples of the nitrile-based solvent include acetonitrile, propionitrile, valeronitrile, and butyronitrile.

Known additives can be blended into the organic developing solution as necessary. Examples of the additive include a surfactant. The surfactant is not particularly limited, and for example, an ionic or non-ionic fluorine-based and/or silicon-based surfactant can be used.

The developing treatment can be performed according to a known developing method, and examples thereof include a method of immersing a support in a developing solution for a certain time (a dip method), a method of raising a developing solution on the surface of a support using the surface tension and maintaining the state for a certain time (a puddle method), a method of spraying a developing solution to the surface of a support (spray method), and a method of continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used.

These organic solvents may be used alone or in combination of two or more kinds thereof. Further, an organic solvent other than the above-described solvents and water may be mixed and used.

The rinse treatment carried out using a rinse solution (washing treatment) can be performed according to a known rinse method. Examples of the method of performing the rinse treatment include a method of continuously ejecting a rinse solution onto a support rotating at a certain rate (rotary coating method), a method of immersing a support in a rinse solution for a certain time (dip method), and a method of spraying a rinse solution to the surface of a support (spray method).

According to the method for forming a resist pattern of the present embodiment described above, since the resist composition described above is used, a resist pattern that can achieve high sensitivity and has favorable lithography characteristics such as roughness reduction can be formed.

It is preferable that various materials that are used in the resist composition according to the above-described embodiment and the pattern forming method according to the above-described embodiment (for example, a resist solvent, a developing solution, a rinse solution, a composition for forming an antireflection film, and a composition for forming a top coat) do not contain impurities such as a metal, a metal salt containing halogen, an acid, an alkali, and a component having a sulfur atom or phosphorus atom. Here, examples of the impurities containing metal atoms include Na, K, Ca, Fe, Cu, Mn, Mg, Al, Cr, Ni, Zn, Ag, Sn, Pb, Li, and salts thereof. The content of the impurities contained in these materials is preferably 200 ppb or less, more preferably 1 ppb or less, still more preferably 100 parts per trillion (ppt) or less, particularly preferably 10 ppt or less, and most preferably substantially zero (less than or equal to the detection limit of the measuring device).

### (Compound)

A compound according to a third aspect of the present invention is a compound represented by General Formula (a0-m) (hereinafter, also referred to as “compound (a0-m)”).

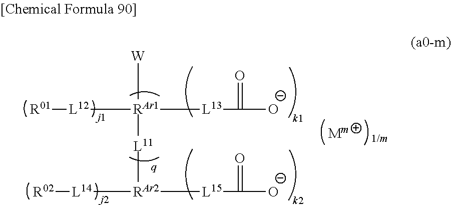

[In the formula, W represents a polymerizable group-containing group. R^{Ar1} and R^{Ar2} each independently represent an aromatic group which may have a substituent. R^{Ar1}-L¹¹- represents R^{Ar1}—O—, R^{Ar1}—S—, R^{Ar1}—NH—, R^{Ar1}-Ak-, R^{Ar1}—C(═O)—O—, R^{Ar1}—C(═O)—NH—, R^{Ar1}—O—C(═O)—, R^{Ar1}—NH—C(═O)—, R^{Ar1}-Ak-O—, R^{Ar1}-Ak-NH—, R^{Ar1}—O—C(═O)-Ak-, or R^{Ar1}— (in a case where L¹¹ represents a single bond). Ak represents an alkylene group or a fluorinated alkylene group (the same applies hereinafter). R^{Ar1}-L¹²- represents R^{Ar1}-Ak-, R^{Ar1}—C(═O)—O-Ak-, R^{Ar1}—C(═O)—NH-Ak-, R^{Ar1}—O—C(═O)-Ak-, R^{Ar1}—NH—C(═O)-Ak-. R^{Ar1}—O—C(═O)—. or R^{Ar1}— (in a case where L¹² represents a single bond). R^{Ar1}-L¹³- represents R^{Ar1}—O—, R^{Ar1}—S—, R^{Ar1}—NH—, R^{Ar1}-Ak-, R^{Ar1}—O-Ak-, or R^{Ar1}— (in a case where L¹³ represents a single bond). R^{Ar2}-L¹⁴- represents R^{Ar2}-Ak-, R^{Ar2}—C(═O)—O-Ak-, R^{Ar2}—C(═O)—NH-Ak-, R^{Ar2}—O—C(═O)-Ak-, R^{Ar2}—NH—C(═O)-Ak-, R^{Ar2}—O—C(═O)—, or R^{Ar2}— (in a case where L¹⁴ represents a single bond). R^{Ar}2-L¹⁵- represents R^{Ar2}—O—, R^{Ar2}—S—, R^{Ar2}—NH—, R^{Ar2}-Ak-, R^{Ar2}—O-Ak-, or R^{Ar2}— (in a case where L¹⁵ represents a single bond). q represents 0 or 1. R⁰¹ and R⁰² each independently represent an iodinated alkyl group, an iodine atom, or a hydrogen atom. j1 and j2 each independently represent an integer of 0 to 5. Here, in a case where q represents 0, R⁰² represents an iodinated alkyl group or an iodine atom, and j2 represents an integer of 1 to 5. In a case where q represents 1, the number of iodine atoms in R^{0′} and R⁰² is 1 or more. k1 and k2 each independently represent 0 or 1. Here, in a case where q represents 0, k2 represents 1. In a case where q represents 1, k1+k2 is 1. M^{m+} represents an m-valent onium cation. m represents an integer of 1 or greater.]

The description of the compound represented by General Formula (a0-m) (compound (a0-m)) is the same as the description of the compound represented by General Formula (a0-m) described in the section of <<Constitutional unit (a0)>> above.

It is preferable that, as the compound (a0-m), W in General Formula (a0-m) represents a polymerizable group-containing group represented by General Formula (a0-w).

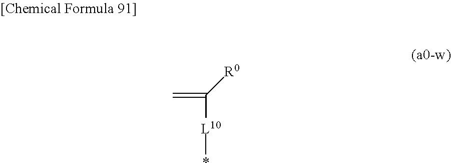

[In the formula, R⁰ represents an alkyl group having 1 to 5 carbon atoms, a halogenated alkyl group having 1 to 5 carbon atoms, or a hydrogen atom. -L¹⁰-* represents —C(═O)—O—*, —C(═O)—NH—*, or -*(in a case where L¹⁰ represents a single bond). * represents a bonding site that is bonded to RA in General Formula (a0-m) or a bonding site that is bonded to R^{Ar2} in a case where q represents 0.]

Suitable examples of the compound (a0-m) include a compound in which, in General Formula (a0-m), W represents a polymerizable group-containing group represented by General Formula (a0-w), q represents 1, k1 represents 1, k2 represents 0, R⁰² represents an iodinated alkyl group or an iodine atom, j2 represents an integer of 1 to 5, and L¹⁰ in General Formula (a0-w) represents a single bond, that is, the compound represented by General Formula (a01-m).

Alternatively, suitable examples of the compound (a0-m) include a compound in which, in General Formula (a0-m), W represents a polymerizable group-containing group represented by General Formula (a0-w), q represents 1, k1 represents 0, k2 represents 1, and L¹⁰ in General Formula (a0-w) represents a single bond, that is, the compound represented by General Formula (a02-m). A compound in which R⁰² represents an iodinated alkyl group or an iodine atom and j2 represents an integer of 1 to 5 is more preferable.

Alternatively, suitable examples of the compound (a0-m) include a compound in which, in General Formula (a0-m), W represents a polymerizable group-containing group represented by General Formula (a0-w), q represents 0, k2 represents 1, R⁰² represents an iodinated alkyl group or an iodine atom, j2 represents an integer of 1 to 5, and L¹⁰ in General Formula (a0-w) represents a single bond, that is, the compound represented by General Formula (a03-m).

Alternatively, suitable examples of the compound (a0-m) include a compound in which, in General Formula (a0-m), W represents a polymerizable group-containing group represented by General Formula (a0-w), q represents 0, k2 represents 1, R⁰² represents an iodinated alkyl group or an iodine atom, j2 represents an integer of 1 to 5, and -L¹⁰-* in General Formula (a0-w) represents —C(═O)—O—* or —C(═O)—NH—* [here, * represents a bonding site that is bonded to R^{Ar2} in General Formula (a0-m)], that is, the compound represented by General Formula (a04″-m).

In the compounds described as the suitable examples above, it is preferable that M′^{m+} in General Formula (a0-m) represents an m-valent sulfonium cation.

Specific examples of the compound (a0-m) are shown below, but the present invention is not limited thereto.

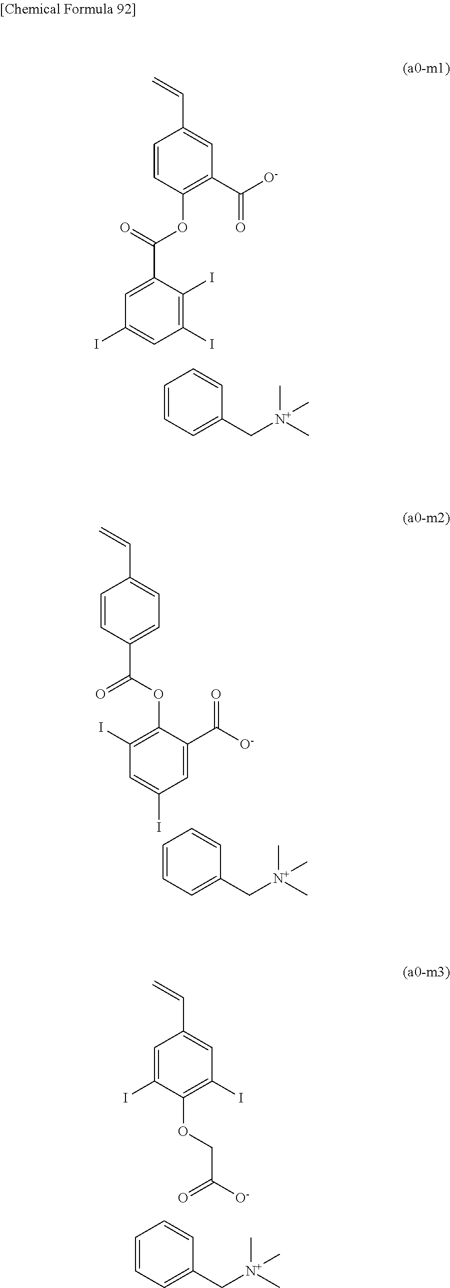 [SMILES: C=Cc1ccc(OC(=O)c2cc(I)cc(I)c2I)c(C(=O)[O-])c1 ; C[N+](C)(C)Cc1ccccc1 ; C=Cc1ccc(C(=O)Oc2c(I)cc(I)cc2C(=O)[O-])cc1 ; C[N+](C)(C)Cc1ccccc1 ; C=Cc1cc(I)c(OCC(=O)[O-])c(I)c1 ; C[N+](C)(C)Cc1ccccc1]

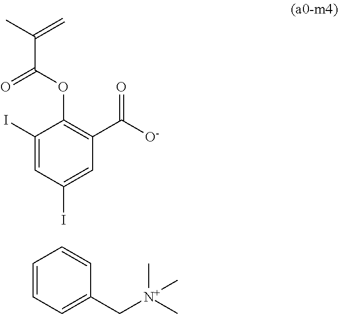 [SMILES: C=C(C)C(=O)Oc1c(I)cc(I)cc1C(=O)[O-] ; C[N+](C)(C)Cc1ccccc1]

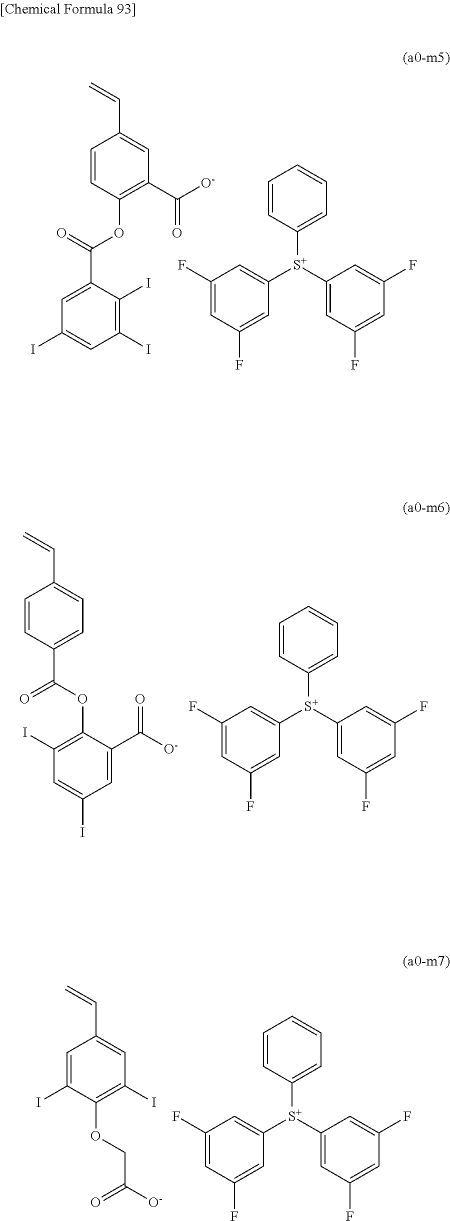 [SMILES: C=Cc1ccc(OC(=O)c2cc(I)cc(I)c2I)c(C(=O)[O-])c1 ; Fc1cc(F)cc([S+](c2ccccc2)c2cc(F)cc(F)c2)c1 ; C=Cc1ccc(C(=O)Oc2c(I)cc(I)cc2C(=O)[O-])cc1 ; Fc1cc(F)cc([S+](c2ccccc2)c2cc(F)cc(F)c2)c1 ; C=Cc1cc(I)c(OCC(=O)[O-])c(I)c1 ; Fc1cc(F)cc([S+](c2ccccc2)c2cc(F)cc(F)c2)c1]

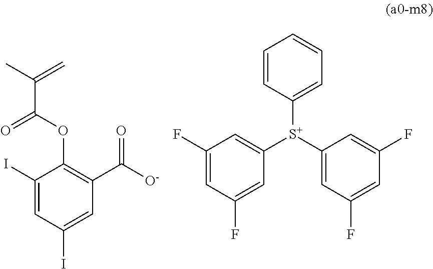 [SMILES: C=C(C)C(=O)Oc1c(I)cc(I)cc1C(=O)[O-] ; Fc1cc(F)cc([S+](c2ccccc2)c2cc(F)cc(F)c2)c1]

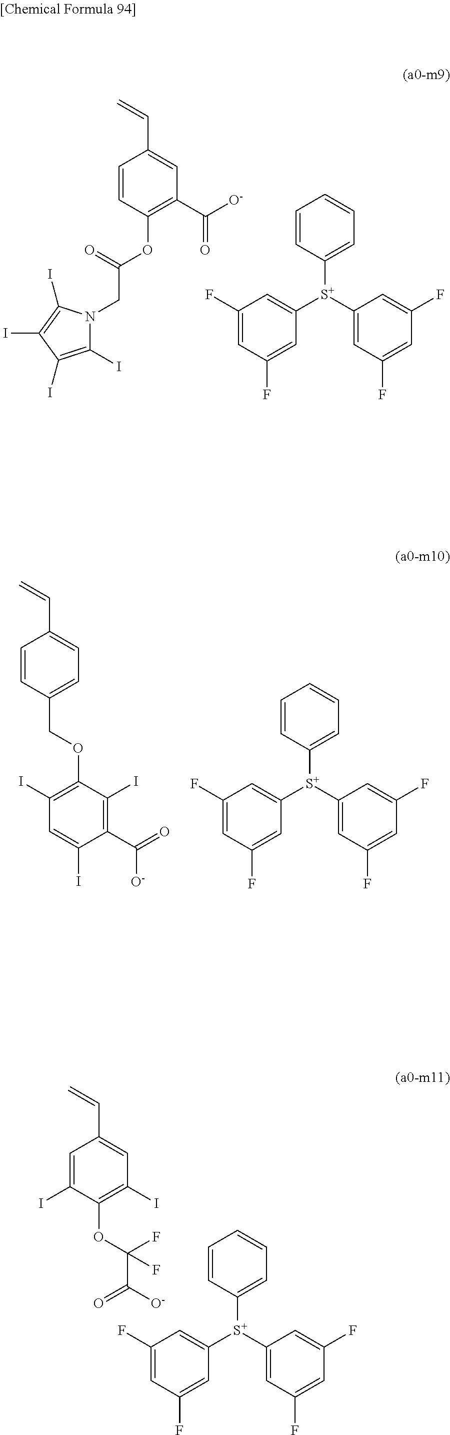 [SMILES: C=Cc1ccc(OC(=O)Cn2c(I)c(I)c(I)c2I)c(C(=O)[O-])c1 ; Fc1cc(F)cc([S+](c2ccccc2)c2cc(F)cc(F)c2)c1 ; C=Cc1ccc(COc2c(I)cc(I)c(C(=O)[O-])c2I)cc1 ; Fc1cc(F)cc([S+](c2ccccc2)c2cc(F)cc(F)c2)c1 ; C=Cc1cc(I)c(OC(F)(F)C(=O)[O-])c(I)c1 ; Fc1cc(F)cc([S+](c2ccccc2)c2cc(F)cc(F)c2)c1]

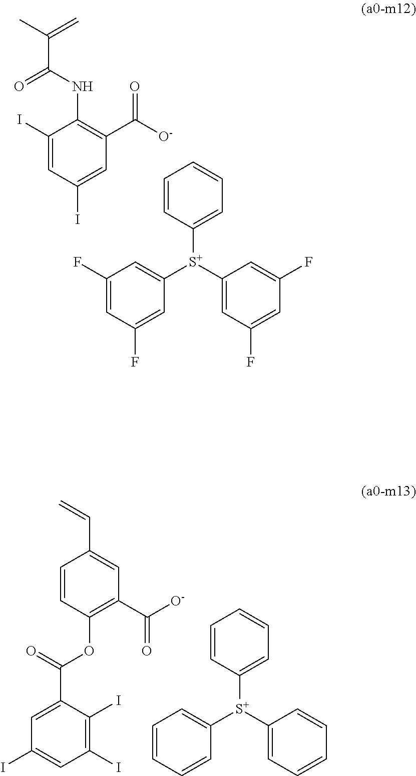 [SMILES: C=C(C)C(=O)Nc1c(I)cc(I)cc1C(=O)[O-] ; Fc1cc(F)cc([S+](c2ccccc2)c2cc(F)cc(F)c2)c1 ; C=Cc1ccc(OC(=O)c2cc(I)cc(I)c2I)c(C(=O)[O-])c1 ; c1ccc([S+](c2ccccc2)c2ccccc2)cc1]

[Method for Producing Compound (a0-m)]

The compound (a0-m) of the present embodiment can be produced by appropriately combining known methods.

The compound represented by General Formula (a01-m) can be produced, for example, by esterifying and hydrolyzing phenols containing a polymerizable group and a tertiary alkyl ester group with an aromatic carboxylic acid substituted with an iodine atom and carrying out salt exchange on the resultant with a desired onium salt, as described in [Synthesis Example 1: synthesis of compound (a0-m1)] below.

The compound represented by General Formula (a02-m) can be produced, for example, by esterifying and hydrolyzing an aromatic carboxylic acid containing a polymerizable group with phenols substituted with an iodine atom and a tertiary alkyl ester group and carrying out salt exchange on the resultant with a desired onium salt, as described in [Synthesis Example 2: synthesis of compound (a0-m2)] below.

The compound represented by General Formula (a03-m) can be produced, for example, by reacting (etherifying) phenols containing a polymerizable group and an iodine atom with an alkylating agent (ethyl bromoacetate or the like) in the presence of a base and carrying out salt exchange on the resultant with a desired onium salt, as described in [Synthesis Example 3: synthesis of compound (a0-m3)] below.

The compound represented by General Formula (a04-m) or (a04″-m) can be produced, for example, by acylating (esterifying) an acylating agent (methacrylic acid chloride) and an aromatic carboxylic acid substituted with a hydroxy group (—OH) and an iodine atom in the presence of a base and carrying out salt exchange on the resultant with a desired onium salt, as described in [Synthesis Example 4: synthesis of compound (a0-m4)] below.

Alternatively, the compound represented by General Formula (a04-m) or (a04″-m) can be produced, for example, by acylating (amidating) an acylating agent (methacrylic acid chloride) and an aromatic carboxylic acid substituted with an amino group (—NH₂) and an iodine atom in the presence of a base and carrying out salt exchange on the resultant with a desired onium salt, as described in [Synthesis Example 4: synthesis of compound (a0-m4)] below.

After completion of each reaction, the compound (a0-m) in the reaction solution may be isolated and purified.

Known methods of the related art can be used for the isolation and the purification, and for example, any one or a combination of two or more of concentrations, solvent extraction, distillation, crystallization, recrystallization, chromatography, and the like can be used.

The structure of the compound obtained as described above can be identified by typical organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), elemental analysis, and X-ray crystal diffraction.

The compound according to the present embodiment can be used as a raw material monomer of a polymer compound according to a fourth aspect described below.

### (Polymer Compound)

The fourth aspect of the present invention relates to a polymer compound having a constitutional unit derived from the compound according to the third aspect of the present invention described above.

The constitutional unit derived from the compound according to the third aspect is a constitutional unit derived from the compound represented by General Formula (a0-m), and is the same as the constitutional unit (a0) described above.

The description of the polymer compound according to the fourth aspect is the same as the description of the component (A1).

The polymer compound according to the fourth aspect, that is, the constitutional unit (constitutional unit (a0)) derived from the compound represented by General Formula (a0-m) can be used as the base material component of the resist composition according to the first aspect. In addition, the polymer compound having the constitutional unit (a0), the constitutional unit (a10) described above, and the constitutional unit (a1) described above is particularly useful as a resist material for lithography, which is exposed to EUV or EB. Alternatively, the polymer compound having the constitutional unit (a0), the constitutional unit (a10) described above, the constitutional unit (a1) described above, and the constitutional unit (a5) described above is particularly useful as a resist material for lithography, which is exposed to EUV or EB.

### EXAMPLES

Hereinafter, the present invention will be described in more detail based on examples, but the present invention is not limited to these examples.

### Synthesis Example of Compound

### Synthesis Example 1: Synthesis of Compound (a0-m1)

100 g of 4-hydroxy-3-tert-butoxycarbonylstyrene, 250 g of 2,3,5-triiodobenzoic acid, and 5.6 g of dimethylaminopyridine were dissolved in 800 g of dichloromethane, and 68.8 g of N,N′-diisopropylcarbodiimide (DIC) was added dropwise to the solution under ice cooling. After the solution was stirred at room temperature for 4 hours, the reaction solution was filtered. 207 g of trifluoroacetic acid (TFA) was added to the filtrate, and the solution was stirred at room temperature for 24 hours. The solvent was distilled off, and 200 g of methanol was added to the residues. The solution was added dropwise to 800 g of t-methylbutyl ether, and the precipitated crystals were filtered and dried, thereby obtaining 205 g of a compound (a0-m1pre).

100 g of the compound (a0-m1pre) was dissolved in 800 g of methyl isobutyl ketone, and 71.2 g of a 40% benzyltrimethylammonium hydroxide aqueous solution and 600 g of ultrapure water were added thereto. After the solution was stirred at room temperature for 2 hours, the aqueous layer was removed, and the organic layer was washed three times with 600 g of ultrapure water. Thereafter, the solvent was distilled off from the organic layer, thereby obtaining 110 g of a compound (a0-m1).

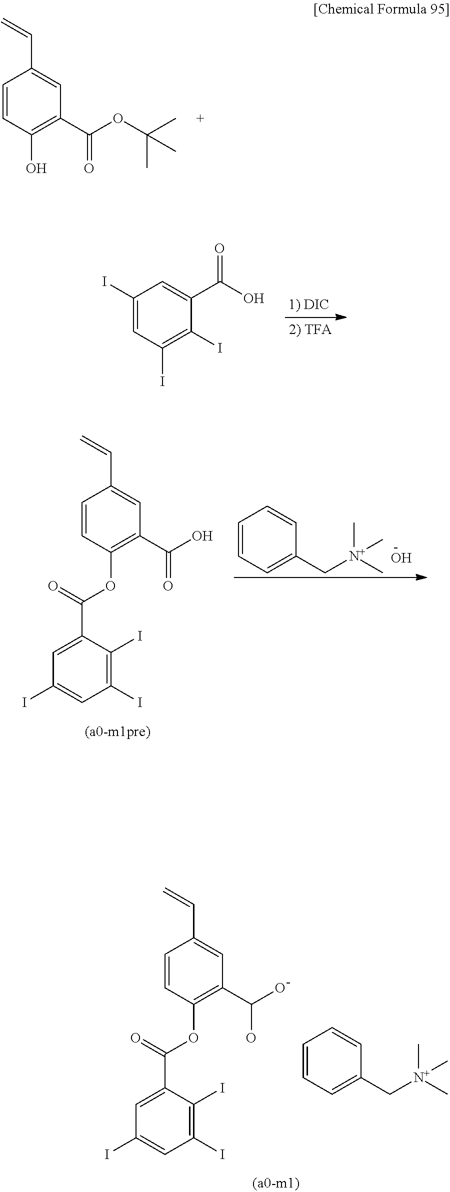

The obtained compound (a0-m1) was subjected to NMR measurement, and the structure thereof was identified from the following results.

¹H-NMR (DMSO-d6, 400 MHz): δ (ppm)=8.30 (s, Ar—H, 2H), 7.60-7.50 (m, Ar—H, 7H), 7.07 (s, Ar—H, 1H), 6.70 (t, —CH═, 1H), 5.72 (d, =CH₂, 1H), 5.18 (d, ═CH₂, 1H), 4.60-4.50 (s, —CH₂—, 2H), 3.04 (s, CH₃, 9H)

### Synthesis Example 2: Synthesis of Compound (a0-m2)

100 g of 4-vinylbenzoic acid, 331 g of 2,4-diiodo-5-tert-butoxycarbonylphenol, and 8.3 g of dimethylaminopyridine were dissolved in 800 g of dichloromethane, and 102 g of N,N′-diisopropylcarbodiimide (DIC) was added dropwise to the solution under ice cooling. After the solution was stirred at room temperature for 4 hours, the reaction solution was filtered. 308 g of trifluoroacetic acid (TFA) was added to the filtrate, and the solution was stirred at room temperature for 24 hours. The solvent was distilled off, and 200 g of methanol was added to the residues. The solution was added dropwise to 800 g of t-methylbutyl ether, and the precipitated crystals were filtered and dried, thereby obtaining 252 g of a compound (a0-m2pre).

100 g of the compound (a0-m2pre) was dissolved in 800 g of methyl isobutyl ketone, and 88.4 g of a 40% benzyltrimethylammonium hydroxide aqueous solution and 600 g of ultrapure water were added thereto. After the solution was stirred at room temperature for 2 hours, the aqueous layer was removed, and the organic layer was washed three times with 600 g of ultrapure water. Thereafter, the solvent was distilled off from the organic layer, thereby obtaining 122 g of a compound (a0-m2).

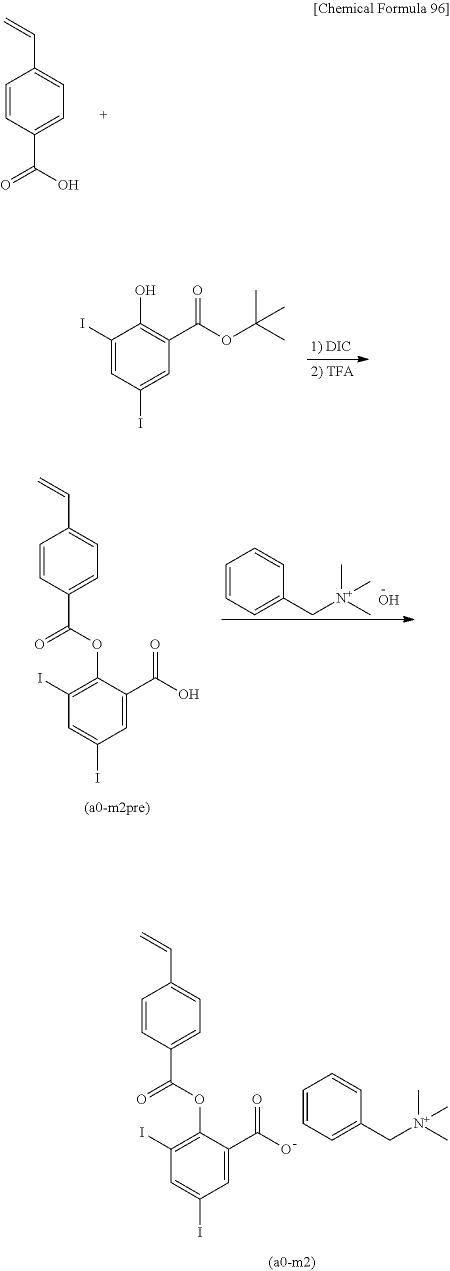

The obtained compound (a0-m2) was subjected to NMR measurement, and the structure thereof was identified from the following results.

¹H-NMR (DMSO-d6, 400 MHz): δ (ppm)=8.10 (d, Ar—H, 2H), 7.85-7.70 (m, Ar—H, 4H), 7.60-7.50 (m, Ar—H, 5H), 6.70 (t, —CH═, 1H), 5.71 (d, ═CH₂, 1H), 5.19 (d, ═CH₂, 1H), 4.60-4.50 (s, —CH₂—, 2H), 3.05 (s, CH₃, 9H)

### Synthesis Example 3: Synthesis of Compound (a0-m3)

100 g of 4-ethenyl-2,6-diiodophenol, 53.9 g of ethyl bromoacetate, 55.7 g of potassium carbonate (K₂CO₃), and 0.1 g of 4-methoxyphenol (methoquinone) were dissolved in 600 g of N,N-dimethylformamide (DMF), and the solution was stirred at 60° C. for 6 hours. Subsequently, 337 g of a 40% benzyltrimethylammonium hydroxide aqueous solution was added thereto, and the solution was stirred at room temperature for 2 hours. Next, the solution was added dropwise to 1200 g of ultrapure water, and the precipitated solid was filtered.

The residues were dissolved in 400 g of methyl isobutyl ketone, and the organic layer was washed once with 500 g of a 10% benzyltrimethylammonium chloride aqueous solution and washed three times with 400 g of ultrapure water. Thereafter, the solvent was distilled off from the organic layer, thereby obtaining 109 g of a compound (a0-m3).

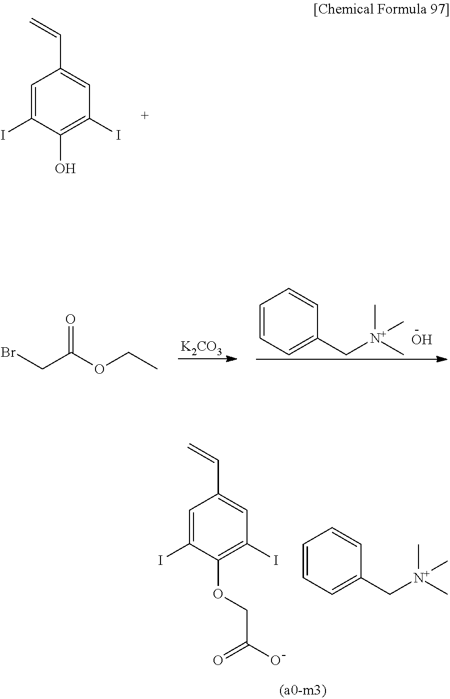

The obtained compound (a0-m3) was subjected to NMR measurement, and the structure thereof was identified from the following results.

¹H-NMR (DMSO-d6, 400 MHz): δ (ppm)=7.85 (s, Ar—H, 2H), 7.61-7.49 (m, Ar—H, 5H), 6.55 (t, —CH═, 1H), 5.72 (d, ═CH₂, 1H), 5.15 (d, ═CH₂, 1H), 4.70 (s, —CH₂—, 2H), 4.61-4.52 (s, —CH₂—, 2H), 3.04 (s, CH₃, 9H)

### Synthesis Example 4: Synthesis of Compound (a0-m4)

100 g of 3,5-diiodosalicylic acid was dissolved in 600 g of dichloromethane, 34.5 g of triethylamine (TEA) was added thereto, and 26.8 g of methacrylic acid chloride was added dropwise thereto under ice cooling. After the solution was stirred at room temperature for 2 hours, the solution was washed twice with 600 g of a 1% HCl aqueous solution and washed three times with 600 g of ultrapure water. Thereafter, the solvent was distilled off from the organic layer, thereby obtaining 106 g of a compound (a0-m4pre).

100 g of the compound (a0-m4pre) was dissolved in 600 g of methyl isobutyl ketone, and 100 g of a 40% benzyltrimethylammonium hydroxide aqueous solution and 600 g of ultrapure water were added thereto. After the solution was stirred at room temperature for 2 hours, the aqueous layer was removed, and the organic layer was washed three times with 600 g of ultrapure water. Thereafter, the solvent was distilled off from the organic layer, thereby obtaining 119 g of a compound (a0-m4).

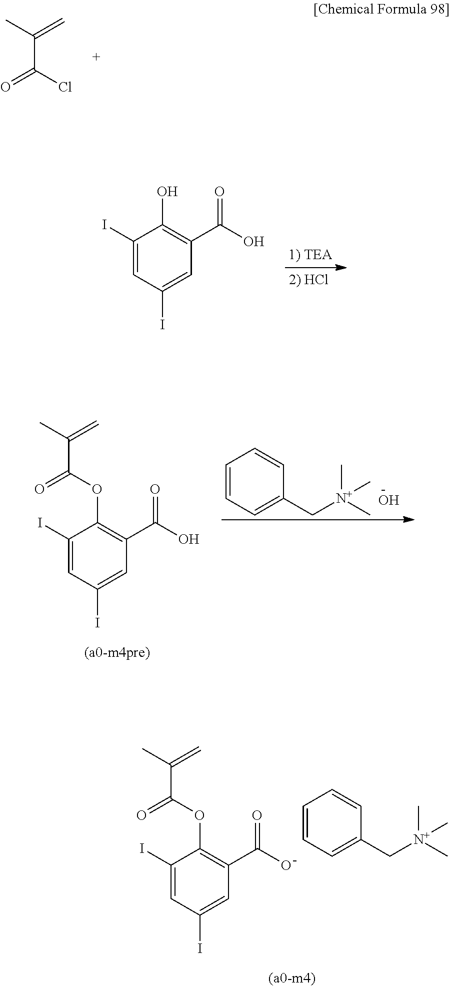

The obtained compound (a0-m4) was subjected to NMR measurement, and the structure thereof was identified from the following results.

¹H-NMR (DMSO-d6, 400 MHz): δ (ppm)=7.80 (d, Ar—H, 2H), 7.60-7.50 (m, Ar—H, 5H), 6.00 (s, ═CH₂, 1H), 5.62 (s, ═CH₂, 1H), 4.59-4.50 (s, —CH₂—, 2H), 3.04 (s, CH₃, 9H), 1.82 (s, CH₃, 3H)

### Synthesis Example 5: Synthesis of Compound (a0-m5)

100 g of the compound (a0-m1) was dissolved in 800 g of dichloromethane, 51.3 g of the compound (C-1) and 800 g of ultrapure water were added thereto, and the solution was stirred at room temperature for 1 hour. After the aqueous layer was removed, the organic layer was washed three times with 800 g of ultrapure water. The solvent was distilled off from the organic layer, thereby obtaining 105 g of a compound (a0-m5).

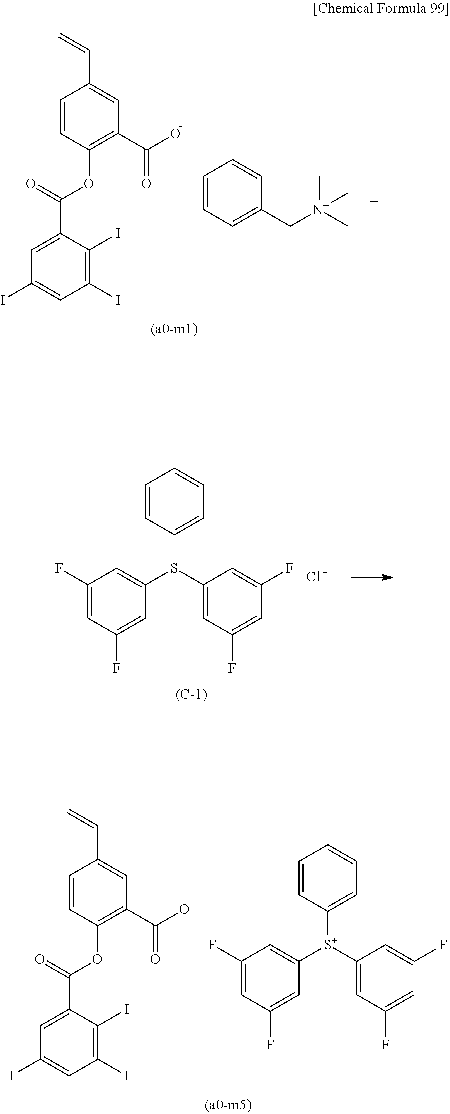

The obtained compound (a0-m5) was subjected to NMR measurement, and the structure thereof was identified from the following results.

¹H-NMR (DMSO-d6, 400 MHz): δ (ppm)=8.30 (s, Ar—H, 2H), 8.00-7.75 (m, Ar—H, 11H), 7.58 (s, Ar—H, 2H), 7.07 (s, Ar—H, 1H), 6.70 (t, —CH═, 1H), 5.72 (d, ═CH₂, 1H), 5.18 (d, ═CH₂, 1H)

### Synthesis Example 6: Synthesis of Compound (a0-m6)

A target compound (a0-m6) was obtained by the same method as in Synthesis Example 5 except that the compound (a0-m2) was used in place of the compound (a0-m1).

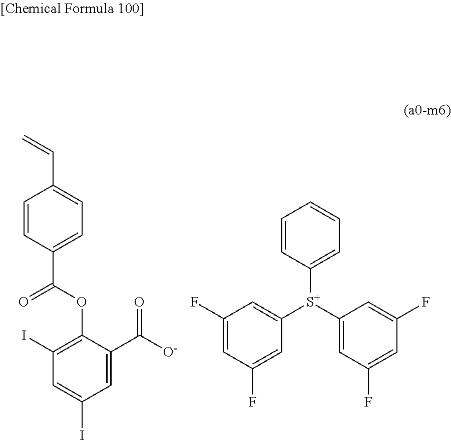 [SMILES: C=Cc1ccc(C(=O)Oc2c(I)cc(I)cc2C(=O)[O-])cc1 ; Fc1cc(F)cc([S+](c2ccccc2)c2cc(F)cc(F)c2)c1]

The obtained compound (a0-m6) was subjected to NMR measurement, and the structure thereof was identified from the following results.

¹H-NMR (DMSO-d6, 400 MHz): δ (ppm)=8.12 (d, Ar—H, 2H), 8.00-7.70 (m, Ar—H, 15H), 6.70 (t, —CH═, 1H), 5.71 (d, ═CH₂, 1H), 5.19 (d, ═CH₂, 1H)

### Synthesis Example 7: Synthesis of Compound (a0-m7)

A target compound (a0-m7) was obtained by the same method as in Synthesis Example 5 except that the compound (a0-m3) was used in place of the compound (a0-m1).

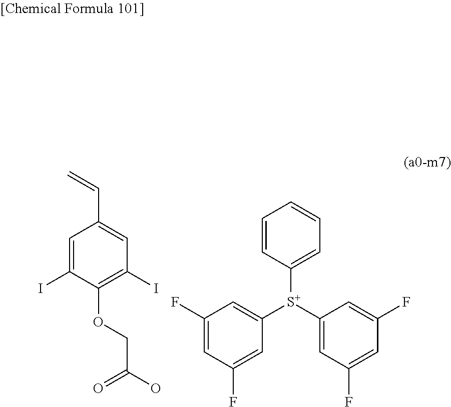 [SMILES: C=Cc1cc(I)c(OCC(=O)O)c(I)c1 ; Fc1cc(F)cc([S+](c2ccccc2)c2cc(F)cc(F)c2)c1]

The obtained compound (a0-m7) was subjected to NMR measurement, and the structure thereof was identified from the following results.

¹H-NMR (DMSO-d6, 400 MHz): δ (ppm)=8.00-7.75 (m, Ar—H, 13H), 6.55 (t, —CH═, 1H), 5.72 (d, ═CH₂, 1H), 5.15 (d, ═CH₂, 1H), 4.70 (s, —CH₂—, 2H)

### Synthesis Example 8: Synthesis of Compound (a0-m8)

A target compound (a0-m8) was obtained by the same method as in Synthesis Example 5 except that the compound (a0-m4) was used in place of the compound (a0-m1).

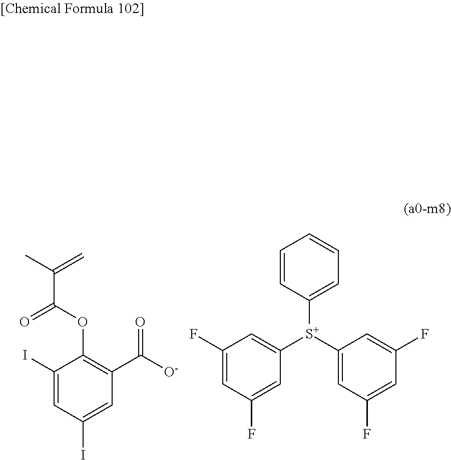 [SMILES: C=C(C)C(=O)Oc1c(I)cc(I)cc1C(=O)[O-] ; Fc1cc(F)cc([S+](c2ccccc2)c2cc(F)cc(F)c2)c1]

The obtained compound (a0-m8) was subjected to NMR measurement, and the structure thereof was identified from the following results.

¹H-NMR (DMSO-d6, 400 MHz): δ (ppm)=8.02-7.70 (d, Ar—H, 13H), 6.00 (s, ═CH₂, 1H), 5.62 (s, ═CH₂, 1H), 1.80 (s, CH₃, 3H)

### Synthesis Example 9: Synthesis of Compound (a0-m9)

A target compound (a0-m9) was obtained by the same method as in Synthesis Example 5 except that the following compound (a0-m9-1) was used in place of 2,3,5-triiodobenzoic acid.

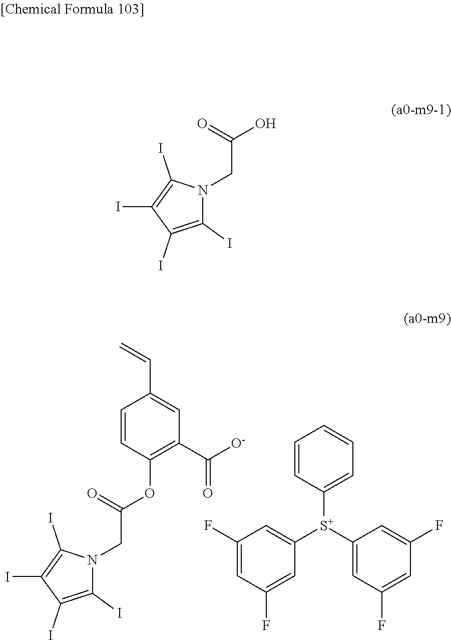 [SMILES: O=C(O)Cn1c(I)c(I)c(I)c1I ; C=Cc1ccc(OC(=O)Cn2c(I)c(I)c(I)c2I)c(C(=O)[O-])c1 ; Fc1cc(F)cc([S+](c2ccccc2)c2cc(F)cc(F)c2)c1]

The obtained compound (a0-m9) was subjected to NMR measurement, and the structure thereof was identified from the following results.

¹H-NMR (DMSO-d6, 400 MHz): δ (ppm)=8.00-7.75 (m, Ar—H, 11H), 7.60 (s, Ar—H, 2H), 7.05 (s, Ar—H, 1H), 4.78 (t, —CH═, 1H), 5.70 (d, ═CH₂, 1H), 5.18 (d, ═CH₂, 1H), 4.78 (s, —CH₂—, 2H)

### Synthesis Example 10: Synthesis of Compound (a0-m10)

A target compound (a0-m10) was obtained by the same method as in Synthesis Example 7 except that the following compound (a0-m10-1) was used in place of 4-ethenyl-2,6-diiodophenol, and the following compound (a0-m10-2) was used in place of ethyl bromoacetate.

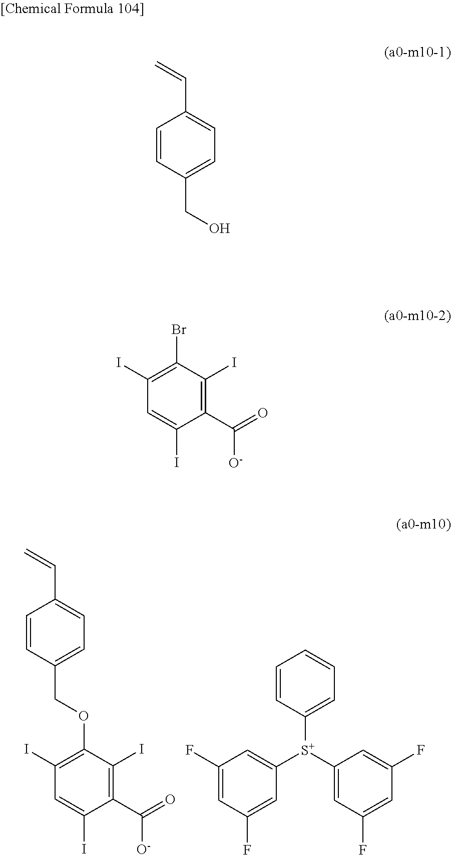

The obtained compound (a0-m10) was subjected to NMR measurement, and the structure thereof was identified from the following results.

¹H-NMR (DMSO-d6, 400 MHz): δ (ppm)=8.10-7.70 (m, Ar—H, 16H), 6.74 (t, —CH═, 1H), 5.70 (d, ═CH₂, 1H), 5.20 (d, ═CH₂, 1H), 4.91 (s, —CH₂—, 2H)

### Synthesis Example 11: Synthesis of Compound (a0-m11)

A target compound (a0-m11) was obtained by the same method as in Synthesis Example 7 except that the following compound (a0-m11-1) was used in place of ethyl bromoacetate.

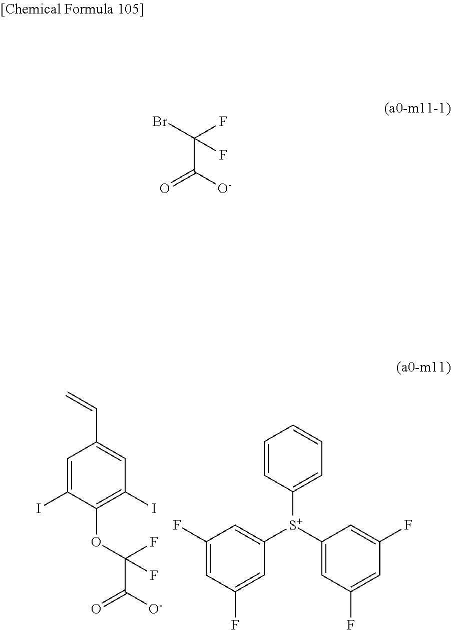 [SMILES: O=C([O-])C(F)(F)Br ; C=Cc1cc(I)c(OC(F)(F)C(=O)[O-])c(I)c1 ; Fc1cc(F)cc([S+](c2ccccc2)c2cc(F)cc(F)c2)c1]

The obtained compound (a0-m11) was subjected to NMR measurement, and the structure thereof was identified from the following results.

¹H-NMR (DMSO-d6, 400 MHz): δ (ppm)=8.03-7.72 (m, Ar—H, 13H), 6.54 (t, —CH═, 1H), 5.70 (d, ═CH₂, 1H), 5.15 (d, ═CH₂, 1H)

### Synthesis Example 12: Synthesis of Compound (a0-m12)

A target compound (a0-m12) was obtained by the same method as in Synthesis Example 8 except that the following compound (a0-m12-1) was used in place of 3,5-diiodosalicylic acid.

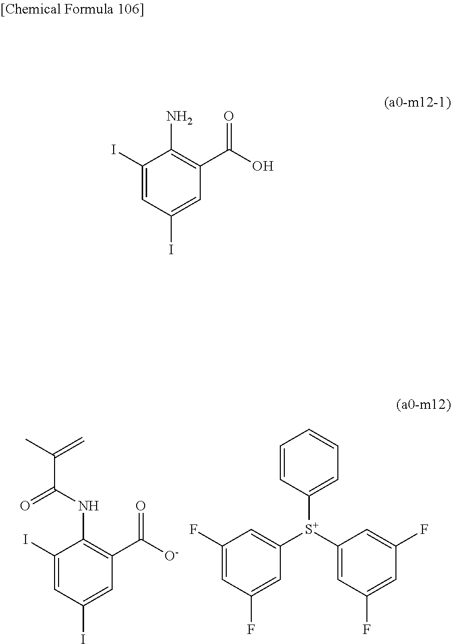 [SMILES: Nc1c(I)cc(I)cc1C(=O)O ; C=C(C)C(=O)Nc1c(I)cc(I)cc1C(=O)[O-] ; Fc1cc(F)cc([S+](c2ccccc2)c2cc(F)cc(F)c2)c1]

The obtained compound (a0-m12) was subjected to NMR measurement, and the structure thereof was identified from the following results.

¹H-NMR (DMSO-d6, 400 MHz): δ (ppm)=8.00-7.71 (d, Ar—H, 13H), 6.01 (s, ═CH₂, 1H), 5.62 (s, ═CH₂, 1H), 1.80 (s, CH₃, 3H)

### Synthesis Example 13: Synthesis of Compound (a0-m13)

100 g of the compound (a0-m1) was dissolved in 800 g of dichloromethane, 41.3 g of the compound (C-2) and 800 g of ultrapure water were added thereto, and the solution was stirred at room temperature for 1 hour. After the aqueous layer was removed, the organic layer was washed three times with 800 g of ultrapure water. The solvent was distilled off from the organic layer, thereby obtaining 97 g of a target compound (a0-m13).

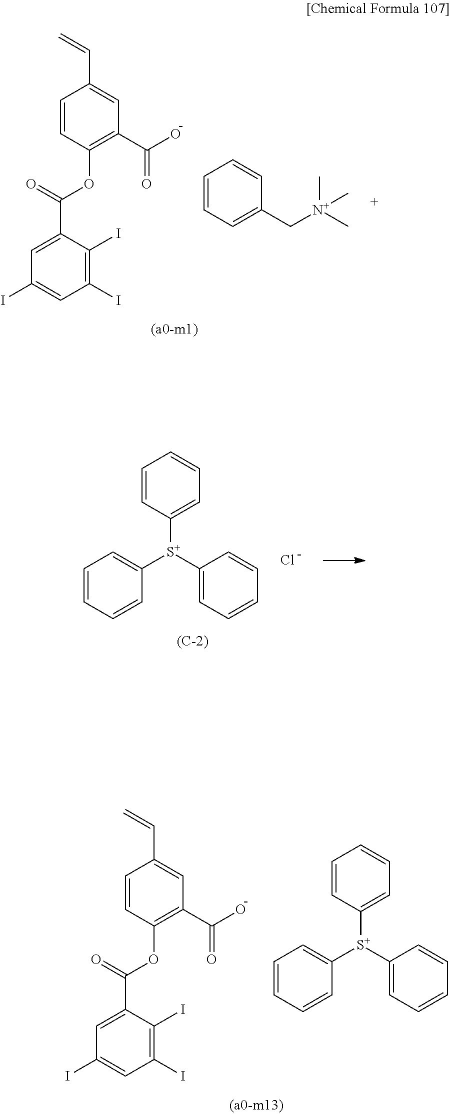

The obtained compound (a0-m13) was subjected to NMR measurement, and the structure thereof was identified from the following results.

¹H-NMR (DMSO-d6, 400 MHz): δ (ppm)=8.30 (s, Ar—H, 2H), 7.58 (s, Ar—H, 2H), 7.49-7.35 (m, Ar—H, 15H), 7.07 (s, Ar—H, 1H), 6.70 (t, —CH═, 1H), 5.72 (d, ═CH₂, 1H), 5.18 (d, ═CH₂, 1H)

### Synthesis Example of Polymer Compound

### Synthesis of Polymer Compound (A1-1)

10.0 g of the compound (a10-m1pre), 16.2 g of the compound (a1-m1), 20.5 g of the compound (a5-m1), 6.9 g of the compound (a0-m1), and 12.6 g of dimethyl azobis(isobutyrate) (V-601) as a polymerization initiator were dissolved in 100 g of methyl ethyl ketone (MEK), and the solution was stirred at 70° C. for 5 hours in a nitrogen atmosphere. Thereafter, the reaction solution was cooled to room temperature.

Next, 3.0 g of acetic acid and 60 g of methanol were added to the obtained polymerization solution, and a deprotection reaction was carried out at 30° C. for 8 hours.

After completion of the reaction, the obtained reaction solution was precipitated in 1,200 g of a mixed solvent of methanol and water and washed. The obtained white solid was filtered and dried overnight under reduced pressure, thereby obtaining a target polymer compound (A1-1).

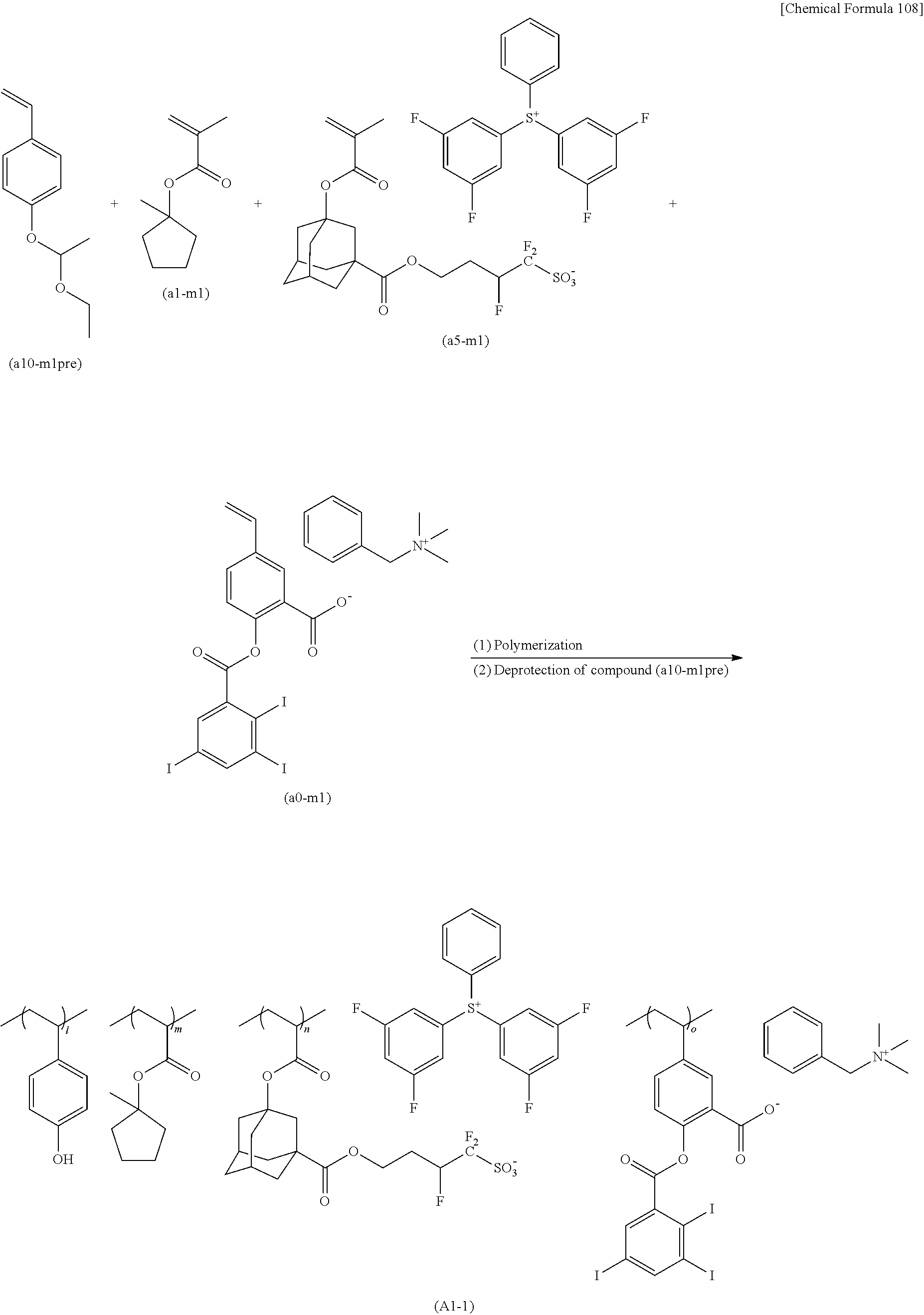

### Synthesis of Polymer Compounds (A1-2) to (A1-12)

Target polymer compounds (AT-2) to (A1-12) were obtained by the same method as the method in [Synthesis of polymer compound (A1-1)] except that each of the compounds (a0-m2) to (a0-m12) was used in place of the compound (a0-m1).

The structures of the obtained polymer compounds (A1-2) to (A1-12) are shown below.

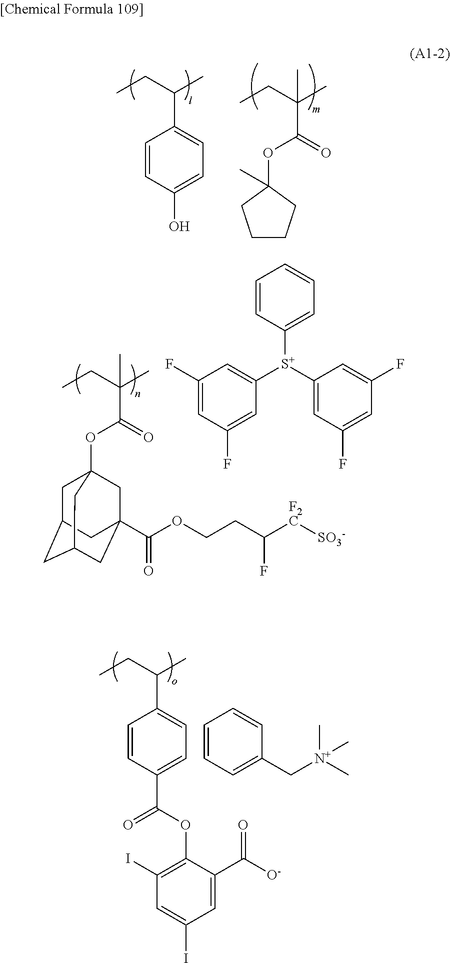

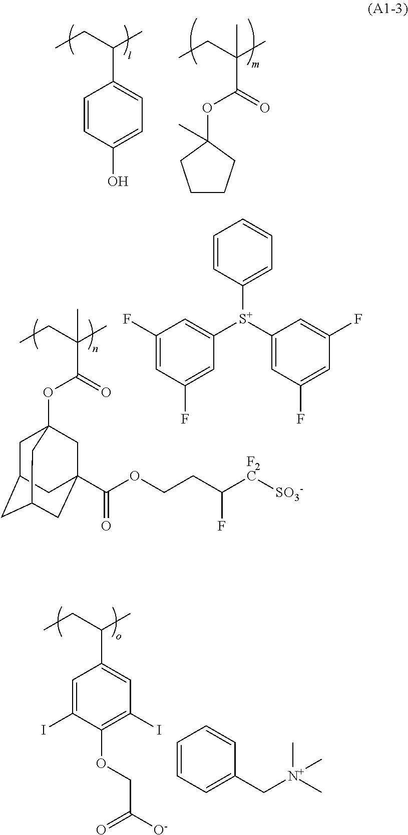

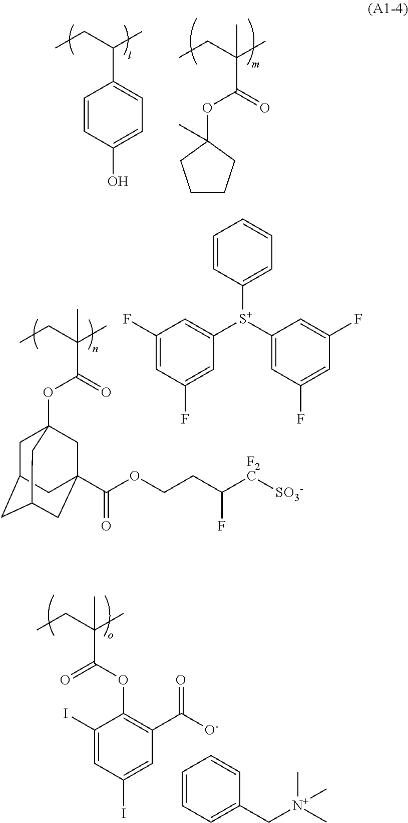

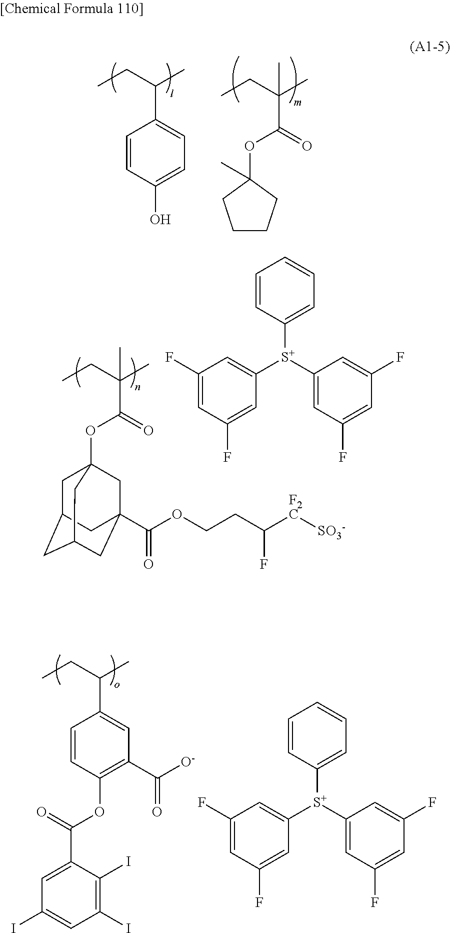

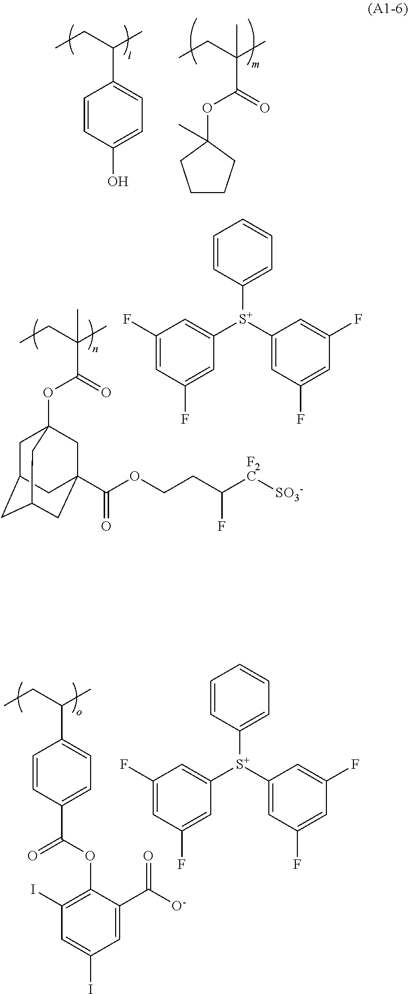

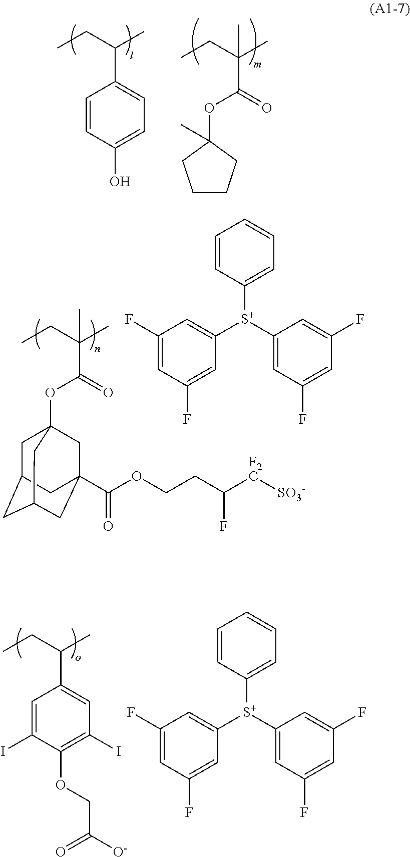

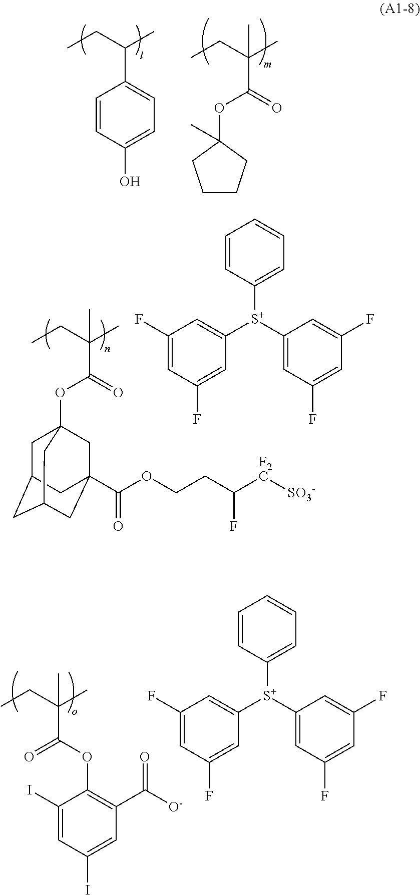

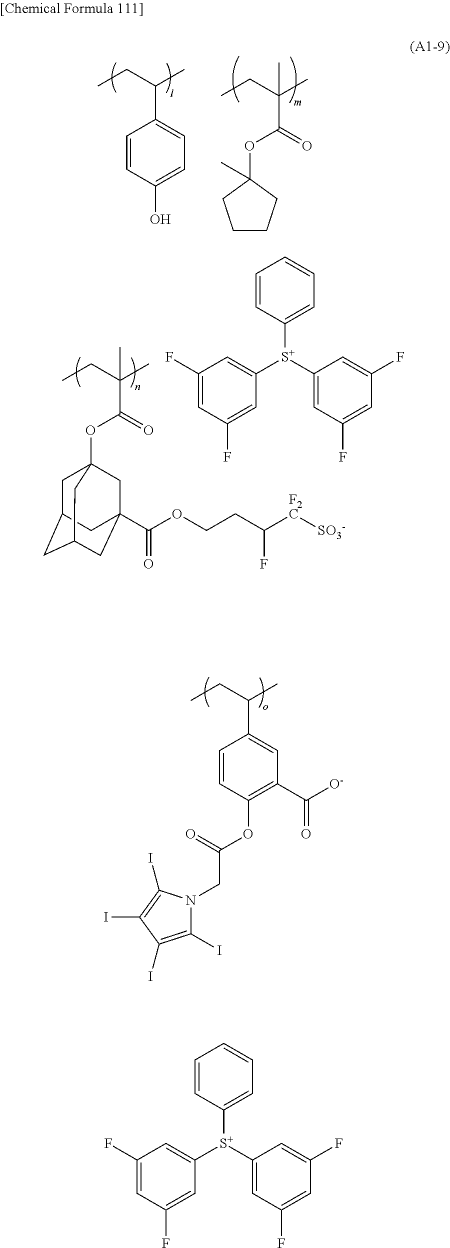

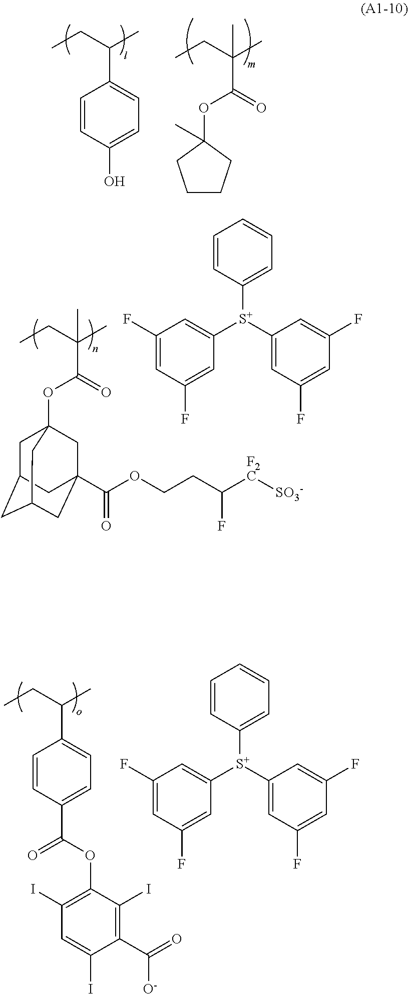

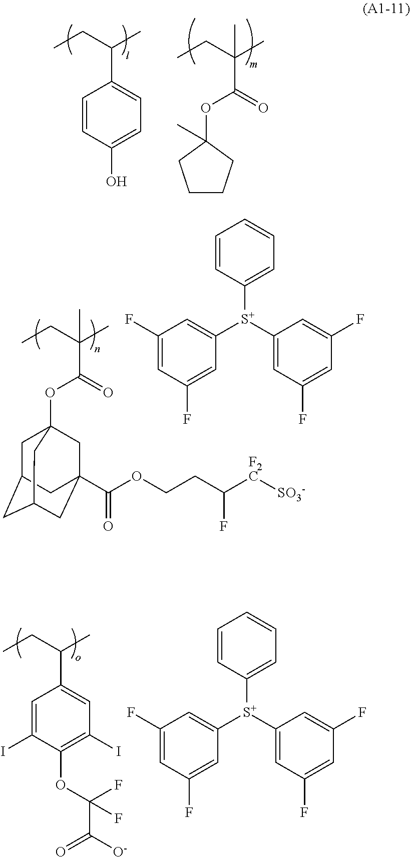

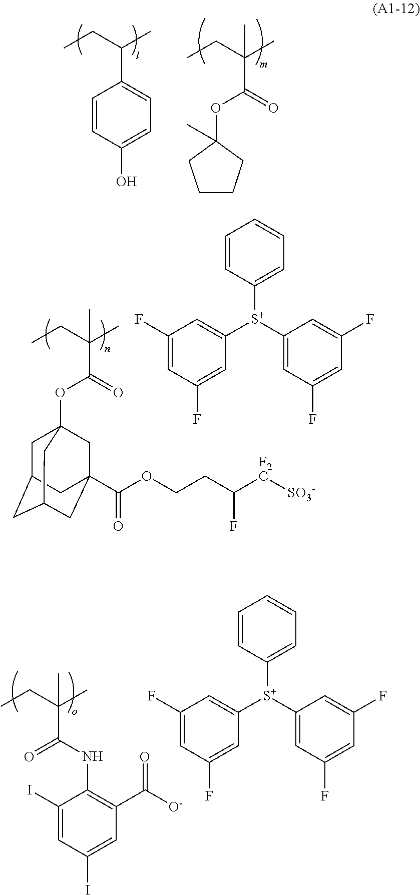

### Synthesis of Polymer Compound (A1-13)

A target polymer compound (A1-13) was obtained by the same method as the method in [Synthesis of polymer compound (A1-1)] except that the compound (a0-m13) was used in place of the compound (a0-m1), and the following compound (a5-m2) was used in place of the compound (a5-m1).

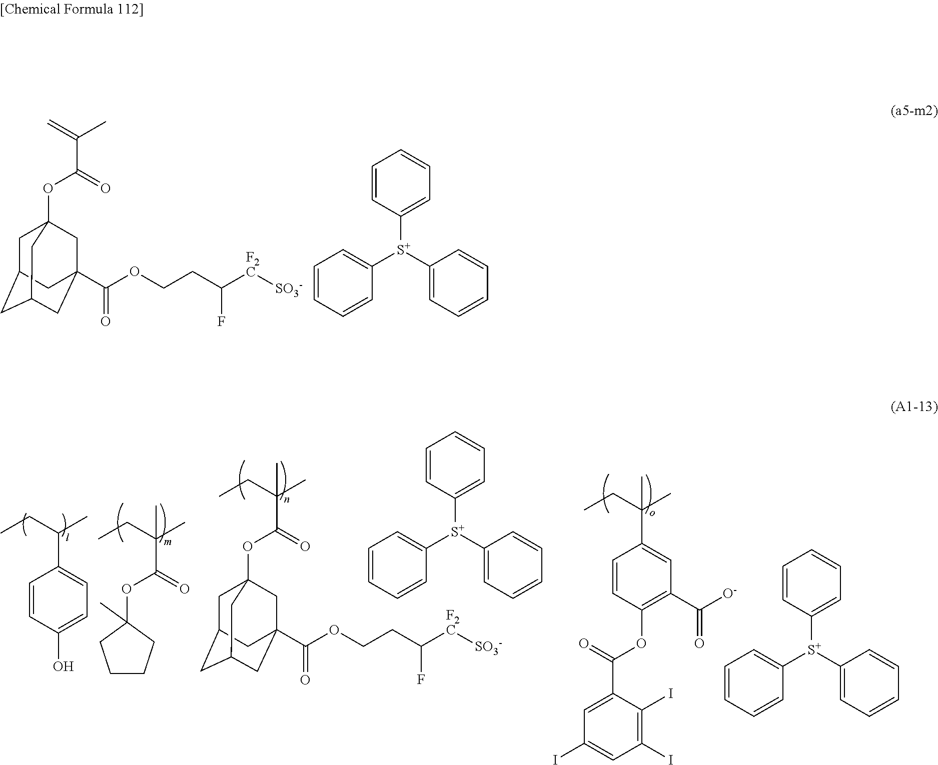

### Synthesis of Polymer Compound (A1-14)

10.0 g of the compound (a10-m1pre), 16.1 g of the compound (a1-m1), 10.2 g of the compound (a0-m5), and 10.9 g of dimethyl azobis(isobutyrate) (V-601) as a polymerization initiator were dissolved in 70 g of methyl ethyl ketone (MEK), and the solution was stirred at 70° C. for 5 hours in a nitrogen atmosphere. Thereafter, the reaction solution was cooled to room temperature.

Next, 3.0 g of acetic acid and 60 g of methanol were added to the obtained polymerization solution, and a deprotection reaction was carried out at 30° C. for 8 hours.

After completion of the reaction, the obtained reaction solution was precipitated in 1,200 g of a mixed solvent of methanol and water and washed. The obtained white solid was filtered and dried overnight under reduced pressure, thereby obtaining a target polymer compound (A1-14).

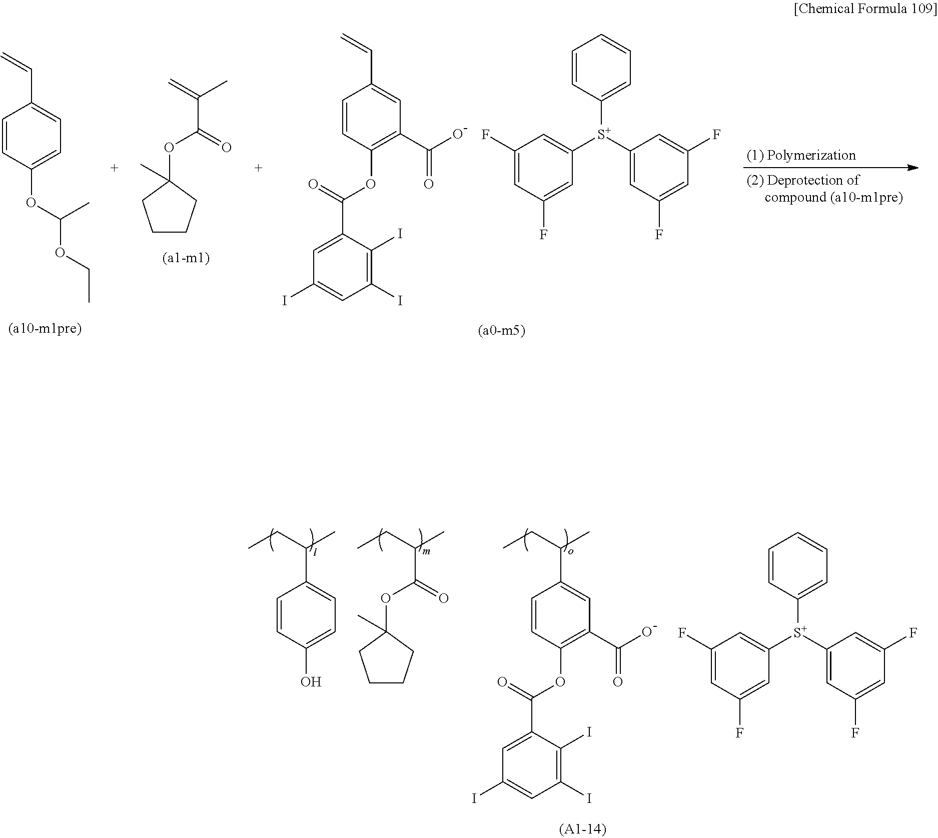

The weight-average molecular weights (Mw) and the polydispersities (Mw/Mn) of the polymer compounds (A1-1) to (A1-14) were determined by GPC measurement (in terms of standard polystyrene).

The copolymer compositional ratio (ratio (molar ratio) between constitutional units in structural formula) of the polymer compounds (A1-1) to (A1-14) was determined by the carbon 13 nuclear magnetic resonance spectrum (600 MHz-¹³C-NMR).
Polymer compound (A1-1): weight-average molecular weight (Mw) of 10100, polydispersity (Mw/Mn) of 1.60, l/m/n/o=30/50/15/5
Polymer compound (A1-2): weight-average molecular weight (Mw) of 9900, polydispersity (Mw/Mn) of 1.58, l/m/n/o=30/50/15/5
Polymer compound (A1-3): weight-average molecular weight (Mw) of 10400, polydispersity (Mw/Mn) of 1.61, l/m/n/o=30/50/15/5
Polymer compound (A1-4): weight-average molecular weight (Mw) of 9800, polydispersity (Mw/Mn) of 1.56, l/m/n/o=30/50/15/5
Polymer compound (A1-5): weight-average molecular weight (Mw) of 9600, polydispersity (Mw/Mn) of 1.54, l/m/n/o=30/50/15/5
Polymer compound (A1-6): weight-average molecular weight (Mw) of 10000, polydispersity (Mw/Mn) of 1.50, l/m/n/o=30/50/15/5
Polymer compound (A1-7): weight-average molecular weight (Mw) of 10300, polydispersity (Mw/Mn) of 1.52, l/m/n/o=30/50/15/5
Polymer compound (A1-8): weight-average molecular weight (Mw) of 9700, polydispersity (Mw/Mn) of 1.51, l/m/n/o=30/50/15/5
Polymer compound (A1-9): weight-average molecular weight (Mw) of 9500, polydispersity (Mw/Mn) of 1.55, l/m/n/o=30/50/15/5
Polymer compound (A1-10): weight-average molecular weight (Mw) of 10100, polydispersity (Mw/Mn) of 1.59, l/m/n/o=30/50/15/5
Polymer compound (A1-11): weight-average molecular weight (Mw) of 9900, polydispersity (Mw/Mn) of 1.58, l/m/n/o=30/50/15/5
Polymer compound (A1-12): weight-average molecular weight (Mw) of 10200, polydispersity (Mw/Mn) of 1.57, l/m/n/o=30/50/15/5
Polymer compound (A1-13): weight-average molecular weight (Mw) of 9700, polydispersity (Mw/Mn) of 1.60, l/m/n/o=30/50/15/5
Polymer compound (A1-14): weight-average molecular weight (Mw) of 9800, polydispersity (Mw/Mn) of 1.55, l/m/o=35/58/7

### Preparation of Resist Composition

### Examples 1 to 14, Comparative Examples 1 to 4

Each of the components shown in Table 1 was mixed and dissolved to prepare a resist composition of each Example.

TABLE 1

| | Component | Component | Component | Component |
|---|---|---|---|---|
| | (A) | (B) | (D) | (S) |
| Example 1 | (A)-1 | — | — | (S)-1 |
| | [100] | | | [2500] |
| Example 2 | (A)-2 | — | — | (S)-1 |
| | [100] | | | [2500] |
| Example 3 | (A)-3 | — | — | (S)-1 |
| | [100] | | | [2500] |
| Example 4 | (A)-4 | — | — | (S)-1 |
| | [100] | | | [2500] |
| Example 5 | (A)-5 | — | — | (S)-1 |
| | [100] | | | [2500] |
| Example 6 | (A)-6 | — | — | (S)-1 |
| | [100] | | | [2500] |
| Example 7 | (A)-7 | — | — | (S)-1 |
| | [100] | | | [2500] |
| Example 8 | (A)-8 | — | — | (S)-1 |
| | [100] | | | [2500] |
| Example 9 | (A)-9 | — | — | (S)-1 |
| | [100] | | | [2500] |
| Example 10 | (A)-10 | — | — | (S)-1 |
| | [100] | | | [2500] |
| Example 11 | (A)-11 | — | — | (S)-1 |
| | [100] | | | [2500] |
| Example 12 | (A)-12 | — | — | (S)-1 |
| | [100] | | | [2500] |
| Example 13 | (A)-13 | — | — | (S)-1 |
| | [100] | | | [2500] |
| Example 14 | (A)-14 | (B)-1 | — | (S)-1 |
| | [80] | [20] | | [2500] |
| Comparative | (A)-15 | — | — | (S)-1 |
| Example 1 | [100] | | | [2500] |
| Comparative | (A)-16 | — | (D)-1 | (S)-1 |
| Example 2 | [90] | | [10] | [2500] |
| Comparative | (A)-17 | — | — | (S)-1 |
| Example 3 | [100] | | | [2500] |
| Comparative | (A)-18 | (B)-1 | — | (S)-1 |
| Example 4 | [80] | [20] | | [2500] |

In Table 1, each abbreviation has the following meaning. The numerical values in the brackets represent the blending amounts (parts by mass; in terms of solid content).
(A)-1 to (A)-14: polymer compounds (A1-1) to (A1-14) shown above
(A)-15: polymer compound (A2-1) shown below Weight-average molecular weight (Mw) of 9800, polydispersity (Mw/Mn) of 1.54, l/m/n/o=30/50/15/5
(A)-16: polymer compound (A2-2) shown below Weight-average molecular weight (Mw) of 9600, polydispersity (Mw/Mn) of 1.48, l/m/n=35/50/15
(A)-17: polymer compound (A2-3) shown below Weight-average molecular weight (Mw) of 9300, polydispersity (Mw/Mn) of 1.56, l/m/n/o=30/50/15/5
(A)-18: polymer compound (A2-4) shown below Weight-average molecular weight (Mw) of 10000, polydispersity (Mw/Mn) of 1.59, l/m/o=35/58/7

The weight-average molecular weight (Mw) is a weight-average molecular weight in terms of standard polystyrene, which is determined by GPC measurement. The copolymer compositional ratio (ratio (molar ratio) between constitutional units in structural formula) was determined by ¹³C-NMR.

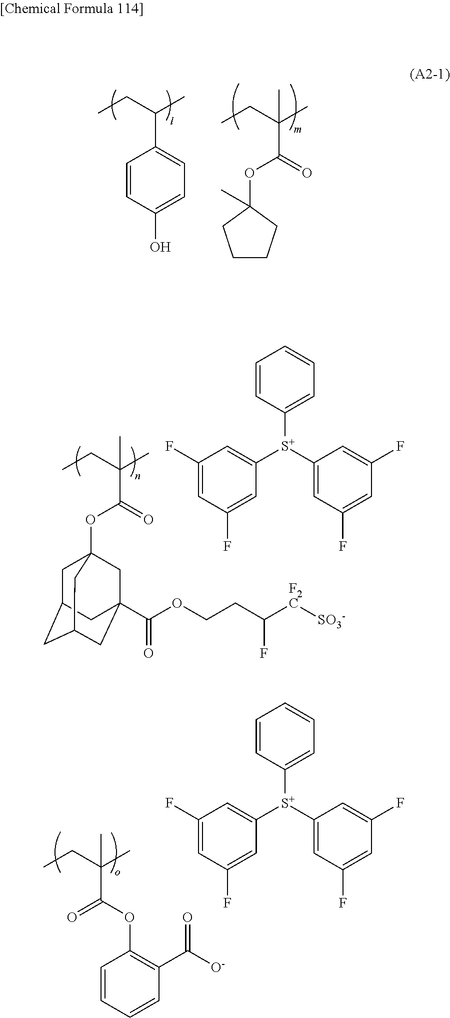

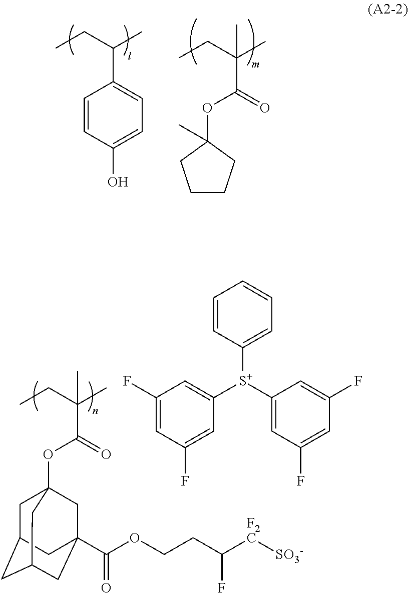

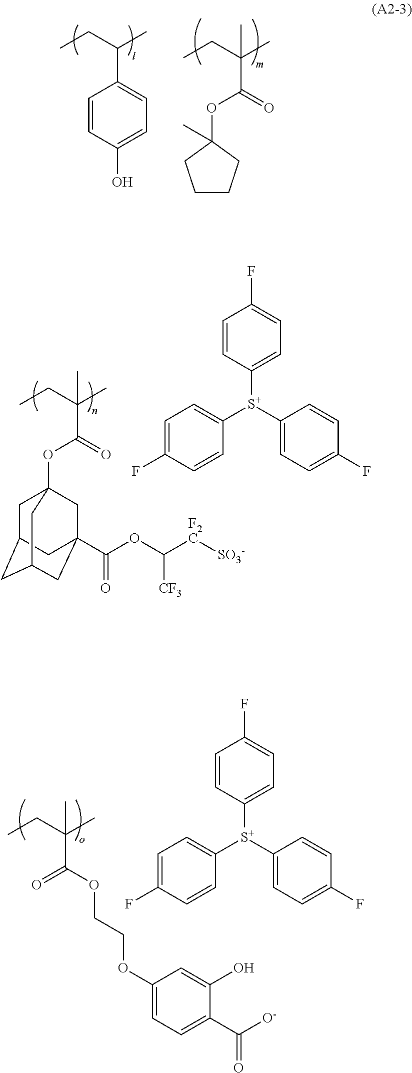

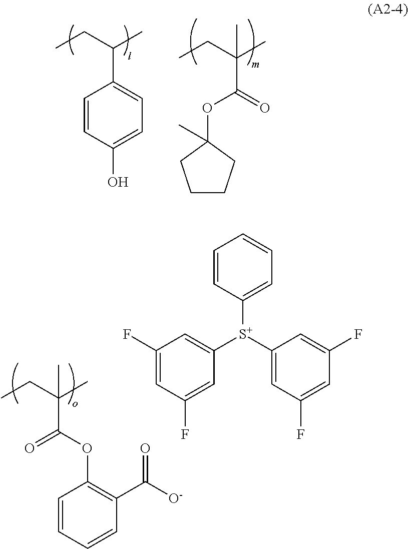

(B)-1: acid generator consisting of compound (B-1) shown below
(D)-1: acid diffusion control agent formed of compound (D1-1) shown below
(S)-1: mixed solvent of propylene glycol monomethyl ether acetate/propylene glycol monomethyl ether=60/40 (mass ratio)

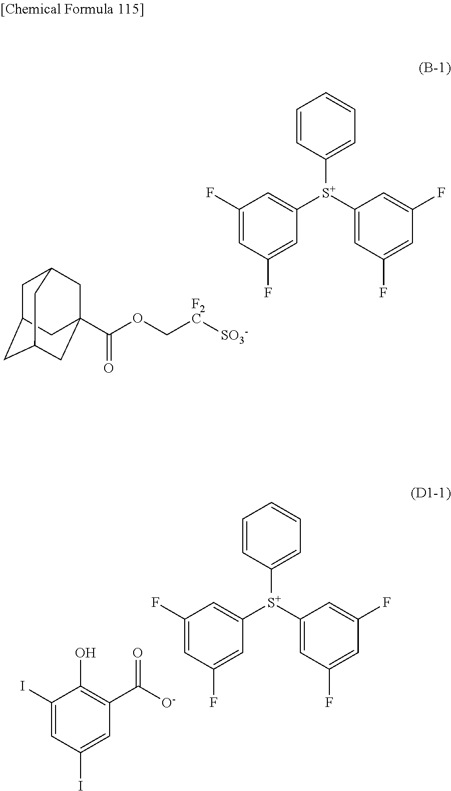

### <Evaluation>

A line-and-space resist pattern (LS pattern) was formed by the method for forming a resist pattern shown below, and the sensitivity and the line width roughness (LWR) were evaluated.

### <<Formation of Resist Pattern>>

### Step of Forming Resist Film:

The resist composition of each Example was applied onto an 8-inch silicon substrate which had been subjected to a hexamethyldisilazane (HMDS) treatment using a spinner, the coated wafer was subjected to a pre-bake (PAB) treatment on a hot plate at a temperature of 110° C. for 60 seconds so that the coated wafer was dried to form a resist film having a film thickness of 50 nm.

### Step of Exposing Resist Film:

Next, the resist film was drawn (exposed) using an electron beam lithography apparatus JEOL JBX-9300FS (manufactured by JEOL Co., Ltd.) at an acceleration voltage of 100 kV (Beam current: 100 pA, Scan step: 4 nm) such that a target size was a 1:1 line-and-space pattern (hereinafter referred to as an LS pattern) having a line width of 50 nm (pitch width: 100 nm).

Thereafter, a post exposure bake (PEB) treatment was performed thereon at 100° C. for 60 seconds.

Step of Developing Resist Film after Light Exposure:

Next, a 2.38 mass % tetramethylammonium hydroxide (TMAH) aqueous solution “NMD-3” (trade name, manufactured by Tokyo Ohka Kogyo Co., Ltd.) was added dropwise to the resist film from an LD-nozzle at 23° C. using a development device (clean track ACT8, manufactured by Tokyo Electron Co., Ltd.) to perform alkali development for 60 seconds.

Thereafter, water rinsing was performed for 15 seconds using pure water.

As a result, in all the examples, each of 1:1 LS patterns having a line width of 50 nm (pitch width of 100 nm) was formed.

### [Evaluation of Optimum Exposure Amount (Eop)]

In <<Formation of resist pattern>> described above, an optimum exposure amount Eop (μC/cm²) in a case where the LS pattern was formed was determined. The results are listed in the columns of “Eop (μC/cm²)” in Table 2.

### [Evaluation of Line Width Roughness (LWR)]

In the LS pattern formed in <<Formation of resist pattern>>, the space width was measured at 400 sites in the longitudinal direction of the space with a CD-SEM (scanning electron microscope, trade name: S—9380, manufactured by Hitachi High-Tech Corporation, acceleration voltage: 300 V). A value of three times (3s) the standard deviation (s) was obtained from the measurement results, and a value obtained by averaging 3s at 400 sites was calculated as a scale indicating the LWR. The results are shown in Table 2 “LWR (nm)”.

The smaller the value of 3s, the smaller the roughness of the line width, and the more uniform width of the LS pattern was obtained.

TABLE 2

| | PAB | PEB | Eop | LWR |
|---|---|---|---|---|
| | (° C.) | (° C.) | (μC/cm²) | (nm) |
| Example 1 | 110 | 100 | 107 | 3.6 |
| Example 2 | 110 | 100 | 109 | 3.9 |
| Example 3 | 110 | 100 | 115 | 4.0 |
| Example 4 | 110 | 100 | 119 | 4.2 |
| Example 5 | 110 | 100 | 92 | 3.4 |
| Example 6 | 110 | 100 | 94 | 3.7 |
| Example 7 | 110 | 100 | 101 | 3.9 |
| Example 8 | 110 | 100 | 105 | 4.2 |
| Example 9 | 110 | 100 | 93 | 3.5 |
| Example 10 | 110 | 100 | 96 | 3.6 |
| Example 11 | 110 | 100 | 100 | 3.8 |
| Example 12 | 110 | 100 | 104 | 4.0 |
| Example 13 | 110 | 100 | 102 | 3.4 |
| Example 14 | 110 | 100 | 98 | 4.4 |
| Comparative | 110 | 100 | 140 | 5.0 |
| Example 1 | | | | |
| Comparative | 110 | 100 | 132 | 5.5 |
| Example 2 | | | | |
| Comparative | 110 | 100 | 138 | 5.2 |
| Example 3 | | | | |
| Comparative | 110 | 100 | 135 | 5.8 |
| Example 4 | | | | |

As shown in the results listed in Table 2, it can be confirmed that the resist compositions of Examples 1 to 14 had higher sensitivity and further reduced roughness of the line width in the LS pattern, as compared with the resist compositions of Comparative Examples 1 to 4.

While preferred examples of the present invention have been described and illustrated above, it should be understood that these are exemplary of the present invention and are not to be considered as limiting. Additions, omissions, substitutions, and other modifications can be made without departing from the scope of the present invention. The present invention is not limited by the description above, but is limited only by the appended claims.

What is claimed is:

1. A resist composition which generates an acid upon light exposure and whose solubility in a developing solution is changed by an action of the acid, the resist composition comprising:
a resin component (A1) whose solubility in a developing solution is changed by the action of the acid,
wherein the resin component (A1) has a constitutional unit (a0) derived from a compound represented by General Formula (a0-m), 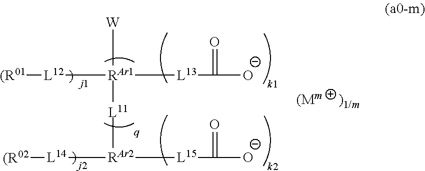
wherein W represents a polymerizable group-containing group, R^{Ar1} and R^{Ar2} each independently represent an aromatic group which may have a substituent, R^{Ar1}-L¹¹-represents R^{Ar1}—O—, R^{Ar1}—S—, R^{Ar1}—NH—, R^{Ar1}-Ak-, R^{Ar1}—C(═O)—O—, R^{Ar1}—C(═O)—NH—, R^{Ar1}—O—C(═O)—, R^{Ar1}—NH—C(═O)—, R^{Ar1}-Ak-O—, R^{Ar1}-Ak-NH—, R^{Ar1}—O—C(═O)-Ak-, or R^{Ar1}- when L¹¹ represents a single bond), Ak represents an alkylene group or a fluorinated alkylene group, and the same applies hereinafter, R^{Ar1}-L¹²⁻ represents R^{Ar1}-Ak-, R^{Ar1}—C(═O)—O-Ak-, R^{Ar1}—C(═O)—NH-Ak-, R^{Ar1}—O—C(═O)-Ak-, R^{Ar1}—NH—C(═O)-Ak-, R^{Ar1}—O—C(═O)—, or R^{Ar1}— when L¹² represents a single bond, R^{Ar1}-L¹³- represents R^{Ar1}—O—, R^{Ar1}—S—, R^{Ar1}—NH—, R^{Ar1}-Ak-, R^{Ar1}—O-Ak-, or R^{Ar1}— when L¹³ represents a single bond, R^{Ar2}-L¹⁴- represents R^{Ar2}-Ak-, R^{Ar2}—C(═O)—O-Ak-, R^{Ar2}—C(═O)—NH-Ak-, R^{Ar2}—O—C(═O)-Ak-, R^{Ar2}—NH—C(═O)-Ak-, R^{Ar2}—O—C(═O)—, or R^{Ar2}— when L¹⁴ represents a single bond), R^{Ar2}-L¹⁵- represents R^{Ar2}—O—, R^{Ar2}—S—, R^{Ar2}—NH—, R^{Ar2}-Ak-, R^{Ar2}—O-Ak-, or R^{Ar2}— when L¹⁵ represents a single bond), q represents 0 or 1, R⁰¹ and R⁰² each independently represents an iodinated alkyl group, an iodine atom, or a hydrogen atom, j1 and j2 each independently represents an integer of 0 to 5, where in a case where q represents 0, R⁰² represents an iodinated alkyl group or an iodine atom, and j2 represents an integer of 1 to 5, in a case where q represents 1, the number of iodine atoms in R⁰¹ and R⁰² is 1 or more, k1 and k2 each independently represents 0 or 1, where in a case where q represents 0, k2 represents 1, and in a case where q represents 1, k1+k2 is 1, M^{m+} represents an m-valent onium cation, and m represents an integer of 1 or greater.

2. The resist composition according to claim 1, wherein W in General Formula (a0-m) represents a polymerizable group-containing group represented by General Formula (a0-w), 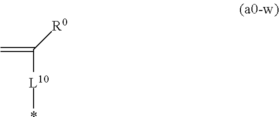
wherein R⁰ represents an alkyl group having 1 to 5 carbon atoms, a halogenated alkyl group having 1 to 5 carbon atoms, or a hydrogen atom, -L¹⁰-* represents —C(═O)—O—*, —C(═O)—NH—*, or -* when L¹⁰ represents a single bond, and * represents a bonding site that is bonded to R^{Ar1} in General Formula (a0-m) or a bonding site that is bonded to R^{Ar2} in when q represents 0.

3. The resist composition according to claim 2,
wherein W in General Formula (a0-m) is the polymerizable group-containing group represented by General Formula (a0-w),
q represents 1,
k1 represents 1,
k2 represents 0,
R⁰² represents an iodinated alkyl group or an iodine atom,
j2 represents an integer of 1 to 5, and
L¹⁰ in General Formula (a0-w) represents a single bond.

4. The resist composition according to claim 3, wherein M^{m+} in General Formula (a0-m) represents an m-valent sulfonium cation.

5. The resist composition according to claim 2,
wherein W in General Formula (a0-m) represents the polymerizable group-containing group represented by General Formula (a0-w),
q represents 1,
k1 represents 0,
k2 represents 1,
R⁰² represents an iodinated alkyl group or an iodine atom,
j2 represents an integer of 1 to 5, and
L¹⁰ in General Formula (a0-w) represents a single bond.

6. The resist composition according to claim 5, wherein M^{m+} in General Formula (a0-m) represents an m-valent sulfonium cation.

7. The resist composition according to claim 2,
wherein W in General Formula (a0-m) represents the polymerizable group-containing group represented by General Formula (a0-w),
q represents 0,
k2 represents 1,
R⁰² represents an iodinated alkyl group or an iodine atom,
j2 represents an integer of 1 to 5, and
L¹⁰ in General Formula (a0-w) represents a single bond.

8. The resist composition according to claim 7, wherein M^{m+} in General Formula (a0-m) represents an m-valent sulfonium cation.

9. The resist composition according to claim 2,
wherein W in General Formula (a0-m) represents the polymerizable group-containing group represented by General Formula (a0-w),
q represents 0,
k2 represents 1,
R⁰² represents an iodinated alkyl group or an iodine atom,
j2 represents an integer of 1 to 5, and
L¹⁰-* in General Formula (a0-w) represents —C(═O)—O—* or —C(═O)—NH—* wherein * represents a bonding site that is bonded to R^{Ar2} in General Formula (a0-m).

10. The resist composition according to claim 9, wherein M^{m+} in General Formula (a0-m) represents an m-valent sulfonium cation.

11. The resist composition according to claim 1,
wherein a content proportion of the constitutional unit (a0) in a total amount (100% by mole) of all constitutional units constituting the resin component (A1) is in a range of 1% to 50% by mole.

12. A method for forming a resist pattern, comprising:
forming a resist film on a support using the resist composition according to claim 1;
exposing the resist film to light; and
developing the resist film exposed to light to form a resist pattern.

13. The method for forming a resist pattern according to claim 12, wherein the resist film is exposed to an extreme ultraviolet ray or an electron beam in exposing the resist film to light.

14. A compound which is represented by General Formula (a0-m), 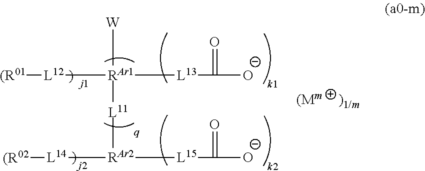
wherein W represents a polymerizable group-containing group, R^{Ar1} and R^{Ar2} each independently represents an aromatic group which may have a substituent, R^{Ar1}-L¹¹- represents R^{Ar1}—O—, R^{Ar1}—S—, R^{Ar1}—NH—, R^{Ar1}-Ak-, R^{Ar1}—C(═O)—O—, R^{Ar1}—C(═O)—NH—, R^{Ar1}—O—C(═O)—, R^{Ar1}—NH—C(═O)—, R^{Ar1}-Ak-O—, R^{Ar1}-Ak-NH—, R^{Ar1}—O—C(═O)-Ak-, or R^{Ar1}— wherein L¹¹ represents a single bond, Ak represents an alkylene group or a fluorinated alkylene group and the same applies hereinafter, R^{Ar1}-L¹²- represents R^{Ar1}-Ak-, R^{Ar1}—C(═O)—O-Ak-, R^{Ar1}—C(═O)—NH-Ak-, R^{Ar1}—O—C(═O)-Ak-, R^{Ar1}—NH—C(═O)-Ak-, R^{Ar1}—O—C(═O)—, or R^{Ar1}— wherein L¹² represents a single bond, R^{Ar1}-L¹³- represents R^{Ar1}—O—, R^{Ar1}—S—, R^{Ar1}—NH—, R^{Ar1}-Ak-, R^{Ar1}—O-Ak-, or R^{Ar1}— wherein L¹³ represents a single bond, R^{Ar2}-L¹⁴- represents R^{Ar2}-Ak-, R^{Ar2}—C(═O)—O-Ak-, R^{Ar2}—C(═O)—NH-Ak-, R^{Ar2}—O—C(═O)-Ak-, R^{Ar2}—NH—C(═O)-Ak-, R^{Ar2}—O—C(═O)—, or R^{Ar2}— wherein L¹⁴ represents a single bond, R^{Ar2}-L¹⁵- represents R^{Ar2}—O—, R^{Ar2}—S—, R^{Ar2}—NH—, R^{Ar2}-Ak-, R^{Ar2}—O-Ak-, or R^{Ar2} wherein L¹⁵ represents a single bond), q represents 0 or 1, R⁰¹ and R⁰² each independently represents an iodinated alkyl group, an iodine atom, or a hydrogen atom, j1 and j2 each independently represents an integer of 0 to 5, wherein when q represents 0, R⁰² represents an iodinated alkyl group or an iodine atom, and j2 represents an integer of 1 to 5, and when q represents 1, the number of iodine atoms in R⁰¹ and R⁰² is 1 or more, k1 and k2 each independently represent 0 or 1, wherein when q represents 0, k2 represents 1, and when q represents 1, k1+k2 is 1, M^{m+} represents an m-valent onium cation, and m represents an integer of 1 or greater.

15. The compound according to claim 14,
wherein W in General Formula (a0-m) represents a polymerizable group-containing group represented by General Formula (a0-w), 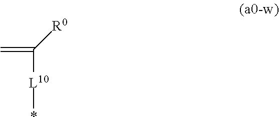
wherein R⁰ represents an alkyl group having 1 to 5 carbon atoms, a halogenated alkyl group having 1 to 5 carbon atoms, or a hydrogen atom, -L¹⁰-* represents —C(═O)—O—*, —C(═O)—NH—*, or -* when L¹⁰ represents a single bond, and * represents a bonding site that is bonded to R^{Ar1} in General Formula (a0-m) or a bonding site that is bonded to R^{Ar2} when q represents 0.

16. The compound according to claim 15,
wherein W in General Formula (a0-m) represents the polymerizable group-containing group represented by General Formula (a0-w),
q represents 1,
k1 represents 1,
k2 represents 0,
R⁰² represents an iodinated alkyl group or an iodine atom,
j2 represents an integer of 1 to 5, and
L¹⁰ in General Formula (a0-w) represents a single bond.

17. The compound according to claim 16, wherein M^{m+} in General Formula (a0-m) represents an m-valent sulfonium cation.

18. The compound according to claim 15,
wherein W in General Formula (a0-m) represents the polymerizable group-containing group represented by General Formula (a0-w),
q represents 1,
k1 represents 0,
k2 represents 1,
R⁰² represents an iodinated alkyl group or an iodine atom,
j2 represents an integer of 1 to 5, and
L¹⁰ in General Formula (a0-w) represents a single bond.

19. The compound according to claim 18, wherein M^{m+} in General Formula (a0-m) represents an m-valent sulfonium cation.

20. The compound according to claim 15,
wherein W in General Formula (a0-m) represents the polymerizable group-containing group represented by General Formula (a0-w),
q represents 0,
k2 represents 1,
R⁰² represents an iodinated alkyl group or an iodine atom,
j2 represents an integer of 1 to 5, and
L¹⁰ in General Formula (a0-w) represents a single bond.

21. The compound according to claim 20, wherein M^{m+} in General Formula (a0-m) represents an m-valent sulfonium cation.

22. The compound according to claim 15,
wherein W in General Formula (a0-m) represents the polymerizable group-containing group represented by General Formula (a0-w),
q represents 0,
k2 represents 1,
R⁰² represents an iodinated alkyl group or an iodine atom,
j2 represents an integer of 1 to 5, and
-L¹⁰-* in General Formula (a0-w) represents —C(═O)—O—* or —C(═O)—NH—*, wherein * represents a bonding site that is bonded to R^{Ar2} in General Formula (a0-m).

23. The compound according to claim 22, wherein M^{m+} in General Formula (a0-m) represents an m-valent sulfonium cation.

24. A polymer compound which has a constitutional unit derived from the compound according to claim 14.
